(19) United States
(12) Patent Application Publication
(10) Pub. No.: US 2005/0054597 A1
(43) Pub. Date: Mar. 10, 2005

(54) Method and system for rapidly conferring a desired trait to an organism

(75) Inventor: Mitsuru Furusawa, Tokyo (JP)

Correspondence Address: SEED INTELLECTUAL PROPERTY LAW GROUP PLLC, 701 FIFTH AVE, SUITE 6300, SEATTLE, WA, 98104-7092, US

(73) Assignees: Neo-Morgan Laboratory Incorporated, Tokyo (JP); Mitsuru Furusawa, Tokyo (JP)

(21) Appl. No.: 10/810,486

(22) Filed: Mar. 26, 2004

Related U.S. Application Data
(63) Continuation-in-part of application No. 10/684,141, filed on Oct. 10, 2003.

(30) Foreign Application Priority Data
Mar. 28, 2003 (JP) 2003-092898

(51) Int. Cl. A61K 48/00; C12N 15/85

(52) U.S. Cl. 514/44; 435/455

(57) ABSTRACT

A method is provided for regulating the conversion rate of a hereditary trait of a cell, comprising the step of regulating the error-prone frequency of gene replication of the cell. A method is provided for producing a cell having a regulated hereditary trait, comprising the step of (a) regulating an error-prone frequency of gene replication of the cell, and (b) reproducing the resultant cell. A method is provided for producing an organism having a regulated hereditary trait, comprising the steps of (a) regulating the error-prone frequency of gene replication of the organism, and (b) reproducing the resultant organism.

This application in a continuation-in-part of U.S. patent application Ser. No. 10/684,141, filed on Oct. 10, 2003, and claims priority to Japanese Patent Application No. 2003-092890, filed on Mar. 28, 2003, each of which is herein incorporated by reference in its entirety for all purposes.

### BACKGROUND OF THE INVENTION

1. Field of the invention

The present invention relates to a method for rapidly modifying a hereditary trait of an organism, and an organism and a product obtained by the method.

2. Description of the Related Art

Humans have tried to modify the hereditary traits of organisms since recorded history. Before the advent of so-called genetic engineering, cross-breeding or the like had been tried to acquire organisms having a desired trait, or alternatively, mutations had been randomly caused by radiation and mutated organisms having a modified hereditary trait had been isolated.

Recent advanced genetic engineering facilitates obtaining organisms having a modified hereditary trait to a greater extent. Genetic engineering has been widely used in production of genetically modified organisms, in which an exogenous gene is introduced into an organism. However, an organism into which an exogenous gene is only introduced does not always acquire a desired hereditary trait. A manipulation different from the natural evolutionary process may lead to unexpected results. Therefore, government authorities regulate foods derived from genetically modified organisms (GMOs) more strictly than conventional foods.

Therefore, there is an increasing demand in this field for a method for conferring a desired hereditary trait to organisms in compliance with natural evolution and a method for producing such organisms.

To date there have been the following known mutagenesis methods.

(1) Natural mutation: mutation occurring when an organism normally grows under ordinary environments is called natural mutation. Major causes for natural mutation are considered to be errors in DNA replication and endogenous mutagens (nucleotide analog) (Maki, “Shizenheni To Shufukukiko [Natural Mutation And Repair Mechanism]”, Seibo Kogaku [Cell Engineering], Vol. 13, No. 8, pp. 663-672, 1994).

(2) Treatment with radiation, mutagens, or the like: DNA is damaged by treatment with radiation, such as ultraviolet light, X-ray, or the like, or treatment with an artificial mutagen, such as an alkylating agent or the like. Such damage may be fixed as a mutation in the course of DNA replication.

(3) Use of PCR (polymerase chain reaction): in PCR, since DNA is amplified in vitro, the PCR system lacks a part of the intracellular mutation suppressing mechanism. Therefore, mutations may be highly frequently induced. If DNA shuffling (Stemmer, Nature, Vol. 370, pp. 389-391, August 1994) is combined with PCR, accumulation of deleterious mutations can be avoided and a plurality of beneficial mutations can be accumulated in genes.

(4) Use of mutating factors (or mutators): in almost all organisms, the frequency of natural mutations is maintained at a considerably low rate by a mutation suppressing mechanism. The mutation suppressing mechanism includes a plurality of stages involved in 10 or more genes. Mutations occur at a high frequency in organisms in which one or more of the genes are destroyed. These organisms are called mutators. These genes are called mutator genes (Maki, supra, and Horst et al., Trends in Microbiology, Vol. 7, No. 1, pp. 29-36, January 1999).

A method using a mutator is a disparity method (Furusawa M. and Doi H., J. Theor. Diol. 157, pp. 127-133, 1992; and Furusawa M. and Doi H., Genetica 103, pp. 333-347, 1998; Japanese Patent Laid-Open Publication 6-163986; Japanese Patent Laid-Open Publication 8-163987; Japanese Patent Laid-Open Publication 9-Z3882; WO00/28015). In the disparity method, it has not been clarified as to whether or not actually produced organisms (particularly, higher organisms (e.g., eukaryotic organisms) exhibit a normal growth curve. In addition, the disparity method has not been demonstrated to accelerate natural evolution.

In simulation of a disequilibrium mutation model for “higher organisms” (e.g., eukaryotic organisms), such as eukaryotic organisms, having diploid or more sets of chromosomes possessing a plurality of sites of replication, there is a possibility that a lethal mutation occurs. It it not clear as to whether or not the disparity method can be applied to actual situations.

In simulation of a disequilibrium mutation model, mutations are randomly introduced into, for example, non-contiguous chains having less replication accuracy. Whether or not such mutations contribute to evolution is not clear.

In drug resistance experiments which have been tried using mutant strains of E. coli having introduced mutators, drug-resistant attains have been obtained. However, no system has even been suggested which can arbitrarily change or control the rate of evolution.

There has been no experiment which determined, by genome-level analysis which provides a measure of the rate of evolution, whether or not mutations were actually inserted in a disequilibrium manner. Considering that sequencing techniques per se can be easily carried out, it can be said that there has been no example which reported that mutation sites were identified.

### SUMMARY OF THE INVENTION

The above-described problems have been solved by the present inventors who found that the rate of evolution of organisms is not a function of time and can be regulated by regulating the error-prone frequency of organisms and demonstrated that real organisms having a modified rate of evolution proliferate at substantially the same rate as that of naturally-evolving organisms. According to the present invention, it could be demonstrated that the error threshold does not substantially influence the evolution of organisms.

In another aspect of the present invention, the present inventors studied the error threshold of quasi species having heterogeneous replication accuracies. The present inventors demonstrated that the coexistence of error-free and error-prone polymerases could. Increase the error threshold without disruptive lose of genetic information. The present inventors also indicated that replicores (replication agents) influence the error threshold. As a result, the present inventors found that quasispecies having heterogeneous replication accuracies reduce genetic costs involved in selective evolution for producing various mutants.

Appropriate evolution requires both genetic diversity and stable reproduction of advantageous mutants. Accurate replication of the genome guarantees stable reproduction, while errors during replication produce genetic diversity. Therefore, one key to evolution is thus inherent in replication accuracy. Replication accuracy depends on nucleotide polymerases. It is believed that intracellular polymerases have homogeneous replication accuracies. Most studies of evolutionary models have also been based on homogeneous replication accuracy. However, it has been demonstrated that error-free and error-prone polymerases coexist in naturally-occurring organisms. The present invention is therefore compatible to nature.

According to an aspect of the present invention, a method is provided for regulating a conversion rate of a hereditary trait of a cell, comprising the step of: (a) regulating an error-prone frequency of gene replication of the cell.

In one embodiment of this invention, at least two kinds of error-prone frequency agents playing a role in the gene replication are present.

In one embodiment of this invention, at least about 30% of the error-prone frequency agents have a lesser error-prone frequency.

In one embodiment of this invention, the agents playing a role in the gene replication have heterogeneous error-prone frequencies.

In one embodiment of this invention, the agent having the lesser error-prone frequency is substantially error-free.

In one embodiment of this invention, the error-prone frequencies are different from each other by at least 10¹.

In one embodiment of this invention, the error-prone frequencies are different from each other by at least 10².

In one embodiment of this invention, the error-prone frequencies are different from each other by at least 10³.

In one embodiment of this invention, the step of regulating the error-prone frequency comprises regulating an error-prone frequency of at least one agent selected from the group consisting of a repair agent capable of removing abnormal bases and a repair agent capable of repairing mismatched base pairs, the agents being present in the cell.

In one embodiment of this invention, the step of regulating the error-prone frequency comprises providing a difference in the number of errors between one strand and the other strand of double-stranded genomic DNA in the cell, in one embodiment of this invention, the step of regulating the error-prone frequency comprises regulating an error-prone frequency of a DNA polymerase of the cell.

In one embodiment of this invention, the DNA polymerase has a proofreading function.

In one embodiment of this invention, the DNA polymerase comprises at least one polymerase selected from the group consisting of DNA polymerase α, DNA polymerase β, DNA polymerase γ, DNA polymerase δ, and DNA polymerase ε of eukaryotic cells, and corresponding DNA polymerases thereto.

In one embodiment of this invention, the step of regulating the error-prone frequency comprises regulating proofreading activity of at least one polymerase selected from the group consisting of DNA polymerase δ and DNA polymerase ε of eukaryotic cells, and corresponding DNA polymerases thereto.

In one embodiment of this invention, the regulating the error-prone frequency comprises regulating a proofreading activity of DNA polymerase δ of a prokaryotic call or DNA polymerase corresponding thereto.

In one embodiment of this invention, the regulating the error-prone frequency comprises introducing a DNA polymerase variant into the cell.

In one embodiment of this invention, the introducing the DNA polymerase variant into the call is performed with a method selected from the group consisting of homologous recombination and transformation using gene introduction or a plasmid.

In one embodiment of this invention, the regulating the error-prone frequency comprises introducing a variant of DNA polymerase δ of a prokaryotic call or DNA polymerase corresponding thereto.

In one embodiment of this invention, the variant of DNA polymerase δ of a prokaryotic call or DNA polymerase corresponding thereto comprises a mutation which deletes a proofreading activity thereof.

In one embodiment of this invention, the step of regulating the error-prone frequency comprises increasing the error-prone frequency higher than that of a wild type of the cell.

In one embodiment of this invention, the proofreading function of the DNA polymerase is lower than that of a wild type of the DNA polymerase.

In one embodiment of this invention, the proofreading function of the DNA polymerase provides at least one mismatched base in a bass sequence, the number of the at least one mismatched base being greater by at least one than that of a wild type of the DNA polymerase.

In one embodiment of this invention, the proofreading function of the DNA polymerase provides at least one mismatched base in a base sequence.

In one embodiment of thin invention, the proofreading function of the DNA polymerase provides at least two mismatched bases.

In one embodiment of this invention, the proofreading function of the DNA polymerase provides at least one mismatched base in a base sequence at a rate of 10⁻⁶.

In one embodiment of this invention, the proofreading function of the DNA polymerase provides at least one mismatched base in a base sequence at a rate of 10⁻³.

In one embodiment of this invention, the proofreading function of the DNA polymerase provides at least one mismatched base in a base sequence at a rate of 10⁻².

In one embodiment of this invention, the cell is a gram-positive or eukaryotic cell.

In one embodiment of this invention, the cell is a eukaryotic cell.

In one embodiment of this invention, the cell is a unicellular or multicellular organism.

In one embodiment of this invention, the cell is an animal, plant, fungus, or yeast cell.

In one embodiment of this invention, the cell is a mammalian cell.

In one embodiment of this invention, after conversion of the hereditary trait, the cell has substantially the same growth as that of a wild type of the cell.

In one embodiment of this invention, the cell naturally has at least two kinds of polymerases.

In one embodiment of this invention, the cell naturally has at least two kinds of polymerases, the at least two kinds of polymerases having a different error-prone frequency.

In one embodiment of this invention, the cell has at least two kinds of polymerases, one of the at least two kinds of polymerases is involved in an error-prone frequency of a lagging strand, and another of the at least two kinds of polymerases is involved in an error-prone frequency of a leading strand.

In one embodiment of this invention, the cell has resistance to an environment, the resistance being not possessed by the call before the conversion.

In one embodiment of this invention, the environment comprises, as a parameter, at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

In one embodiment of this invention, the cell includes a cancer cell.

In one embodiment of this invention, the cell constitutes a tissue.

In one embodiment of this invention, the cell constitutes an organism.

In one embodiment of this invention, the method further comprises differentiating the cell to a tissue or an organism after conversion of the hereditary trait of the cell.

In one embodiment of this invention, the error-prone frequency is regulated under a predetermined condition.

In one embodiment of this invention, the error-prone frequency is regulated by regulating at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

According to another aspect of the present invention, a method in provided for producing a cell having a regulated hereditary trait, comprising the step of: (a) regulating an error-prone frequency of gene replication of the cell; and (b) reproducing the resultant cell,

In one embodiment of this invention, the method further comprises screening for the reproduced cell having a desired trait.

In one embodiment of this invention, at least two kinds of error-prone frequency agents playing a role in the gene replication are present.

In one embodiment of this invention, at least about 30% of the error-prone frequency agents have a lesser error-prone frequency.

In one embodiment of this invention, the agents playing a role in the gene replication have heterogeneous error-prone frequencies.

In one embodiment of this invention, the agent having the lesser error-prone frequency is substantially error-free.

In one embodiment of this invention, the error-prone frequencies are different from each other by at least 10¹.

In one embodiment of this invention, the error-prone frequencies are different from each other by at least 10².

In one embodiment of this invention, the error-prone frequencies are different from each other by at least 10³.

In one embodiment of this invention, the step of regulating the error-prone frequency comprises regulating an error-prone frequency of at least one agent selected from the group consisting of a repair agent capable of removing abnormal bases and a repair agent capable of repairing mismatched base pairs, the agents being present in the cell.

In one embodiment of this invention, the step of regulating the error-prone frequency comprises providing a difference in the number of errors between one strand and the other strand of double-stranded genomic DNA in the cell.

In one embodiment of this invention, the step of regulating the error-prone frequency comprises regulating an error-prone frequency of a DNA polymerase of the cell.

In one embodiment of this invention, the DNA polymerase has a proofreading function.

In one embodiment of this invention, the DNA polymerase comprises at least one polymerase selected from the group consisting of DNA polymerase α, DNA polymerase β, DNA polymerase γ, DNA polymerase δ, and DNA polymerase ε of eukaryotic cells, and corresponding DNA polymerases thereto.

In one embodiment of this invention, the step of regulating the error-prone frequency comprises regulating proofreading activity of at least one polymerase selected from the group consisting of DNA polymerase δ and DNA polymerase ε of eukaryotic cells, and corresponding DNA polymerases thereto.

In one embodiment of this invention, the regulating the error-prone frequency comprises regulating a proofreading activity of DNA polymerase δ of a prokaryotic cell or DNA polymerase corresponding thereto.

In one embodiment of this invention, the regulating the error-prone frequency comprises introducing a DNA polymerase variant into the cell.

In one embodiment of this invention, the introducing the DNA polymerase variant into the cell is performed with a method selected from the group consisting of homologous recombination and transformation using gene introduction or a plasmid.

In one embodiment of this invention, the regulating the error-prone frequency comprises introducing a variant of DNA polymerase δ of a prokaryotic cell or DNA polymerase corresponding thereto.

In one embodiment of this invention, the variant of DNA polymerase δ of a prokaryotic cell or DNA polymerase corresponding thereto comprises a mutation which deletes only a proofreading activity thereof.

In one embodiment of this invention, the step of regulating the error-prone frequency comprises increasing the error-prone frequency higher than that of a wild type of the cell.

In one embodiment of this invention, the proofreading function of the DNA polymerase is lower than that of a wild type of the DNA polymerase.

In one embodiment of this invention, the proofreading function of the DNA polymerase provides at least one mismatched base in a base sequence, the number of the at least one mismatched bass being greater by at least one than that of a wild type of the DNA polymerase.

In one embodiment of this invention, the proofreading function of the DNA polymerase provides at least one mismatched base in a bass sequence.

In one embodiment of this invention, the proofreading function of the DNA polymerase provides at least two mismatched bases.

In one embodiment of this invention, the proofreading function of the DNA polymerase provides at least one mismatched base in a base sequence at a rate of 10⁻⁶.

In one embodiment of this invention, the proofreading function of the DNA polymerase provides at least one mismatched base in a base sequence at a rate of 10⁻³.

In one embodiment of this invention, the proofreading function of the DNA polymerase provides at least one mismatched base in a base sequence at a rate of 10⁻².

In one embodiment of this invention, the cell is a gram-positive or eukaryotic cell.

In one embodiment of this invention, the cell is a eukaryotic cell.

In one embodiment of this invention, the cell is a unicellular or multicellular organism.

In one embodiment of this invention, the cell is an animal, plant, fungus, or yeast cell.

In one embodiment of this invention, the cell is a mammalian cell.

In one embodiment of this invention, after conversion of the hereditary trait, the cell has substantially the same growth as that of a wild type of the cell.

In one embodiment of this invention, the cell naturally has at least two kinds of polymerases.

In one embodiment of this invention, the cell naturally has at least two kinds of polymerases, the at least two kinds of polymerases having a different error-prone frequency.

In one embodiment of this invention, the cell has at least two kinds of polymerases, one of the at least two kinds of polymerases is involved in an error-prone frequency of a lagging strand, and another of the at least two kinds of polymerases is involved in an error-prone frequency of a leading strand.

In one embodiment of this invention, the cell has resistance to an environment, the resistance being not possessed by the cell before the conversion.

In one embodiment of this invention, the environment comprises, as a parameter, at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

In one embodiment of this invention, the cell includes a cancer cell.

In one embodiment of this invention, the cell constitutes a tissue.

In one embodiment of this invention, the cell constitutes an organism.

In one embodiment of this invention, the method further comprises differentiating the cell to a tissue or an organism after conversion of the hereditary trait of the cell.

In one embodiment of this invention, the error-prone frequency is regulated under a predetermined condition.

In one embodiment of this invention, the error-prone frequency is regulated by regulating at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotic, natural substances, mental stress and physical stress, or a combination thereof.

According to another aspect of the present invention, a method is provided for producing an organism having a regulated hereditary trait, comprising the steps of: (a) regulating the error-prone frequency of gene replication of the organism; and (b) reproducing the resultant organism.

According to another aspect of the present invention, a cell is provided, which has a regulated hereditary trait, produced by the above-described method.

In one embodiment of this invention, the cell has substantially the same growth as that of a wild type of the cell.

According to another aspect of the present invention, an organism is provided, which has a regulated hereditary trait, produced by the above-described method.

In one embodiment of this invention, the organism has substantially the same growth as that of a wild type of the organism.

According to another aspect of the present invention, a method is provided for producing a nucleic acid molecule encoding a gene having a regulated hereditary trait, comprising the steps of: (a) changing an error-prone frequency of gene replication of an organism; (b) reproducing the resultant organism; (c) identifying a mutation in the organism; and (d) producing a nucleic acid molecule encoding a gene having the identified mutation.

According to another aspect of the present invention, a nucleic acid molecule is provided, which is produced by the above-described method.

According to another aspect of the present invention, a method is provided for producing a polypeptide encoded by a gone having a regulated hereditary trait, comprising the steps of: (a) changing an error-prone frequency of gene replication of an organism: (b) reproducing the resultant organism; (c) identifying a mutation in the organism; and (d) producing a polypeptide encoded by a gone having the identified mutation.

According to another aspect of the present invention, a polypeptide is provided, which is produced by the above-described method.

According to another aspect of the present invention, a method is provided for producing a metabolite of an organism having a regulated hereditary trait, comprising the steps of: (a) changing an error-prone frequency of gene replication of an organism; (b) reproducing the resultant organism; (c) identifying a mutation in the organism; and (d) producing a metabolite having the identified mutation.

According to another aspect of the present invention, a metabolite is provided, which is produced by the above-described method.

According to another aspect of the present invention, a nucleic acid molecule is provided for regulating a hereditary trait of an organism, comprising a nucleic acid sequence encoding a DNA polymerase having a regulated error-prone frequency.

In one embodiment of this invention, the DNA polymerase is DNA polymerase δ or ε of eukaryotic organisms, or DNA polymerase corresponding thereto of gram-positive bacteria.

In one embodiment of this invention, the DNA polymerase is a variant of DNA polymerase δ or ε of eukaryotic organisms, or DNA polymerase corresponding thereto of gram-positive bacteria, the variant comprising a mutation which deletes only a proofreading activity thereof.

In one embodiment of this invention, the DNA polymerase in a variant of DNA polymerase δ of eukaryotic organisms, or DNA polymerase corresponding thereto of gram-positive bacteria, the variant comprising a mutation which deletes only a proofreading activity thereof.

According to another aspect of the present invention, a vector is provided, comprising the above-described nucleic acid molecule.

According to another aspect of the present invention, a cell is provided comprising the above-described nucleic acid molecule.

In one embodiment of this invention, the cell is a eukaryotic cell.

In one embodiment of this invention, the eukaryotic cell is selected from the group consisting of plants, animals, and yeasts.

In one embodiment of this invention, the cell is a gram-positive bacterial cell.

In one embodiment of this invention, the cell is used for regulating a conversion rate of a hereditary trait.

According to another aspect of the present invention, an organism is provided, comprising the above-described nucleic acid molecule.

According to another aspect of the present invention, a product substance is provided, which is produced by the above-described cell or a part thereof.

According to another aspect of the present invention, a nucleic acid molecule is provided, which is contained in the above-described cell or a part thereof.

In one embodiment of this invention, the nucleic acid molecule encodes a gene involved in the regulated hereditary trait.

According to another aspect of the present invention, a method is provided for testing a drug, comprising the steps of: testing an effect of the drug using the above-described cell as a model of disease; testing an effect to the drug using a wild type of the cell as a control; and comparing the model of disease and the control.

According to another aspect of the present invention, a method is provided for testing a drug, comprising the steps of: testing an effect of the drug using the above-described organism as a model of disease; testing an effect to the drug using a wild type of the organism as a control; and comparing the model of disease and the control.

According to another aspect of the present invention, a set of at least two kinds of polymerases is provided for use in regulating a conversion rate of a hereditary trait of an organism, wherein the polymerases have a different error-prone frequency.

In one embodiment of this invention, one of the at least two kinds of polymerases is involved in an error-prone frequency of a lagging strand, and another of the at least two kinds of polymerases is involved in an error-prone frequency of a leading strand.

In one embodiment of this invention, the set of polymerases are derived from the same species.

According to another aspect of the present invention, a set of at least two kinds of polymerases is provided for use in producing an organism having a regulated hereditary trait, wherein the polymerases have a different error-prone frequency.

In one embodiment of this invention, one of the at least two kinds of polymerases is involved in an error-prone frequency of a lagging strand, and another of the at least two kinds of polymerases is involved in an error-prone frequency of a leading strand.

In one embodiment of this invention, the set of polymerases are derived from the same organism species.

According to another aspect of the present invention, use of at least two kinds of polymerases is provided for regulating a conversion rate of a hereditary trait of an organism, wherein the polymerases have a different error-prone frequency.

According to another aspect of the present invention, use of at least two kinds of polymerases for producing an organism having a regulated hereditary trait, wherein the polymerases have a different error-prone frequency.

Thus, the invention described herein makes possible the advantage of providing a method for conferring a desired hereditary trait to organisms in compliance with natural evolution.

These and other advantages of the present invention will become apparent to those skilled in the art upon reading and understanding the following detailed description with reference to the accompanying figures.

### BRIEF DESCRIPTION OF THE DRAWINGS

FIG. 1 shows that a mutant of Example 1 of the present invention and its wild type have substantially the same growth curves.

FIG. 2 shows Example 1 of the present invention in which high temperature resistance to conferred.

FIG. 3A shows a photograph of Example 1 of the present invention in which high temperature resistance is conferred. A mutant strain capable of growing at high temperature was isolated from the pol3 mutant strain (DNA polymerase δ lacking exonuclease). Mark * indicates the parent strain (AMY128-1) and the seven other colonies are high temperature resistant strains.

FIG. 3B shows another photograph of Example 1 of the present invention in which high temperature resistance is conferred. A mutant strain capable of growing at high temperature was isolated from the pol2 mutant strain (DNA polymerase ε lacking exonuclease). Mark * indicates the parent strain (AMY2-6) and the seven other colonies are high temperature resistant strains.

FIG. 4A shows a photograph of Example 1 of the present invention in which high temperature resistance is conferred. Arrows indicate cells which were dead and had bubbles. High temperature resistant strains 1 and 2 were subjected to separate experiments. In the parent strain, no cell could survive at 41° C. The high temperature resistant strain obtained by the method of the present invention could live at 41° C.

FIG. 4B show another photograph of Example 1 of the present invention in which high temperature resistance in conferred. A mutant strain capable of growing at such a high temperature that yeast cannot be considered to survive at 41° C., was isolated from a pol2 mutant strain (DNA polymerase ε lacking exonuclease activity) of S. cerevisiae. Top shows the parent strain (AMY2-6), and the other seven colonies are high temperature resistant mutant strains.

FIG. 5 shows examples of quasi species having homogeneous replication accuracy and heterogeneous replication accuracies.

FIG. 6 shows error catastrophe.

FIG. 7 shows an error threshold as a function of the relative concentration of error-free polymerase at various numbers of replication agents.

FIG. 8 shows an example of a permissible error rate based on the parameters of E. coli.

FIG. 9 schematically shows a vector to be introduced into a transgenic mouse.

FIG. 10 shows the PCR process for confirming foreign genes. From the left, with mPGK2 Tg, without mPGK2 Tg, with Fth117 Tg, a mPGK2 Tg vector, and Bluescript only (control) (transgenic mouse #1 for each), and without #2 mouse Tg, with #2 mouse Tg, a #2Tg vector, and pBluescript (transgenic mouse #2 for each). The marker is shown at the right end.

FIG. 11 shows expression of a Cre recombinase in the mouse testis. a shows mPGK2, b shows Fth117, and c shows a control. The bar represents 50 μm.

FIG. 12 shows am expression region by a mPGK2 promoter.

FIG. 13 shows an expression region by a Fth117 promoter.

FIG. 14 schematically shows a targeting vector.

FIG. 15 schematically shows a tissue-specific recombination reaction.

FIG. 16 schematically shows a screening method using calli.

FIG. 17 schematically shows a vector used in an experiment for ES cells in Example 8.

FIG. 18 schematically shows a recombinant (targeting) vector using Cre recombinase.

### DESCRIPTION OF SEQUENCES

SEQ ID NO. 1: yeast DNA polymerase δ nucleic acid sequence
SEQ ID NO. 2: yeast DNA polymerase δ amino acid sequence
SEQ ID NO. 3: yeast DNA polymerase ε nucleic acid sequence
SEQ ID NO. 4: yeast DNA polymerase ε amino acid sequence
SEQ ID NO. 5: DnaQ partial sequence (Escherichia coli)
SEQ ID NO. 6: DnaQ partial sequence (Haemophilus influenzae)
SEQ ID NO. 7: DnaQ partial sequence (Salmonella typhimurium)
SEQ ID NO. 8: DnaQ partial sequence (Vibrio cholerae)
SEQ ID NO. 9: DnaQ partial sequence (Pseudomonas aeruginosa)
SEQ ID NO. 10: DnaQ partial sequence (Neisseria meningitides)
SEQ ID NO. 11: DnaQ partial sequence (Chlamydia trachomatis)
SEQ ID NO. 12: DnaQ partial sequence (Streptomyces coelicolor)
SEQ ID NO. 13: DnaQ partial sequence (Shigella flexneri 2a str. 301)
SEQ ID NO. 14: PolC partial sequence (Staphylococcus aureus)
SEQ ID NO. 15: PolC partial sequence (Bacillus subtilis)
SEQ ID NO. 16: PolC partial sequence (Mycoplasma pulmonis)
SEQ ID NO. 17: PolC partial sequence (Mycoplasma genitalium)
SEQ ID NO. 18: PolC partial sequence (Mycoplasma pnuemoniae)
SEQ ID NO. 19: Pol III partial sequence (Saccharomyces cerevisiae)
SEQ ID NO. 20: Pol II partial sequence (Saccharomyces cerevisiae)
SEQ ID NO. 21: Polδ partial sequence (mouse)
SEQ ID NO. 22: Polε partial sequence (mouse)
SEQ ID NO. 23: Polδ partial sequence (human)
SEQ ID NO. 24: Polε partial sequence (human)
SEQ ID NO. 25: Polδ partial sequence (rice)
SEQ ID NO. 26: Polδ partial sequence (Arabidopsis thaliana)
SEQ ID NO. 27: Pol ε partial sequence (Arabidopsis thaliana)
SEQ ID NO. 28: Polδ partial sequence (rat)
SEQ ID NO. 29: Polδ partial sequence (bovine)
SEQ ID NO. 30: Polδ partial sequence (soybean)
SEQ ID NO. 31: Polδ partial sequence (fruit fly)
SEQ ID NO. 32: Polε partial sequence (fruit fly)
SEQ ID NO. 33: Polδ yeast modified nucleic acid sequence
SEQ ID NO. 34: Polδ yeast modified amino acid sequence
SEQ ID NO. 35: Polε yeast modified nucleic acid sequence
SEQ ID NO. 36: Polε yeast modified amino acid sequence
SEQ ID NO. 37: Polδ forward primer
SEQ ID NO. 38: Polδ reverse primer
SEQ ID NO. 39: Polε forward primer
SEQ ID NO. 40: Polε reverse primer
SEQ ID NO. 41: Escherichia coli DnaQ nucleic acid sequence
SEQ ID NO. 42: Escherichia coli DnaQ amino sequence
SEQ ID NO. 43: Bacillus subtilis PolC nucleic acid sequence
SEQ ID NO. 44: Bacillus subtilis PolC amino sequence
SEQ ID NO. 45: Arabidopsis thaliana Polδ amino sequence
SEQ ID NO. 46: Arabidopsis thaliana Polε amino sequence
SEQ ID NO. 47: rice Polδ nucleic acid sequence
SEQ ID NO. 48: rice Polδ amino sequence
SEQ ID NO. 49: soybean Polδ nucleic acid sequence
SEQ ID NO. 50: soybean Polδ amino sequence
SEQ ID NO. 51: human Polδ nucleic acid sequence
SEQ ID NO. 52: human Polδ amino sequence
SEQ ID NO. 53: human Polε nucleic acid sequence
SEQ ID NO. 54: human Polε amino sequence
SEQ ID NO. 55: mouse Polδ nucleic acid sequence
SEQ ID NO. 56: mouse Polδ amino sequence
SEQ ID NO. 57: mouse Polε nucleic acid sequence
SEQ ID NO. 58: mouse Polε amino sequence
SEQ ID NO. 59: rat Polδ nucleic acid sequence
SEQ ID NO. 60: rat Polδ amino sequence
SEQ ID NO. 61: bovine Polδ nucleic acid sequence
SEQ ID NO. 62: bovine Polδ amino sequence
SEQ ID NO. 63: fruit fly Polδ nucleic acid sequence
SEQ ID NO. 64: fruit fly Polδ amino sequence
SEQ ID NO. 65: fruit fly Polε nucleic acid sequence
SEQ ID NO. 66: fruit fly Polε amino sequence
SEQ ID NO.: 67: 5′ terminal primer SpeI-5′ Pold1 of the Pold1 gene
SEQ ID NO.: 68: 3′ terminal primer EcoRI-3′ Pold1 of the Pold1 gene
SEQ ID NO.: 69: primer sequence for introducing a mutation into the Pold1 gene (Example 4)
SEQ ID NO.: 70: mutant cDNA sequence of the Pold1 gene (Example 4)
SEQ ID NO.: 71: 5′ mPGK2-sacII primer of mPGK2
SEQ ID NO.: 72: 3′ mPGK2-SpeI primer of mPGK2
SEQ ID NO.: 73: 5Fth117-sacII primer of Fth117
SEQ ID NO.: 74: 3′ Fth117-SpeI primer of Fth117
SEQ ID NO.: 75: Cre-F primer of transgenic mouse #1
SEQ ID NO.: 76: Cre-R primer of transgenic mouse #1
SEQ ID NO.: 77: Neo-F primer of transgenic mouse #2
SEQ ID NO.: 78: Neo-R primer of transgenic mouse #2
SEQ ID NO.: 79: Neo-F primer for confirming expression of mRNA in Example 4
SEQ ID NO.: 80: Neo-R primer for confirming expression of mRNA in Example 4
SEQ ID NO.: 81: about 5.7 kbp sequence upstream of Fth117
SEQ ID NO. 82: Xbal-42120-F for amplifying Arabidopsis thaliana-derived Polδ
SEQ ID NO.: 83: 2g42120-Sacl-R for amplifying Arabidopsis thaliana-derived polδ
SEQ ID NO.: 84: 2g42120-D316A-F for amplifying mutant polδ gene polδ (D316A)
SEQ ID NO.: 85: 2g42120R for amplifying mutant polδ gene polδ (D316A)
SEQ ID NO.: 86: Pold1 gene (nucleic acid sequence) containing Kozak sequence derived from mouse testis
SEQ ID NO.: 87: Pold1 gene (amino acid sequence) containing Kozak sequence derived from mouse testis
SEQ ID NO.: 88: nucleic acid sequence of mouse polδ gene mutant (D400A)
SEQ ID NO. 89: amino acid sequence of mouse polδ gene mutant (D400A)
SEQ ID NO.: 90: nucleic acid sequence of polδ (At1g42120)
SEQ ID NO.: 91: amino acid sequence of polδ (At1g42120)
SEQ ID NO. 92: mutant polδ gene polδ (D316A) (nucleic acid sequence)
SEQ ID NO.: 93: mutant polδ gene polδ (D316A) (amino acid sequence)
SEQ ID NO.: 94: 455-bp mPGK2 promoter fragment
SEQ ID NO.: 95: 5725-bp DNA fragment upstream of the Fth117 gene

These and other advantages of the present invention will be apparent from the drawings and a reading of the detailed description thereof.

### DESCRIPTION OF THE PREFERRED EMBODIMENTS

Hereinafter, the present invention will be described by way of illustrative examples with reference to the accompanying drawings.

It should be understood throughout the present specification that the singular forms “a”, “an” and “the” include plural referents unless the context clearly dictates otherwise. It should be also understood that the terms as used herein have definitions typically used in the art unless otherwise mentioned.

(Terms)

In this specification and in the claims which follow, reference will be made to a number of terms which shall be defined to have the following meanings:

The term “organism” is herein used in its broadest sense in the art and refers to a body carrying on processes of life, which has various properties, such as, representatively, cellular structure, proliferation (self reproduction), growth, regulation, metabolism, repair ability, and the like. Typically, organisms possess basic attributes, such as heredity controlled by nucleic acids and proliferation in which metabolism controlled by proteins is involved. Organisms include viruses, prokaryotic organisms, eukaryotic organisms (e.g., unicellular organisms (e.g., yeast, etc.) and multicellular organisms (e.g., plants, animals, etc.)), and the like. It will be understood that the method of the present invention may be applied to any organisms, including higher organisms, such as gram-positive bacteria, eukaryotic organisms, and the like.

The term “eukaryotic organism” Is herein used in its ordinary sense and refers to an organism having a clear nuclear structure with a nuclear envelope. Examples of eukaryotic organisms include, but are not limited to, unicellular organisms (e.g., yeast, etc.), plants (e.g., rice, wheat, maize, soybean, etc.), animals (e.g., mouse, rat, bovine, horse, swine, monkey, etc.), insects (e.g., fly, silkworm, etc.), and the like. Yeast, nematode, fruit fly, silkworm, rice, wheat, soybean, maize, Arabidopsis thaliana, human, mouse, rat, bovine, horse, swine, frog, fish (e.g., zebra fish, etc) may be used herein as models, but use is not limited thereto.

As used herein, the term “prokaryotic organism” is used herein in its ordinary sense and refers to an organism composed of cell(s) having no clear nuclear structure. Examples of prokaryotic organisms include gram-negative bacteria (e.g., E. coli, Salmonella, etc.), gram-positive bacteria (e.g., Bacillus subtilis, actinomycete, Staphylococcus, etc.), cyanobacteria, hydrogen bacteria, and the like. Representatively, in addition to E. coli, gram-positive bacteria may be used herein, but use is not limited thereto.

The term “unicellular organism” is used herein in its ordinary sense and refers to an organism consisting of one cell. Unicellular organisms include both eukaryotic organisms and prokaryotic organism. Examples of unicellular organisms include, but are not limited to, bacteria (e.g., E. coli, Bacillus subtilis, etc.), yeast, cyanobacteria, and the like.

As used herein, the term “multicellular organism” refers to an individual organism consisting of a plurality of cells (typically, a plurality of cells of different types). Since a multicellular organism is composed of cells of different types, the maintenance of the life of the organism requires a high level of mechanism for homeostasis as is different from unicellular organisms. Most eukaryotic organisms are multicellular organisms. Multicellular organisms include animals, plants, insects, and the like. It should be noted that the present invention can be surprisingly applied to multicellular organisms.

The term “animal” is used herein in its broadest sense and refers to vertebrates and invertebrates (e.g., arthropods). Examples of animals include, but are not limited to, any of the class Mammalia, the class Aves, the class Reptilia, the class Amphibia, the class Pisces, the class Insecta, the class Vermes, and the like. Preferably, the animal may be, but is not limited to, a vertebrate (e.g., Myxiniformes, Petronyzoniformes, Chondrichthyes, Osteichthyes, amphibian, reptilian, avian, mammalian, etc.). In a certain embodiment, the animal may be, but is not limited to, a mammalian (e.g., monotremata, marsupialia, edentate, dermoptera, chiroptera, carnivore, insectivore, proboscides, perissodactyla, artiodactyla, tubulidentata, pholidota, sirenia, cetacean, primates, rodentia, lagomorpha, etc.). More preferably, the animal may be, but is not limited to, a primate (e.g., a chimpanzee, a Japanese monkey, a human) or any of the species which may be used as a model animal (e.g., perissodactyla, artiodactyla, rodentia (mouse, etc.), lagomorpha, etc.). The present invention is the first to demonstrate that the method of the present invention can be applied to any organism. It should be understood that any organism may be used in the present invention.

As used herein, the term “plant” refers to any organism belonging to the kingdom Plantae, characterized by chlorophylis hard cell walls, presence of rich perpetual embryotic tissues, and lack of the power of locomotion. Representatively, the term “plant” refers to a flowering plant capable of formation of cell walls and assimilation by chlorophylis. The term “plant” refers to any of monocotyledonous plants and dicotyledonous plants. Preferable plants include, but are not limited to, useful plants, such as monocotyledonous plants of the rice family (e.g., wheat, maize, rice, barley, sorghum, etc.). Examples of preferable plants include tobacco, green pepper, eggplant, melon, tomato, sweet potato, cabbage, leek, broccoli, carrot, cucumber, citrus, Chinese cabbage, lettuce, peach, potato, and apple. Preferable plants are not limited to crops and include flowering plants, trees, lawn, weeds, and the like. Unless otherwise dictated, the term “plant” refers to any of plant body, plant organ, plant tissue, plant cell, and seed. Examples of plant organ include root, leave, stem, flower, and the like. Examples of plant cell include callus, suspended culture cell, and the like. The present invention is the first to demonstrate that the method of the present invention can be applied to any organism. It should be understood that any organism may be used in the present invention.

In a certain embodiment, examples of types of plants that can be used in the present invention include, but are not limited to, plants in the families of Solanaceae, Poaceae, Brassicaceae, Rosaceae, Leguminosae, Cucurbitaceae, Lamiaceae, Liliaceae, Chenopodiaceae, and Umbelliferae.

As used herein, the term “hereditary trait”, which is also called genotype, refers to a morphological element of an organism controlled by a gene. An example of a hereditary trait includes, but is not limited to, resistance to a parameter of environment, such as, for example, temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, other organisms, chemical agents, antibiotics, natural substances, mental stress, physical stress, and the like.

As used herein, the term “gene” refers to a nucleic acid present in cells having a sequence of a predetermined length. A gene may or may not define a genetic trait. As used herein, the term “gene” typically refers to a sequence present in a genome and may refer to a sequence outside chromosomes, a sequence in mitochondria, or the like. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, for example, the term “DNA polymerase gene” typically refers to the structural gene of a DNA polymerase and its transcription and/or translation regulating sequences (e.g., a promoter). In the present invention, it will be understood that regulatory sequences for transcription and/or translation as well as structural genes are useful as genes targeted by the present invention. As used herein, “gene”may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context.

As used herein, the term “replication” in relation to a gene means that genetic material, DNA or RNA, reproduces a copy of itself, wherein a parent nucleic acid strand (DNA or RNA) is used as a template to form a new nucleic acid molecule (DNA or RNA, respectively) having the same structure and function as the parent nucleic acid. In eukaryotic cells, a replication initiating complex comprising a replication enzyme (DNA polymerase α) in formed to start replication at a number of origins of replication on a double-stranded DNA molecule, and replication reactions proceed in opposite directions from the origin of replication. The initiation of replication is controlled in accordance with a cell cycle. In yeast, an autonomously replicating sequence is regarded as an origin of replication. In prokaryotic cells, such as E. coli and the like, an origin of replication (or) is present on a genomic double-stranded circular DNA molecule. A replication initiating complex is formed at the ori, and reactions proceed in opposite directions from the ori. The replication initiating complex has a complex structure comprising 10 or more protein elements including a replication enzyme (DNA polymerase III). In the replication reaction, the helical structure of double-stranded DNA is partially rewound; a short DNA primer is synthesized; a new DNA strand is elongated from the 3′—OH group of the primer; Okazaki fragments are synthesized on a complementary strand template; the Okazaki fragments are ligated; proofreading is performed to compare the newly replicated strand with the template strand; and the like. Thus, the replication reaction is performed via a number of reaction steps.

The replication mechanism of genomic DNA which stores the genetic information of an organism is described in detail in, for example, Kornberg A. and Baker T., “DNA Replication”, New York, Freeman, 1992. Typically, an enzyme that uses one strand of DNA as a template to synthesize the complementary strand, forming a double-stranded DNA, is called DNA polymerase (DNA replicating enzyme). DNA replication requires at least two kinds of DNA polymerases. This is because typically, a leading strand and a lagging strand are simultaneously synthesized. DNA replication is started from a predetermined position an DNA, which is called an origin of replication (ori). For example, bacteria have at least one bi-directional origin of replication on their circular genomic DNA. Thus, typically, four DNA polymerases need to simultaneously act on one genomic DNA during its replication. In the present invention, preferably, replication error may be advantageously regulated on only one of a leading strand and a lagging strand, or alternatively, there may be advantageously a difference in the frequency of replication errors between the two strands.

As used herein, the term “replication error” refers to introduction of an incorrect nucleotide during replication of a gene (DNA, etc.). Typically, the frequency of replication errors is as low as one in 10⁸ to 10¹² pairings. The reason the replication error frequency is low is that nucleotide addition is determined by complementary base pairing between template DNA and introduced nucleotides during replication; the 3′→5′ exonuclease activity (proofreading function) of an enzyme, such as DNA polymerase δ, ε, or the like, identifies and removes mispaired nucleotides which are not complementary to the template; and the like. Therefore, in the present invention, the regulation of error-prone frequency in replication can be carried out by interrupting formation of specific base pairs, the proofreading function, and the like.

As used herein, the term “conversion rate” in relation to a hereditary trait refers to a rate at which a difference occurs in the hereditary trait between an original organism and its progenitor after reproduction or division of the original organism. Such a conversion rate can be represented by the number of organisms having a change in the hereditary trait per division or generation, for example. Such conversion of a hereditary trait may be herein alternatively referred to as “evolution”.

As used herein, the term “regulate” in relation to the conversion rate of a hereditary traits means that the conversion rate of the hereditary trait is changed by an artificial manipulation not bra naturally-occurring factor. Therefore, regulation of the conversion rate of a hereditary trait includes slowing and accelerating the conversion rate of a hereditary trait. By slowing the conversion rate of a hereditary trait of an organism, the organism does not substantially change the hereditary trait. In other words, by slowing the conversion rate of a hereditary trait of an organism, the evolution speed of the organism is lowered. Conversely, by accelerating the conversion rate of a hereditary trait of an organism, the organism changes the hereditary trait more frequently than normal levels. In other words, by accelerating the conversion rate of a hereditary trait of an organism, the evolution speed of the organism is increased.

As used herein, the term “error-free” refers to a property that there is little or substantially no errors in replication of a gene (DNA, etc.). Error-free levels are affected by the accuracy of the proofreading function of a proofreading enzyme (e.g., DNA polymerases δ and ε, etc.).

As used herein, the term “error-prone” refers to a property that an error is likely to occur in replication of a gene (DNA, etc.) (i.e., a replication error is likely to occur). Error-prone levels are affected by the accuracy of the proofreading function of a proof reading enzyme (e.g., DNA polymerases δ and ε, etc.).

Error-prone states and error-free states can be absolutely separated (i.e., can be determined with the level of an error-prone frequency or the like), or alternatively, can be relatively separated (i.e., when two or more agents playing a role in gene replication are separated, agents having a higher error-prone frequency are categorized into error-prone genes while agents having a lower error-prone frequency are categorized into error-free agents).

As used herein, the term “error-prone frequency” refers to a level of an error-prone property. Error-prone frequency can be represented by the absolute number of mutations (the number of mutations themselves) in a gene sequence or the relative number of mutations in a gene sequence (the ratio of the number of mutations to the full length), for example. Alternatively, when mentioning a certain organism or enzyme, the error-prone frequency may be represented by the absolute or relative number of mutations in a gene sequence per one reproduction or division thereof. Unless otherwise mentioned, error-prone frequency is represented by the number of errors in a gene sequence in one replication process. Error-prone frequency may be herein referred to as “accuracy” as an inverse measure. Uniform error-prone frequency means that when agents (polymerases, etc.) playing a role in replication of a plurality of genes are mentioned, their error-prone frequencies are substantially equal to one another. Conversely, heterogeneous error-prone frequency means that a significant difference in error-prone frequency is present among a plurality of agents (polymerases, etc.) playing a role in replication of a plurality of genes.

As used herein, the term “regulate” in relation to error-prone frequency means that the error-prone frequency is changed. Such regulation of error-prone frequency includes an increase and decrease in error-prone frequency. Examples of a method for regulating error-prone frequency include, but are not limited to, modification of a DNA polymerase having a proofreading function, insertion of an agent capable of inhibiting or suppressing polymerization or elongation reactions during replication, inhibition or suppression of factors promoting these reactions, deletion of one or more bases, lack of duplex DNA repair enzyme, modification of a repair agent capable of removing abnormal bases, modification of a repair agent capable of repairing mismatched base pairs, reduction of the accuracy of replication itself, and the like. Regulation of error-prone frequency may be carried out on both strands or one strand of double-stranded DNA. Preferably, regulation of error-prone frequency may be advantageously carried out on one strand. This is because adverse mutagenesis is reduced.

As used herein, the term “DNA polymerase” or “Pol” refers to an enzyme which releases pyrophosphoric acid from four deoxyribonucleoside 5′-triphosphate so as to polymerize DNA. DNA polymerase reactions require template DNA, a primer molecule, Mg²⁺, and the like. Complementary nucleotides are sequentially added to the 3′—OH terminus of a primer to elongate a molecule chain.

It is known that E. coli possesses at least three DNA polymerases I, II, and III. DNA polymerase I is involved in repair of damaged DNA, gene recombination, and DNA replication. DNA polymerases II and III are said to have an auxiliary function. These enzymes each have a subunit structure comprising several proteins and are divided into a core enzyme or a holoenzyme in accordance with the structure. A core enzyme is composed of a α, ε, and θ subunits. A holoenzyme comprises τ, γ, δ, and β components in addition to α, ε, and θ subunits. It is known that eukaryotic cells have a plurality of DNA polymerases. In higher organisms, there are a number of DNA polymerases α, β, δ, γ, ε, and the like. In animals, there are known polymerases: DNA polymerase α which to involved in replication of nuclear DNA and plays a role in DNA replication in a call growth phase); DNA polymerase β which is involved in DNA repair in nuclei and plays a role in repair of damaged DNA in the growth phase and the quiescent phase, and the like), DNA polymerase γ which is involved in replication and repair of mitochondrial DNA and has exonuclease activity); DNA polymerase δ which is involved in DNA elongation and has exonuclease activity: DNA polymerase ε which is involved in replication of a gap between lagging strands and has exonuclease activity; and the like.

In DNA polymerases having a proofreading function in gram-positive bacteria, gram-negative bacteria, eukaryotic organisms, and the like, it is believed that amino acid sequences having an ExoI motif play a role in 3′→5′ exonuclease activity center and have an influence on the accuracy of the proofreading function,
SEQ ID NO. 5: DnaQ: 8-QIVLDTETTGMN-19 (Escherichia coli);
SEQ ID NO. 6: DnaQ: 7-QIVLDTETTGMN-18 (Haemophilus influenzae):
SEQ ID NO. 7: DnaQ: 8-QIVLDTETTGMN-19 (Salmonella typhirmurium);
SEQ ID NO. 8: DnaQ: 12-IVVLDTETTGMN-23 (Vibrio cholerae);
SEQ ID NO. 9: DnaQ: 3-SVVLDTETTGMP-14 (Pseudomonas aeruginosa);
SEQ ID NO. 10: DnaQ: 5-QIILDTETTGLY-16 (Neisseria meningitides);
SEQ ID NO. 11: DnaQ: 9-FVCLDCETTGLD-20 (Chlamydia trachomatis);
SEQ ID NO. 12: DnaQ: 9-LAAFDTETTGVD-20 (Streptomyces coelicolor);
SEQ ID NO. 13: dnaQ: 11-QIVLDTETTGMN-22 (Shigella flexneri 2a str. 301);
SEQ ID NO. 14: PolC: 420-YVVFDVETTGLS-431 (Staphylococcus aureus);
SEQ ID NO. 15: PolC: 421-YVVFDVETTGLS-432 (Bacillus subtilis);
SEQ ID NO. 16: PolC: 404-YVVYDIETTGLS-415 (Mycoplasma pulmonis);
SEQ ID NO. 17: PolC: 416-FVIFDIETTGLH-427 (Mycoplasma genitalium);
SEQ ID NO. 18: PolC: 408-FVIFDIETTGLH-419 (Mycoplasma pneumoniae);
SEQ ID NO. 19: Pol III: 317-IMSFDIECAGRI-328 (Saccharomyces cerevisiae);
SEQ ID NO. 20: Pol II: 286-VMAFDIETTKPP-297 (Saccharomyces cerevisiae);
SEQ ID NO. 21: Pol δ: 310-VLSFDIECAGRK-321 (mouse);
SEQ ID NO. 22: Pol ε: 271-VLAFDIETTKLP-282 (mouse);
SEQ ID NO. 23: Pol δ: 312-VLSFDIECAGRK-323 (human);
SEQ ID NO. 24: Pol ε: 271-VLAFDIETTKLP-282 (human);
SEQ ID NO. 25: Pol δ: 316-ILSFDIECAGRK-327 (rice);
SEQ ID NO. 26: Pol ε: 306-VLSFDIECAGRK-317 (Arabidopsis thaliana);
SEQ ID NO. 27: Pol ε: 235-VCAFDIETVKLP-246 (Arabidopsis thaliana);
SEQ ID NO. 28: Pol δ: 308-VLSFDIECAGRK-319 (rat);
SEQ ID NO. 29: Pol δ: 311-VLSFDIECAGRK-322 (bovine);
SEQ ID NO. 30: Pol δ: 273-ILSFDIECAGRK-284 (soybean);
SEQ ID NO. 31: Pol δ: 296-ILSFDIECAGRK-307 (fruit fly); and
SEQ ID NO. 32: Pol ε: 269-VLAFDIETTKLP-280 (fruit fly).

Clearly, DNA polymerases having a proofreading function have well conserved aspartic acid (e.g. position 316 in human DNA polymerase δ) and glutamic acid (e.g., position 318 in human DNA polymerase δ). Regions containing such an aspartic acid and glutamic acid may be herein regarded as a proofreading function active site.

In gram-negative bacteria, such as E. coli, there are two DNA polymerase proteins, i.e., a molecule having exonuclease activity and a molecule having DNA synthesis activity. Therefore, by regulating exonuclease activity, the proofreading function can be regulated.

However, in gram-positive bacteria (e.g., B. subtilis, etc.) as well as eukaryotic organisms (e.g., yeast, animals, plants, etc.), one DNA polymerase has both DNA synthesis activity and exonuclease activity. Therefore, a molecule which regulates exonuclease activity while retaining normal DNA synthesis activity to regulate a proofreading function, is required. The present invention provides a variant of a DNA polymerase of eukaryotic organisms and gram-positive bacteria, which is capable of regulating exonuclease activity while maintaining normal DNA synthesis activity and which can be used in evolution of the organisms. Thereby, an effect which is different from that of E. coli and is not expected was achieved. Therefore, the present invention can be said to be achieved in part by the finding that the above-described proofreading function active site was unexpectedly specified in eukaryotic organisms and gram-positive bacteria, especially in eukaryotic organisms. Moreover, the significant effect of the present invention is acquisition of a hereditary trait which is unexpectedly shown in examples below.

A number of error-prone DNA polymerases have been found in bacteria and the like as well as humans. A number of replicative DNA polymerases typically have a proofreading function, i.e., remove errors by 3′→5′ exonuclease activity to perform error-free replication. However, error-prone DNA polymerases do not have a proofreading function and cannot bypass DNA damage, thus results in mutations. The presence of error-prone DNA polymerases is involved with the onset of cancer, evolution, antibody evolution, and the like. A number of DNA polymerases have the possibility of becoming error-prone. By disrupting their proofreading function, these DNA polymerases can be made error-prone. Therefore, the accuracy of replication can be regulated by modifying the above-described proofreading function active site. By using this model, a new property which has been once acquired can be advantageously evolved without abnormality. In this regard, an unexpected disadvantage and effect can be obtained in the present invention as compared to original disparity model.

In the quasispecies theory, Eigen advocates an evolution model in which only error-prone replication is taken into consideration (M. Eigen, Naturwissenschaften 58, 465(1971), etc.). The quasispecies theory uses various modifications. Quasispecies can be defined as a stable ensemble of the fittest sequence and its mutants are distributed around the fittest sequence in sequence space with selection. Natural selection appears to occur in not a single sequence but rather an entire quasispecies distribution. The evolution of quasispecies occurs as follows: a mutant with a higher fitness than the master sequence appears in the quasispecies, this mutant replaces the old master sequence with selection, and then a new quasispecies distribution organizes around the mutant.

The quasispecies theory expected and concluded that there exists an error threshold for maintaining genetic information. Therefore, conventionally, it is believed that quasispecies may only evolve below this threshold (M. Eigen at al., Adv. Chem. Phys. 75, 149 (1989)). This means that the upper limit of evolution rate is limited by the upper limit of the error threshold. The quasispecies theory seems to be proved in studies of RNA viruses, which evolve at a high rate near the error threshold. However, an agent with an increase in error rate in the phenotype of a mutated agent is believed to play an important role in this process.

Whereas the genomes of bacteria have a single origin of replication, the genomes of eukaryotic organisms have a plurality of origins of replication. This means that the sequence of the genome contains a plurality of replication units (replication agent, replicore). Therefore, a plurality of polymerases simultaneously participate in genomic replication. In the present invention, an influence of the number of replication agents on the error threshold may be taken into consideration.

In one preferred embodiment, by introducing a mutation capable of disrupting the 3′→5′ exonuclease activity into a gene (DNA polymerase gene) encoding a DNA polymerase, a nucleic acid molecule and polypeptide encoding a DNA polymerase having a reduced proofreading function i.e., a higher error-prone frequency) can be provided. Note that in a single DNA polymerase gene (PolC, POL2, CDC2, etc.), the 3′→5′ exonuclease activity (proofreading function) is contained in a molecule having DNA polymerization activity (e.g., eukaryotic organisms, gram-positive bacteria, etc.), or is encoded by a gene (e.g., dnaQ) different from a gene encoding DNA polymerization activity (e.g., dnaE) (e.g., gram-negative bacteria, etc.) (Kornberg A. and Baker T., “DNA Replication”, New York, Freeman, 1992). Based of the understanding of the above-described properties, those skilled in the art can regulate error-prone frequency according to the present invention. For example, in eukaryotic organisms, it is preferable to introduce a mutation, which changes a proofreading function but substantially not DNA polymerization activity, into a DNA polymerase. In this case, two acidic amino acids involved with the above-described proofreading function are modified (preferably, non-conservative substitution (e.g., substitutions of alanine, valine, etc.)) (Derbyshire et al., EMBO J. 10, pp. 17-24, January 1991; Fijalkowska and Schaaper, “Mutants in the Exo I motif of Escherichia coli dnaQ: Defective proofreading and inviability due to error catastrophe”, Proc. Natl. Acad. Sci. USA, Vol. 93, pp, 2856-2861, April 1996). The present invention is not limited to this.

As used herein, the term “proofreading function” refers to a function which detects and repairs a damage and/or an error in DNA of a cell. Such a function may be achieved by inserting bases at apurinic sites or apyrimidinic sites, or alternatively, cleaving one strand with an apurinic-apyrimidinic (A-P) endonuclease and then removing the sites with a 5′→3′ exonuclease. In the removed portion, DNA is synthesized and supplemented with a DNA polymerase, and the synthesized DNA is ligated with normal DNA by a DNA ligase. This reaction is called excision repair. For damaged DNA due to chemical modification by an alkylating agent, abnormal bases, radiation, ultraviolet light, or the like, the damaged portion is removed with a DNA glycosidase before repair is performed by the above-described reaction (unscheduled DNA synthesis). Examples of a DNA polymerase having such a proofreading function include, but are not limited to, DNA polymerase δ, DNA polymerase ε, etc. of eukaryotic organisms, and the like. As used herein, the term “fidelity” may also be used to represent the level of a proofreading function. The term “fidelity” refers to DNA replication accuracy. Normal DNA polymerases typically have a high level of fidelity. A DNA polymerase having a reduced proofreading function due to modification may have a low level of fidelity.

The above-described proofreading function of DNA polymerases is described in, for example, Kunkel, T. A.: J. Biol. Chem., 260, 12866-12874 (1985)1 Kunkel, T. A., Sabotino, R. D. & Bambara, R. A. Proc. Natl. Acad. Sci. USA, 84, 4865-4869 (1987): Wu, C. I. & Maeda, N.: Nature. 327, 167-170 (1987); Roberts, J. D. & Kunkel, T. A.: Proc. Natl. Acad. Sci. USA, 85, 7064-7068(1988): Thomas, D. C., Fitzgerald, M. P. & Kunkel, T. A.: Basic Life Sciences, 52, 287-297(1990); Trinh, T. Q. & Siden, R. R., Nature, 352, 544-547 (1991); Weston-Hafer, K. & Berg, D. E., Genetics, 127, 649-655(1991); Veaute, X. & Fuchs, R. P. P.: Science, 261, 598-600 (1993); Roberts, J. D., Izuta, S. Thomas, D. C. & Kunkel, T. A.: J. Biol. Chem., 269, 1711-1717 (1994): Roche, W. A., Trinh, T. Q. & Siden, R. R., J. Bacteriol., 177, 4385-4391 (1995); Sang, S., Jaworski, A., Ohshirna K. & Wells, Nat. Genet., 10, 213-218 (1995); Fijalkowska, I. J., Jonczyk, P., Maliszewska-Tkaczyk, M., Bialoskorska, M. & Schaeper, R. M., Proc. Natl. Acad. Sci. USA., 95, 10020-10025 (1998); Malisewska-Tkaczyk, M., Jonezyk, P., Bialoskorska, M., Schaaper, M. & Fijalkowska, I.: Proc. Natl. Aced. Sat. USA, 97, 12678-12683 (2000); Gwel, D., Jonezyk, P., Bialoskorska, M., Schaaper, R. M. & Fijalkowska, I. J.: Mutation Research, 501, 129-136 (2002) Roberts, J. D., Thomas, D. C. & Kunkel, T. A.: Proc. Natl. Acad. Sci. USA, 88, 3465-3469 (1991); Roberts, J. D., Nguyen, D. & Kunkel, T. A.: Biochemistry, 32, 4083-4089 (1993); Francino, M. P., Chao, L., Riley, M. A. & Ochman, H.: Science, 272, 107-109 (1996); A. Boulet, M. Simon, G. Faye, G. A. Bauer & P. M. Burgers, EMBO J., 8, 1849-1854, (1989); Morrison A., Araki H., Clark A. B., Hamatake R. K., & Sugino A., Cell, 62(6), 1143-1151 (1990), etc.

As used herein, the term “DNA polymerase δ” of eukaryotic organisms refers to an enzyme involved in DNA elongation, which is said to have exonuclease activity leading to a proofreading function. A representative DNA polymerase δ has sequences set forth in SEQ ID Nos. 1 and 2 (a nucleic acid sequence and an amino acid sequence, respectively; polδ: X61920 gi/171411/gb/M61710.1/YSCDPB2[171411]). The proofreading function of this DNA polymerase δ can be regulated by modifying an amino acid at position 322 of the amino acid sequence set forth in SEQ ID NO. 2. The DNA polymerase δ is described in Simon, M. et al., EMBO J. 10, 2163-2170, 1991, whose contents are incorporated herein by reference. Examples of the DNA polymerase δ include, but are not limited to, those of Arabidopsis thaliana (SEQ ID NO. 45), rice (SEQ ID NOs. 47 and 48), soybean (SEQ ID NOs. 49 and 50), human (SEQ ID NOs. 51 and 52), mouse (SEQ ID NO. 55 and 56), rat (SEQ ID NOs. 59 and 60), bovine (SEQ ID NOs. 61 and 62), fruit fly (SEQ ID NOs. 63 and 64), and the like.

As used herein, the term “DNA polymerase ε” of eukaryotic organisms refers to an enzyme involved with replication of a gap between lagging strands, which is said to have exonuclease activity leading to a proofreading function. A representative DNA polymerase ε has sequences set forth in SEQ ID NOs. 3 and 4 (a nucleic acid sequence and an amino acid sequence, respectively: pol ε; M60416 gi/171408/gb/M60416.1/YSCDNA POL[171408]). The proofreading function of the DNA polymerase ε can be regulated by modifying an amino acid at position 391 of the amino acid sequence set forth in SEQ ID NO. 4. The DNA polymerase ε is described in, for example, Morrison, A. et al., MGG.242, 289-296, 1994: Araki H., et al., Nucleic Acids Res. 19, 4857-4872, 1991: and Ohya T., et al., Nucleic Acids Res. 28, 3846-3852, 2000, whose contents are incorporated herein by reference. Examples of the DNA polymerase ε include, but are not limited to, those of Arabidopsis thaliana (SEQ ID NO. 46), human (SEQ ID NOs. 53 and 54), mouse (SEQ ID NOs. 57 and 58), fruit fly (SEQ ID NOs. 65 and 66), and the like.

DNA polymerases δ and ε are referred to as POLD1/POL3 and POLE/POL2, respectively, according to the HUGO categories. Both nomenclatures may be used herein.

Other DNA polymerases are described in, for example, Lawrence C. W. et al., J. Mol. Biol., 122, 1-21, 1978; Lawrence C. W. et al., Genetics 92, 397-408; Lawrence C. W. et al., MGG. 195, 457-490, 1984; Lawrence C. W. et al., MGG. 200, 86-91, 1985 (DNA polymerase β and DNA polymerase ξ); Maher V. M. et al., Nature 261, 593-595, 1976; McGregor, W. G. et al., Mol. Cell. Biol. 19, 147-154, 1999 (DNA polymerase η); Strand M. et al., Nature 365, 275-276, 1993; Prolla T. A., et al., Mol. Cell. Biol. 15, 407-415, 1994; rat A., et al., Proc. Natl. Acad. Sci. USA 90, 6424-6428 Bhattacharyya N. P., et al., Proc. Natl. Acad. Sci. USA 91, 6319-6323, 1994; Faber F. A., et al., Hum. Mol. Genet. 3, 253-256, 1994; Eshleman, J. R., et al., Oncogene 10, 33-37, 1995; Morrison A., et al., Proc. Natl. Aced. Sci. USA 68, 9473-9477, 1991; Morrison A., et al., EMBO J. 12, 1467-1473, 1993; Foury F., at al., EMBO J. 11, 2717-2726, 2992 (DNA polymerase λ, DNA polymerase μ, etc.); and the like, whose contents are incorporated herein by reference.

As used herein, the term “wild type” in relation to genes encoding DNA polymerases and the like and organisms (e.g., yeast, etc.) refers in its broadest sense, to a type that is characteristic of most members of a species from which naturally-occurring genes encoding DNA polymerases and the like and organisms (e.g., yeast, etc.) are derived. Therefore, typically, the type of genes encoding DNA polymerases and the like and organisms (e.g., yeast, etc.) Which are first identified in a certain species can be said to be a wild type. Wild type is also referred to as “natural standard type”. Wild type DNA polymerase δ has sequences set forth in SEQ ID NOs. 1 and 2. Wild type DNA polymerase ε has sequences set forth in SEQ ID NOs. 3 and 44. DNA polymerases having sequences set forth in SEQ ID NOs. 41 to 66 are also of wild type. Wild type organisms may have normal enzyme activity, normal traits, normal behavior, normal physiology, normal reproduction, and normal genomes.

As used herein, the term “lower than wild type” in relation to a proofreading function of an enzyme or the like means that the proofreading function of the enzyme is lower than that of the wild type enzyme (i.e., the number of mutations remaining after the proofreading process of the enzyme is greater than that of the wild type enzyme). Comparison with wild types can be carried out by relative or absolute representation. Such comparison can be carried out using error-prone frequency or the like.

As used herein, the term “mutation” in relation to a gene means that the sequence of the gene is altered or refers to a state of the altered nucleic acid or amino acid sequence of the gene. For example, the term “mutation” herein refers to a change in the sequence of a gene leading to a change in the proofreading function. Unless otherwise defined, the terms “mutation” and “variation” have the same meaning throughout the specification.

Mutagenesis is most commonly performed for organisms in order to produce their useful mutants. The term “mutation” typically refers to a change in a base sequence encoding a gene, encompassing a change in a DNA sequence. Mutations are roughly divided into the following three groups in accordance with the influence thereof on an individual having the mutation: A) neutral mutation (most mutations are categorized into this group, and there is substantially no influence on the growth and metabolism of organisms); B) deleterious mutation (Its frequency is lower than that of neutral mutations. This type of mutation inhibits the growth and metabolism of organisms. The deleterious mutation encompasses lethal mutations which disrupt genes essential for growth. In the case of microorganisms, the proportion of deleterious mutations is typically about 1/10 to 1/100 of the total of mutations, though varying depending on the species); and C) beneficial mutation (this mutation is beneficial for breeding of organisms. The occurrence frequency is considerably low compared to neutral mutations. Therefore, a large population of organisms and a long time period are required for obtaining individual organisms having a beneficial mutation. An effect sufficient for breeding of organisms is rarely obtained by a single mutation and often requires accumulation of a plurality of beneficial mutations.)

As used herein, the term “growth” in relation to a certain organism refers to a quantitative increase in the individual organism. The growth of an organism can be recognized by a quantitative increase in a measured value, such as body size (body height), body weight, or the like. A quantitative increase in an individual depends on an increase in each cell and an increase in the number of cells.

As used herein, the term “substantially the same growth” in relation to an organism moans that the growth rate of the organism is not substantially changed as compared to a reference organism (e.g., an organism before transformation). An exemplary range in which the growth rate is considered not to be substantially changed, includes, but is not limited to, a range of 1 deviation in a statistical distribution of typical growth. In the organism of the present invention, the term “substantially the same growth” means, for example, (1) the number of progenitors is not substantially changed; (2) although the morphology is changed, substantially no disorder is generated as is different from typical artificial mutations. Despite a considerably high rate of mutations, appearance is appreciated as being “beautiful” (although this feature is not directly related to growth, the feature is characteristic to mutants created by the method of the present invention); and (3) a trait, genotype, or phenotype which has been once acquired does not regress.

As used herein, the term “drug resistance” refers to tolerance or resistance to drugs including physiologically active substances, such as bacteriophages, bacteriocins, and the like. Drug resistance is acquired by sensitive hosts when a receptor thereof for a drug is altered or one or more of the various processes involved in the action of a drug is altered. Alternatively, when sensitive hosts acquire ability to inactivate antibiotics themselves, drug resistance may be obtained. In drug resistant organisms, a mutation in chromosomal DNA may alter an enzyme and/or a ribosome protein on which a drug acts on, so that the drug having an ordinary concentration is no longer effective. Alternatively, an organism may acquire a drug resistant plasmid (e.g., R plasmid) from other organisms, so that enzyme activity to inactivate a drug is obtained. Alternatively, the membrane permeability of a drug may be reduced to acquire resistance to the drug. The present invention is not limited to this.

As used herein, the term “cancer cell” has the same meaning as that of the term “malignant tumor cell” including sarcoma and refers to a cell which has permanent proliferating ability and is immortal. Cancer cells acquire permanent proliferating ability and become immortal in the following fashion. A certain irreversible change is generated in a normal cell at the gene level. As a result, the normal cell is transformed into an abnormal cell, i.e., a cancer cell.

As used herein, the term “production” in relation to an organism means that the individual organism is produced.

As used herein, the term “reproduction” in relation to an organism means that a new individual of the next generation is produced from a parent individual. Reproduction includes, but is not limited to, natural multiplication, proliferation, and the like; artificial multiplication, proliferation, and the like by artificial techniques, such as cloning techniques (nuclear transplantation, etc.). Examples of a technique for reproduction include, but are not limited to, culturing of a single cell; grafting of a cutting; rooting of a cutting; and the like, in the case of plants. Reproduced organisms typically have hereditary traits derived from their parents. Sexually reproduced organisms have hereditary traits derived from typically two sexes. Typically, these hereditary traits are derived from two sexes in substantially equal proportions. Asexually reproduced organisms have hereditary traits derived from their parents.

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be naturally-occurring cells or artificially modified cells (e.g., fusion calls, genetically modified cells, etc.). Examples of a source for calls include, but are not limited to, a single cell culture, the embryo, blood, or body tissue of a normally grown transgenic animal, a cell mixture, such as cells from a normally grown cell line, and the like.

Cells for use in the present invention may be derived from any organism (e.g., any unicellular organism (e.g., bacteria, yeast, etc.) or any multicellular organism (e.g., animals (e.g., vertebrates, invertebrates), plants (e.g., monocotyledonous plants, dicotyledonous plants, etc.), etc.)). For example, cells derived from vertebrates (e.g., Myxiniformes, Petronyzoniformes, Chondrichthyes, Osteichthyes, amphibian, reptilian, avian, mammalian, etc.) are used. Specifically, cells derived from mammals (e.g., monotremata, marsupialia, edentate, dermoptera, chiroptera, carnivore, insectivore, proboscides, perissodactyla, artiodactyla, tubulidentata, pholidota, sirenia, cetacean, primates, rodentia, lagomorpha, etc.). In one embodiment, cells derived from primates (e.g., chimpanzees, Japanese monkeys, humans, etc.), especially humans may be used. The present invention is not limited to this. Cells for use in the present invention may be stem cells or somatic cells. The above-described cells may be used for the purpose of implantation. Cells derived from flowering plants (monocotyledons or dicotyledons) may be used. Preferably, is dicotyledonous plant cells are used. More preferably, cells from the family Gramineae, the family Solanaceae, the family Cucurbitaceae, the family Cruciferae, the family Umbelliferae, the family Rosaceae, the family Leguminosae, and the family Boraginaceae are used. Preferably, cells derived from wheat, maize, rice, barley, sorghum, tobacco, green pepper, eggplant, melon, tomato, strawberry, sweet potato, Brassica, cabbage, leek, broccoli, soybean, alfalfa, flax, carrot, cucumber, citrus, Chinese cabbage, lettuce, peach, potato, Lithospermum eythrohizon, Coptis Rhizome, poplar, and apple, are used. Plant cells may be a part of plant body, an organ, a tissue, a culture cell, or the like. Techniques for transforming cells, tissues, organs or individuals are well known in the art. These techniques are well described in the literature cited herein and the like. Nucleic acid molecules may be transiently or stably introduced into organism cells. Techniques for introducing genes transiently or stably are well known in the art. Techniques for differentiating cells for use in the present invention so as to produce transformed plants are also well known in the art. It will be understood that these techniques are well described in literature cited herein and the like. Techniques for obtaining seeds from transformed plants are also well known in the art. These techniques are described in the literature mentioned herein.

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cell, tissue-specific stem cell, or somatic stem cell). A stem cell may be an artificially produced cell as long as it can have the above-described abilities. The term “embryonic stem cell” refers to a Pluripotent stem cell derived from early embryos. As are different from embryonic stem cells, the direction of differentiation of tissue stem cells is limited. Embryonic stem cells are located at specific positions in tissues and have undifferentiated intracellular structures. Therefore, tissue stem cells have a low level of pluripotency. In tissue stem cells, the nucleus/cytoplasm ratio is high, and there are few intracellular organelles. Tissue stem cells generally have pluripotency and the cell cycle to long, and can maintain proliferation ability beyond the life of an individual. Stem cell used herein may be embryonic stem cells or tissue stem cells as long as they are capable of regulating the error-prone frequency of gene replication.

Tissue atom cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreas (common) stem cells, liver stem cells, and the like. Tissue stem cells in the bone marrow system include hematopoietic stem cells, mesenchymal stem cells, and the like. Tissue stem cells in the nervous system include neural stem cslls, retina stem cells, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they are capable of regulating the error-prone frequency of gene replication.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoietic stem cells, mesenchymal stem cells, and the like. Stem cells of mesoderm origin are mostly present in organs, including liver stem cells, pancreas stem cells, and the like. Somatic cells as used herein may be derived from any germ layer as long as they are capable of regulating the error-prone frequency of gene replication.

As used herein, the term “isolated” indicates that at least a naturally accompanying substance in a typical environment is reduced, preferably substantially excluded. Therefore, the term “isolated cell” refers to a cell which contains substantially no naturally accompanying substance in a typical environment (e.g., other cells, proteins, nucleic acids, etc.). The term “isolated” in relation to a nucleic acid or a polypeptide refers to a nucleic acid or a polypeptide which contains substantially no cellular substance or culture medium when is is produced by recombinant DNA techniques or which contains substantially no precursor chemical substance or other chemical substances when it is chemically synthesized, for example. Preferably, isolated nucleic acids do not contain a sequence which naturally flanks the nucleic acid in organisms (the 5′ or 3′ terminus of the nucleic acid).

As used herein, the term “established” in relation to cells refers to a state of a cell in which a particular property (pluripotency) of the cell is maintained and the cell undergoes stable proliferation under culture conditions. Therefore, established stem cells maintain pluripotency.

As used herein, the term “differentiated cell” refers to a cell having a specialized function and form (e.g., muscle cells, neurons, etc.). Unlike stem cells, differentiated cells have no or little pluripotency. Examples of differentiated cells include epidermic cells, pancreatic parenchymal cello, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cello, and the like. Cells used herein may be any of the above-described cells as long as they are capable of regulating the error-prone frequency of gene replication. As used herein, the terms “differentiation” or “cell differentiation” refers to a phenomenon that two or more types of calls having qualitative differences in form and/or function occur in a daughter cell population derived from the division of a single cell. Therefore, “differentiation” includes a process during which a population (family tree) of cells which do not originally have a specific detectable feature acquire a feature, such as production of a specific protein, or the like.

As used herein, the term “state” in relation to a cell, an organism, or the like, refers to a condition or mode of a parameter (e.g., a cell cycle, a response to an exogenous agent, signal transduction, gene expression, gene transcription, etc.) of the cell, the organism, or the like. Examples of such a state include, but are not limited to, a differentiated state, an undifferentiated state, a response of a cell to an exogenous agent, a cell cycle, a proliferation state, and the like. The responsiveness or resistance of an organism of interest with respect to the following parameters of, particularly, environments of the organism may be used herein as a measure of the state of the organism: temperature, humidity (e.g., absolute humidity, relative humidity, etc.), pH, salt concentration (e.g., the concentration of all salts or a particular salt), nutrients (e.g., the amount of carbohydrate, etc.), metals (e.g., the amount or concentration of all metals or a particular metal (e.g., a heavy metal, etc.)), gas (e.g., the amount of all gases or a particular gas), organic solvent (e.g., the amount of all organic solvents or a particular organic solvent (e.g., ethanol, etc.)), pressure (e.g., local or global pressure, etc.), atmospheric pressure, viscosity, flow rate (e.g., the flow rate of a medium in which an organism is present, etc.), light intensity (e.g., the quantity of light having a particular wavelength, etc.), light wavelength (e.g., visible light, ultraviolet light, infrared light, etc.), electromagnetic waves, radiation, gravity, tension, acoustic waves, organisms other than an organism of interest (e.g., parasites, pathogenic bacteria, etc.), chemicals (e.g., pharmaceuticals, etc.), antibiotics, naturally-occurring substances, metal stresses, physical stresses, and the like.

As used herein, the term “environment” (or “Umgebung” in Germany) in relation to an entity refers to a circumstance which surrounds the entity. In an environment, various components and quantities of state are recognized, which are called environmental factors. Examples of environmental factors include the above-described parameters. Environmental factors are typically roughly divided into non-biological environmental factors and biological environmental factors. Non-biological environmental factors (inorganic environment factors) may be divided into physical factors and chemical factors, or alternatively, climatic factors and soil factors. Various environmental factors do not always act on organisms independently, but may be associated with one another. Therefore, environment factors may be herein observed one by one or as a whole (a whole of various parameters).

As used herein, the term “tissue” refers to an aggregate of cells having substantially the same function and/or form in a multicellular organism. “Tissue” is typically an aggregate of cells of the same origin, but may be an aggregate of cells of different origins as long as the cells have the same function and/or form. Therefore, when a stem cell of the present invention is used to regenerate a tissue, the tissue may be composed of an aggregate of cells of two or more different origins. Typically, a tissue constitutes a part of an organ. Animal tissues are separated into epithelial tissue, connective tissue, muscular tissue, nervous tissue, and the like, on a morphological, functional, or developmental basis. Plant tissues are roughly separated into meristematic tissue and permanent tissue according to the developmental stage of the calls constituting the tissue. Alternatively, tissues may be separated into single tissues and composite tissues according to the type of cells constituting the tissue. Thus, tissues are separated into various categories. Any tissue may be herein used as long as the error-prone frequency of gene replication can be regulated therein.

Any organ or a part thereof may be used in the present invention. Tissues or cells to be injected in the present invention may be derived from any organ. As used herein, the term “organ” refers to a morphologically independent structure localized at a particular portion of an individual organism in which a certain function is performed. In multicellular organisms (e.g., animals, plants), an organ consists of several tissues spatially arranged in a particular manner, each tissue being composed of a number of cells. An example of such an organ includes an organ relating to the vascular system. In one embodiment, organs targeted by the present invention include, but are not limited to, skin, blood vessel, cornea, kidney, heart, liver, umbilical cord, intestine, nerve, lung, placenta, pancreas, brain, peripheral limbs, retina, and the like. Any organ or a part thereof may be used in the present invention as long as the error-prone frequency of gene replication can be regulated therein.

As used herein, the term “product substance” refers to a substance produced by an organism of interest or a part thereof. Examples of such a product substance include, but are not limited to, expression products of genes, metabolites, excrements, and the like. According to the present invention, by regulating the conversion rate of a hereditary trait, an organism of interest is allowed to change the type and/or amount of the product substance. It will be understood that the present invention encompasses the thus-changed product substance. Preferably, the product substance may be, but is not limited to, a metabolite.

As used herein, the term “model of disease” in relation to an organism refers to an organism model in which a disease, a symptom, a disorder, a condition, or the like specific to the organism can be recreated. Such a model of disease can be produced by a method of the present invention. Examples of such a model of disease include, but are not limited to, animal models of cancer, animal models of a heart disease (e.g., myocardiac infarction, etc.), animal models of a cardiovascular disease (e.g., arterial solerosis, etc.), animal models of a central nervous disease (e.g., dementia, cerebral infarction, etc.), and the like.

(General Biochemistry and Molecular Biology)

(General Techniques)

Molecular biological techniques, biochemical techniques, microorganism techniques, and cellular biological techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987). Current Protocols in Molecular Biology, Greene Pub. Associates and Wiley-Interscience; Ausubel, F. M. (1989) Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates: Innis, M. A. at al. (1995), PCR Strategies, Academic Press; Ausubel, F. M. (1999). Short Protocols in Molecular Biology: A Compendium of Methods from Current Protocols in Molecular Biology, Wiley, and annual updates: Sninsky, J. J. et al. (1999), PCR Applications: Protocols for Functional Genomics, Academic Press; Special issue, Jikken Igaku [Experimental Medicine]“Indenshi Donyu & Hatsugen Kaiseki Jikkenho [Experimental Methods for Gene introduction & Expression Analysis]”, Yodo-sha, 1997, and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approac, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate techniques, Academic Press; and the like, related portions of which are herein incorporated by reference.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art for example. The gene product of the present invention is typically in the form of a polypeptide. A product substance of the present invention in the form of a polypeptide may be useful as a pharmaceutical composition or the like.

The terms “polynucleotide”, “oligonucleotide” and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide to substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes 8:91-98(1994)). The gene of the present invention is typically in the form of a polynucleotide. The gene or gone product of the present invention in the form of a polynucleotide is useful for the method of the present invention.

As used herein, the term “nucleic acid molecule” is also used interchangeably with the terms “nucleic acid”, “oligonucleotide”, and “polynucleotide”, including cDNA, mRNA, genomic DNA, and the like. As used herein, nucleic acid and nucleic acid molecule may be included by the concept of the term “gene”. A nucleic acid molecule encoding the sequence of a given gene includes “splice mutant (variant)”. Similarly, a particular protein encoded by a nucleic acid encompasses any protein encoded by a splice variant of that nucleic acid. “Splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, a gene of the present invention may include the splice mutants herein.

As used herein, “homology” of a gene (e.g., a nucleic acid sequence, an amino acid sequence, or the like) refers to the proportion of identity between two or more gene sequences. As used herein, the identity of a sequence (a nucleic acid sequence, ah amino acid sequence, or the like) refers to the proportion of the identical sequence (an individual nucleic acid, amino acid, or the like) between two or more comparable sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, “similarity” of a gene (e.g. a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using PSI-BLAST (sequence analyzing tool) with the default parameters. Otherwise, FASTA (using default parameters) may be used instead of PSI-BLAST.

As used herein, the term “amino acid” may refer to a naturally-occurring or nonnaturally-occurring amino acid as long as it satisfies the purpose of the present invention. The term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers, although embodiments using D-amino acids are within the scope of the present invention. The term “nonnaturally-occurring amino acid” refers to an amino acid which is ordinarily not found in nature. Examples of nonnaturally-occurring amino acids include norleucine, para-nitrophenylalanine, homophenylalanine, para-fluorophenylalanine, 3-amino-2-benzil propionic acid, D- or L-homoarginine, and D-phenylalanine. The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site (e.g., a range which provides a proofreading function of a DNA polymerase) and similarly contributes to catalytic activity. For example, in the case of antisense molecules, the term refers to a similar portion in an ortholog corresponding to a particular portion of the antisense molecule. Corresponding amino acids and nucleic acids can be identified using alignment techniques known in the art. Such an alignment technique is described in, for example, Needleman, S. B. and Wunsch, C. D. J. Mol. Biol. 48, 443-453, 1970.

As used herein, the term “corresponding” gene (e.g., a polypeptide or polynucleotide molecule) refers to a gene (e.g., a polypeptide or polynucleotide molecule) in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to a mouse DNA polymerase gene and the like can be found in other animals (human, rat, pig, cattle, and the like). Such a corresponding gene can be identified by techniques well known in the art. Therefore, for example, a corresponding gene in a given animal can be found by searching a sequence database of the animal (e.g., human, rat) using the sequence of a reference gene (e.g., mouse DNA polymerase genes, and the like) as a query sequence.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs ate well known in the art. Examples of such nucleotide derivatives and nucleotide analogs include, but are not limited to, phosphorothioate, phosphoramidate, methylphosphonate, chiral-methylphosphonate, 2-O-methyl ribonucleotide and peptide-nucleic acid (PNA).

As used herein, the term “fragment” refers to a polypeptide or polynucleotide having a sequence length ranging from 1 to n-1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification. The length of useful fragment may be determined depending on whether or not at least one function (e.g., specific interaction with other molecules, etc.) is maintained among the functions of a full-length protein which is a reference of the fragment.

As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like. As used herein, the “agent” may be any substance or other agent (e.g., energy, such as light, radiation, heat, electricity, or the like) as long as the intended purpose can be achieved. Examples of such a substance include, but are not limited to, proteins, polypeptides, oligopeptides, peptides, polynucleotides, oligonucleotides, nucleotides nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like. These agents may be herein useful for regulation of the error-prone frequency of organisms.

As used herein, the term “low molecular weight organic molecule” refers to an organic molecule having a relatively small molecular weight. Usually, the low molecular weight organic molecule refers to a molecular weight of about 1,000 or less, or may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecule include, but are not limited to, hormones, ligands, information transfer substances, synthesized by combinatorial chemistry, pharmaceutically acceptable low molecular weight molecules (e.g., low molecular weight ligands and the like), and the like. These agents may be herein useful for regulation of the error-prone frequency of organisms.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “antigen” refers to any substrate to which an antibody molecule may specifically blind. As used herein, the term “immunogen” refers to an antigen capable of initiating activation of the antigen-specific immune response of a lymphocyte.

As used herein, the term “single chain antibody” refers to a single chain polypeptide formed by linking the heavy chain fragment and the light chain fragment of the Fv region via a peptide crosslinker.

As used herein, the term “composite molecule” refers to a molecule in which a plurality of molecules, such as polypeptides, polynucleotides, lipids, sugar, low molecular weight molecules, and the like, are linked together. Examples of such a composite molecule include, but are not limited to, glycolipids, glycopeptides, and the like. These molecules can be used herein as genes or products thereof (e.g., DNA polymerases, etc.) or as the agent of the present invention as long as the molecules have substantially the same function as those of the genes or products thereof (e.g., DNA polymerases, etc.) or the agent of the present invention.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “expression” of a gene product, such as a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

As used herein, the term “reduction of expression” of a gene, a polynucleotide, a polypeptide, or the like indicates that the level of expression is significantly reduced in the presence of the action of the agent of the present invention, as compared to when the action of the agent is absent. Preferably, the reduction of expression includes a reduction in the amount of expression of a polypeptide (e.g., a DNA polymerase and the like). As used herein, the term “increase of expression” of a gene, a polynucleotide, a polypeptide, or the like indicates that the level of expression is significantly increased in the presence of the action of the agent of the present invention, as compared to when the action of the agent is absent. Preferably, the increase of expression includes an increase in the amount of expression of a polypeptide (e.g., a DNA polymerase and the like). As used herein, the term “induction of expression” of a gene indicates that the amount of expression of a gene is increased by applying a given agent to a given cell. Therefore, the induction of expression includes allowing a gene to be expressed when expression of the gene is not otherwise observed, and increasing the amount of expression of the gene when expression of the gene is observed. The increase or reduction of these genes or gene products (polypeptides or polynucleotides) may be useful in regulating error-prone frequencies in replication, for example, in the present invention.

As used herein, the term “specifically expressed” in the case of genes indicates that a gene is expressed in a specific site or for a specific period of time at a level different from (preferably higher than) that in other sites or periods of time. The term “specifically expressed” indicates that a gene may be expressed only in a given site (specific site) or may be expressed in other sites. Preferably, the term “specifically expressed” indicates that a gene is expressed only in a given site. Therefore, according to an embodiment of the present invention, a DNA polymerase may be expressed specifically or locally in a desired portion.

As used herein, the term “biological activity” refers to activity possessed by an agent (e.g., a polynucleotide, a protein, etc.) within an organism, including activities exhibiting various functions (e.g., transcription promoting activity). For example, when two agents interact with each other (e.g., a DNA polymerase binds to a sequence specific thereto), the biological activity includes linkage between the DNA polymerase and the specific sequence, a biological change caused by the linkage (e.g., a specific nucleotide polymerization reaction, occurrence of replication errors errors nucleotide removing ability; recognition of mismatched base pairs; etc.). For example, when a given agent is an enzyme, the biological activity thereof includes the emzymatic activity thereof. In another example, when a given agent is a ligand, the biological activity thereof includes binding of the agent to a receptor for the ligand. Such biological activity can be measured with a technique well known in the art.

As used herein, the term “antisense (activity)” refers to activity which permits specific suppression or reduction of expression of a target gene. The antisense activity is ordinarily achieved by a nucleic acid sequence having a length of at least 8 contiguous nucleotides, which is complementary to the nucleic acid sequence of a target gene (e.g., a DNA polymerase and the like). Such a nucleic acid sequence preferably have a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 30 contiguous nucleotides, a length of at least 40 contiguous nucleotides, and a length of at least 50 contiguous nucleotides. These nucleic acid sequences include nucleic acid sequences having at least 70% homology thereto, more preferably at least 80%, even more preferably at least 90%, and still even more preferably at least 95%. The antisense activity is preferably complementary to a 5′ terminal sequence of the nucleic acid sequence of a target gene. Such an antisense nucleic acid sequence includes the above-described sequences having one or several, or at least one, nucleotide substitutions, additions, and/or deletions. Molecules having such antisense activity may be herein useful for regulation of an error-prone frequency in organisms.

As used herein, the term “RNAi” is an abbreviation of RNA interference and refers to a phenomenon that an agent for causing RNAi, such as double-stranded RNA (also called dsRNA), is introduced into cells and mRNA homologous thereto is specifically degraded, so that synthesis of gene products is suppressed, and a technique using the phenomenon. As used herein, RNAi may have the same meaning as that of an agent which causes RNAi.

As used herein, the term “an agent causing RNAi” refers to any agent capable of causing RNAi. As used herein, “an agent causing RNAi for a gene” indicates that the agent causes RNAi relating to the gene and the effect of RNAi is achieved (e.g., suppression of expression of the gene, and the like). Examples of such an agent causing RNAi include, but are not limited to, a sequence having at least about 70% homology to the nucleic acid sequence of a target gene or a sequence hybridizable under stringent conditions, RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g. 2-4 nucleotides in length). RNAi may be herein useful for regulation of an error-prone frequency in organisms.

As used herein, “polynucleotides hybridizing under stringent conditions” refers to conditions commonly used and well known in the art. Such a polynucleotide can be obtained by conducting colony hybridization, plaque hybridization, southern blot hybridization, or the like using a polynucleotide selected from the polynucleotides of the present invention. Specifically, a filter on which DNA derived from a colony or plaque is immobilized is used to conduct hybridization at 65° C. In the presence of 0.7 to 1.0 M NaCl. Thereafter, a 0.1 to 2-fold concentration SSC (saline-sodium citrate) solution (I-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A (adenine) or T (thymine). “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 95%.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.0015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, N.Y., 1989); Anderson et al., Nucleic Acid Hybridization: A Practical Approach Ch. 4 (IRL Press Limited) (Oxford Express). More stringent conditions (such as higher temperature, lower ionic strength, higher formamide, or other denaturing agents) may be optionally used. Other agents may be included in the hybridization and washing buffers for the purpose of reducing non-specific and/or background hybridization. Examples are 0.1% bovine serum albumin, 0.1% polyvinylpyrrolidone, 0.1% sodium pyrophosphate, 0.1% sodium dodecylsulfate (NaDodSO₄ or SDS), Ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are ordinarily carried out at pH 6.8-7.4; however, at typical ionic strength conditions, the rate of hybridization is nearly independent of pH. See Anderson et al., Nucleic Acid Hybridization: A Practical Approach Ch. 4 (IRL Press Limited, Oxford UK).

Agents affecting the stability of DNA duplex include base composition, length, and degree of base pair mismatch. Hybridization conditions can be adjusted by those skilled in the art in order to accommodate these variables and allow DNAs of different sequence relatedness to form hybrids. The melting temperature of a perfectly matched DNA duplex can be estimated by the following equation:
Tm(° C.)=81.5+16.6(log[Na⁺])+0.41 (% G+C)−600/N−0.72 (% formamide)
where N is the length of the duplex formed, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example a “moderately stringent” conditions” of 50° C. In 0.015 M sodium ion will allow about a 21% mismatch.

It will be appreciated by those skilled in the art that there is no absolute distinction between “highly stringent conditions” and “moderately stringent conditions”. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, those skilled in the art can simply lower the temperature or raise the ionic strength.

A good estimate of the melting temperature in 1 M NaCl for oligonucleotide probes up to about 20 nucleotides is given by:
Tm=(2° C. per A−T base pair)+(4° C. per G−C base pair).

Note that the sodium ion concentration in 6×salt sodium citrate (SSC) is 1 M. See Suggs et al., Developmental Biology Using Purified Genes 683 (Brown and Fox, eds., 1981).

A naturally-occurring nucleic acid encoding a DNA polymerase protein is readily isolated from a cDNA library having PCR primers and hybridization probes containing part of a nucleic acid sequence indicated by, for example, SEQ ID NO. 1, 3, 41, 43, 47, 49, 51, 53, 55, 57, 59, 61, 63, 65, or the like. A preferable nucleic acid encoding a DNA polymerase, or variants or fragments thereof, or the like is hybridizable to the whole or part of a sequence as set forth in SEQ ID NO. 1, 3, 41, 43, 47, 49, 51, 53, 55, 57, 59, 61, 63, 65, or the like under low stringent conditions defined by hybridization buffer essentially containing 1% bovine serum albumin (BSA): 500 mM sodium phosphate (NaPO₄); 1 mM EDTA; and 7% SDS at 42° C., and wash buffer essentially containing 2×SSC (600 mM NaCl; 60 mM sodium citrate); and 0.1% SDS at 50° C., more preferably under low stringent conditions defined by hybridization buffer essentially containing 1% bovine serum albumin (BSA); 500 mM sodium phosphate (NaPO₄); 15% formamide; 1 mM EDTA; and 7% SDS at 50° C., and wash buffer essentially containing 1×SSC (300 mM NaCl; 30 mM sodium citrate); and 1% SDS at 50° C., and most preferably under low stringent conditions defined by hybridization buffer essentially containing 1% bovine serum albumin (BSA); 200 mM sodium phosphate (NaPO₄); 15% formamide; 1 mM EDTA; and 7% SDS at 50° C., and wash buffer essentially containing 0.5×SSC (150 mM NaCl: 15 mM sodium citrate); and 0.1% SDS at 65° C.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotide, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 225: 403-410 (1990)), FASTA (Pearson & Lipman, Proc. Natl. Acad. Sci., USA 85:2444-2448 (1988)), Smith and Waterman method (5 Smith and Waterman, J. Mol. Biol. 1472195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane or the like or a microarray (microassay) in which genomic DNA is attached to a glass plate under stringent hybridization, PCR and in situ hybridization, and the like. It is herein intended that a DNA polymerase and the like used in the present invention include corresponding genes identified by such an electronic or biological search.

As used herein, the “percentage of sequence identity, homology or similarity (amino acid, nucleotide, or the like)” is determined by comparing two optimally aligned sequences over a window of comparison, wherein the portion of a polynucleotide or polypeptide sequence in the comparison window may comprise additions or deletions (i.e. gaps), as compared to the reference sequences (which does not comprise additions or deletions (if the other sequence includes an addition, a gap may occur)) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e. the window size) and multiplying the results by 100 to yield the percentage of sequence identity. When used in a search, homology is evaluated by an appropriate technique selected from various sequence comparison algorithms and programs well known in the art. Examples of such algorithms and programs include, but are not limited to, TBLASTN, BLASTP, FASTA, TFASTA and CLUSTALW (Pearson and Lipman, 1988, Proc. Natl. Acad. Sci. USA 85(8); 2444-2448, Altschul et al., 1990, J. Mol. Biol. 215(3): 403-410, Thompson et al., 1994, Nucleic Acids Ras. 22(2): 4673-4680, Higgins et al., 1996, Methods Enzymol. 266:383-402, Altschul et al., 1990, J. Mol. Biol. 215(3): 403-410, Altschul et al., 1993, Nature Genetics 3:266-272). In a particularly preferable embodiment, the homology of a protein or nucleic acid sequence is evaluated using a Basic Local Alignment Search Tool (BLAST) well known in the art (e.g., see Karlin and Altschul, 1990, Proc. Natl. Acad. Sol, USA 87: 2267-2268, Altschul at al., 1990, J. Mol. Biol. 215:403-410, Altschul et al., 1993, Nature Genetics 3:266-272, Altschul at al., 1997, Nuc. Acids Res. 25:3389-3402). Particularly, 5 specialized-BLAST programs may be used to perform the following tacit to achieve comparison or search:
(1) comparison of an amino acid query sequence with a protein sequence database using BLASTP and BLAST3;
(2) comparison of a nucleotide query sequence with a nucleotide sequence database using BLASTN;
(3) comparison of a conceptually translated product in which a nucleotide query sequence (both strands) is converted over 6 reading frames with a protein sequence database using BLASTX;
(4) comparison of all protein query sequences converted over 6 reading frames (both strands) with a nucleotide sequence database using TBLASTN; and
(5) comparison of nucleotide query sequences converted over 6 reading frames with a nucleotide sequence database using TBLASTX.

The BLAST program identifies homologous sequences by specifying analogous segments called “high score segment pairs” between amino acid query sequences or nucleic acid query sequences and test sequences obtained from preferably a protein sequence database or a nucleic acid sequence database. A large number of the high score segment pairs are preferably identified (aligned) using a scoring matrix well known in the art. Preferably, the scoring matrix is the BLOSUM62 matrix (Gonnet et al., 1992, Science 256:1443-1445, Henikoff and Henikoff, 1993, Proteins 17:49-61). The PAM or PAM250 matrix may be used, although they are not as preferable as the BLOSUM62 matrix (e.g., see Schwartz and Dayhoff, eds., 1978, Matrices for Detecting Distance Relationships: Atlas of Protein Sequence and Structure, Washington: National Biomedical Research Foundation). The BLAST program evaluates the statistical significance of all identified high score segment pairs and preferably selects segments which satisfy a threshold level of significance independently defined by a user, such as a user set homology. Preferably, the statistical significance of high score segment pairs is evaluated using Karlin's formula (see Karlin and Altschul, 1990, Proc. Natl. Acad. Sci. USA 87:2267-2268).

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such a primer design is well known in the art and may be performed manually or using a computer program (e.g., LASERGENE, Primer Select, DNAStar).

As used herein, the term “epitope” refers to an antigenic determinant whose detailed structure may not be necessarily defined as long as it can elicit an antigen-antibody reaction. Therefore, the term “epitope” includes a set of amino acid residues which are involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the feature of a molecule (e.g., primary, secondary and tertiary peptide structure, and charge) that forms a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and two-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method for rapidly synthesizing peptides to determine the location of immunogenic epitopes in a given antigen); U.S. Pat. No. 4,708,871 (procedures for identifying and chemically synthesizing epitopes of antigens); and Geysen at al., Molecular Immunology (1986) 23: 709 (technique for identifying peptides with high affinity for a given antibody). Antibodies that recognize the same epitope can be identified in a simple immunoassay. Thus, methods for determining an epitope including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and at least 25 amino acids. Epitopes may be linear or conformational.

(Modification of Genes)

In a given protein molecule (e.g., a DNA polymerase, etc.), a given amino acid contained in a sequence may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness. Alternatively, a nucleic acid sequence encoding a DNA polymerase may be modified so that the proofreading function of the DNA polymerase is modified.

When the above-described modifications are designed, the hydrophobicity indices of amino acids may be taken into consideration. The hydrophobic amino acid indices play an important role in providing a protein with an interactive biological function, which to generally recognized in the art (Kyte. J and Doolittle, R. F., J. Mol. Biol. 157(1): 105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows isoleucine (+4.5); valine (+4.2); leucine (+3.8); phenylalanine (+2.8)1 cysteine/cystine (+2.5): methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient.

Hydrophilicity indexes may be taken into account in modifying genes in the present invention. As described in U.S. Pat. No. 4,554,101, amino acid residues are given the following hydrophilicity indices: arginine (+3.0); lysine (+3.0); aspartic acid (+3.0±1): glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, conservative substitution is carried out between amino acids having a hydrophilicity or hydrophobicity index of within ±2, preferably within ±1, and more preferably within ±0.5. Examples of conservative substitution include, but are not limited to, substitutions within each of the following residue pairs: arginine and lysine: glutamic acid and aspartic acids; serine and threonine: glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion with respect to a reference nucleic acid molecule or polypeptide. Variant may or may not have the biological activity of a reference molecule (e.g., a wild-type molecule, etc.). Variants may be conferred additional biological activity, or may lack a part of biological activity, depending on the purpose. Such design can be carried out using techniques well known in the art. Alternatively, variants, whose properties are already known, may be obtained by isolation from organisms to produce the variants and the nucleic acid sequence of the variant may be amplified so as to obtain the sequence information. Therefore, for host cells, corresponding genes derived from heterologous species or products thereof are regarded as “variants”.

As used herein, the term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has a lost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining much a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gone are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of a method for such modification of a base sequence include cleavage using a restriction enzyme or the like; ligation or the like by treatment using DNA polymerase. Klenow fragments, DNA ligase, or the like; and a site specific base substitution method using synthesized oligonucleotides (specific-site directed mutagenesis; Mark Zoller and Michael Smith, Methods in Enzymology, 100, 465-500(1983)). Modification can be performed using methods ordinarily used in the field of molecular biology.

In order to prepare functionally equivalent polypeptides, amino acid additions, deletions, and/or modifications can be performed in addition to amino acid substitutions. Amino acid substitution(s) refers to the replacement of at least one amino acid of an original peptide chain with different amino acids, such as the replacement of 1 to 10 amino acids, preferably 1 to 5 amino acids, and more preferably 1 to 3 amino acids with different amino acids. Amino acid addition (s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the terms “peptide analog” or “peptide derivative” refer to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same an the function of the original peptide (e.g., a similar pKa value, a similar functional group, a similar binding manner to other molecules, a similar water-solubility, and the like). Such a peptide analog can be prepared using a technique well known in the art. Therefore, a peptide analog may be a polymer containing an amino acid analog.

Similarly, as used herein, the terms “polynucleotide analog” or “nucleic acid analog” refer to a compound which is different from a polynucleotide or nucleic acid, but has at least one chemical or biological function equivalent to the polynucleotide or nucleic acid. Therefore, a polynucleotide or nucleic acid analog includes one that has at least one nucleotide analog or nucleotide derivative addition or substitution with respect to the original polynucleotide or nucleic acid.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function. Such a gene is known in the art and can be used in the present invention.

The above-described nucleic acid can be obtained by a well-known PCR method, i.e., chemical synthesis. This method may be combined with, for example, site-directed mutagenesis, hybridization, or the like.

As used herein, the term “substitutions”, “addition” or “deletion” for a polypeptide or a polynucleotide refers to the substitution, addition or deletion of an amino acid or its substitute, or a nucleotide or its substitute, with respect to the original polypeptide or polynucleotide, respectively. This is achieved by techniques well known in the art, including a site-directed mutagenesis technique and the like. A polypeptide or a polynucleotide may have any number (>0) of substitutions, additions, or deletions. The number can be as large as a variant having such a number of substitutions, additions or deletions which maintains an intended function (e.g., the information transfer function of hormones and cytokines, etc.). For example, such a number may be one or several, and preferably within 20% or 10% of the full length, or no more than 100, no more than 50, no more than 25, or the like.

(Genetic Engineering)

Proteins, such as DNA polymerases and fragments and variants thereof, and the like, as used herein can be produced and introduced into cello by genetic engineering techniques.

When a gene is mentioned herein, the term “vector” or “recombinant vector” refers to a vector capable of transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for cloning is referred to as “cloning vector”. Such a cloning vector ordinarily contains a multiple cloning site containing a plurality of restriction sites. Restrictions sites and multiple cloning sites are well known in the art and may be appropriately or optionally used depending on the purpose. The technology is described in references an described herein (e.g., Sambrook et al. (supra)). Such vectors include, for example, plasmids.

As used herein; the term “plasmid” refers to a hereditary factor which is present apart from chromosomes and autonomously replicates. When specifically mentioned, DNA contained in mitochondria, chloroplasts, and the like of cell nuclei is generally called organelle DNA and is distinguished from plasmids, i.e., is not included in plasmids.

Examples of plasmids include, but are not limited to:
E. coli: pET (TAKARA), pUC (TOYOBO), pBR322 (TOYOBO), pBluescriptII (TOYOBO);
yeast: pAUR (TAKARA), pESP (TOYOBO), pESC (TOYOBO);
Bacillus subtilis: pHY300PLK(TAKARA);
mycosis: pPRTI (TAKARA), pAUR316(TAKARA);
animal cells: pCMV (TOYOBO), pBK-CMV(TOYOBO); and the like.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and silencers and/or enhancers. It is well known to those skilled in the art that the type of organism (e.g., a plant) expression vector and the type of regulatory element may vary depending on the host cell. By introducing a specific promoter into cells, the error-prone frequency of the cells can be regulated under certain conditions.

As used herein, a “recombinant vector” for prokaryotic cells includes, for example, pcDNA 3(+), pBluescript-SK(+/−), pGEM-T, pEF-BOS, pEGFP, pHAT, pUC18, pFT-DEST™, 42GATEWAY (Invitrogen), and the like.

As used herein, a “recombinant vector” for animal cells includes, for example, pcDNA I/Amp, pcDNA I, pCDM8 (all commercially available from Funakoshi, Tokyo, Japan), pAGE107 [Japanese Laid-Open Publication No. 3-229 (Invitrogen), pAGE103 [J. Biochem., 101, 1307(1987)], pAMo, pAMoA [J. Biol. Chem., 268, 22782-22787 (1993)], retroviral expression vectors based on Murine Stem Cell Virus (MSCV), pEF-BOS, pEGFP, and the like.

Examples of recombinant vectors for use in plant cells include Ti plasmid, a tobacco mosaic virus vector, a cauliflower mosaic virus vector, a gemini virus vector, and the like.

Examples of recombinant vectors for use in insect cells include a baculo virus vector, and the like.

As used herein, the term “terminator” refers to a sequence which is located downstream of a protein-encoding region of a gene and which is involved in the termination of transcription when DNA is transcribed into mRNA, and the addition of a poly A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. Therefore, a portion of a given gene which functions as a promoter is herein referred to as a “promoter portion”. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “enhancer” refers to a sequence which is used so as to enhance the expression efficiency of a gene of interest. Such an enhancer is well known in the art. One or more enhancers may be used, or no enhancer may be used.

As used herein, the term “silencer” refers to a sequence having a function of suppressing or ceasing expression of a gene. In the present invention, any silencer having such a function may be used, or alternatively, no silencer may be used.

As used herein, the term “operatively linked” indicates that a desired sequence to located such that expression (operation) thereof is under control of a transcription and translation regulatory sequence (e.g., a promoter, an enhancer, and the like) or a translation regulatory sequence. In order for a promoter to be operatively linked to a gone, typically, the promoter is located immediately upstream of the gene. A promoter in not necessarily adjacent to a structural gene.

Any technique may be used herein for introduction of a nucleic acid molecule encoding a DNA polymerase having a modified proofreading function or the like into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed. (2001), Cold Spring Harbor Laboratory Press, Cold Spring Harbor, N.Y.: Special issue, Jikken Igaku [Experimental Medicine] “Experimental Method for Gene introduction & Expression Analysis”, Yodo-sha, 1997; and the like. Gene introduction can be confirmed by methods as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, ouch as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serrattia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like.

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon cancer cell line, and the like. The mouse myeloma cell includes ps20, NSO, and the like. The rat myeloma cell includes YB2/0 and the like. A human embryo kidney call includes HEK293 (ATCC:CRL-1573) and the like. The human leukemia cell includes BALL-1 and the like. The African green monkey kidney cell includes COS-1, COS-7, and the like. The human colon cancer call line includes HCT-15, and the like. A human neuroblastoma includes SK-N-SH, SK-N-SH-5Y, and the like. A mouse neuroblastoma includes Neuro2A, and the like.

Any method for introduction of DNA can be used herein as a method for introduction of a recombinant vector, including, for example, a calcium chloride method, an electroporation method (Methods. Enzymol., 194, 182 (1990)), a lipofection method, a spheroplast method (Proc. Natl. Acad. Sci. USA, 84, 1929 (1978)), a lithium acetate method (J. Bacteriol., 153, 163 (1983)), a method described in Proc. Natl. Acad. Sci. USA, 75, 1929 (1978), and the like.

A retrovirus infection method as used herein is well known in the art as described in, for example, Current Protocols in Molecular Biology (supra) (particularly, Units 9.9-9.14), and the like. Specifically, for example, embryonic stem cells are trypsinized into a single-cell suspension, followed by co-culture with the culture supernatant of virus-producing cell. (packaging cell lines) for 1-2 hours, thereby obtaining a sufficient amount of infected cells.

When the present invention is applied to plants, plant expression vectors may be introduced into plant cells using methods well known in the art, such as a method using an Agrobacterium and a direct inserting method. An example of the method using Agrobacterium may include a method described in, for example, Nagel et al. (1990). Microbiol. Lett., 67, 325). In this method, for example, an expression vector suitable for plants are inserted into Agrobacterium by electroporation and the transformed Agrobacterium is introduced into plant cells by a method described in, for example, Gelvin at al., eds, (1994), Plant Molecular Biology Manual (Kluwer Academic Press Publishers)). Examples of a method for introducing a plant expression vector directly into cells include electroporation (Shimamoto at al. (1989), Nature, 338: 274-276; and Rhodes et al. (1989), Science, 240: 204-207), a particle gun method (Christou et al. (1991), Bio/Technology 9: 957-962), and a polyethylene glycol method (PEG) method (Datta et al. (1990), Bio/Technology 8: 736-740). These methods are well known in the art, and among them, a method suitable for a plant to be transformed may be appropriately selected.

In the present invention, a nucleic acid molecule (introduced gene) of interest may or may not be introduced into a chromosome of transformants. Preferably, a nucleic acid molecule (introduced gone) of interest is introduced into a chromosome of transformants, more preferably into a pair of chromosomes.

Transformed cells may be differentiated by methods well known in the art to plant tissues, plant organs, and/or plant bodies.

Plant cells, plant tissues, and plant bodies are cultured, differentiated, and reproduced using techniques and media known in the art. Examples of the media include, but are not limited to, Murashige-Skoog (MS) medium, Gamborg B5(B) medium, White medium, Nitech & Nitsch medium, and the like. Those media are typically supplemented with an appropriate amount of a plant growth regulating substance (plant hormone) or the like.

As used herein, the term “redifferentiation” or “redifferentiate” in relation to plants refers to a phenomenon in which a whole plant is restored from a part of an individual plant. For example, a tissue segment, such as a cell, a leaf, a root, or the like, can be redifferentiated into an organ or a plant body.

Methods of redifferentiating a transformant into a plant body are well known in the art. These methods are described in, for example, Rogers et al., Methods in Enzymology 118: 627-640 (1986): Tabata et al., Plant Cell Physiol., 28: 73-82 (1987): Shaw, Plant Molecular Biology: A practical approach, IRL press (1988); Shimamoto et al., Nature 338: 274 (1989): Maliga et al., Methods in Plant Molecular Biology: A laboratory course, Cold Spring Harbor Laboratory Press (1995); and like. Therefore, the above-described well-known methods can be appropriately selected and employed, depending on a transformed plant of interest, by those skilled in the art to redifferentiate the plant. The transformed plant has an introduced gene of interest. The introduced gene can be confirmed by methods described herein and other well-known common techniques, such as northern blotting, western blotting analysis, and the like.

Seeds may be obtained from transformed plants. Expression of an introduced gene can be detected by northern blotting or PCR. Expression of a gene product protein may be confirmed by, for example, western blotting, if required.

It is demonstrated that the present invention can be applied to any organism and is particularly useful for plants. The present invention can also be applied to other organisms. Molecular biology techniques for use in the present invention are well known and commonly used in the art, and are described in, for example, Ausubel F. A., et al., ads. (1988), Current Protocols in Molecular Biology, Wiley, New York, N.Y.; Sambrook J., et al, (1987), Molecular Cloning: A Laboratory Manual, Ver. 2 and Ver. 3, Cold Spring Harbor Laboratory Press, Cold Spring Harbor, N.Y.; Special issue, Jikken Igaku [Experimental Medicine]“Idenshi Donyu & Hatsugen Kaiseki Jikkenho [Experimental Methods for Gene introduction & Expression Analysis]”, Yodo-sha, 1997; and the like.

Gene expression (e.g., mRNA expression, polypeptide expression) may be “detected” or “quantified” by an appropriate method, including mRNA measurement and immunological measurement method. Examples of the molecular biological measurement method include a Northern blotting method, a dot blotting method, a PCR method, and the like. Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like. A gene analysis method using an array (e.g., a DNA array, a protein array, etc.) may be used. The DNA array is widely reviewed in Saibo-Kogaku [Cell Engineering], special Issue, “DNA Microarray and Up-to-date PCR Method”, edited by Shujun-sha. The protein array is described in detail in Nat Genet. 2002 December; 32 Suppl: 526-32. Examples of a method for analyzing gene expression include, but are not limited to, an RT-PCR method, a RACE method, an SSCP method, an immuno precipitation method, a two-hybrid system, an in vitro translation method, and the like in addition to the above-described techniques. Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

As used herein, the term “amount of expression” refers to the amount of a polypeptide or mRNA expressed in a subject cell. The amount of expression includes the amount of expression at the protein level of a polypeptide of the present invention evaluated by any appropriate method using an antibody of the present invention, including immunological measurement methods (e.g., an ELISA method, a RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method. Thus, according to the present invention, an error-prone frequency can be regulated by changing the amount of expression of a certain agent (e.g., DNA polymerase, etc.).

As used herein, the term “upstream” in reference to a polynucleotide means that the position is closer to the 5′ terminus than a specific reference point.

As used herein, the term “downstream” in reference to a polynucleotide means that the position is closer to the 3′ terminus than a specific reference point.

As used herein, the term “base paired” and “Watson & Crick base paired” have the same meaning and refer to nucleotides which can be bound together by hydrogen bonds based on the sequence identity that an adenine residue (A) is bound to a thymine residue (T) or a uracil residue (U) via two hydrogen bonds and a cytosine residue (C) is bound to a guanine reside (G) via three hydrogen bonds, as seen in double-stranded DNA (see Stryer, L., Biochemistry, 4th edition, 1995).

As used herein, the term “complementary” or “complement” refers to a polynucleotide sequence such that the whole complementary region thereof is capable of Watson-Crick base paring with another specific polynucleotide. In the present invention, when each base of a first polynucleotide pairs with a corresponding complementary base, the first polynucleotide is regarded as being complementary to a second polynucleotide. Complementary bases are generally A and T (or A and U) or C and G. As used herein, the term “complement” is used as a synonym for the terms “complementary polynucleotide”, “complementary nucleic acid” and “complementary nucleotide sequence”. These terms are applied to a pair of polynucleotides based on the sequence, but not a specific set of two polynucleotides which are virtually bound together.

Production and analysis of transgenic animals and knockout animals via homologous recombination of embryotic stem (ES) cells provide important means. Transgenic animals or knockout mammals can be produced by, for example, a positive-negative selection method using homologous recombination (see, U.S. Pat. Nos. 5,464,764; 5,487,992; 5,627,059; Proc. Natl. Acad. Sci. USA, Vol. 86, 8932-8935, 1989; Nature, Vol. 342, 435-438, 1989; and the like). Production of knockout animals (also called gene targeting) is reviewed in, for example, Masami Murayama, Masashi Yamamoto, eds. Jikken Igaku Bessatsu [Special Issue of Experimental Medicine], “Shintei Idenshi Kogaku Handobukku [Newly Revised Genetic Engineering Handbook]”, Ver. 3, 1999, Yodo-sha, particularly pp. 239-256; Shinichi Aizawa, (1995), Jikken Igaku Bessatsu [Special Issue of Experimental Medicine], “Jintagettingu—ES Saibo Wo Motiita Heni Mausu No Sakusei [Gene Targeting—Production of Mutant Mouse Using ES Cell]; and the like. Transgenic animals or knockout mammals have been widely used. In the present invention, the above-described methods are employed if required.

For example, in the case of higher organisms, recombinants are efficiently screened for by positive selection using a neomycin resistant gene and negative selection using a thymidine kinase gene of HSV or a diphtheria toxin gene. Knockout PCR or Southern blotting is used to screen homologous recombinants. Specifically, a part of a target gene is substituted with a neomycin resistant gene or the like for positive selection and an HSVTK gene or the like for negative selection is linked to a terminus thereof, resulting in a targeting vector. The targeting vector is introduced into ES cells by electroporation. The ES cells are screened in the presence of G418 and ganciclovir. Surviving colonies are isolated, followed by PCR or Southern blotting to screen for homologous recombinants.

In the above-described method, a targeted endogenous gene is disrupted to obtain a transgenic or knockout (target gene recombinant, gene disrupted) mouse lacking, or having a reduced level of, the corresponding function. The method is useful for analysis of gene functions since a mutation is introduced only into a targeted gene.

After a desired homologous recombinant is selected, the resultant recombinant ES cell is mixed with a normal embryo by a blastocyst injection method or am aggregation chimera method to produce a chimeric mouse of the ES cell and the host embryo. In the blastocyst injection method, an ES cell is injected into a blastocyst using a glass pipette. In the aggregation chimera method, a mass of ES cells are attached to a 8-cell stage embryo without zona pellucida. The blastocyst having the introduced ES cell is implanted into the uterus of a pseudopregnant foster mother to obtain a chimeric mouse. ES cells have totipotency and can be differentiated in vivo into any kind of cell including germ cello. If chimeric mice having a germ cell derived from an ES cell are crossbred with normal mice, mice having the chromosome of the ES cell heterozygously are obtained. The resultant mice are crossbred with each other, knockout mice having a homozygous modified chromosome of the ES cell are obtained. To obtain knockout, mice having the modified chromosome homozygously from the chimeric mice, male chimeric mice are crossbred with female wild type mice to produce F1 heterozygous mice. The resultant male and female heterozygous mice are crossbred and F2 homozygous mice are selected. Whether or not a desired gene mutation is introduced into F1 and F2 may be determined using commonly used methods, such as Southern blotting, PCR, base sequencing, and the like, as with assays for recombinant ES cells.

As another technique for overcoming the problem that various gene functions cannot be selectively analyzed, a conditional knockout technique has attracted attention, in which the cell type-specific expression of Cre recombinase is combined with the site-specific recombination of Cre-loxP. To obtain conditional knockout mice using Cre-loxP, a neomycin resistant gene is introduced into a site which does not inhibit expression of a target gene; a targeting vector is introduced into ES cells, in which a loxP sequence is incorporated in such a manner that an exon, which will be removed later, breaks in the loxP sequence; and thereafter, the homologous recombinants are isolated. Chimeric mice are obtained from the isolated clones. Thus, genetically modified mice are produced. Next, a transgenic mouse in which P1 phage-derived site-specific recombinant enzyme Cre of E. coli is expressed in tissue-specific manner is crossbred with the mouse. In this case, genes are disrupted only in a tissue expressing Cre (Cre specifically recognizes the loxP sequence (34 bp), and a sequence between two loxP sequences is subjected to recombination and is disrupted). Cre can be expressed in adults by crossbreeding with a transgenic mouse having a Cre gene linked to an organ-specific promoter, or by using a viral vector having the Cre gene (Stanford W. L., et al., Nature Genetics 2: 756-768(2001)).

Thus, organisms of the present invention can be produced.

(Polypeptide Production Method)

A transformant derived from a microorganism, an animal cell, or the like, which is produced by a method of the present invention, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium as long as the medium contains a carbon source, a nitrogen source, inorganic salts, and the like which an organism of the present invention can assimilate and the medium allows efficient culture of the transformant.

The carbon source includes any carbon source that can be assimilated by the organism, such as carbohydrates (e.g., glucose, fructose, sucrose, molasses containing these, starch, starch hydrolysate, and the like), organic acids (e.g., acetic acid, propionic acid, and the like), alcohols (e.g., ethanol, propanol, and the like), and the like.

The nitrogen source includes ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), and the like.

Salts of inorganic acids, such as potassium (I) phosphate, potassium (II) phosphate, magnesium phosphate, sodium chloride, iron (I) sulfate, manganese sulfate, copper sulfate, calcium carbonate, and the like, can be used. Culture is performed under aerobic conditions for shaking culture, deep aeration agitation culture, or the like.

Culture temperature is preferably 15 to 40° C., and other temperatures can be used. Particularly, if temperature resistant organisms or cells are produced according to the present invention, the other temperature may be most suitable. Culture time is ordinarily 5 hours to 7 days. The pH of culture medium is maintained at 3.0 to 9.0. Particularly, if acid or alkali resistant organisms or calls are produced according to the present invention, other pH may be most suitable. The adjustment of pH is carried out using inorganic or organic acid, alkali solution, urea, calcium carbonate, ammonia, or the like. An antibiotic, such as ampicillin, tetracycline, or the like, may be optionally added to the culture medium during cultivation.

When culturing a microorganism which has been transformed using an expression vector containing an inducible promoter, the culture medium may be optionally supplemented with an inducer. For example, when a microorganism, which has been transformed using an expression vector containing a lac promoter, is cultured, isopropyl-β-D-thiogalactopyranoside or the like may be added to the culture medium. When a microorganism, which has been transformed using an expression vector containing a trp promoter, is cultured, indole acrylic acid or the like may be added to the culture medium. A cell or an organ into which a gene has been introduced can be cultured in a large volume using a jar fermenter. Examples of culture medium include, but are not limited to, commonly used MurashigeMurashige-Skoog (MS) medium, White medium, or these media supplemented with a plant hormone, such as auxin, cytokines, or the like.

For example, when an animal cell is used, a culture medium of the present invention for culturing the call includes a commonly used RPMI1640culture medium (The Journal of the American Medical Association, 299, 519 (1967)), Eagle's MEM culture medium (Science, 122, 501 (1952)), DMEM culture medium (Virology, 8, 396 (1959)), 199 culture medium (Proceedings of the Society for the Biological Medicine, 73, 1 (1950)) or these culture media supplemented with fetal bovine serum or the like.

Culture is normally carried out for 1 to 7 days in media of pH 6 to 8, at 25 to 40° C., in an atmosphere of 5% CO₂, for example. An antibiotic, such as kanamycin, penicillin, streptomycin, or the like may be optionally added to culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain the soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Chemical Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.), and the like.

When a polypeptide of the present invention is accumulated in a dissolved form within a transformant cell of the present invention for producing the polypeptide, the culture is subjected to centrifugation to collect cells in the culture. The cells are washed, followed by pulverization of the cells using an ultrasonic pulverizer, a French press, MANTON GAULIN homogenizer, Dinomil, or the like, to obtain a cell-free extract solution. A purified specimen can be obtained from a supernatant obtained by centrifuging the call-free extract solution or by a technique, such as solvent extraction salting-out/desalting with ammonium sulfate or the like, precipitation with organic solvent, anion exchange chromatography with a resin (e.g. diethylaminoethyl (DEAN)-Sepharose, DIAION HPA-75 (Mitsubishi Chemical Corporation), etc.), cation exchange chromatography with a resin (e.g., S-Sepharose FF (Pharmacia), etc.) hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.), and the like.

When the polypeptide of the present invention has been expressed and has formed insoluble bodies within calls, the cells are harvested, pulverized, and centrifuged. From the resulting precipitate fraction, the polypeptide of the present invention is collected using a commonly used method. The insoluble polypeptide is a solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler at al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231(1989), Genes Develop., 4, 1288(1990)), a fusion protein of the polypeptide of the present invention with protein A is produced, followed by purification with affinity chromatography using immunoglobulin G.

A fusion protein of the polypeptide of the present invention with a FLAG peptide is produced, followed by purification with affinity chromatography using anti-FLAG antibodies (Proc. Natl. Aced. Sci., USA, 86, 8227(1989), Genes Develop., 4, 1288 (1990)).

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

The polypeptide of the present invention can also be produced by a chemical synthesis method, such as the Fmoc method (fluorenylmethyloxycarbonyl method), the tBoc method (t-buthyloxycarbonyl method), or the like, based on the amino acid information thereof. The peptide can be chemically synthesized using a peptide synthesizer (manufactured by Advanced ChemTech, Applied Biosystems, Pharmacia Biotech, Protein Technology instrument, Synthecell-Vega, PerSeptive, Shimazu, or the like).

The structure of the purified polypeptide of the present invention can be carried out by methods commonly used in protein chemistry (see, for example, Hisashi Hirano. “Protein Structure Analysis for Gene Cloning”, published by Tokyo Kagaku Dojin, 1993). The physiological activity of a novel ps020-like peptide of the present invention can be measured by known measuring techniques (Cell, 75, 1389(1993): J. Cell Bio., 1146, 233(1999); Cancer Res. 58, 1238(1998); Neuron 17, 1157(1996): Science 289, 1197(2000); etc.).

(Screening)

As used herein, the term “screening” refers to selection of a target, such as an organism, a substance, or the like, with a given specific property of interest from a population containing a number of elements using a specific operation/evaluation method. For screening, an agent (e.g., an antibody), a polypeptide or a nucleic acid molecule of the present invention can be used. Screening may be performed using libraries obtained in vitro, in vivo, or the like (with a system using a real substance) or alternatively in silico (with a system using a computer). It will be understood that the present invention encompasses compounds having desired activity obtained by screening. The present invention is also intended to provide drugs which are produced by computer modeling based on the disclosures of the present invention.

The screening or identifying methods are well known in the art and can be carried out with, for example, microtiter plates; arrays or chips of molecules, such as DNA, proteins, or the like; or the like. Examples of a subject containing samples to be screened include, but are not limited to, gene libraries, compound libraries synthesized using combinatorial libraries, and the like.

Therefore, in a preferred embodiment of the present invention, a method for identifying an agent capable of regulating a disorder or a disease is provided. Such a regulatory agent can be used as a medicament for the diseases or a precursor thereof. Such a regularoty agent, a medicament containing the regulatory agent, and a therapy using the same are encompassed by the present invention.

Therefore, it is contemplated that the present invention provides drugs obtained by computer modeling in view of the disclosure of the present invention.

In another embodiment of the present invention, the present invention encompasses compounds obtained by a computer-aided quantitative structure activity relationship (QSAR) modeling technique, which is used as a tool for screening for a compound of the present invention having effective regulatory activity. Here, the computer technique includes several substrate templates prepared by a computer, pharmacophores, homology models of an active portion of the present invention, and the like. In general, a method for modeling a typical characteristic group of a substance, which interacts with another substance, based on data obtained in vitro includes a recent CATALYST™ pharmacophore method (Ekins et al., Pharmacogenetics, 9:477 to 489, 1999; Ekins et al., J. Pharmacol. & Exp. Ther., 288;21 to 29, 1999: Ekins et al., J. Pharmacol. & Exp. Ther., 290:429 to 438, 1999; Ekins et al., J. Pharmacol. Exp. Ther., 291.424 to 433, 1999), a comparative molecular field analysis (CoMFA) (Jones et al., Drug Metabolism & Disposition, 24:1 to 6, 1996), and the like. In the present invention, computer modeling may be performed using molecule modeling software (e.g. CATALYST™ Version 4 (Molecular Simulations, inc., San Diego, Calif.), etc.).

The fitting of a compound with respect to an active site can be performed using any of various computer modeling techniques known in the art. Visual inspection and manual operation of a compound with respect to an active site can be performed using a program, such as QUANTA (Molecular Simulations, Burlington, Mass., 1992), SYBYL (Molecular Modeling Software, Tripos Associates, inc., St. Louis, Mo., 1992), AMBER (Weiner et al., J. Am. Chem. Soc., 106:765-784, 1984), CHARMM (Brooks et al., J. Comp. Chem., 4:187 to 217, 1983), or the like. In addition, energy minimization can be performed using a standard force field, such as CHARMM, AMBER, or the like. Examples of other specialized computer modeling methods include GRID (Goodford et al., J. Med. Chem., 28:849 to 857, 1985), MCSS (Miranker and Karplus, Function and Genetics, 11:29 to 34, 1991), AUTODOCK (Goodsell and Olsen, Proteins: Structure, Function and Genetics, 8:195 to 202, 1990), DOCK (Kuntz at al., J. Mol. Biol., 161:269 to 288, 1982), and the like. Further, structural compounds can be newly constructed using an empty active site, an active site of a known small molecule compound with a computer program, such as LUDI (Bohm, J. Comp. Aid. Molec. Design, 6:61 to 78, 1992). LEGEND (Nishibata and Itai, Tetrahedron, 47:8985, 1991), LeapFrog (Tripos Associates, St. Louis, Mo.), or the like. The above-described modeling methods are commonly used in the art. Compounds encompassed by the present invention can be appropriately designed by those skilled in the art based on the disclosure of the present specification.

(Diseases)

The present invention may target diseases and disorders which an organism of interest may suffer from (e.g., production of model animals, etc.).

In one embodiment, diseases and disorders targeted by the present invention may be related to the circulation system (blood cells, etc.). Examples of the diseases or disorders include, but are not limited to, anemia (e.g., aplastic anemia (particularly, severe aplastic anemia), renal anemia, cancerous anemia, secondary anemia, refractory anemia, etc.), cancer or tumors (e.g., leukemia); and after chemotherapy therefor, hematopoietic failure, thrombocytopenia, acute myelocytic leukemia (particularly, a first remission (high-risk group), a second remission and thereafter), acute lymphocytic leukemia (particularly, a first remission, a second remission and thereafter), chronic myelocytic leukemia (particularly, chronic period, transmigration period), malignant lymphoma (particularly, a first remission (high-risk group), a second remission and thereafter), multiple myeloma (particularly, an early period after the onset), and the like.

In another embodiment, diseases and disorders targeted by the present invention may be related to the nervous system. Examples of such diseases or disorders include, but are not limited to, dementia, cerebral stroke and sequela thereof, cerebral tumor, spinal injury, and the like.

In another embodiment, diseases and disorders targeted by the present invention may be related to the immune system. Examples of such diseases or disorders include, but are not limited to, T-cell deficiency syndrome, leukemia, and the like.

In another embodiment, diseases and disorders targeted by the present invention may be related to the motor organ and the skeletal system. Examples of such diseases or disorders include, but are not limited to, fracture, osteoporosis, luxation of joints, subluxation, sprain, ligament injury, osteoarthritis, osteosarooma, Ewing's sarcoma, osteogenesis imperfecta, osteochondrodysplasia, and the like.

In another embodiment, diseases and disorders targeted by the present invention may be related to the skin system. Examples of such diseases or disorders include, but are not limited to, atrichia, melanoma, cutis matignant lympoma, hemangiosarooma, histiocytosis, hydroa, pustulosis, dermatitis, eczema, and the like.

In another embodiment, diseases and disorders targeted by the present invention may be related to the endocrine system. Examples of such diseases or disorders include, but are not limited to, hypothalamus/hypophysis diseases, thyroid gland diseases, accessory thyroid gland (parathyroid) diseases, adrenal cortex/medulla diseases, saccharometabolism abnormality, lipid metabolism abnormality, protein metabolism abnormality, nucleic acid metabolism abnormality, inborn error of metabolism (phenylketonuria, galactosemia, homocystinuria, maple syrup urine disease), analbuminemia, lack of ascorbic acid sysnthetic ability, hyperbilirubinemia, hyperbilirubinuria, kallikrein deficiency, mast cell deficiency, diabetes insipidus, vasopressin secretion abnormality, dwarf, Wolman's disease (acid lipase deficiency)), mucopolysaccharidosis VI, and the like.

In another embodiment diseases and disorders targeted by the present invention may be related to the respiratory system. Examples of such diseases or disorders include, but are not limited to, pulmonary diseases (e.g., pneumonia, lung cancer, etc.), bronchial diseases, and the like.

In another embodiment, diseases and disorders targeted by the present invention may be related to the digestive system. Examples of such diseases or disorders include, but are not limited to, esophagus diseases (e.g., esophagus cancer, etc.), stomach/duodenum diseases (e.g., stomach cancer, duodenum cancer, etc.), small intestine diseases/large intestine diseases (e.g., polyp of colon, colon cancer, rectum cancer, etc.), bile duct diseases, liver diseases (e.g., liver cirrhosis, hepatitis (A, B, C, D, E, etc.), fulminant hepatitis, chronic hepatitis, primary liver cancer, alcoholic liver disorders, drug induced liver disorders, etc.), pancreas diseases (acute pancreatitis, chronic pancreatitis, pancreas cancer, cystic pancreas diseases, etc.), peritoneum/abdominal wall/diaphragm diseases (hernia, etc.), Hirscheprung's disease, and the like.

In another embodiment, diseases and disorders targeted by the present invention may be related to the urinary system. Examples of such diseases or disorders include, but are not limited to, kidney diseases (e.g., renal failure, primary glomerulus diseases, renovascular disorders, tubular function abnormality, interstitial kidney diseases, kidney disorders due to systemic diseases, kidney cancer, etc.), bladder diseases (e.g., cystitis, bladder cancer, etc.), and the like.

In another embodiment, diseases and disorders targeted by the present invention may be related to the genital system. Examples of such diseases or disorders include, but are not limited to, male genital organ diseases (e.g., male sterility, prostatomegaly, prostate cancer, testis cancer, etc.), female genital organ diseases (e.g., female sterility, ovary function disorders, hysteromyoma, adenomyosis uteri, uterus cancer, endometriosis, ovary cancer, villosity diseases, etc.), and the like.

In another embodiment, diseases and disorders targeted by the present invention may be related to the circulatory system. Examples of such diseases or disorders include, but are not limited to, heart failure, angina pectoris, myocardial infarct, arrhythmia, valvulitis, cardiac muscle/pericardium disease, congenital heart disease (e.g., atrial septal defect, arterial canal patency, tetralogy of Fallot, etc.), artery diseases (e.g., arteriosclerosis, aneurysm), vein diseases (e.g., phlebeurysm, etc.), lymphoduct diseases (e.g., lymphedema, etc.), and the like.

Diseases (damages) and disorders targeted by the present invention may include diseases and disorders of plants. Examples of diseases and disorders include, but are not limited to, rice blast, disorders due to cold weather, and the like.

When a product substance or the like obtained according to the present invention is used as a medicament, the medicament may further comprise a pharmaceutically acceptable carrier. Any pharmaceutically acceptable carrier known in the art may be used in the medicament of the present invention.

Examples of a pharmaceutical acceptable carrier or a suitable formulation material include, but are not limited to, antioxidants, preservatives, colorants, flavoring agents, diluents, emulsifiers, suspending agents, solvents, fillers, bulky agents, buffers, delivery vehicles, and/or pharmaceutical adjuvants. Representatively, a medicament of the present invention is administered in the form of a composition comprising adiponectin or a variant or fragment thereof, or a variant or derivative thereof with at least one physiologically acceptable carrier, excipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are nontoxic to recipients and are preferably inert at the dosages and concentrations employed, and preferably include phosphate, citrate, or other organic acids; ascorbic acid, α-tocopherol: low molecular weight polypeptides; proteins (e.g., serum albumin, gelatin, or immunoglobulins): hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

Hereinafter, commonly used preparation methods of the medicament of the present invention will be described. Note that animal drug compositions, quasi-drugs, marine drug compositions, food compositions, cosmetic compositions, and the like can be prepared using known preparation methods.

A product substance and the like of the present invention can be mixed with a pharmaceutically acceptable carrier and can be orally or parenterally administered as solid formulations (e.g., tablets, capsules, granules, abstracts, powders, suppositories, etc.) or liquid formulations (e.g., syrups, injections, suspensions, solutions, spray agents, etc.). Examples of pharmaceutically acceptable carriers include excipients, lubricants, binders, disintegrants, disintegration inhibitors, absorption promoters, absorbers, moisturizing agents, solubilizing agents, stabilizers and the like in solid formulations; and solvents, solubilizing agents, suspending agents, isotonic agents, buffers, soothing agents and the like in liquid formulations. Additives for formulations, such as antiseptics antioxidants, colorants, sweeteners, and the like can be optionally used. The composition of the present invention can be mixed with substances other than the product substance, and the like of the present invention. Examples of parenteral routes of administration include, but are not limited to, intravenous injection, intramuscular injection, intranasal, rectum vagina, transdermal, and the like.

Examples of excipient in solid formulations include glucose, lactose, sucrose, D-mannitol, crystallized cellulose, starch, calcium carbonate, light silicic acid anhydride, sodium chloride, kaolin, urea, and the like.

Examples of lubricants in solid formulations include, but are not limited to, magnesium stearate, calcium stearate, boric acid powder, colloidal silica, talc, polyethylene glycol, and the like.

Examples of binders in solid formulations include, but are not limited to, water, ethanol, propanol, saccharose, D-mannitol, crystallized cellulose, dextran, methylcellulose, hydroxypropylcellulose, hydroxypropylmethylcellulose, carboxymethylcellulose, starch solution, gelatin solution, polyvinylpyrrolidone, calcium phosphate, potassium phosphate, shellac, and the like.

Examples of disintegrants in solid formulations include, but are not limited to, starch, carboxymethylcellulose, carboxymethylcellulose calcium, agar powder, laminarin powder, croscarmellose sodium, carboxymethyl starch sodium, sodium alginate, sodium hydrocarbonate, calcium carbonate, polyoxyethylene sorbitan fatty acid esters, sodium lauryl sulfate, starch, monoglyceride stearate, lactose, calcium glycolate cellulose, and the like.

Examples of disintegration inhibitors in solid formulations include, but are not limited to, hydrogen-added oil, saccharose, stearin, cacao butter, hydrogenated all, and the like.

Examples of absorption promoters in solid formulations include, but are not limited to, quaternary ammonium salts, sodium lauryl sulfate, and the like.

Examples of absorbers in solid formulations include, but are not limited to, starch, lactose, kaolin, bentonite, colloidal silica, and the like.

Examples of moisturizing agents in solid formulations include, but are not limited to, glycerin, starch, and the like.

Examples of solubilizing agents in solid formulations include, but are not limited to, arginine, glutamic acid, aspartic acid, and the like.

Examples of stabilizers in solid formulations include, but are not limited to, human serum albumin, lactose, and the like.

When tablets, pills, and the like are prepared as solid formulations, they may be optionally coated with a film of a substance dissolvable in the stomach or the intestine (saccharose, gelatin, hydroxypropylcellulose, hydroxypropylmethylcellulose phthalate, etc.). Tablets include those optionally with a typical coating (e.g., dragees, gelatin coated tablets, enteric coated tablets, film coated tablets or double tablets, multilayer tablets, etc.). Capsules include hard capsules and soft capsules. When tablets are molded into the form of a suppository, higher alcohols, higher alcohol esters, semi-synthesized glycerides, or the like can be added in addition to the above-described additives. The present invention is not so limited.

Preferable examples of solutions in liquid formulations include injection solutions, alcohols, propyleneglycol, macrogol, sesame oil, corn oil, and the like.

Preferable examples of solubilizing agents in liquid formulations include, but are not limited to, polyethyleneglycol, propyleneglycol, D-mannitol, benzyl benzoate, ethanol, trisaminomethane, cholesterol, triethanolamine, sodium carbonate, sodium citrate, and the like.

Preferable examples of suspending agents in liquid formulations include surfactants (e.g., stearyltriethanolamine, sodium lauryl sulfate, lauryl amino propionic acid, lecithin, benzalkonium chloride, benzethonium chloride, glycerin monostearate, etc.), hydrophilic macromolecule (e.g., polyvinyl alcohol, polyvinylpyrrolidone, carboxymethylcellulose sodium, methylcellulose, hydroxymethylcellulose, hydroxyethylcellulose, hydroxypropylcellulose, etc.), and the like.

Preferable examples of isotonic agents in liquid formulations include, but are not limited to, sodium chloride, glycerin, D-mannitol, and the like.

Preferable examples of buffers in liquid formulations include, but are not limited to, phosphate, acetate, carbonate, citrate, and the like.

Preferable examples of soothing agents in liquid formulations include, but are not limited to, benzyl alcohol, benzalkonium chloride, procaine hydrochloride, and the like.

Preferable examples of antiseptics in liquid formulations include, but are not limited to, parahydroxybenzoate ester, chlorobutanol, benzyl alcohol, 2-phenylethylalcohol, dehydroacetic acid, sorbic acid, and the like.

Preferable examples of antioxidants in liquid formulations include, but are not limited to, sulfite, ascorbic acid, α-tocopherol, cysteine, and the like.

When liquid agents and suspensions are prepared as injections, they are sterilized and are preferably isotonic with the blood. Typically, these agents are made aseptic by filtration using a bacteria-retaining filter or the like, mixing with a bactericide or, irradiation, or the like. Following these treatments, these agents may be made solid by lyophilization or the like. Immediately before use, sterile water or sterile injection diluent (lidocaine hydrochloride aqueous solution, physiological saline, glucose aqueous solution, ethanol or a mixture solution thereof, etc.) may be added.

The pharmaceutical composition of the present invention may further comprise a colorant, a preservative, a flavor, an aroma chemical, a sweetener, or other drugs.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, to within the skill of the art. Administration methods may herein include oral administration and parenteral administration (e.g., intravenous, intramuscular, subcutaneous, intradermal, mucosal, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing a sugar chain composition having the desired degree of purity with optional physiologically acceptable carriers, excipients, or stabilizers (Japanese Pharmacopeia 14th Edition or the latest edition: Remington's Pharmaceutical Sciences, 18th Edition, A. R. Gennaro, ed., Mack Publishing Company, 1990; and the like), in the form of lyophilized cake or aqueous solutions.

Various delivery systems are known and can be used to administer a compound of the present invention (e.g., liposomes, microparticles, microcapsules). Methods of introduction include, but arm not limited to, intradermal, intramuscular, intraperitoneal, intravenous, subcutaneous, intranasal, epidural, and oral routes. The compounds or compositions may be administered by any convenient routs (e.g., by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the present invention into the central nervous system by any suitable route (including intraventricular and intrathecal injection; intraventricular injection may be facilitated by an intraventricular catheter, for example, attached to a reservoir, such as an Ommaya reservoir). Pulmonary administration can also be employed, e.g., by use of an inhaler or nebulizer, and formulation with an aerosolizing agent.

In a specific embodiment, it may be desirable to administer a product substance of the present invention or a composition comprising the same locally to the area in need of treatment (e.g., the central nervous system, the brain, etc.); this may be achieved by, for example, and not by way of limitation, local infusion during surgery, topical application (e.g., in conjunction with around dressing after surgery), by infection, by means of a catheter, by means of a suppository, or by means of an implant (the implant being of a porous, non-porous, or gelatinous material, including membranes, such as sialastic membranes, or fibers). Preferably, when administering a protein, including an antibody, of the present invention, care must be taken to use materials to which the protein does not absorb.

In another embodiment, the compound or composition can be delivered in a vesicle, in particular a liposome (see Langer, Science 249: 1527-1533 (1990); Treat et al., Liposomes in the Therapy of infectious Disease and Cancer, Lopez-Berestein and Fidler (eds.), Lies, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14: 201 (1987); Buchwald et al., surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Pres., Boca Raton, Fla. (1974): Controlled Drug Bioavailability, Drug Product Design and Performance, Smolen and Ball (eds.), Wiley, New York (1984): Ranger and Peppas, J., Macromol. Sci. Rev. Macromol. Chem. 23: 61 (1983) see also Levy et al., Science 228: 190 (1985): During et al., Ann. Neurol. 25: 351 (1989); Howard et al., J. Neurosurg. 71:105 (1989)).

In yet another embodiment, a controlled release system can be placed in proximity to the therapeutic target, i.e., the brain, thus requiring only a fraction of the systemic dose (see, e.g., Goodson, in Medical Applications of Controlled Release, Supra, vol. 2, pp. 115-138 (1984)).

Other controlled release systems are discussed in the review by Langer (Science 249: 1527-1533 (1990)).

The amount of a compound used in the treatment method of the present invention can be easily determined by those skilled in the art with reference to the purpose of use, target disease (type, severity, and the like), the patient's age, weight, sex, and case history, the form or type of the calls, and the like. The frequency of the treatment method of the present invention which is applied to a subject (patient) is also determined by those skilled in the art with respect to the purpose of use, target disease (type, severity, and the like), the patient's age, weight, sex, and case history, the progression of the therapy, and the like. Examples of the frequency include once per day to once per several months (e.g., once per week to once per month). Preferably, administration is performed once per week to once per month with reference to the progression.

The doses of the product substance or the like of the present invention vary depending on the subject's age, weight and condition or administration method or the like, including, but not limited to, ordinarily 0.01 mg to 10 g per day for an adult in the case of oral administration, preferably 0.1 mg to 1 g, 1 mg to 100 mg, 0.1 mg to 10 mg, and the like; in the parenteral administration, 0.01 mg to 1 g, preferably 0.01 mg to 100 mg, 0.1 mg to 100 mg, 1 mg to 100 mg, 0.1 mg to 10 mg, and the like. The present invention is not so limited.

As used herein, the term “administer” means that the polypeptides, polynucleotides or the like of the present invention or pharmaceutical compositions containing them are incorporated into cell tissue of an organism either alone or in combination with other therapeutic agents. Combinations may be administered either concomitantly (e.g., as an admixture), separately but simultaneously or concurrently; or sequentially. This includes presentations in which the combined agents are administered together as a therapeutic mixture, and also procedures in which the combined agents are administered separately but simultaneously (e.g. as through separate intravenous lines into the same individual). “Combination” administration further includes the separate administration of one of the compounds or agents given first, followed by the second.

As used herein, “instructions” describe a method of administering a medicament of the present invention, a method for diagnosis, or the like for persons who administer, or are administered, the medicament or the like or persons who diagnose or are diagnosed (e.g. physicians, patients, and the like). The instructions describe a statement indicating an appropriate method for administrating a diagnostic, medicament, or the like of the present invention. The instructions are prepared in accordance with a format defined by an authority of a country in which the present invention is practiced (e.g., Health, Labor and Welfare Ministry in Japan, Food and Drug Administration (FDA) in U.S., and the like), explicitly describing that the instructions are approved by the authority. The instructions are so-called package insert and are typically provided in paper media. The instructions are not so limited and may be provided in the form of electronic media (e.g., web sites and electronic mails provided on the Internet).

The judgment of termination of treatment with a method of the present invention may be supported by a result of a standard clinical laboratory using commercially available assays or instruments or extinction of a clinical symptom characteristic to a disease of interest. Treatment can be resumed with the relapse of a disease of interest.

The present invention also provides a pharmaceutical package or kit comprising one or more containers loaded with one or more pharmaceutical compositions. A notice in a form defined by a government agency which regulates the production, use or sale of pharmaceutical products or biological products may be arbitrarily attached to such a container, representing the approval of the government agency relating to production, use or sale with respect to administration to humans.

### DESCRIPTION OF PREFERRED EMBODIMENTS OF THE INVENTION

Hereinafter, the present invention will be described by way of examples. Examples described below are provided only for illustrative purposes. Accordingly, the scope of the present invention is not limited except as by the appended claims.

In one aspect of the present invention, a method for regulating the conversion rate of a hereditary trait of an organism or a cell is provided. The method comprises the steps of: (a) regulating an error-prone frequency in replication of a gene of the organism or the cell. In this case, the error-prone frequency can be regulated by regulating a proofreading function of a DNA polymerase, for example, or alternatively, by increasing errors in polymerization reactions of the DNA polymerase. Such error-prone frequency regulation can be carried out using techniques well known in the art. The error-prone frequency regulation can provide rapid mutagenesis to an extent which cannot be conventionally achieved, and near-natural evolution. In addition, deleterious mutations which occur more frequently than beneficial mutations can be substantially reduced as compared to any mutagenesis method known in the art using UV, chemicals, or the like. This is because in the method of the present invention, introduced mutations are the same phenomena as that in naturally-occurring evolution phenomena.

In the method of the present invention for evolving cells or organisms, the step of regulating an error-prone frequency and the stop of screening cells or organisms obtained for a desired trait can be carried out separately. By carrying out the two steps separately, the error-prone frequency (or the rate of evolution) can be regulated under conditions that do not exert selection pressure; the number of individuals can be increased to a certain number; and the variants are screened for and identified. These steps are similarly repeated at the second time and thereafter, so that evolved cells or organisms of interest can be efficiently and effectively obtained.

In conventional methods, the occurrence frequency of beneficial mutations is increased with an increase in the mutation frequency of an organism or a cell. At the same time, however, deleterious mutations also take place. Typically, the occurrence frequency of deleterious mutations is high so that the occurrence frequency of beneficial mutations can be substantially reduced as compared to the occurrence frequency of deleterious mutations provided by any mutagenesis method known in the art using UV, chemicals, or the like. Therefore, in conventional methods, it is not possible to induce a plurality of beneficial mutations in an organism or a cell while the occurrence frequency of deleterious mutations can be substantially reduced as compared to any mutagenesis method known in the art using UV chemicals, or the like.

In some conventional mutagenesis methods, natural mutation is employed. However, in this case, the occurrence frequency of natural mutations is considerably low (e.g., 10⁻¹⁰ mutations (per base per replication) for E. coli, etc.). Therefore, the rate of natural mutation is poorly practical. In addition, beneficial mutation rarely occurs in nature. Therefore, breeding relying on natural mutation requires a large organism population and a long time period. Unlike the method using natural mutation, the method of the present invention only requires a small organism population and a time corresponding to about one to several generations. The effect of the present invention is great.

In site-directed mutagenesis, only a predetermined mutation can be induced. Although the reliability is excellent, site-directed mutagenesis is not suited to large scale use and a mutated property does not have an influence on the entire organism. Thus, site-directed mutagenesis does not necessarily cause a beneficial mutation. Therefore, site-directed mutagenesis cannot be said to mimic natural evolution and has a disadvantage in that an adverse effect due to gene recombination is accompanied thereto. The present invention can provide substantially the same mutagenesis as natural mutagenesis, but not artificial mutagenesis.

As other mutagenesis methods, there are methods using radiation, mutagens, and the like. These methods can generate mutations at a higher frequency than that of natural mutations. However, an effective dose of radiation or an effective concentration of mutagene may kill most of the treated cells. In other words, deleterious mutations are lethal to organisms, in the methods using mutagens, it is not possible to induce mutagenesis without deleterious mutations. By the method of the present invention, the occurrence frequency of deleterious mutations can be substantially reduced as compared to those of the above-described methods such as UV, chemicals, or the like. The method of the present invention only requires a small organism population and a time corresponding to about one to several generations.

In the method for regulating the conversion rate of a hereditary trait using the disparity theory according to the present invention, by utilizing a DNA polymerase having a regulated proofreading function, a larger number of mutations are introduced into one strand of double-stranded genomic DNA than into the other strand. The present invention is the first to demonstrate at the experimental level that a plurality of beneficial mutations can be accumulated without accumulation of deleterious mutations. Therefore, the present invention disproves the disparity theory that a number of mutations are expected to be introduced into an organism, but the normal growth (metabolism, etc.) of the organisms would not be maintained. Thus, the present invention is an epoch-making invention. Particularly, a eukaryotic organism has a plurality of bi-directional origins of replication. If genomic DNA has a bi-directional origin of replication, the disparity method cannot accumulate a plurality of beneficial mutations without accumulation of deleterious mutations. According to the method of the present invention, it was demonstrated that even in eukaryotic organisms, a plurality of beneficial mutations can be accumulated without accumulation of deleterious mutations.

In a preferred embodiment, it may be advantageous to introduce a DNA polymerase having an altered proofreading function into only one of a lagging strand and a leading strand.

Satisfactory breeding achieved by the present invention is considered to achieve high-speed organism evolution. High-speed organism evolution typically requires large genetic diversity of a population and stable expansion of beneficial mutants. Stable expansion is achieved by accurate DNA replication, while mutations caused by errors during DNA replication produce genetic diversity.

An effect of the present invention is that high-speed evolution can be achieved even in eukaryotic organisms. Eukaryotic organisms have a definite nuclear structure and their genomes are composed of a plurality of chromosomes, as is different from E. coli. Therefore, the present invention can be said to have an effect which cannot be unexpected from conventional techniques. Even if the evolution speed could be regulated in E. coli, it could not have been expected that evolution speed can be regulated in eukaryotic organisms or gram-positive bacteria until this was demonstrated in an example herein.

In a preferred embodiment, agents playing a role in gene replication include at least two kinds of error-prone frequency agents. The two error-prone frequency agents are preferably DNA polymerases. These DNA polymerases have a different error-prone frequency. In a preferred embodiment, the error-prone frequency agents may advantageously include at least about 30% of agents having a lesser error-prone frequency, more preferably at least about 20%, and even more preferably at least about 15%. With this feature, there is an increasing probability that a mutant is generated with dramatic evolution while stable replication is carried out.

In another preferred embodiment, agents (e.g., DNA polymerases, etc.) playing a role in gene replication according to the present invention advantageously have heterogeneous error-prone frequency. Non-uniform error-prone frequency allows an increase in the rate of evolution compared to conventional techniques and removal of the upper limit of the error threshold.

In a preferred embodiment, agents having a low error-prone frequency are substantially error-free. However, agents having error-prone frequency such that there is substantially no error per genome may be preferably used.

Therefore, in a preferred embodiment, at least two kinds of error-prone frequencies are typically different from each other by at least 10¹, preferably at least 10², and more preferably at least 10³. With such a frequency difference, the rate of evolution can be more efficiently regulated.

In one embodiment of the present invention, the step of regulating error-prone frequency comprises regulating the error-prone frequency of a DNA polymerase of an organism. The error-prone frequency of a DNA polymerase of an organism of interest may be regulated by directly modifying a DNA polymerase present in the organism, or alternatively, by introducing a DNA polymerase having a modified error-prone frequency externally into the organism. Such modification of a DNA polymerase may be carried out by biological techniques well known in the art. The techniques are described in other portions of the present specification. In a non-limiting example, direct modification of a DNA polymerase can be carried out by crossing organism lines into which mutations have already been introduced.

In another embodiment, a DNA polymerase has a proofreading function. In an organism of interest, a DNA polymerase having a proofreading function is typically present. Examples of such a DNA polymerase having a proofreading function include, but are not limited to, DNA polymerases δ and ε. DnaQ, DNA polymerases β, θ, and λ which have a repair function, and the like. The proofreading function of a DNA polymerase may be regulated by directly modifying a DNA polymerase present in the organism, or alternatively, by introducing a DNA polymerase having a modified proofreading function externally into the organism. Such modification of a DNA polymerase may be carried out by biological techniques well known in the art. The techniques are described in other portions of the present specification. In a non-limiting example, direct modification of a DNA polymerase can be carried out by crossing organism lines into which mutations have already been introduced. Preferably, a nucleic acid molecule encoding a modified DNA polymerase is incorporated into a plasmid, and the plasmid is introduced into an organism, so that the nucleic acid molecule is transiently expressed. Due to the transient expression property of a plasmid or the like, the plasmid or the like is vanished. Thus, after regulation of the conversion rate of a hereditary trait is no longer required, the same conversion rate as that of a wild type can be restored.

In another embodiment, a DNA polymerase of the present invention includes at least one polymerase selected from the group consisting of DNA polymerase δ and DNA polymerase ε of eukaryotic organisms and DNA polymerases corresponding thereto. In still another preferred embodiment, only one DNA polymerase for use in the present invention selected from the group consisting of DNA polymerase δ and DNA polymerase ε of eukaryotic organisms and DNA polymerases corresponding thereto, may be modified. By modifying the error-prone frequency of only one DNA polymerase, a genotype (including a wild type) which has once appeared is conserved; a high rate of mutation may be allowed; a wide range (genes) in a genome can be improved original traits can be guaranteed and diversity can be increased; evolution may be accelerated to a rate exceeding conventional levels; and mutated traits are stable.

In another embodiment of the present invention, the step of regulating an error-prone frequency comprises regulating at least one polymerase selected from the group consisting of DNA polymerase δ and DNA polymerase ε of eukaryotic organisms and DNA polymerases corresponding thereto. Such proofreading activity can be regulated by modifying the 3′→5′ exonuclease activity center of the polymerase (alternatively, ExoI motif, proofreading function active site) (e.g., aspartic acid at position 316 and glutamic acid at position 318 and sites therearound of human DNA polymerase δ), for example. The present invention is not limited to this.

In a preferred embodiment of the present invention, the step of regulating an error-prone frequency comprises increasing the error-prone frequency to a level higher than that of the wild type. By increasing an error-prone frequency to a level higher than that of the wild type, the hereditary trait conversion rate (i.e., the rate of evolution) of organisms was increased without an adverse effect on the organisms. Such an achievement was not conventionally expected. The present invention has an excellent effect.

In another preferred embodiment, a DNA polymerase for use in the present invention has a proofreading function lower than that of the wild type. Such a DNA polymerase may be naturally-occurring, or alternatively, may be a modified DNA polymerase.

In one embodiment, a (modified) DNA polymerase for use in the present invention advantageously has a proofreading function which provides mismatched bases (mutations), the number of which is greater by at least one than that of the wild type DNA polymerase. By providing mismatched bases (mutations), the number of which is greater by at least one than that of the wild type DNA polymerase, the hereditary trait conversion rate (i.e., the rate of evolution) of organisms was increased without an adverse effect on the organisms. The hereditary trait conversion rate tends to be increased if the number of mutated bases is greater than that of the wild type DNA polymerase. Therefore, to increase the conversion rate, a proofreading function is preferably further lowered. Methods for assaying a proofreading function are known in the art. For example, products obtained by an appropriate assay system suitable for a DNA polymerase of interest (determination by sequencing replicated products; determination by measuring proofreading activity) are directly or indirectly sequenced (e.g., by a sequencer or a DNA chip).

In another preferred embodiment, a DNA polymerase for use in the present invention advantageously has a proofreading function which provides at least one mismatched base (mutation). Typically, wild type DNA polymerases often provide no mutation the base sequence of a resultant product. Therefore, in such a case, a DNA polymerase variant for use in the present invention may need to have a lower level of proofreading function which provides at least one mismatched base (mutation). Such a proofreading function can be measured by the above-described assay system. More preferably, a DNA polymerase for use in the present invention has a proofreading function which provides at least two mismatched bases (mutations), more preferably at least 3, 4, 5, 6, 7, 8, 9, and 10 mismatched bases, and more preferably at least 15, 20, 25, 50, and 100 mismatched bases. It is considered that the hereditary trait conversion rate (i.e., the rate of evolution) of organisms is increased with a decrease in the level of a proofreading function, i.e., an increase in the number of mismatched bases (mutations) in a base sequence.

In another embodiment, a DNA polymerase for use in the present invention has a proofreading function which provides a mismatched base (mutation) in a base sequence at a rate of 10⁻⁶. Typically, mutations are induced at a rate of 10⁻¹² to 10⁻⁶ in naturally-occurring organisms. Therefore, in the present invention, it is preferable to employ a DNA polymerase having a significantly lowered proofreading function. More preferably, a DNA polymerase for use in the present invention has a proofreading function which provides a mismatched base (mutation) in a bane sequence at a rate of 10⁻³, and even more preferably at a rate of 10⁻². It is considered that the hereditary trait conversion rate (i.e., the rate of evolution) of organisms is increased with a decrease in the level of a proofreading function, i.e., an increase in the number of mismatched bases (mutations) in a base sequence.

In a certain embodiment, an organism targeted by the present invention may be a eukaryotic organism. Eukaryotic organisms have a mechanism conferring a proofreading function, which is different from that of E. coli. Therefore, the rate of evolution is discussed or explained in a manner different from when E. coli is used as a model. Unexpectedly, the present invention demonstrated that the hereditary trait conversion rate (i.e., the rate of evolution) of all organisms including eukaryotic organisms can be modified. Therefore, the present invention provides an effect which cannot be predicted by conventional techniques. Particularly, since the rate of evolution can be regulated in eukaryotic organisms by the present invention, the following various applications were achieved: elucidation of the mechanism of evolution; elucidation of the relationship between a genome and traits; improvement of various higher organisms including animals and plants; investigation of the evolution ability of existing organisms; prediction of future organisms; production of animal models of diseases; and the like. Examples of eukaryotic organisms targeted by the present invention include, but are not limited to, unicellular organisms (e.g., yeast, etc.) and multicellular organisms (e.g., animals and plants). Examples of such organisms include, but are not limited to, Myxiniformes, Petronyzoniformes, Chondrichthyes, Osteichthyes, the class Mammalia (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.), the class Aves, the clans Reptilia, the class Amphibia, the class Pisces, the class Insecta, the class Vermes, dicotyledonous plants, monocotyledonous plants (e.g., the family Gramineae, such as wheat, maize, rice, barley, sorghum, and the like), Pteridophyta, Bryophyta, Eumycetes, cyanobacteria, and the like. Preferably, an organism targeted by the present invention may be a multicellular organism. In another preferred embodiment, an organism targeted by the present invention may be a unicellular organism. In another preferred embodiment, an organism targeted by the present invention may be an animal, a plant, or yeast. In a more preferred embodiment, an organism targeted by the present invention may be, but is not limited to, a mammal.

In another embodiment, an organism or a cell for use in the present invention naturally has at least two kinds of polymerases. If at least two kinds of polymerases are present, it is easy to provide an environment where heterogeneous error-prone frequency is provided. More preferably, it is advantageous that an organism or a cell naturally has at least two kinds of polymerase and the error-prone frequencies thereof are different from one another. Such an organism or cell can be used to provide a modified organism or call.

In a preferred embodiment, a modified organism or cell obtained by a method of the present invention has substantially the same growth as the wild type after a desired trait has been transformed. This feature is obtained only after the present invention provides regulation of the conversion rate of a hereditary trait without an adverse affect. The feature cannot be achieved by conventional mutagenesis methods. Thus, the feature is an advantageous effect provided by the present invention. Organisms or cells having substantially the same growth as the wild types can be handled in the same manner as the wild types.

In another embodiment, an organism or a call modified by a method of the present invention has resistance to an environment to which the organism or the cell has not had resistance before modification (i.e., the wild type). Examples of ouch an environment include at least one agent, as a parameter, selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, organisms (e.g., parasites, etc.) other than the organism, chemical agents, antibiotics, natural substances, mental stress, and physical stress, and any combination thereof. Thus, any combination of theme agents may be used. Any two or more agents may be combined.

Examples of temperature include, but are not limited to, high temperature, low temperature, very high temperature (e.g., 95° C. etc.), very low temperature (e.g., −80° C., etc.), a wide range of temperature (e.g., 150 to −270° C., etc.), and the like.

Examples of humidity include, but are not limited to, a relative humidity of 100%, a relative humidity of 0%, an arbitrary point from 0% to 100%, and the like.

Examples of pH include, but are not limited to, an arbitrary point from 0 to 14, and the like.

Examples of salt concentration include, but are not limited to, a NaCl Concentration (e.g., 3%, etc.), an arbitrary point of other salt concentrations from 0 to 100%, and the like.

Examples of nutrients include, but are not limited to, proteins, glucose, lipids, vitamins, inorganic salts, and the like.

Examples of metals include, but are not limited to, heavy metals (e.g., mercury, cadmium, etc.), lead, gold, uranium, silver, and the like.

Examples of gas include, but are not limited to, oxygen, nitrogen, carbon dioxide, carbon monoxide, and a mixture thereof, and the like.

Examples of organic solvents include, but are not limited to, ethanol, methanol, xylene, propanol, and the like.

Examples of pressure include, but are not limited to, an arbitrary point from 0 to 10 ton/cm², and the like.

Examples of atmospheric pressure include, but are not limited to, an arbitrary point from 0 to 100 atmospheric pressure, and the like.

Examples of viscosity include, but are not limited to the viscosity of any fluid (e.g., water, glycerol, etc.) or a mixture thereof, and the like.

Examples of flow rate include, but are not limited to an arbitrary point from 0 to the velocity of light.

Examples of light intensity include, but are not limited to, a point between darkness and the level of sunlight.

Examples of light wavelength include, but are not limited to visible light, ultraviolet light (UV-A, UV-B, UV-C, etc.), infrared light (far infrared light, near infrared light, etc.), and the like.

Examples of electromagnetic waves include ones having an arbitrary wavelength.

Examples of radiation include ones having an arbitrary intensity.

Examples of gravity include, but are not limited to, an arbitrary gravity on the Earth or an arbitrary point from zero gravity to a gravity on the Earth, or an arbitrary gravity greater than or equal to a gravity on the Earth.

Examples of tension include ones having an arbitrary strength.

Examples of acoustic waves include ones having an arbitrary intensity and wavelength.

Examples of organisms other than an organism of interest include, but are not limited to, parasites, pathogenic bacteria, insects, nematodes, and the like.

Examples of chemicals include, but are not limited to hydrochloric acid, sulfuric acid, sodium hydroxide, and the like.

Examples of antibiotics include, but are not limited to, penicillin, kanamycin, streptomycin, quinoline, and the like.

Examples of naturally-occurring substances include, but are not limited to, puffer toxin, snake venom, akaloid, and the like.

Examples of mental stress include, but are not limited to starvation, density, confined spaces, high places, and the like.

Examples of physical stress include, but are not limited to vibration, noise, electricity, impact, and the like.

In another embodiment, an organism or a cell targeted by a method of the present invention has a cancer cell. An organism or cell model of cancer achieved by the present invention generates cancer according to the same mechanism as that of naturally-occurring cancer, as is different from conventional methods. Thus, the organism or cell model of cancer can be regarded as an exact organism or cell model of cancer. Therefore, the organism or cell model of cancer is particularly useful for development of pharmaceuticals.

In another aspect of the present invention, a method for producing an organism or a cell having a regulated hereditary trait is provided. The method comprises the steps of: (a) regulating or changing an error-prone frequency of replication of a gene in an organism or a cell; and (b) reproducing the resultant organism or cell. In this case, techniques relating to regulation of the conversion rate of a hereditary trait are described above. Therefore, the above-described techniques can be utilized in the step of changing an error-prone frequency of replication of a gene in an organism or a cell. Organisms or cells as described above in relation to the method for regulating the conversion rate of a hereditary trait may be used in the step of regulating an error-prone frequency.

The step of reproducing the resultant organism or cell may be carried out using any method known in the art if the organism or cell has a regulated hereditary trait. Reproduction techniques include, but are not limited to, natural phenomena, such as multiplication, proliferation, and the like; artificial techniques, such as cloning techniques; reproduction of individual plants from cultured cells; and the like. Whether or not such a technique was used can be confirmed by, for example, confirmation by determination of base sequences; identification of antigenicity or the like: detection of vectors when vectors are used, a trait restoring test; and confirmation of compatibility of a high rate of mutation and non-disruption. These tests can be easily carried out by those skilled in the art based on the present specification.

In a preferred embodiment, the organism or cell reproducing method for the present invention further comprises screening reproduced organisms or cells for an individual having a desired trait. Such an individual having a desired trait may be screened for based on a hereditary trait of organisms or cells (e.g., resistance to the above-described various environments, etc.), or at the gene or metabolite level. The results of screening can be confirmed by various techniques, including, not being limited to, visual inspection, sequencing, various biochemical tests, microscopic observation, staining, immunoassay, behavior analysis, and the like. These techniques are known in the art and can be easily carried out by those skilled in the art in view of the present specification.

In another aspect of the present invention, an organism or a call produced according to the present invention, whose hereditary trait is regulated, is provided. The organism or cell is obtained at a high rate of evolution which cannot be achieved by conventional techniques. Therefore, the presence per so of the organism or cell is clearly novel. The organism or cell is characterized by, for example: compatibility of a high rate of mutation and non-disruption; biased distribution of SNPs (single nucleotide polymorphism); mutations tend to be accumulated in different modes even in the same region of a genome, depending on individuals (particularly, this tendency is significant in a region which is not subject to selection pressure); the distribution of mutations in a particular region (especially, a redundant region) of the genome of the same individual is not random and is significantly biased; and the like. The organism or cell of the present invention preferably has substantially the same growth as that of the wild type. Typically, it is not possible that organisms which have undergone rapid mutagenesis have the same growth as that of the wild type. However, the organism or cell of the present invention can have substantially the same growth as that of the wild type. Therefore, the present invention has much a remarkable effect. Experiments for confirming such a property are known in the art and can be easily carried out by those skilled in the art in view of the present specification.

In another aspect of the present invention, a method for producing a nucleic acid molecule encoding a gene having a regulated hereditary trait is provided. The method comprises the steps of: (a) changing the error-prone frequency of gene replication of an organism or a cell; (b) reproducing the resultant organism or cells (c) identifying a mutation in the organism or cell; and (d) producing a nucleic acid molecule encoding a gene containing the identified mutation. In this case, techniques for changing an error-prone frequency and for reproducing resultant organisms or cell are described above and can be appropriately carried out by those skilled in the art in view of the present specification. Embodiments of the present invention can be carried out using these techniques.

Mutations in organisms or cells can be identified using techniques well known in the art. Examples of the identifying techniques include, but are not limited to, molecular biological techniques (e.g., sequencing, PCR, Southern blotting, etc.), immunochemical techniques (e.g., western blotting, etc.), microscopic observation, visual inspection, and the like.

Once a gene carrying a mutation has been identified, a nucleic acid molecule encoding the identified gene carrying the mutation can be produced by those skilled in the art using techniques well known in the art. Examples of the production method include, but are not limited to, synthesis using a nucleotide synthesizer; semi-synthesis methods (e.g. PCR, etc.), and the like. Whether or not synthesized nucleic acid molecules have a sequence of interest can be determined by sequencing or a DNA chip using techniques well known in the art.

Therefore, the present invention provides nucleic acid molecules produced by the method of the present invention. These nucleic acid molecules are genes derived from organisms or cells which are obtained at a rate of evolution which cannot be achieved by conventional techniques. Therefore, the presence per so of the nucleic acid molecule encoding the gone is clearly novel. The nucleic acid molecule is characterized by, but is not limited to: the distribution of SNPs is biased; regions having a large number of mutations accumulated and other regions tend to be distributed in a mosaic pattern in a genome: mutations tend to be accumulated in different modes even in the same region of a genome, depending on individuals (particularly, this tendency is significant in a region which is not subject to selection pressure); the distribution of mutations in a particular region (especially, a redundant region) of the genome of the same individual is not random and is significant biased; and the like. Experiments for confirming such properties are known in the art and can be easily carried out by those skilled in the art in view of the present specification.

In another aspect of the present invention, a method for producing a polypeptide encoding a gene having a regulated hereditary trait is provided. The method comprises the steps of: (a) changing the error-prone frequency of gene replication of an organism or a cell; (b) reproducing the resultant organism or cell; (c) identifying a mutation in the organism or cell; and (d) producing a polypeptide encoding a gene containing the identified mutation, in this case, techniques for changing an error-prone frequency and for reproducing resultant organisms or cells are described above and can be appropriately carried out by those skilled in the art in view of the present specification. Embodiments of the present invention can be carried out using these techniques.

Mutations in organisms or cells can be identified using techniques well known in the art. Examples of the identifying techniques include, but are not limited to, molecular biological techniques (e.g., sequencing, PCR, Southern blotting, etc.), immunochemical techniques (e.g., western blotting, etc.), microscopic observation, visual inspection, and the like.

Once a gene carrying a mutation has been identified, a polypeptide encoded by the identified gene carrying the mutation can be produced by those skilled in the art using techniques well known in the art. Examples of the production method include, but are not limited to, synthesis using a peptide synthesizer; a nucleic acid molecule encoding the above-described gene is synthesized using gene manipulation techniques, cells are transformed using the nucleic acid molecule, the gone is expressed, and an expressed product is recovered polypeptides are purified from modified organisms or cells; and the like, whether or not the resultant polypeptide has a sequence of interest can be determined by sequencing, a protein chip, or the like using techniques well known in the art.

In another aspect of the present invention, polypeptides produced by the method of the present invention are provided. These polypeptides are encoded by genes derived from organisms or cells which are obtained at a rate of evolution which cannot be achieved by conventional techniques. Therefore, the presence per se of the polypeptide encoded by the gene is clearly novel. The polypeptide is characterized by, for example, an amino acid sequence having the following hereditary trait: the distribution of SNPs is biased; regions having a large number of mutations accumulated and other regions tend to be distributed in a mosaic pattern in a genome; mutations tend to be accumulated in different modes even in the same region of a genome, depending on individuals (particularly, this tendency is significant in a region which is not subject to selection pressure); the distribution of mutations in a particular region (especially, a redundant region) of the genomes of sperm of the same individual is not random and is significantly biased, and the like. The present invention is not limited to this. Experiments for confirming such properties are known in the art and can be easily carried out by those skilled in the art in view of the present specification.

In another aspect of the present invention, a method for producing a metabolite of an organism having a regulated hereditary trait is provided. The method comprises the steps of: (a) changing the error-prone frequency of gone replication of an organism or a cell; (b) reproducing the resultant organism or cell; (c) identifying a mutation in the organism or cell; and (d) producing a metabolite containing the identified mutation. In this case, techniques for changing an error-prone frequency and for reproducing resultant organisms or cells are described above and can be appropriately carried out by those skilled in the art in view of the present specification. Embodiments of the present invention can be carried out using these techniques.

As used herein, the term “metabolite” refers to a molecule which is obtained by activity (metabolism) for survival in cells. Examples of metabolites include, but are not limited to, compounds, such as amino acids, fatty acids and derivatives thereof, steroids, monosaccharides, purines, pyrimidines, nucleotides, nucleic acids, proteins, and the like. In addition, substances obtained by hydrolysis of these polymer compounds or oxidation of carbohydrates or fatty acids are also called metabolites. Metabolites may be present in cells or may be excreted from cells.

In the method of the present invention, mutations in organisms or cells can be identified using techniques well known in the art. Examples of the identifying techniques include, but are not limited to, identification of metabolites (component analysis), molecular biological techniques (e.g., sequencing, PCR, southern blotting, etc.), immunochemical techniques (e.g., western blotting, etc.), microscopic observation, visual inspection, and the like. Metabolite identifying techniques can be appropriately selected by those skilled in the art, depending on a metabolite.

In another aspect of the present invention, metabolites produced by the method of the present invention are provided. These metabolites are also derived from organisms or cells obtained at a rate of evolution which cannot be achieved by conventional techniques, and the presence per se of the metabolites is clearly novel. The metabolite is characterized by, but is not limited to: being less toxic to self; preemption of spontaneously evolved metabolites; and the like. Experiments for confirming such properties are known in the art and can be easily carried out by those skilled in the art in view of the present specification.

In another aspect of the present invention, a nucleic acid molecule for regulating a hereditary trait of an organism or a cell is provided. The nucleic acid molecule comprises a nucleic acid sequence encoding a DNA polymerase having a modified error-prone frequency. The DNA polymerase may be at least one polymerase selected from the group a consisting of DNA polymerase δ and DNA polymerase ε of eukaryotic organisms and DNA polymerases corresponding thereto, whose proofreading activity is regulated. The proofreading activity can be regulated by modifying the 3′→5′ exonuclease activity center of the polymerase (alternatively, ExoI motif, proofreading function active site) (e.g., aspartic acid at position 316 and glutamic acid at position 318 and sites therearound of human DNA polymerase δ), for example. The present invention is not limited to this.

Preferably, the sequence encoding the DNA polymerase contained in the nucleic acid molecule of the present invention advantageously encodes DNA polymerase δ or ε. This is because these DNA polymerases naturally possess a proofreading function and the function is relatively easily modified.

In another aspect of the present invention, a vector comprising a nucleic acid molecule for regulating a hereditary trait of an organism or a cell according to the present invention is provided. The vector may be a plasmid vector. The vector may preferably comprise a promoter sequence, an enhancer sequence, and the like if required. The vector may be incorporated into a kit for regulating a hereditary trait of organisms or calls, or may be sold.

In another aspect of the present invention, a cell comprising a nucleic acid molecule for regulating a hereditary trait of an organism or a cell according to the present invention is provided. The nucleic acid molecule of the present invention may be incorporated into the cell in the form of a vector. The present invention is not limited to this. The cell may be incorporated into a kit for regulating a hereditary trait of organisms or cells, or may be sold. In a preferred embodiment, the cell may be advantageously, but is not limited to, a eukaryotic cell. If the cell is used only so as to amplify a nucleic acid molecule, a prokaryotic cell may be preferably used.

In another aspect of the present invention, an organism or a cell comprising a nucleic acid molecule for regulating a hereditary trait of an organism or a cell according to the present invention is provided. The organism may be incorporated into a kit for regulating a hereditary trait of organisms or cells.

In another aspect, the present invention provides a product substance produced by an organism or a cell or apart thereof (e.g., an organ, a tissue, a cell, etc.) obtained by the method of the present invention is provided. Organisms or parts thereof obtained by the present invention are not obtained by conventional methods, and their product substances may include a novel substance.

In another aspect of the present invention, a method for testing a drug is provided, which comprises the steps of: testing an affect of the drug using an organism or a cell of the present invention as a model of disease; testing the effect of the drug using a wild type organism or cell as a control; and comparing the model of disease and the control. Such a model of disease is a spontaneous disease process model which cannot be achieved by conventional methods. Therefore, by using such a model of disease in a method for testing a drug, the result of the test is close to that of a test performed in a natural condition which cannot be realized by conventional methods, resulting in a high level of reliability of the test. Therefore, it is possible to reduce the development period of pharmaceuticals and the like. Alternatively, it may be possible to obtain more accurate information, such as side effects and the like, in test results.

In another aspect, the present invention relates to a set of at least two kinds of polymerases for use in regulation of the conversion rate of a hereditary trait of an organism or a cell, where the polymerases have a different error-prone frequency. Such a set of polymerases have not been conventionally used in the above-described method and is very novel. Any polymerase may be used as long as they function in an organism or a cell into which they are introduced. Therefore, polymerases may be derived from two or more species, preferably from the same animal species. Polymerases for use in the above-described application may be introduced into organisms or cells via gene introduction.

In another aspect of the present invention, a set of at least two kinds of polymerases for use in production of an organism or a cell having a modified hereditary trait, where the polymerases have a different error-prone frequency, are provided. Such a set of polymerases have not been conventionally used in the above-described method and is very novel. Any polymerases may be used as long as they function in an organism or a cell into which they are introduced. Therefore, polymerases may be derived from two or more species, preferably from the same animal species. Polymerases for use in the above-described application may be introduced into organisms via gene introduction.

In another aspect, the present invention relates to use of a set of at least two kinds of polymerases for use in regulation of the conversion rate of a hereditary trait of an organism or a cell, where the polymerases have a different error-prone frequency. Polymerases for use in the above-described application are described above and are used and produced in examples below.

In another aspect, the present invention relates to use of a set of at least two kinds of polymerases for use in production of an organism or a cell having a modified hereditary trait, where the polymerases have a different error-prone frequency. Polymerases for use in the above-described application are described above and are used and produced in examples below.

(Disparity Quasispecies Hybrid Model)

A. Mutant Distribution of Quasispecies with Heterogeneous Replication Accuracy

In another aspect of the present invention, a quasispecies consists of a population of genomes, assuming that each is represented by a binary base sequence of length n, which has 2ⁿ possible genotypes (or sequence space). A sequence with the best fitness is herein called “master sequence”. The population size is selected to be very large and stable. The replication of one template sequence produces one direct copy sequence, and thus the replication error in fixed to a mutation by one step. Only base substitutions occur, and hence the sequence length is constant. Sequence degradation is neglected. For easy handling, the present inventors classify the sum of all i-error mutants of the master sequence (I₀) into a mutant class Iᵢ (i=0, 1, . . . , n). The corresponding sum of relative concentrations is denoted by xᵢ. The rate of change in xᵢ is represented by:

$${x}_{i} = ( {A}_{i} {Q}_{ii} - f ) {x}_{i} + {\sum }_{j \neq i} {A}_{j} {Q}_{ij} {x}_{j} \qquad ( 1 )$$

where Aᵢ is the replication rate constant (or fitness) of the mutant class Iᵢ; f keeps the total concentration constant; and is then Σᵢ Aⱼ xᵢ; Qᵢᵢ is the replication accuracy or the probability of producing Iᵢ by complete error-free replication of Iⱼ; and Qᵢⱼ is the probability of Iᵢ by misreplication of Iⱼ.

The genome sequence is replicated by a polymerase. Eₖ indicates that p kinds of polymerases with different accuracies (k=1, 2, . . . , p). The relative concentration of Eₖ is denoted by cₖ. Single-base accuracy of polymerase Eₖ is represented by 0≦qₖ≦1, so that the per bass error rate is 1−qₖ. Because of the consistent replication of one sequence by the same polymerase, the per base error rate Eₖ is n(1−qₖ). The per genome mean error rate of the quasispecies is then represented by nΣₖcₖ(1−qₖ)=m. By transforming the homogeneous replication accuracy (e.g., M. Eigen, 1971 (supra)), the heterogeneous replication accuracy to obtained by:

$$\begin{matrix}{Q}_{ij} = {\sum }_{k} {c}_{k} {q}_{k}^{n} {\sum }_{h = 0}^{i} {( \frac{1 - {q}_{k}}{{q}_{k}} )}^{2 h + \vert j - i \vert} ( \begin{matrix}n - j \\ h + \frac{1}{2} ( \vert j - i \vert - j + i )\end{matrix} ) \times \\ ( \begin{matrix}j \\ h + \frac{1}{2} ( \vert j - i \vert + j - i )\end{matrix} ) , & ( 2 ) \\ with 1 = [ \frac{1}{2} ( min \{ i + i , 2 n - ( j + i ) \} - \vert j - i \vert ) ] . & ( 3 )\end{matrix}$$

The stationary mutant distribution, limₜ₋ₓxᵢ=yᵢ, is a quasispecies. This is represented by the eigenVectors of the matrix W={AⱼQᵢⱼ}. FIG. 5 shows examples of the quasispecies with homogeneous and heterogeneous replication accuracies. Here, a simple single-peaked fitness space was used. A replication rate constant A₀ is assigned to the master sequence, and all other mutant classes have the same fitness.

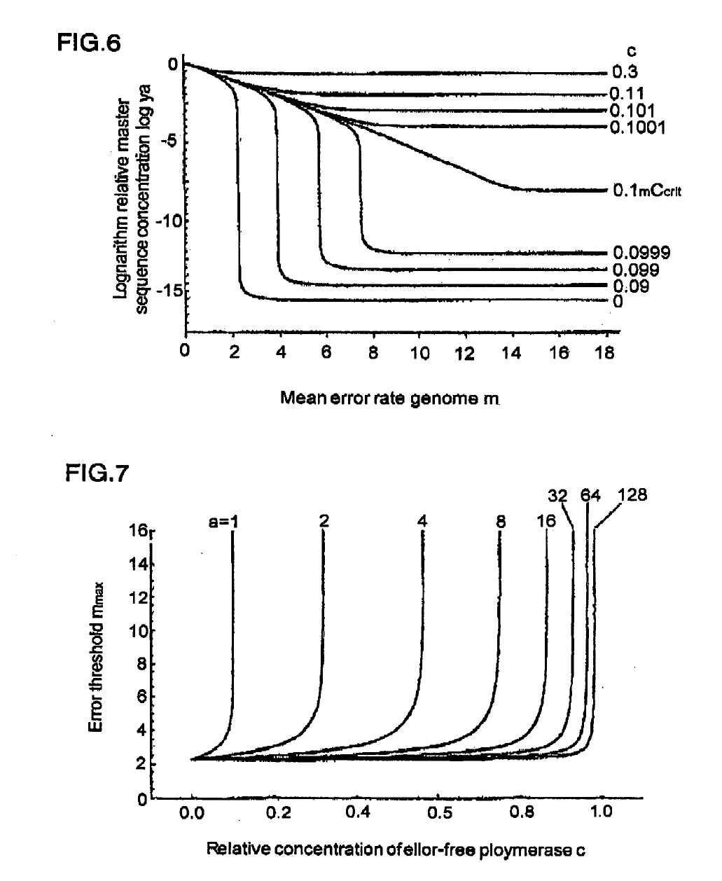

Parity quasispecies with a homogeneous replication accuracy below the error threshold localizes around the master sequence ((a) of FIG. 5). At the error threshold near m=2.3, the transition is very sharp, and the relative concentration of the master sequence decreases over about 10 orders of magnitude (at c=0, FIG. 6). Such a phenomenon is called an error catastrophe. Above the error threshold, quasispecies localization is replaced by a uniform distribution, in which individual concentrations are extremely small (e.g., yᵢ=8.88×10⁻¹⁶). In a real, finite population, it is more difficult to maintain the genetic information of the master sequence by selection as errors are accumulated. Only below the error threshold can the quasispecies evolve, and the rate of evolution appears to reach its maximum near the error threshold.

It is assumed that disparity models of the present invention ((b) to (d) in FIG. 5) have two kinds of polymerases, each with different accuracy. Polymerase E₁ is error-free, q₁=1 and E₂ is error-prone, 0≦q₂≦1; each is present at a relative concentration of c and 1−c. The assumption of a complete error-frees polymerase appears not to be realistic, however, the error rate of the proofreading polymerase in DNA-based microorganisms is very small, 0.003 errors per genome per replication, thus it is negligible in this case.

When the relative concentration of error-free polymerase is low, 0<c<1, the error threshold is shifted to a higher mean error rate with increasing c, and the magnitude of the error catastrophe decreases ((b) of FIG. 5 and FIG. 6). At c=0.1, the error threshold vanishes ((c) of FIG. 5). The relative concentration of the master sequence gradually decreases and finally levels off at a 10⁷ times higher concentration than the parity uniform distribution (at c=0.1 in FIG. 6). When c>0.1, independent of the mean error rate, the master sequence is present in a sufficient concentration ((d) of FIG. 5 and FIG. 6). FIG. 6 shows the dramatic change of the quasispecies dynamics near c_{crit}=0.1. In the disparity quasispecies model, mutants far distant from the master sequence can be present without incurring the loss of quasispecies localization. This means that the rate of evolution can increase without error catastrophe.

B. Error Threshold for Quasispecies with a Plurality of Replication Agents

Considering the error threshold for the disparity model, the present inventors encountered the following two difficulties: (i) the genome size in nature is too large; virus n>10³, bacteria: n>10⁶, to do exact calculations; and (ii) the genome replication in nature to partitioned into more than one unit (replication agent) and more than one polymerase participates at the same time. The multiple replication agents appear to influence the error threshold. The present inventors calculated the error threshold by using an approximation of the relative stationary concentration of the master sequence.

$${y}_{0} \approx \frac{{A}_{0} {Q}_{00} - {A}_{i \neq 0}}{{A}_{0} - {A}_{i \neq 0}} , \qquad ( 4 )$$

where A₀ is the replication rate constant of the master sequence and Aᵢ₌₀ is the overall average of other mutant sequences; Q₀₀ is the replication accuracy for complete error-free replication of the master sequence. This approximation relies on the negligence of considering back mutations from mutants to the master sequence in expression (1). Agreement with the exact solution increases with increasing genome size. The relative stationary concentration of the master sequence vanishes for a critical error rate that fulfils:

$${( {Q}_{00} )}_{min} = \frac{{A}_{i \neq 0}}{{A}_{0}} = {s}^{- 1} , \qquad ( 5 )$$

where s is the selective superiority of the master sequence. To obtain Q₀₀ for the disparity model with a plurality of replication agents, the present inventors assume that there are two kinds of polymerases E₁ and E₂, each present at a relative concentration of c and 1−c. The error rate of the proofreading polymerase is a very small and negligible. Thus, polymerase E₁ is error-free, q₁=1, and E₂ is error-prone, 0≦q₂≦1. The per genome mean error rate is then:
m=n(i−c)(1−q₂). (6)

The probability of replicating the genome by error-prone polymerase E₂ is obtained from a binominal distribution. The nonerror probability by the error-prone polymerase E₂ is obtained from a Poisson approximation, in which the genome size is assumed to be very large compared to the number of replication agents. Multiplying them, we have:

$$\begin{matrix}{Q}_{00} = {\sum }_{b = 0}^{a} ( \begin{matrix}a \\ b\end{matrix} ) {{c}^{a - b} ( 1 - c )}^{b} {ⅇ}^{- mb / a ( 1 - c )} \\ = {[ c + ( 1 - c ) {ⅇ}^{- m / a ( 1 - c )} ]}^{a} ,\end{matrix} \qquad ( 7 )$$

where a is the number of all replication agents in the genome. Combining expressions (5) and (7), we have the error threshold for the disparity model:

$${m}_{max} = a ( 1 - c ) ln ( \frac{1 - c}{{s}^{- 1 / a} - c} ) . \qquad ( 8 )$$

FIG. 7 shows the error threshold as a function of the relative concentration of error-free polymerase at various numbers of replication agents. The error threshold for the parity model, c=0, is not influenced by the number of replication agents. In the disparity model, c>0, the singularity occurring at the critical concentration of the error-free polymerase,
c_{crit}=s^{-1/a}, (9)
leads to a very sharp increase of error threshold. This means that in c≧c_{crit}, the error threshold vanishes. c_{crit} increases with increasing number of replication agents.

The permissible error rate is thus obtained from expressions (6) and (8):

$${m}_{pms} = \{ \begin{matrix}< a ( 1 - c ) ln ( \frac{1 - c}{{s}^{- 1 / a} - c} ) , c < z \\ \leq n ( 1 - c ) ( 1 - {q}_{min} ) , c \geq z\end{matrix} , \\ z = \frac{exp ( {nq}_{min} / a ) - exp ( n / a ) {s}^{- 1 / a}}{exp ( {nq}_{min} / a ) - exp ( n / a )} = {s}^{- 1 / a} \qquad ( 10 )$$

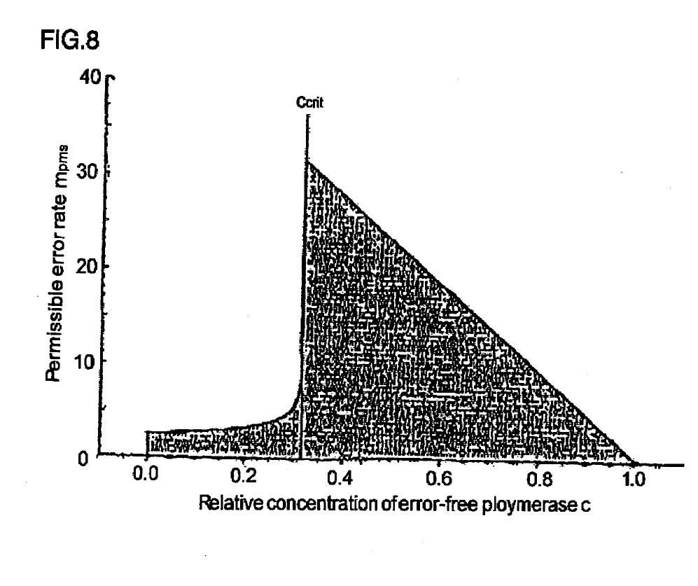

When c≧c_{crit}, there are two constraints: (i) the genome size n is finite; and (ii) the error-prone polymerase has a nonzero accuracy qₘᵢₙ in real organisms. The error rate of the complete proofreading-free DNA polymerase of Escherichia coli is assumed to be 1-qₘᵢₙ=10⁻⁵. FIG. 8 shows an example of the permissible error rate based on the parameters of E. coli. The plot resembles a λ transition in shape. For s=10, the maximum of mₚₘₛ of E. coli becomes 31 errors per genome per replication. This error rate is sufficiently high compared to the error threshold of the parity model (ln(s)=2.3).

The present inventors provide a disparity-quasispecies hybrid model in which error-free and error-prone polymerases exist. As a result, it was demonstrated that the dynamics of a quasispecies may be determined not only by the error rate but also by the proportion of polymerases with different accuracies and by the number of replication agents changing the genome. One notable finding to emerge was that the coexistence of the error-free and error-prone polymerases could greatly increase the error threshold for quasispecies compared to conventional parity models. This in an effect of the present invention which has not been revealed by conventional techniques.

A number of organisms it nature live in a continuously changing environment. This is especially true for microbial pathogens and cancer cells dodging the host immune system. The chance of finding an advantageous mutant will increase with increasing Hamming distance from the master sequence, because of the large increase in the number of mutants, and hence possible candidates, with increasing distance.

A simple homogeneous increase in the error rate would incur a considerable cost of deleterious mutations, even if it were transient. So small is the error threshold of the parity quasispecies that the distribution range of mutants is limited to a short distance from the master sequence. The parity quasispecies would be trapped in a local low peak and could never reach the higher peaks far from the master sequence. The disparity quasispecies, on the other hand, could increase the error threshold without losing genetic information, and hence produce a large number of advantageous mutants with increasing distance from the master sequence. The disparity quasispecies could search long distances across the sequence space and finally find a higher peak.

The processivity of the error-prone polymerases seems to be much lower than that of the major replicative polymerases with proofreading ability. The disparity model with a plurality of replication agents takes this observation into account. In this model, errors are concentrated within regions of a plurality of replication agents in which error-prone polymerases participate. If error-prone replication is restricted within a specific gene region, the error rate of the region greatly increases as the cost for other genes is kept to a minimum.

Therefore, according to the present invention, it was demonstrated that if DNA replication agents (e.g., Polymerases) capable of achieving at least two kinds of error-prone frequencies are provided in organisms, the organisms can exhibit the rate of evolution which is significantly increased as compared to conventional techniques while keeping the individual organisms normal. Such an effect has not been conventionally achieved.

All patents, patent applications, journal articles and other references mentioned herein are incorporated by reference in their entireties.

The present invention is heretofore described with reference to preferred embodiment to facilitate understanding of the present invention. Hereinafter, the present invention will be described by way of examples. Examples described below are provided only for illustrative purposes. Accordingly, the scope of the present invention is not limited except as by the appended claims.

### EXAMPLES

Hereinafter, the present invention will be described in more detail by ways of examples. The present invention is not limited to the examples below. Reagents, supports, and the like used it the examples below were available from Sigma (St. Louis, USA), Wako Pure Chemical industries (Osaka, Japan), and the like, with Some exceptions. Animals were treated and tested in accordance with rules defined by Japanese Universities.

### Example 1

### Production of Drug Resistant Strain and High Temperature Resistant Strain of Yeast

In Example 1, yeast was used as a representative eukaryotic organism to demonstrate that the conversion rate of a hereditary trait can be regulated in disparity mutating yeast according to the present invention.

To confirm the usefulness of disparity mutation for the field of breeding, yeast having drug resistance and/or high temperature resistance was produced.

Mutations were introduced into the proofreading function of DNA polymerase δ and DNA polymerase ε to regulate the proofreading function (Alan Morrison & Akio Sugino, Mol. Gen. Genet. (1994) 242: 289-296).

(Materials)

In Example 1, yeast (Saccharomyces cerevisiae) was used as an organism of interest. As a normal strain, AMY52-3D:MATα, ura3-52leu2-1ade2-1 his1-7hom3-10 trp1-289 canR (available from Prof. Sugino (Osaka University)) was used.

As a normal yeast strain, MYA-868(CG378) was obtained from the American Type Culture Collection (ATCC).

Error-prone frequency was regulated by changing the proofreading function of DNA polymerase δ or ε. The proofreading function was changed by producing disparity mutant strains which had a deletion in the proofreading portion of DNA polymerase δ or ε. To produce mutant strains, site-directed mutagenesis was used to perform base substitutions at a specific site of DNA polymerases polδ or polε of the normal strain (Morrison A, & Sugino A., Mol. Gen. Genet. (1994)242: 289-296) using common techniques (Sambrook et al., Molecular Cloning: A Laboratory Manual, Ver. 2, Cold Spring Harbor Laboratory (Cold Spring Harbor, N.Y., 1989), supra). Specifically, conversion was performed: in polδ, 322(D)→(A) and 324(E)→(A); and in polε, 291(D)→(A) and 293(E)→(A). These mutants were a DNA polymerase δ mutant strain (AMY128-1: Pol3-01 MATα, ura3-52leu2-1 lys1-1ade2-1 his1-7 hom3-10 trp-289 canR: available from Prof. Sugino (Osaka University) and a DNA polymerase ε mutant strain (AMY2-6: pol2-4 MATα, ura3-52 leu2-1 lys1-1 ade2-6 his1-7 hom3-10 try1-289 canR; available from Prof. Sugino (Osaka University). It will be understood that equivalents of such strains can be produced by those skilled in the art using site directed mutagenesis to introduce mutations, such as 322(D)→(A) and 324(E)→(A) in polδ; and 291(D)→(A) and 293(E)→(A) in polε.

(Method of Producing Drug Resistant Strains)

The above-described three strains were plated on agar plates containing complete medium (YPD mediums 10 g of Yeast Extract (Difco), 20 g of BactoPepton (Difco), and 20 g of Glucose (Wako)). 5 single colonies were randomly collected for each strain. The strain was inoculated into 3 ml of YPD liquid medium, followed by shaking culture at 30° C. to a final concentration of about 1×10⁶.

The strain was diluted and inoculated onto YPD plates containing 1 mg/L cycloheximide (Sigma, St. Louis, Mo., USA). As a control, the strain Was inoculated onto YPD plates containing no drug. The strain was cultured at 30° C. for 2 days. Resultant colonies were counted.

(Method of Obtaining High Temperature Resistant Strains)

The above-described 3 strains were transferred from single colonies to liquid medium, followed by acclimation culture while gradually increasing culture temperature. Acclimation culture protocol was the following:

37° C., 2 days→28° C., 1 day→38° C., 2 days→28° C., 1 day→39° C., 2 days→28° C., 1 day→40° C., 2 days→28° C., 1 day: the last culture was stored refrigerated (“acclimated culture”).

Acclimation culture was continued as follows:

37° C., 2 days→28° C., 1 day→38° C., 2 days→25° C., 1 day→39° C., 2 days→28° C., 1 day→40° C., 2 days→28° C., 1 day→41° C., 2 days→28° C., 1 day: the last culture was stored refrigerated (“acclimated culture II”).

(Measurement for Growth Curve)

Shaking culture was carried out in complete liquid medium (YPD). Growth (i.e., cell density) was measured based on the optical density (OD) at 530 nm. The optical density was determined using a spectrophotometer (Hitachi). The normal strain and the drug resistant mutant were tested at 28° C. to obtain a growth curve while the high temperature resistant strain was tested at 38.5° C.

(Results of Drug Resistant Strains)

Among DNA polymerase δ and DNA polymerase ε mutants, cycloheximide resistant bacteria emerged during the time when the cells were grown in medium without any drug, but not among the wild type.

TABLE 1
Numbers of cycloheximide-resistant colonies

| | Number of colonies* | | | | | Mean* |
|---|---|---|---|---|---|---|
| polδ | 60 | 81 | 81 | 111 | 744 | 215 |
| polyε | 3 | 39 | 138 | 0 | 0 | 36 |
| WT | 0 | 0 | 0 | 0 | 0 | 0 |

*unit: ×10⁶

It was observed that resistant strains obtained from polδ mutants could grow in up to 10 ml/L cycloheximide.

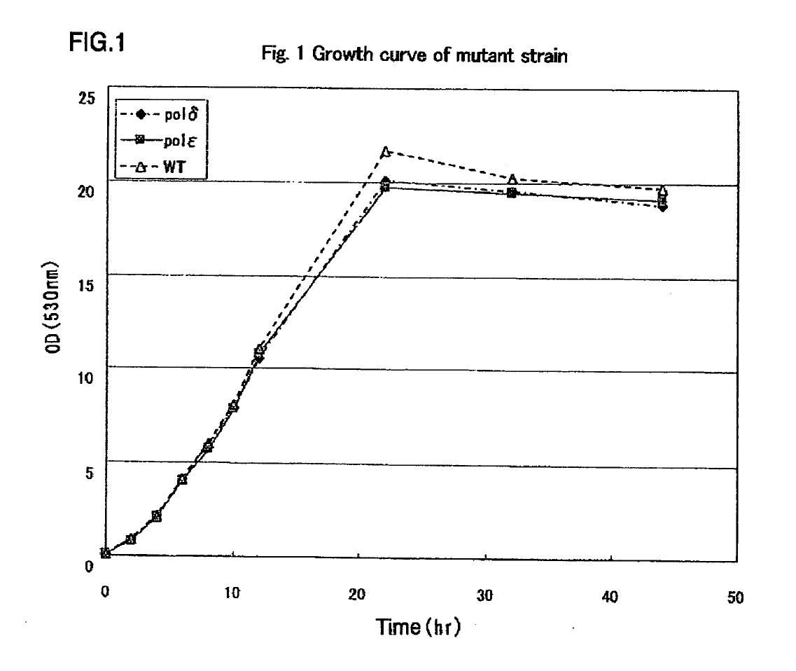

The growth characteristics of the wild type and the mutants were compared. Substantially no difference in the growth rate was found (Table 2 and FIG. 1).

TABLE 2
Growth curves of polδ and polε mutants

| Growth time | polδ | polε | WT |
|---|---|---|---|
| 0 | 0.13 | 0.13 | 0.13 |
| 2 | 0.9 | 0.6 | 0.9 |
| 4 | 2.2 | 2.1 | 2.1 |
| 6 | 4.1 | 4.0 | 4.1 |
| 8 | 5.9 | 5.7 | 6.0 |
| 10 | 7.9 | 7.8 | 8.1 |
| 12 | 10.5 | 10.8 | 11.1 |
| 22 | 20.1 | 19.8 | 21.7 |
| 32 | 19.6 | 19.5 | 20.3 |
| 44 | 18.9 | 19.2 | 19.8 |

(hr)
OD: 530 nm

(Results of High Temperature Resistant Strains)

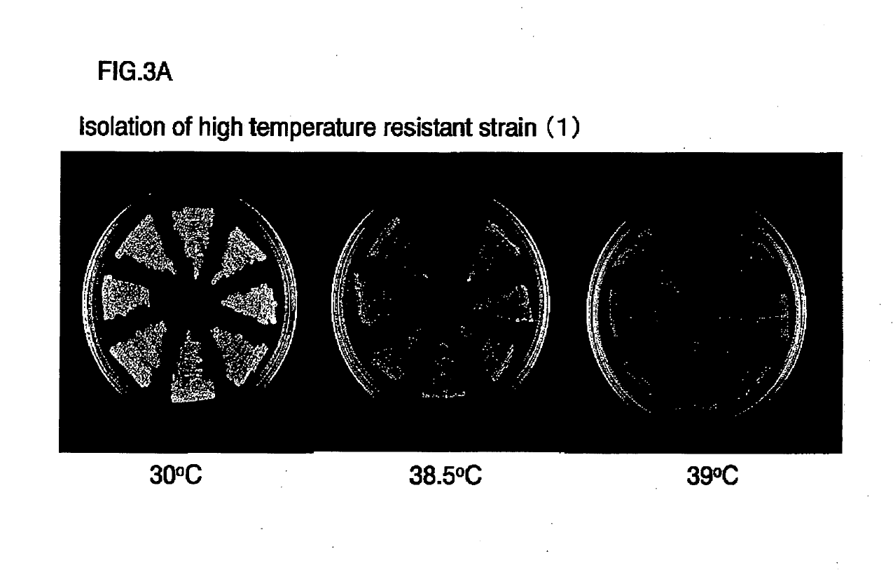

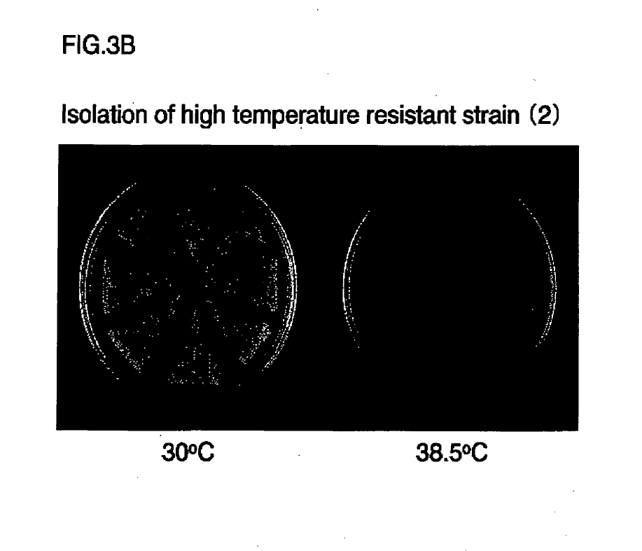

The acclimated culture was cultured for two days at 40° C. and was then inoculated onto agar plates, followed by culture at 38.5° C. Although the parent strains could not grow at high temperature, the mutants were confirmed to be able to grow at high temperature (FIGS. 3A and 3B (photographs)).

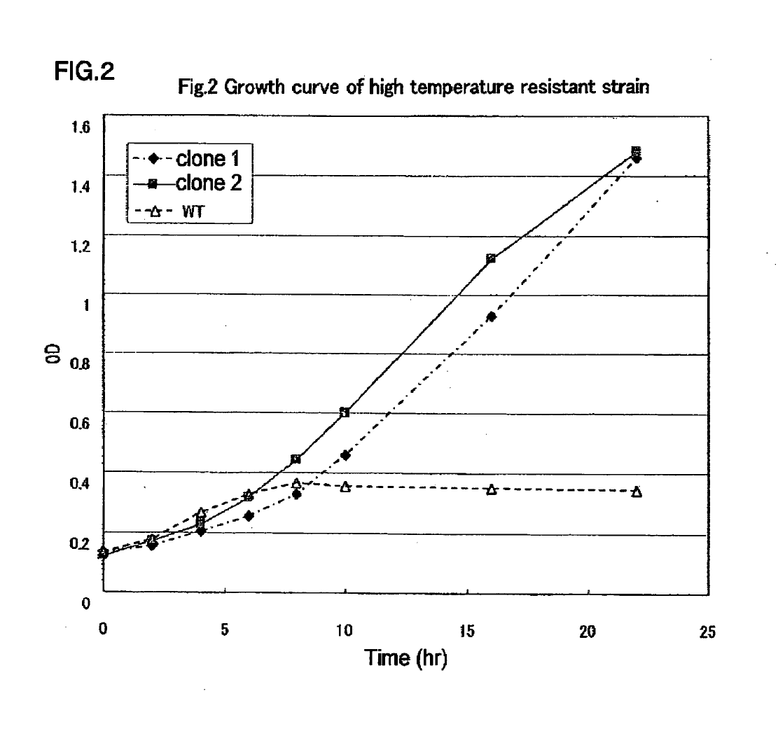

The growth characteristics of the wild type strains and the mutants under high temperature conditions were compared. It was confirmed that the growth of the wild type strains had ceased (Table 3 and FIG. 2).

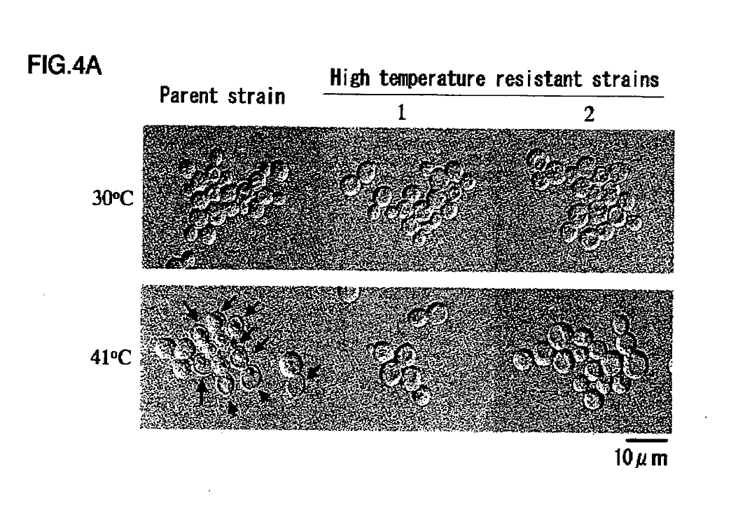

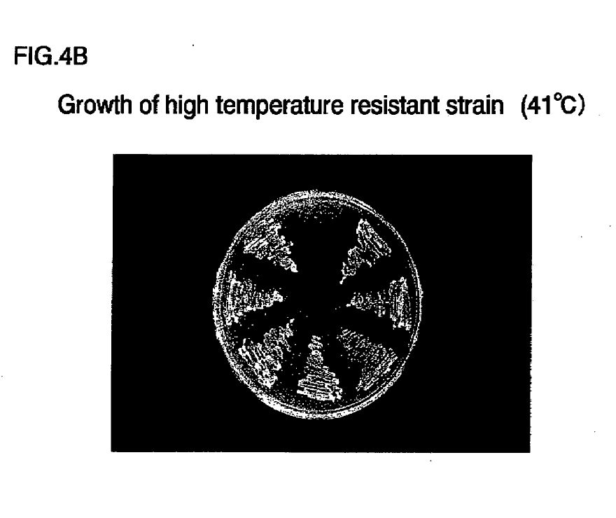

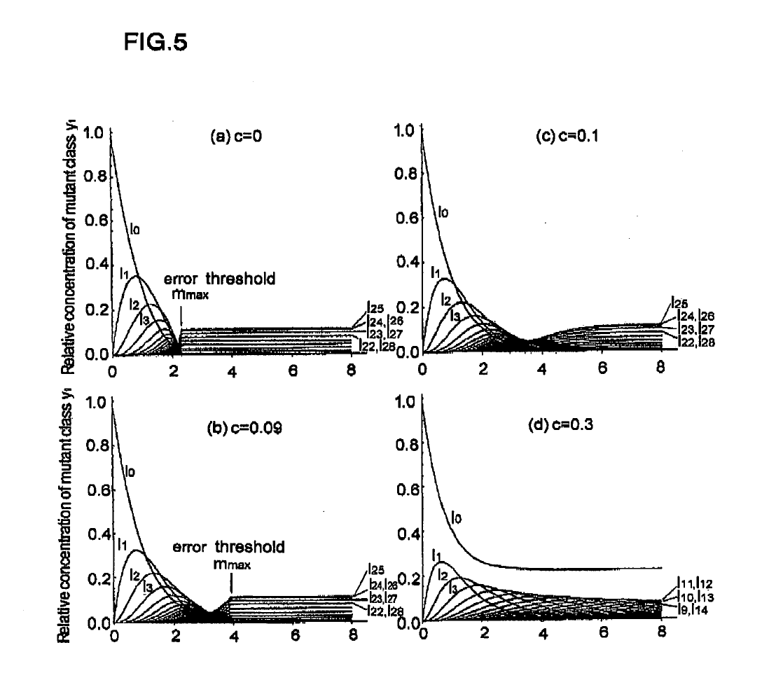

Further, the acclimated culture was continued at 41° C. As a result, it was found that mutants capable of growing at 41° C. were generated (FIGS. 4A and 4B).

TABLE 3
Growth curves of high-temperature resistant strains

| Growth time | Clone 1 | Clone 2 | WT |
|---|---|---|---|
| 0 | 0.131 | 0.125 | 0.134 |
| 2 | 0.154 | 0.174 | 0.177 |
| 4 | 0.203 | 0.227 | 0.264 |
| 6 | 0.258 | 0.314 | 0.327 |
| 8 | 0.327 | 0.447 | 0.365 |
| 10 | 0.462 | 0.6 | 0.358 |
| 12 | 0.93 | 1.12 | 0.352 |
| 22 | 1.463 | 1.486 | 0.346 |

(hr)
OD: 530 nm

Clone 1: Resistant strain derived from polδ
Clone 2: Resistant strain derived from polε

Yeast has a gene replication mechanism different from that of gram-negative bacteria, such as E. coli. Therefore, it had been unclear as to whether or not the error-prone frequency of yeast can be regulated without influencing the survival of the organism by regulating the conversion rate of a hereditary trait according to the present invention.

In Example 1, it was demonstrated that the error-prone frequency of yeast, i.e., a eukaryotic organism, can be regulated without influencing the survival of the organism by regulating the conversion rate of a hereditary trait.

### Example 2

### Mutation Introduction Using Plasmids

In Example 2, it was demonstrated that the conversion rate of a hereditary trait of eukaryotic organisms can be regulated using plasmid vectors (“disparity mutagenesis plasmid”.

The proofreading function was regulated by introducing mutations into the proofreading functions of DNA polymerase δ and DNA polymerase ε similar to Example 1 (Alan Morrison & Akio Sugino, Mol. Gen. Genet. (1994) 242: 289-296).

Plasmid vectors capable of expressing mutant DNA polymerase (pol) δ or DNA polymerase ε were produced. Yeast cells were transformed by transfection with the vector to produce mutant cells. The mutants were cultured in plate medium containing a drug, such as cycloheximide or the like. Emerging drug resistant colonies were counted.

(Materials)

In Example 2, yeast (Saccharomyces cerevisiae) was used as an organism of interest. As a normal strain, AMY52-3D:MATα, ura3-52 leu2-1 ade2-1 his1-7hom3-10 trp1-289 canR (ATCC, supra) was used. The error-prone frequency of the yeast was regulated by introducing mutant DNA polymerase δ or ε into the wild type normal strain.

Sequences encoding mutant DNA polymerase δ or ε were produced using a DNA polymerase δ mutant strain (AMY128-1:

Pol3-01 MATα, ura3-52 leu2-1 lys1 ade2-6 his1-7 hom3-10 trp1-289 canR) or a DNA polymerase ε mutant strain (AMY2-6: pol2-4 MATα, ura3-52 leu2-1 lys1-1 ade2-6 his1-7 hom3-10 try1-289 canR)) as used in Example 1.

The plasmid vector contained a promoter Gal and nucleic acid sequences (SEQ ID Nos. 33 and 35) encoding mutant DNA polymerase δ and ε, respectively. The nucleic acid sequences wore operatively linked to the promoter.

(Methods)

(Production of Vectors)

Molecular biological techniques used herein are described in, for example, Sambrook, J., et al. (supra). The pol sites of polδ and polε mutant strains (a DNA polymerase δ mutant strain (AMY128-1: Pol3-01MATα, ura3-52leu2-1lys1-1 ade2-1 his1-7 hom3-1 trp1-289 canR) and a DNA polymerase ε mutant strain (AMY2-6: pol2-4 MATα, ura3-52 leu2-1 lys1-1 ade2-6 his1-7 hom3-10 try1-289 canR)) were amplified by PCR, and polδ and polε wore recovered. Primers used for recovery of pol altos have the following sequences:

polδ (forward):
SEQ ID NO. 37: 5′-CCCGAGCTCATGAGTGAAAAAAGATCCCTT-′3 (δ);
pol3 (reverse):
SEQ ID NO. 38: 5′-CCCGCGGCCGCTTACCATTTGCTTAATTGT-′3 (δ);
polε (forward):
SEQ ID NO. 39: 5′-CCCGAGCTCATGATGTTTGGCAAGAAAAAA-′3 (ε); and
pol2 (reverse):
SEQ ID NO. 40: 5′-CCCGCGGCCGCTCATATGGTCAAATCAGCA-′3 (Ε).

The PCR products were incorporated into vectors having a GAL promoter.

(Transformation)

The normal yeast strain was transfected with the plasmid vector using a potassium phosphate method.

(Mutation Introduction)

The transformed yeast was cultured in liquid medium containing galactose at 28° C. for 48 to 72 hours while shaking.

(Confirmation of Drug Resistance)

The cells were cultured in plate medium containing cycloheximide (supplemented with galactose) at 28° C. for 24 hours. Colonies grown were counted.

(Results)

Among DNA polymerase δ and DNA polymerase ε mutants, cycloheximide resistant bacteria emerged during the time when the cells were grown in medium without any drug, but not among the wild type.

### Example 3

### Production of Mutant Organisms Including Mouse and the Like as Animals)

In Example 3, mice (animals) were used as representative eukaryotic organisms to produce disparity mutant organisms.

Mice having a replication complex having heterogeneous DNA replication proofreading abilities were produced using gene targeting techniques.

The replication proofreading function was regulated by regulating the proofreading function of a DNA polymerase δ (SEQ ID NO. 55 (nucleic acid sequence) and 56 (amino acid sequence)) and/or a DNA polymerase ε (SEQ ID NO. 57 (nucleic acid sequence) and 58 (amino acid sequence)). Mutation was performed as follows: in polδ, 315(D)→(A), 317(E)→(A); and in polε, 275(D)→(A), 277(E)→(A).

(Gene Targeting Techniques)

Gene targeting techniques are described in, for example, Yagi T. et al., Proc. Natl. Acad. Sci. USA, 87: 9918-9922, 1990; “Gintagettingu no Saishingijyutsu [Up-to-date Gene Targeting Technology]”, Takeshi Yagi, ed., Special issue, Jikken Igaku [Experimental Medicine], 2000, 4. Homologous recombinant mouse ES cells were produced using targeting vectors having mutant pol.

The recombinant ES cell was introduced into a mouse early embryo to form a blastocyst. The blastocyst was implanted into pseudopregnant mice to produce chimeric mice.

The chimeric mice wore crossbred. Mice having a germ cell in which a mutation had boon introduced were selected. Crossbreeding was continued until mice having homologous mutations were obtained.

In Example 3, a trait of interest was selected as a measure of the onset of cancer.

(Protocol)

(1. Preparation of ES Cells)

Mouse ES cells prepared from a cell mass in an embryo (available from the Center for Animal Resources and Development, Kumamoto University, Kumamoto, Japan) were cultured using feeder cells (mouse fetal fibroblasts; available from Prof. Yagi, Osaka University) in Dulbecco's Modified Eagle Medium (DMEM) supplemented with 20 to 30% bovine fetus serum at 37° C. In 5% CO₂.

The feeder cells were prepared using techniques described in, for example, “Gintagettingu no Saishingijyutsu [Up-to-date Gene Targeting Technology]”, Takeshi Yagi, ed., Special issue, Jikken Igaku [Experimental Medicine], 2000, 4. The feeder cells were obtained from primary culture of mouse fetal fibroblasts.

(2. Homologous Recombination of Pol Genes Using Targeting Vectors)

Targeting vectors were prepared by a positive/negative method (Evans, M. J., Kaufman, M. H., Nature, 292, 154-156 (1951)) so as to efficiently obtain homologous recombinant ES cell (Capecchi, M., R., Science 244: 1288-1292 (1989)).

Preparation of targeting vectors at targeting vectors were prepared by techniques described in, for example, Molecular Cloning, 2nd edition, Sambrook, J., et al, supra, and Ausubel, F. M., Current Protocols in Molecular Biology, Green Publishing Associates and Wiley-Interscience, NY, 1987, supra.

In the targeting vector, mutation polδ and/or pol ε genes were inserted between a positive gene and a negative gene. Neomycin resistant gene was used as the positive gene while diphtheria toxin was used as the negative gene.

For Pol mutations, one-base mutation was introduced into the proofreading activity sites (SEQ ID Nos. 55 and 56 (δ): SEQ ID Nos. 57 and 58 (ε)) of both polδ and polε to delete proofreading activity: in polδ, 315(D)→(A), 317(E)→(A); and in polε, 275(D)→(A), 277(E)→(A) (Morrison A. & Sugino A., Mol. Gen. Genet. 242: 289-296, Z994; Goldsby R. E., et al., Proc. Natl. Acad. Sci. USA, 99: 15560-15565, 2002).

(3. Introduction of Vectors into Es Cells)

The vector was introduced into ES cells by electroporation. Culture was performed using DMEM medium (Flow Laboratory) containing G418 (Sigma, St. Louis, Mo., USA).

(4. Recovery of Recombinant ES Cells)

After culture in the presence of G418, emerging colonies were transferred to plates (DMEM medium: Flow Laboratory).

(5. Confirmation of Homologous Recombinants)

Genomic DNA was extracted from the ES cells. Whether or not mutant pol was successfully introduced into the ES cells was determined by Southern blotting and/or PCR.

(6. Preparation of Chimeric Mice—Introduction of Recombinant ES Cells into Embryos)

The above-described recombinant cells are introduced into blastocysts by a microinjection method. As the blastocysts, host mouse embryos different from the ES cells are selected by a common method described in, for example, “Gintagettingu no Saishingijyutsu [Up-to-date Gene Targeting Technology]”, Takeshi Yagi, ed., Special issue, Jikken Igaku [Experimental Medicine], 2000, 4.

(7. Production of Chimeric Mice—Implantation of Embryos into Pseudopregnant Mice)

When the ES cell is derived from a 129-line mouse, the ES cell is injected into the blastocyst of C57BL/6 mice. When the ES cell is a TT-2 cell, the ES cell is injected into 8-cell stage embryos of ICR mice to produce pseudopregnant mice. The mouse embryo having the injected ES cell is implanted into the uterus or oviduct of a foster to produce chimeric mice

(8. Production of Chimeric Mice—Crossbreeding of Mice)

The chimeric mice are crossbred. Whether or not mutant pol is successfully introduced into germ cells is determined by PCR and/or DNA sequencing, and the like. Crossbreeding is continued until mice having homologous mutant pol are produced.

(Results)

From the mice prepared in Example 3, mice having cancer are selected. The mice naturally produce cancer at a rate significantly higher than that of conventional techniques. The modified cells have substantially the same growth rate as that of naturally-occurring cells, however, the mutation rate of the modified cell is two or more per generation, which is significantly different from that of conventional mutations.

(Other Traits)

Similarly, screening is performed with respect to diabetes, hypertension, arteriosclerosis, obesity, dementia, neurological disorders, or the like. The present invention can provide models, in which the onsets of these diseases were extremely expedited, but each disease was naturally generated. Therefore, the method of the present invention can be applied to animals.

(Other Animals)

Next, similar experiments were carried out using rats as models. Rat models of cancer can be rapidly prepared by introducing mutations into pol δ (in an amino acid sequence as set forth in SEQ ID NO. 60, D at position 315 and E at position 317 are substituted with alanine).

### Example 4

### Production of Mutant Organisms Using Other Procedures)

Next, another mouse model was used to determine whether or not a mutant organism can be produced. The procedure used is described below.

(Materials and Methods)

Preparation of cDNA of Pold1

mRNA was extracted from the testes of four-week old neonatal C57BL/6 mice (Charles River Japan) using-TRIzol Reagent (Invitrogen). Total cDNA of mouse testis was produced by reverse transcription of the extracted mRNA using SuperScript III (Invitrogen) and an Oligo-dT primer. With the total cDNA, the cDNA fragment of the Pold1 gene was amplified by the PCR using the 5′-terminal primer, SpeI-5′ Pold1 (GACTAGTGGCTATCTTGTGGCGGGAA) (SEQ ID NO.: 67) and the 3′-terminal primer, EcoRI-3′ Pold1 (GGAATTCCTTGTCCCGTGTCAGGTCA) (SEQ ID NO.: 68) of the Pold1 gene (SEQ ID NO.: 86(nucleic acid sequence) and SEQ ID NO.: 87 (amino acid sequence)), which were designed to contain the Kozak sequence. In this manner, cDNA of wild-type Pold1 was obtained. Mutation (D400A) was introduced into the cDNA to delete the 3′-5′ exonuclease activity from the Pold1 gene (SEQ ID NO.: 88 (nucleic acid sequence) and SEQ ID NO.: 89 (amino acid sequence)). To achieve this, a mutation introducing primer sequence (CAGAACTTTGCCCTCCCATACCTC) (SEQ ID NO.: 69) and a primer complementary thereto were subjected to PCR ligation to produce cDNA of a Pold1 mutant. The full-length sequence of cDNA (SEQ ID NO.: 70) was read with an ABI3100 Sequencer (Applied Biosystems, CA. USA) and was compared to a database to find the same sequence. The cDNA was used for all experiments. PCR for preparing the wild-type and mutant-type Pold1 cDNAs was performed using a KOD DNA polymerase (TOYOBO, Osaka, Japan).

Cloning of Promoter Sequence

A mPGK2 promoter fragment (SEQ ID NO.: 94) of mPGK2:455-bp was cloned by utilizing a 5′ mPGK2-sacII primer (TCCCCGCGGCTGCAGAGGATTTTCCACAG) (SEQ ID NO.: 71) and a 3′ mPGK2-SpeI primer (GGACTAGTATGGTATGCACAACAGCCTC) (SEQ ID NO.: 72) of the genomic DNA of C57BL/6 mouse. The PCR was performed using KOD DNA polymerase (TOYOBO, Osaka, Japan).

A DNA fragment (SEQ ID NO.: 95), which is an upstream sequence of Fth117:5725-bp was cloned by utilizing a 5′ Fth117-sacII primer (TCCCCGCGGAGTGGTTGTGGGAGACTTAC) (SEQ ID NO.: 73) and 3′ Fth117-SpeI primer (GGACTAGTCAGTCCCACAGTCCCAAAGT) (SEQ ID NO.: 74). PCR was performed using a LA Taq polymerase (TAKARA) and a GC buffer (provided by the manufacturer).

Production of Transgenic Mice

Vector DNA (2 ng/μl) prepared for production of transgenic mice was injected into the pronuclei of fertilized eggs of C57BL/6 mice using a micromanipulator. Among the fertilized eggs into which the gone was introduced, embryos in the 2-cell stage (the following day) were transplanted into the oviducts of pseudopregnant female ICR mice, thereby producing transgenic mice.

Confirmation of the Presence or Absence of Transgene

The tails of mice were cut into small pieces, which were in turn placed into a solubilizing buffer (50 mM Tris-HCl, 10 mM EDTA, 200 mM NaCl, 1% SDS) containing proteinase K (Nacali Tesque) and incubated at 55° C. overnight. Thereafter, the genomic DNA of the mice was prepared by performing twice phenol/chloroform extraction and ethanol precipitation. For the genomic DNA of each mouse, the presence or absence of a transgene was determined by PCR for transgenic mouse #1 using a Cre-F primer (CTGAGAGTGATGAGGTTC) (SEQ ID NO.: 75) and a Cre-R primer (CTAATCGCCATCTTCCAGCAG) (SEQ ID NO.: 76) and for transgenic mouse #2 using a Neo-F primer (GCTCGACGTTGTCACTGAAG) (SEQ ID NO.: 77) and a Neo-R primer (CCAACGCTATGTCCTGATAG) (SEQ ID NO.: 78). PCR was performed using an Ex-Taq polymerase (TAKARA, Kyoto, Japan).

Immunostaining

F₀-generation transgenic mice of mPGK2 (postnatal 14 weeks old) and Fth117 (postnatal 13 weeks old) were used for experiments. The mice were anesthetized with Nembutal (50 mg/ml, Dainippon Pharmaceutical) and abdominal incisions were performed. Initially, one of the two epididymes was cut off. Thereafter, the mice were perfusion fixed with 4% paraformaldehyde. The two epididymes were extracted and immersed in 4% paraformaldehyde for 4 hours. The epididymes were briefly washed with PBS (NaCl 8 g, Na₂HPO₄ 1.15 g, KCl 0.2 g, and KH₂PO₄ 0.2 g in water; final volume: 1 L), and were immersed in 20% sucrose phosphate buffer (0.1 M phosphate (sodium) buffer (pH 7.3), 20% sucrose) at 4° C. overnight. Thereafter, the tissue was immersed in an OCT compound (Tissue-Tek, Sakura Fineteck Japan) and immediately cooled. The tissue was out into 5-μm thick slices using a cryostat. The slices Were incubated in PBS containing 20% Blocking one (Nacali Tesque) and 0.05% Tween 20. Thereafter, the slice was incubated with a mouse anti-Cre recombinase monoclonal antibody (MAB3120, Chemicon) 4000-fold diluted. As a secondary antibody, a biotinylated anti-mouse IgG antibody (Vector Laboratories inc.) was used. Color development was performed using 3,3-diaminobenzidine (DAB) (Dojindo Laboratories) and peroxidase (Nacali Tesque). After color development with DAB, comparative staining was performed using methyl green (Merck).

Artificial Insemination

Pregnant mare's serum gonadotrophin (PMSG) (CALBIOCHEM) was intraperitoneally injected into female C57BL/6 mice (Charles River Japan) (5 IU per mouse). 46 to 48 hours later, human chorionic gonadotropin (hCG) (Teikoku Hormone MFG.) was intraperitoneally injected into the mice (5 IU per mouse) similarly to PMSG. 12 hours later, the mice were euthanized by cervical dislocation, and an egg mass was extracted. The extracted egg mass was incubated in M2 medium containing 0.3 mg/ml hyaluronidase (SIGMA) at 37° C. fof 10 minutes, and unfertilized eggs were collected. Epididymes were extracted from the transgenic mice of mPGK2 and Fth117used for immunostaining before perfusion fixation. Sperm was collected from the tail portion of the epididymes. The sperm collected was placed and activated in TYH medium (in vitro fertilization medium) at 37° C. In a 5% CO₂ incubator. Thereafter, the sperm was added to TYH medium containing the unfertilized eggs. The mixture was allowed to stand in the same 5% CO₂ incubator for 6 hours. Thereafter, the eggs were washed and transferred to embryo culture medium WM, followed by incubation at 37° C. In a 5% CO₂ incubator overnight. The following day, only eggs in the 2-cell stage were transplanted into the oviducts of pseudopregnant ICR mice.

Confirmation of Gene Expression Using mRNA

TRIzol Reagent (Invitrogen) was used to extract mRNA from the tail of transgenic mouse #2. cDNA was obtained by reverse transcription of the extracted mRNA using SuperScript III (Invitrogen) and an Oligo-dT primer, followed by PCR using a Neo-F primer (GCTCGACGTTGTCACTGAAG) (SEQ ID NO.: 79) and a Neo-R primer (CCAACGCTATQTCCTGATAG) (SEQ ID NO.: 80). Thereby, the presence or absence of mRNA expression was determined. PCR was performed using an Ex-Tag polymerase (TAKARA).

Analysis of Recombination Efficiency Using Cre Recombinase

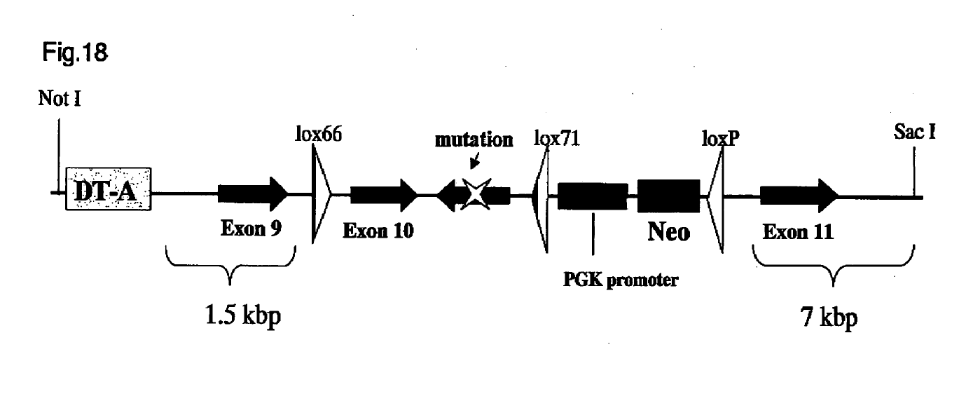

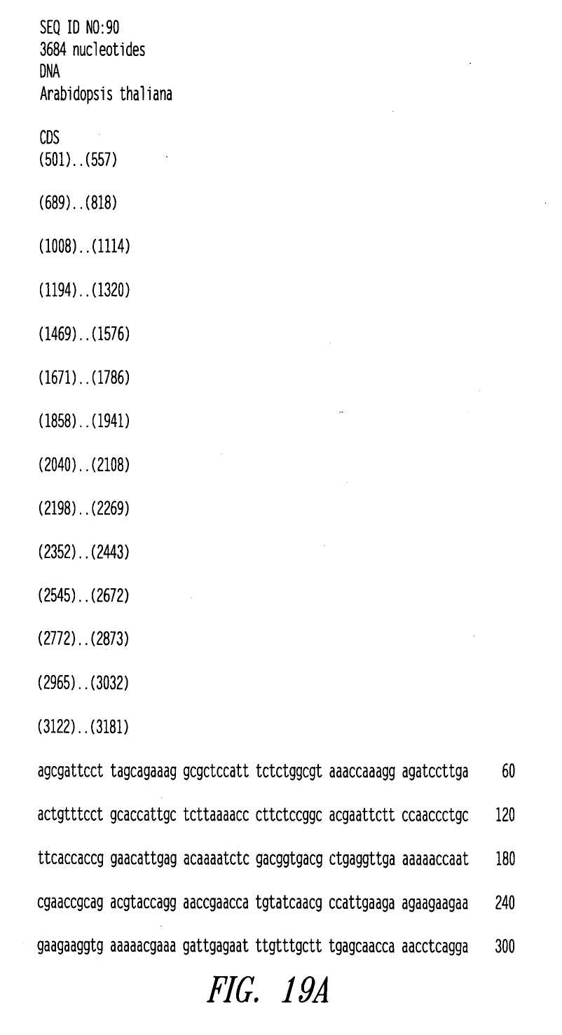

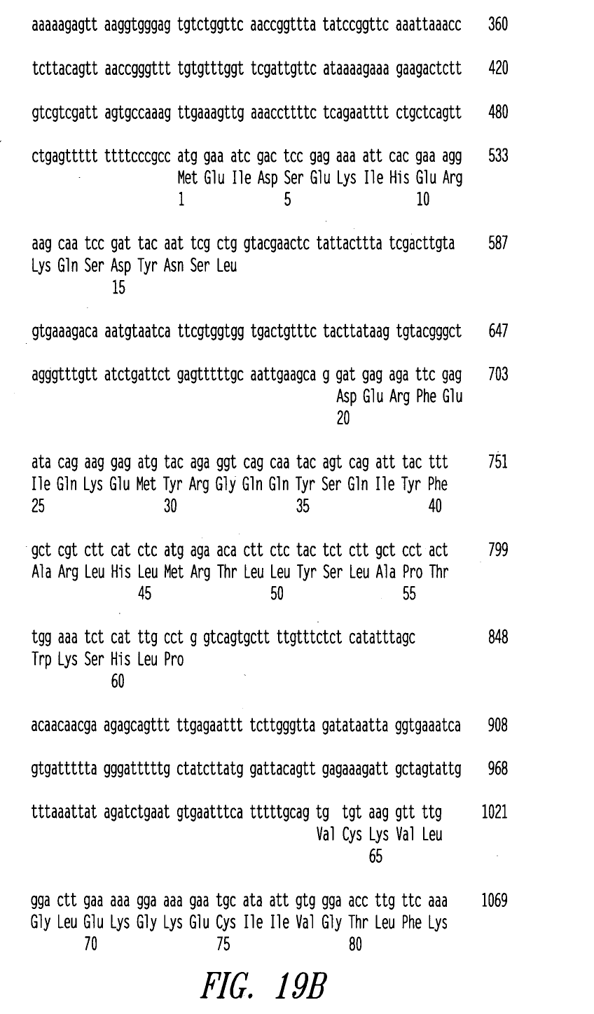

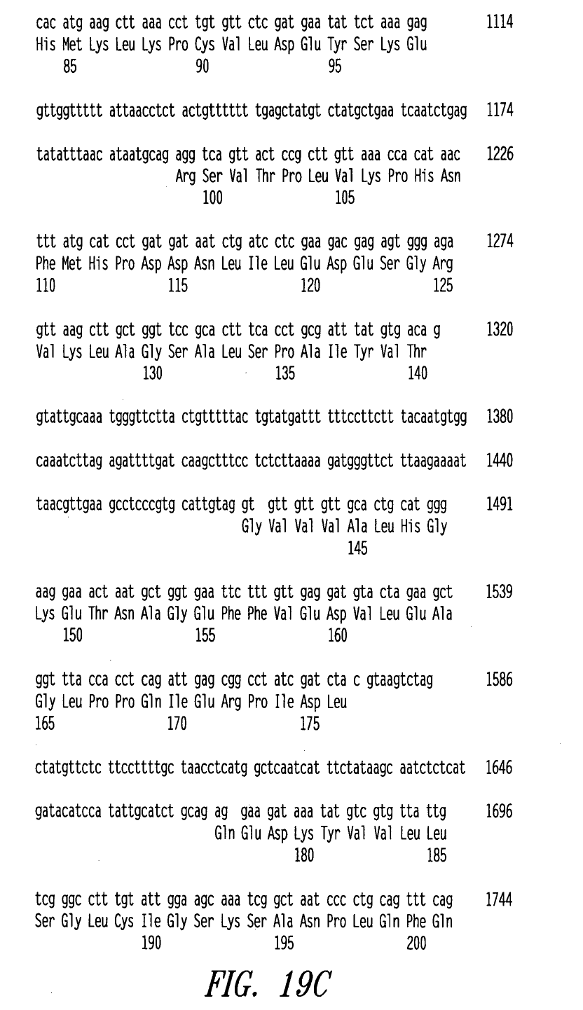

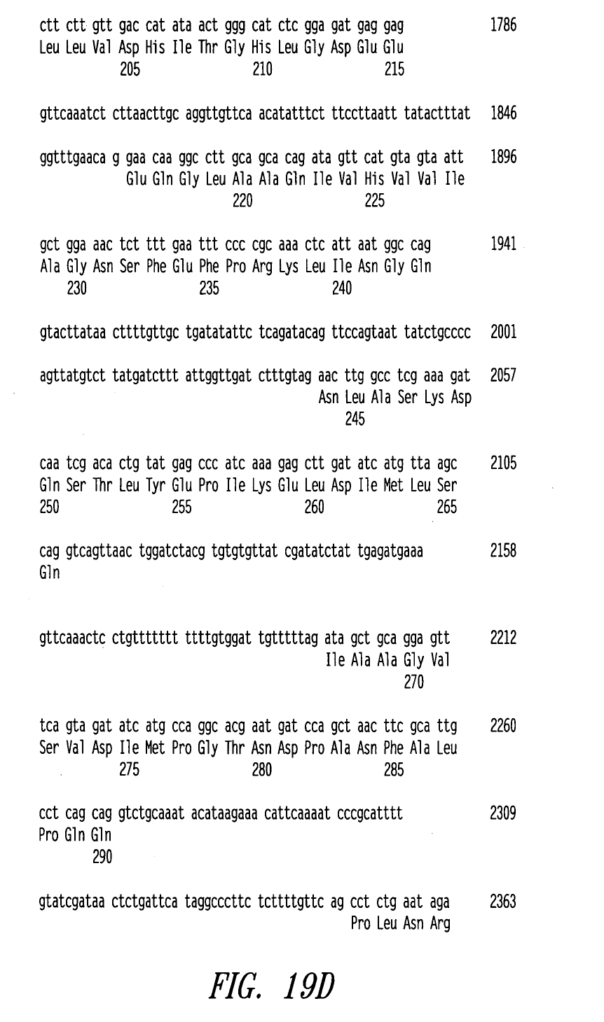

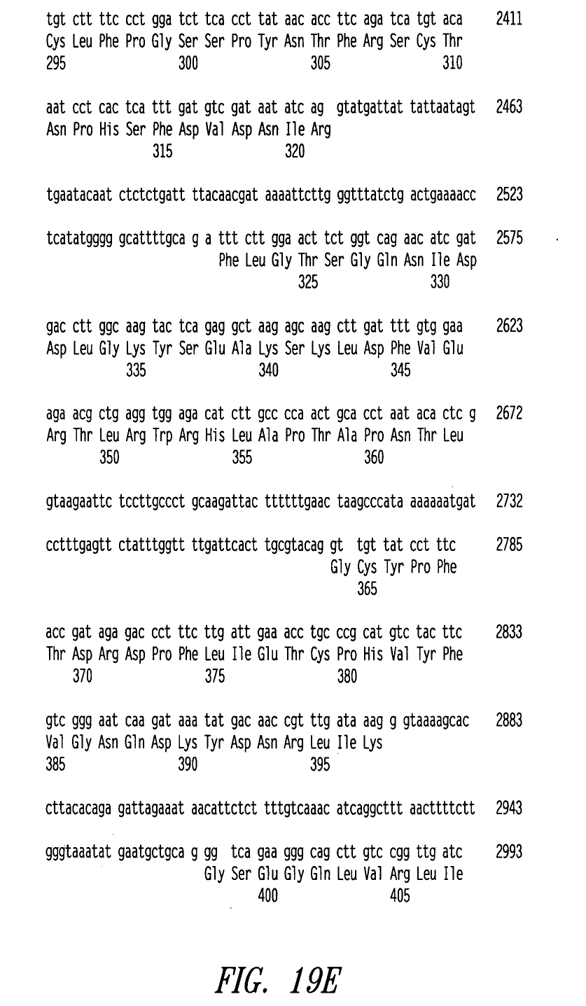

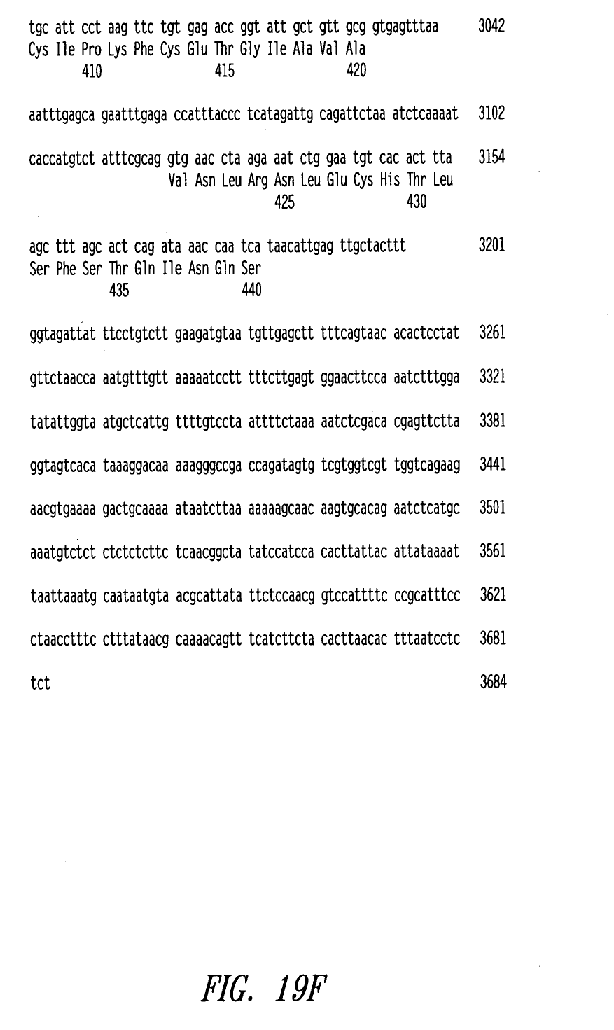

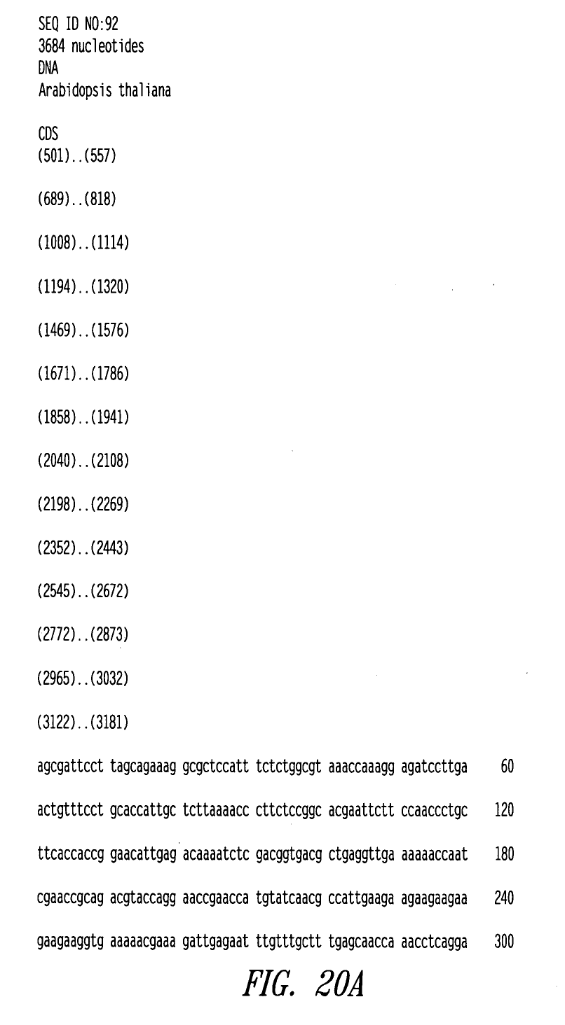

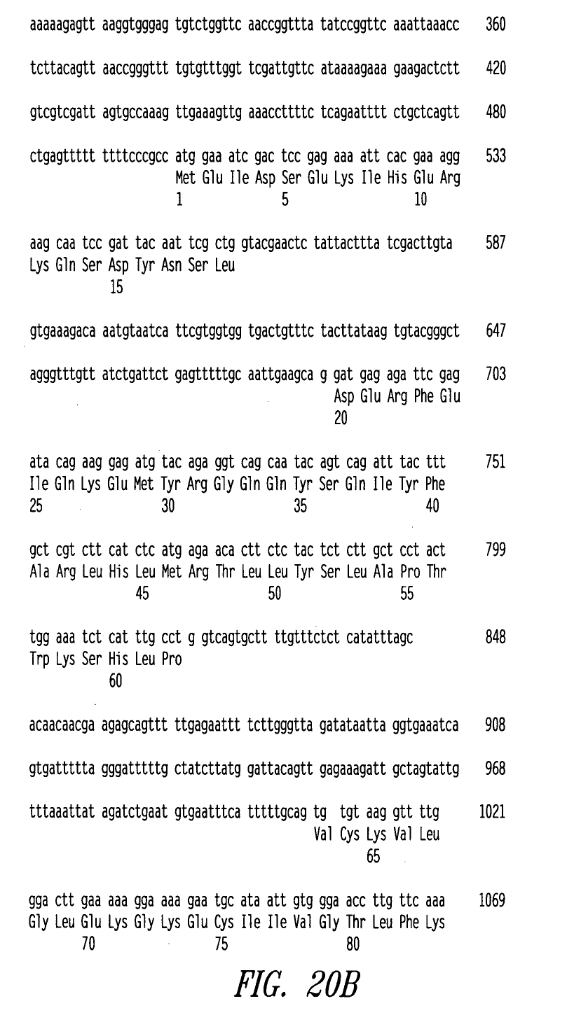

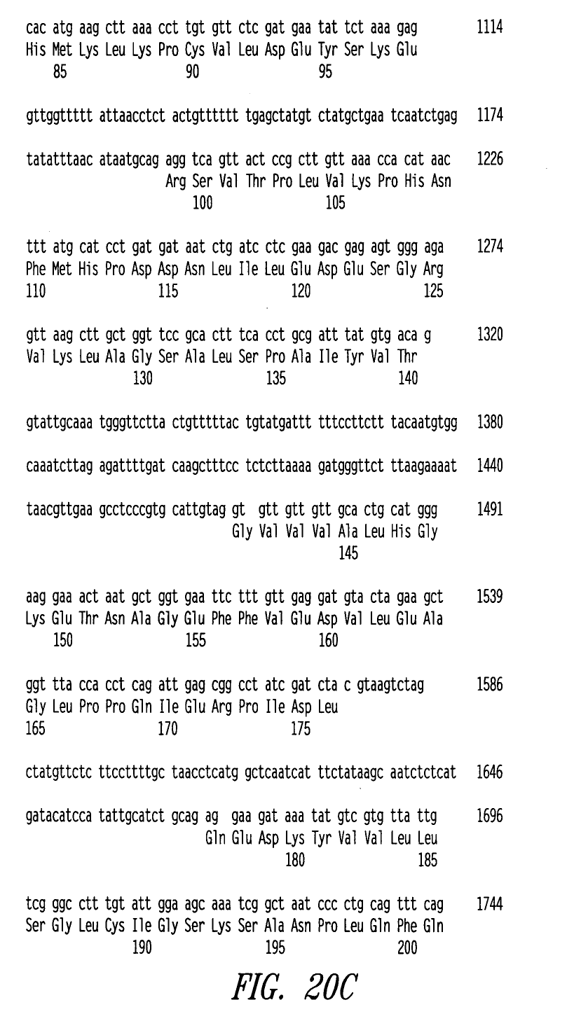

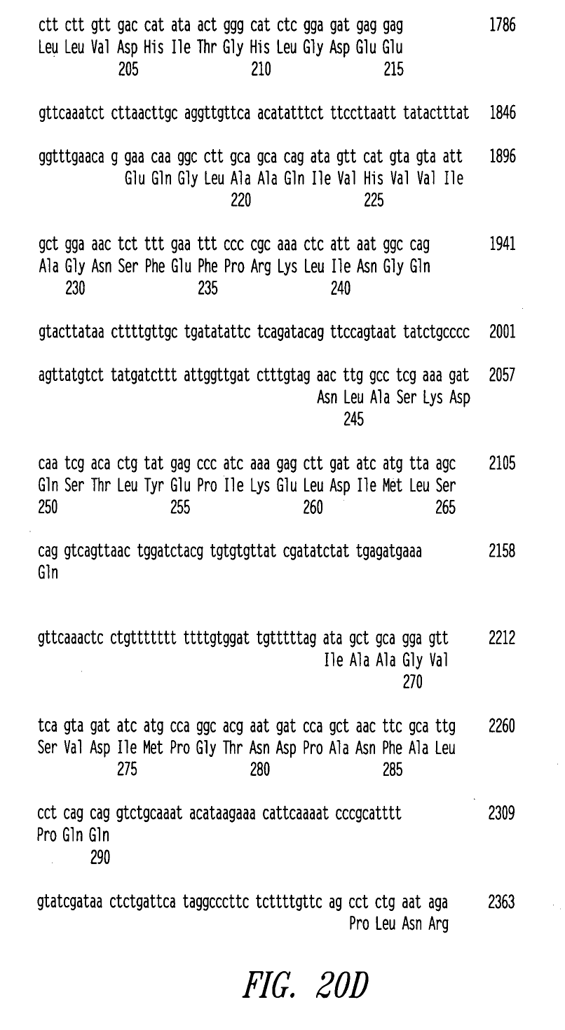

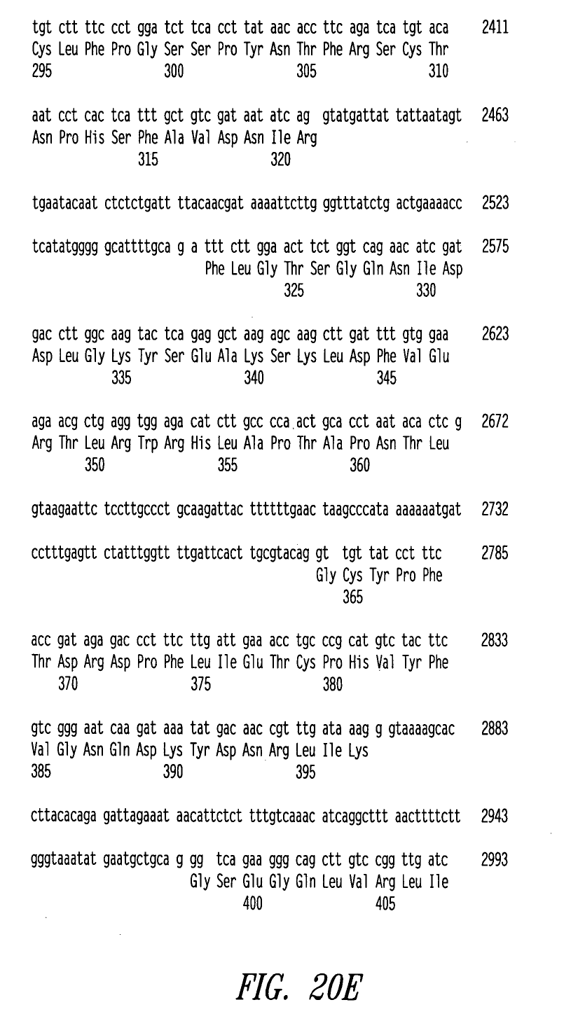

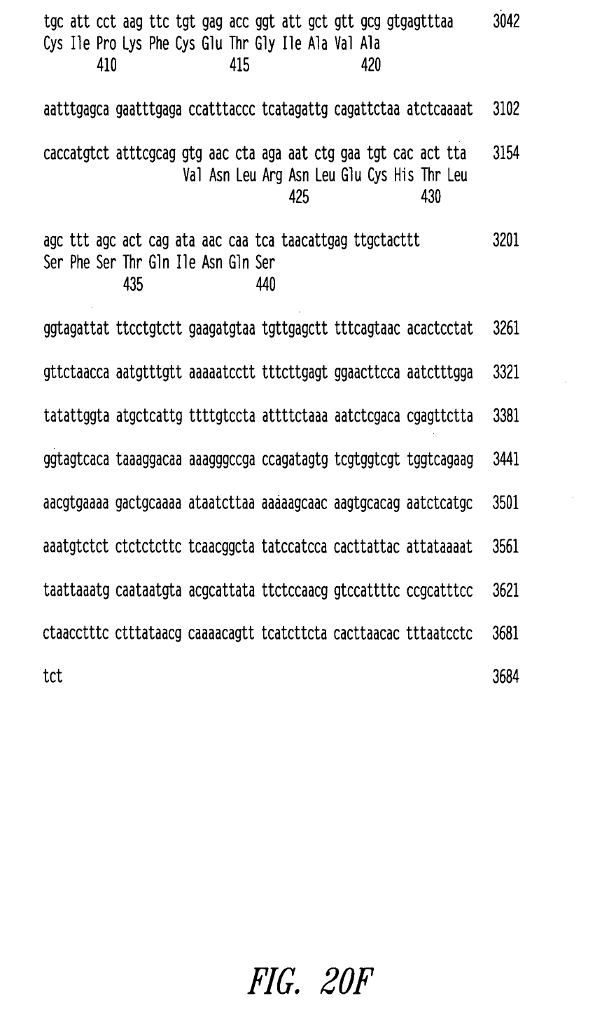

As a targeting vector (FIG. 18), the sequence of a region between lox66 and lox71, was produced on pBluescript II. 200 ng of the vectors produced were reacted with a Cre recombinase (BD Biosciences) in Cre reaction Buffer (BD Biosciences) in the presence of 1 mg/ml BSA at room temperature for 2 hours. After reaction, incubation was performed at 70° C. for 5 minutes to inactivate the Cre recombinase. The reaction solution was subjected to heat shook to transform the cells onto competent cells. The transformed cells were plated onto LB-Amp plates (1.5% agar powder (Nacali Tesque) was added to LB medium, followed by autoclaving, and then was supplemented with 100 μg/mL ampicillin (SIGMA)). On the following day, colonies were picked up. The colonies were cultured in LB-Amp medium, followed by extraction of plasmids. Recombination was confirmed based on the results of sequencing the plasmids using ABI sequencer 3100.

An object of producing transgenic mice is to determine whether or not the rate of evolution can be regulated by over expression of a mutant-type Pold1 specific to the spermatogenesis stage.

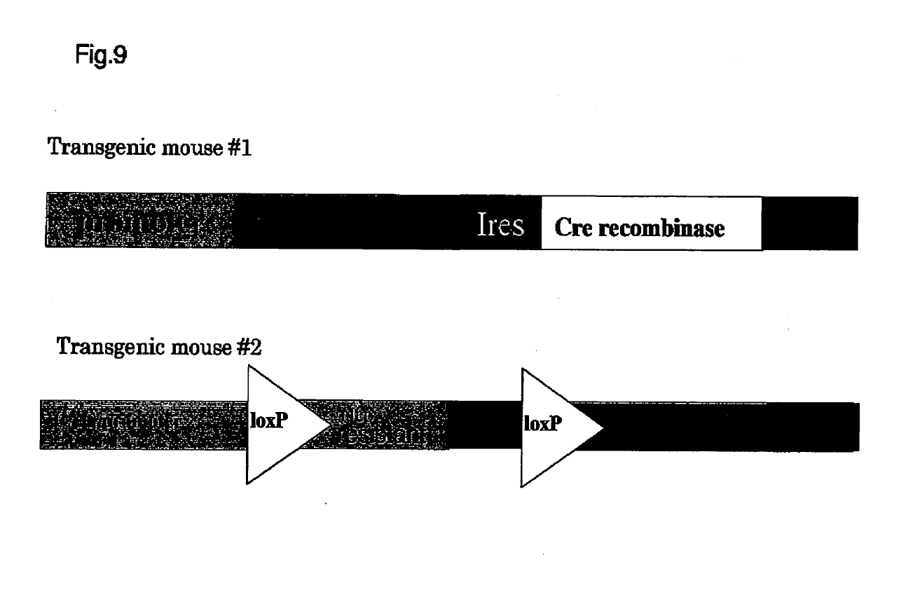

Further, it was considered that by expressing the Cre recombinase, expression specific to the spermatogenesis stage can be controlled in mice having the loxP sequence. Therefore, an attempt was made to produce two transgenic mice: transgenic mouse #1 which can express both a mutant-type Pold2 and the Cre recombinase specifically in the spermatogenesis stage, and transgenic mouse #2 which allows tissue-specific overexpression of a mutant-type Pold1 by utilizing the loxP sequence (FIG. 9).

(a) Transgenic Mouse #1

Transgenic mouse #1 elicits expression of a mutant-type Pold1 and the Cre recombinase specifically in the spermatogenesis stage. To produce such a mouse, it is important to select a promoter which elicits gene expression in the spermatogenesis stage. It has been suggested that in the testis of mice, DNA polymerase δ is expressed in the spermatogonium stage and from the primary spermatocyte stage until the first half of meiosis (Dia Kamel, et al., (1997) Biology of Reproduction, 57, 1367-1374).

Therefore, it was conceived to utilize a promoter which elicits expression in the spermatogonium stage or in the primary spermatocyte Stage. A mouse phosphoglycerate kinase 2 (mPGK2) gene promoter is often used for overexpression in primary spermatocytes (Nadia A. Higgy, et al., (1995) Dev. Genetics, 16, 190-200). The mPGK2 promoter was used as a candidate for a promoter which elicits expression specifically in the spermatogenesis stage. It was also conceived to utilize a promoter which promotes expression in the spermatogonium stage of spermatogenesis earlier than that of the mPGK2 promoter. However, substantially no promoters capable of expression specific to the spermatogonium stage or the primary spermatocyte stage have been reported. Therefore, an attempt was made to develop a novel promoter specific to the spermatogenesis stage by cloning a sequence upstream of a gene which had been said to express specifically in spermatognia by PCR. Among the genes that express specifically in spermatognia and had been found by the cDNA subtraction method (P. Jeremy Wang, et al., (2001), Nature genetics, 27, 422-426), the Ferritin heavy polypeptide-like 17 (Fth117) gene was selected, A sequence of about 5.7 kbp (SEQ ID NO.: 81) located upstream of the gene was utilized as a promoter which expresses specifically in the spermatogonium stage. The above-described two promoters specific to the spermatogenesis stage were used to produce vectors for transgenic mouse #1. FIG. 9 schematically shows a vector actually produced. A vector was produced, in which a mutant-type Pold1 gene and the Cre recombinase were linked via a sequence of IRES (internal ribosome entry site) and the genes were simultaneously expressed by a promoter which was expected to elicit expression specifically in the spermatogenesis stage.

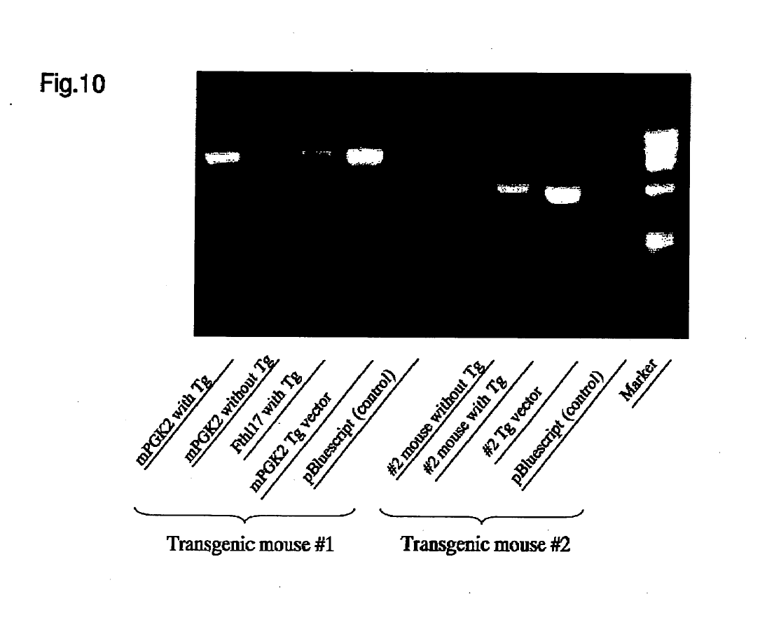

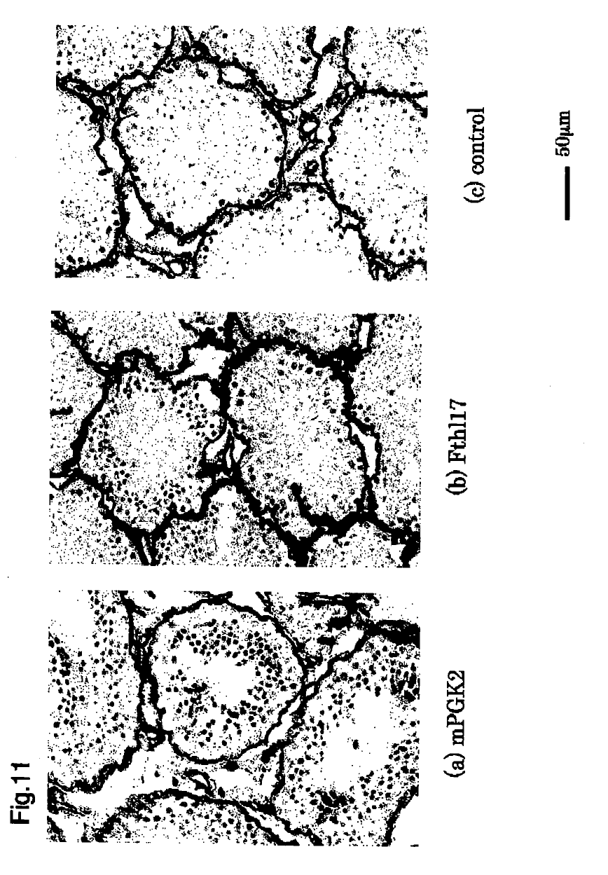

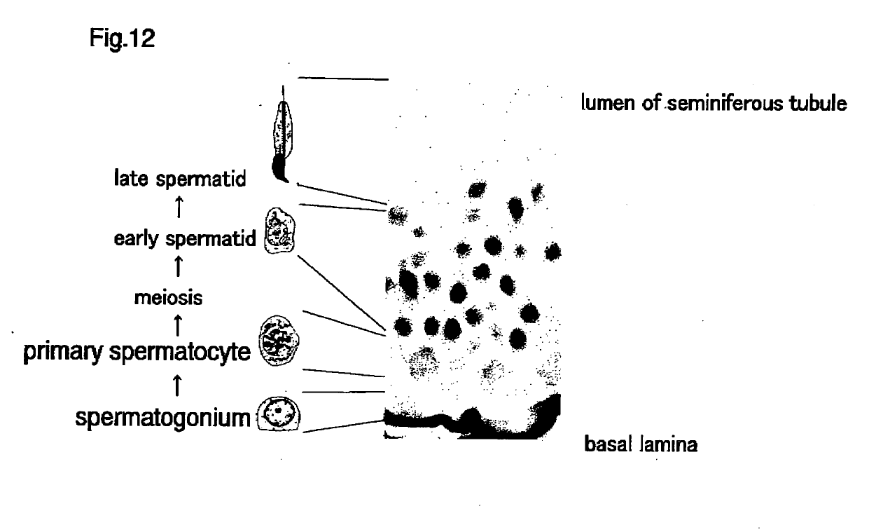

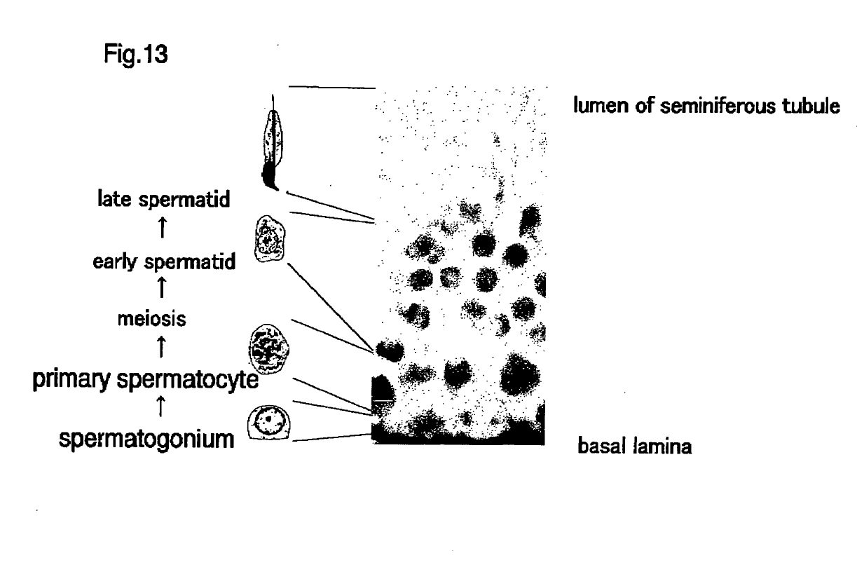

The DNA of the vector produced was microinjected into the pronuclei of fertilized eggs to produce transgenic mice. The presence or absence of the transgene in newborn mice was determined by PCR using a primer specific to the Cre recombinase (FIG. 10). As a result, there were two lines of transgenic mice for the mPGK2 promoter (in 46 neonates), while there was one line of transgenic mouse for the sequence upstream of Fth117 (in 27 neonates). The newborn transgenic mice could not be distinguished from normal mice in their appearance. In order to analyze the expression regions of the promoters, the testes of transgenic mice in the F₀ generations of mPGK2 (postnatal 14 weeks old) and Fth117 (postnatal 13 weeks old) were extracted, followed by immuno staining using a mouse anti-Cre recombinase monoclonal antibody (FIG. 11). In FIG. 11, DAB was used for color development of a secondary antibody (black brown), followed by comparative staining with methyl green for staining RNA present in cells (blue green). Also in controls, strong black blown color development was observed in the basal lamina of the seminiferous tubules. This was background since the primary antibody was of mouse. In the results of this immunostaining, the black brown color development within the seminiferous tubules indicates the expression site of the foreign Cre recombinase. According to FIG. 11, it was confirmed that the Cre recombinase was expressed in the seminiferous tubules of the testes of both the transgenic mouse using the mPGK2 promoter and the transgenic mouse using the sequence upstream of Fth117. Thus, it suggested the possibility that the 5.7-kbp region sequence upstream of Fth117 has promoter activity. In addition, the color development was weaker in the result of staining the Cre recombinase in the testis of the transgenic mouse using the sequence upstream of Fth117 than when the mPGK2 promoter was used. It is thus suggested that the sequence upstream of Fth117 has a promoter activity (expression ability) lower than that of the mPGK2 promoter. Russel et al. conducted histological analysis of the testes of mice, rats, and dogs and summarized criteria for distinguishing stages of spermatogenesis from each other (Russell L D, Ettlin R A, Hikim A P S, Cleggand E D. (1990), Histological and Histopathological Evaluation of Testis., Clearwater, Fla.: Cache River Press). According to this, further analysis was performed so as to determine at what stage of spermatogenesis the above-described two promoters were expressed in the staining images of transgenic mouse #1. In the case of the mPGK2 promoter, expression was observed mainly in the second stage of the primary spermatocyte (FIG. 12). This expression was observed in a region different from the conventionally considered region. In the case of the sequence upstream of Fth117 used as a promoter, expression was observed from the primary spermatocyte stage to the spermatogonium stage (FIG. 13). According to the results of staining, it was difficult to distinguish the expression in the spermatogonium from the background (stained basal lamina), so that the presence or absence of the expression could not be determined.

The above-described newborn F₀ generation included transgenic mice using the mPGK2 promoter (male 1, female 2) and a transgenic mouse using the sequence upstream of Fth117 (male 1). The testis of each male was used as a sample for immunostaining. The F₀ generation males used for immunostaining started mating for reproduction from the age of 9 weeks postnatal. The results of actual mating are summarized in Table 4(A).

TABLE 4(A)

| | Number of mated | Number of pregnant | Pregnancy |
|---|---|---|---|
| | females | females | rate |
| mPGK2 | 10 | 0 | 0% |
| Fthl17 | 10 | 6 | 60% |

In Table 4(A), females whose abdomen was enlarged were counted as pregnant females. In the case of the male transgenic mice using the mPGK2 promoter, although some females were confirmed to be pregnant on the day after mating, the females eventually gave birth to no newborns. When immunostaining was further performed, the transgenic mouse was anesthetized and its epididymis was extracted before perfusion fixation. Sperm obtained from the epididymis was used to try artificial insemination (Table 4(B)).

TABLE 4(B)

| | Number of | | |
|---|---|---|---|
| | unfertilized | Number of 2-cell | Number of |
| | eggs | stage | newborns |
| mPGK2 | 23 | 0 | 0 |
| Fth117 | 22 | 8 | 7 |

The sperm collected from ether of the mice invaded an unfertilized egg. No abnormality was found in any of the sperm observed. In the case of the transgenic mice using the sequence upstream of Fth117, some eggs proceeded to the 2-cell stage on the day after artificial insemination at a rate which wag lower than usual. Newborns were confirmed to be born to surrogate mothers into which the eggs had been transplanted. However, in the case of the transgenic mice using the mPGK2 promoter, there were some fertilized eggs which proceeded to the pronucleus stage after artificial insemination, but no eggs reached the 2-cell stage on the day after artificial insemination. Therefore, the possibility was suggested that the male transgenic mice using the mPGK2 promoter used for immunostaining had an abnormality in spermatogenesis. Note that no abnormality was particularly found in the females of the F₀ generation using the mPGK2 promoter, which gave birth to newborns in a manner similar to normal mice.

(b) Transgenic Mouse #2

Transgenic mouse #2 was obtained by mating with a mouse expressing the Cre recombinase in a tissue-specific manner, so that a mutant-type Pold1 was overexpressed in a tissue-specific manner. To achieve this, a vector was produced, whose sequence comprised a CAG promoter for overexpression in the whole body, a neomycin resistant gene sandwiched by two loxP sequences, and a mutant-type Pold1 linked thereto (FIG. 9). A polyA signals which indicates termination of transcription, was added to the end of the neomycin resistant gene. Therefore, the expression of the mutant-type Pold1 can be started by the tissue-specific expression of the Cre recombinase. The transgenic mouse #2 was produced as in transgenic mouse #1. With PCR using a primer specific to the neomycin resistant gene (FIG. 10), 4 lines (in 20 newborns) were confirmed to be transgenic mice. Among the four lines of transgenic mice, the F₀ generation mice of three lines exhibited growth similar to that of normal mice. Therefore, it can be said that substantially no abnormality occurred in the mice even if the conversion rate of a hereditary trait was regulated according to the present invention.

mRNA was extracted from the tails of the 3 surviving lines of transgenic mice #2, followed by RT-PCR using a primer specific to the neomycin resistant gene. As a result, the expression of the neomycin resistant gene was confirmed.

(Production of Targeting Mice)

An attempt was made to produce conditional targeting mice, in which normal Pold1 genes were replaced with a mutant-type Pold1 gene in a tissue- or time-specific manner, and the expression manner of the original DNA polymerase δ was maintained as much as possible. In the case of recombination using the Cre recombinase, if two loxP sequences are linked so that they are oriented toward each other, recombination occurs between the two loxP sequence, so that a region sandwiched by the loxP sequences can be reversed, i.e., replaced with the reversed region. However, if the two loxP sequences are only oriented toward each other, the replacement is a reversible reaction process. To cause the recombination reaction process to be irreversible, a mutation may be introduced into a portion of the loxP sequence (lox66, lox71) as described in Kimi Araki, et al., (1997), Nucleic Acids Res., 25, 868-872.

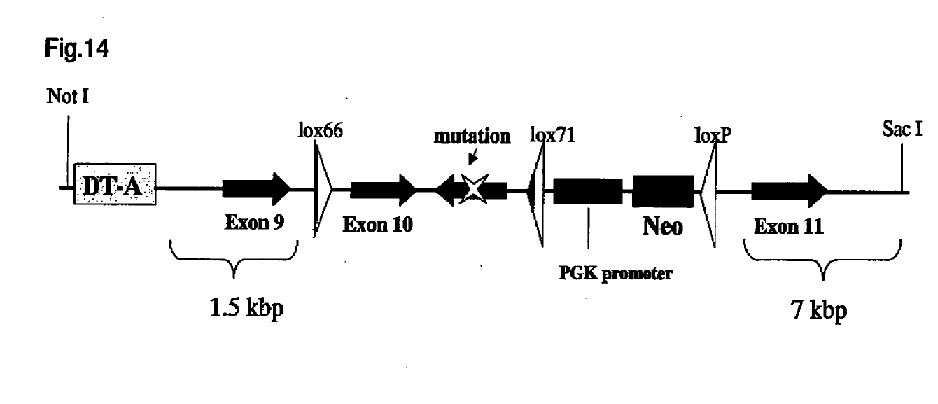

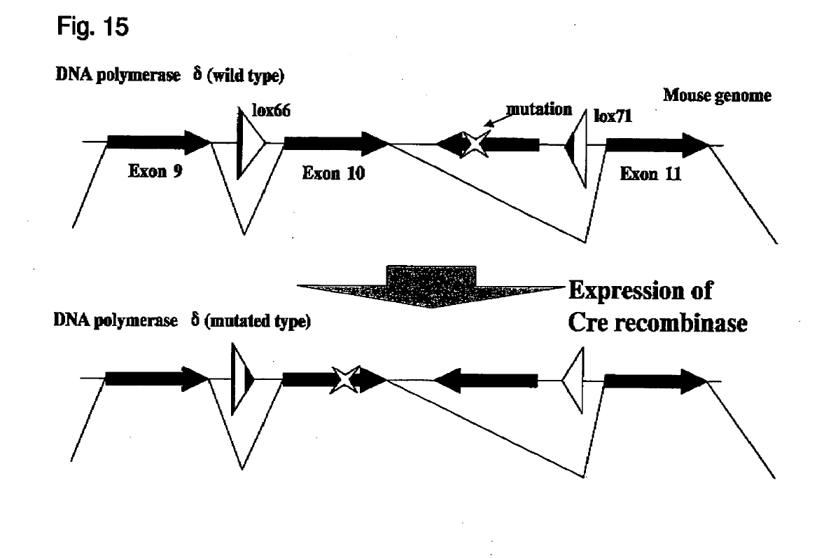

Conditional targeting mice were produced using the mutated loxP sequences. Lox66 and lox71 were provided and oriented toward each other. The sequence of normal exon 10 and a sequence complementary to a mutant-type exon 10 containing a mutation site of a mutant-type Pold1 were linked in sequence (FIG. 14). Such a vector was used to produce targeting mice. It was expected that if recombination occurs between the two lox sequences due to expression of the Cre recombinase, exon 10 used in splicing would be changed from is the normal type to the mutant-type (FIG. 15). Thereby, the normal type endogenous DNA polymerase δ would be replaced with the mutant-type due to expression of the Cre recombinase. When the targeting vector was produced, exon 10 was prepared so that the intron portions at the opposite ends thereof contained sequences essential for splicing.

However, the lox66 and lox71 sequences contained a mutation. Therefore, it was considered that the recombination efficiency due to the Cre recombinase would be lower than when the normal loxP sequence was used. In order to investigate whether or not the reaction appropriately occurred when the lox66 and lox71 sequences were oriented toward each other, the Cre recombinase itself was used to perform recombination. To achieve this, a sequence containing two axon 10 between lox66 and lox71 (referred to as a lox66-71 recombinant sequence) was produced on pBluescript II. By reacting the sequence with the Cre recombinase, recombination efficiency was investigated. As an experiment for a positive control with respect to the occurrence of a reaction, the vector sequence for transgenic mouse #2 was used. As a result, the reaction using the Cre recombinase caused recombination in 50% of the plasmids in 15 minutes and 100% in 2 hours. When the reaction was carried out for two hours with respect to the lox66-71 recombinant sequence, normal recombination was confirmed at a low frequence (⅓). Thus, it was confirmed that recombination occurred in the lox66-71 recombinant sequence.

(Regulation of Conversion Rate of Hereditary Trait)

When these mice were exposed to the step of converting hereditary traits (e.g., high temperature, high humidity, high salt concentration, etc.), the number of individuals which could adapt to the environment was significantly increased as compared to normal mice.

According to the method of this example, it was revealed that by deleting the proofreading activity of DNA polymerase δ, disequilibrium mutations can be accumulated on both leading and lagging DNA chains. It was also revealed that by expressing DNA polymerase δ having a mutation specifically in the spermatogenesis stage, the rate of mutations occurring in the whole body of mice can be reduced as much as possible. It is also revealed that secondary influences due to genetic manipulation or the like can be suppressed as much as possible. In this example, disequilibrium evolution mice satisfying the above-described requirements were achieved.

In the case of transgenic mouse #1, it was possible to investigate promoters which are expressed specifically in the spermatogenesis stage. Most of the promoters, which are currently known to be expressed specifically in the spermatogenesis stage, are expressed specifically in the spermatid stage after meiosis. In this example, it was intended to utilize a promoter which is expressed specifically in male germ cells in the spermatogonium stage or the primary spermatocyte stage where the DNA chain is replicated. The mPGK2 promoter was the only promoter that satisfied the conditions. Therefore, in this example, an attempt was made to utilize the sequence upstream of the Fth117 gone as a novel promoter. As a result, expression was confirmed in at least the primary spermatocyte stage. For spermatognia, transgenic mouse #1 was mated with an available CAG-CAT-GFP transgenic mouse (a transgenic mouse produced by using a vector having a structure similar to that of transgenic mouse #2 produced herein and in this mouse, expression of GFP is started by expression of the Cre recombinase), so that GFP was considered to be expressed in regions of transgenic mouse #1 in which the Cre recombinase is expressed. Therefore, by combining the results of the GFP expression regions and the Cre recombinase expression regions, it is possible to analyze the expression regions of the promoter of this example. Note that the sequence upstream of Fth117 did not contain a basic transcription factor binding sequence, such as a TATA box or the like.

In the expression of the mPGK2 promoter, the expression after the spermatogenesis stage was not observed, which was the later stage compared to conventional reports.

It was suggested that the two transgenic mice produced with transgenic mouse #1 had different expression regions. Therefore, it is considered to be useful that these mice are used to compare the expression efficiencies of various regions in order to regulate the conversion rate. Production of transgenic mice which express the Cre recombinant specifically in the spermatogenesis stage makes it possible to obtain regulatory gene deficient mice by utilizing recombination of the loxP sequence which occurs in a tissue-specific manner. Therefore, such mice can be used as materials for studying germ calls.

Transgenic mouse #2 can be mated with mice which express the Cre recombinase in a tissue-specific manner to achieve overexpression of a mutant-type Pold1 in a tissue-specific manner. In transgenic mouse #1, when the expression of the promoter is stopped, the expression of the mutant-type Pold1 no longer occurs. By the above-described mating, the expression of the mutant-type Pold01 can be continued after the end of the expression of the promoter. In addition, by mating with a transgenic mouse in which the Cre recombinase is expressed specifically in a tissue, such as, for example, the brain, the liver, or the like, an influence of the overexpression can be investigated at the somatic level.

According to the results of this example, it will be understood that the conversion rate of hereditary traits can be regulated in knockout mice.

### Example 5

### Production of Mutant Organisms Using Rice as a Plant

Next, in Example 5, rice (plant) is used as a representative eukaryotic organism to produce a disparity mutant organism.

Gene targeting techniques are described in, for example, Yagi T. et al., Proc. Natl. Acad. Sci. USA, 87: 9918-9922, 1990; “Gintagettingu no Saishingijyutsu [Up-to-date Gene Targeting Technology]”, Takeshi Yagi, ad, Special issue, Jikken Igaku [Experimental Medicine], 2000, 4. In Example 4, plants having a replication complex having disparity DNA replication proofreading abilities (Morrison, A., et al., Mol. Gen. Genet., 242: 289-296, 1994) are produced.

Hereditary traits to be modified are disease resistance (rice blest) and low-temperature resistance.

(Gene Targeting Techniques)

Targeting vectors having a mutant DNA polymerase (pol) (Morrison, A., et al., Mol. Gen. Genet., 242: 289-296, 1994) are prepared. Plant cells, such as callus or the like, are subjected to homologous recombination with respect to the pol gene of the plant cells. Thereafter, the cells are allowed to differentiate into plant bodies.

(Protocol)

(1. Preparation of Callus Cells)

Callus cells are prepared in well known techniques described in, for example, Plant Tissue Culture: Theory and Practice, Bhojwani, S. S. and Razdan, N. K., Elsevier, Amsterdam, 1983. Specifically, callus cells are prepared from plant bodies (Davies, R., 1981, Nature, 291: 531-532 and Luo, Z., et al., Plant Mol. Bio. Rep., 7: 69-77, 1989).

(2. Homologous Recombination of Pol Genes)

To obtain homologous recombinant cells efficiently, homologous recombination is carried out using a gene targeting method for mice, i.e., a positive/negative method (Yagi, T., at al., Proc. Natl. Acad. Sci. USA, 87: 9918-9922, 1990; Capecchi M. R., Science, 244(16), 1288-1292, 1989).

Preparation of targeting vectors: targeting vectors were prepared by techniques described in, for example, Molecular Cloning, 2nd edition, Sambrook, J., et al, supra, and Ausubel, F. M., Current Protocols in Molecular Biology, Green Publishing Associates and Wiley-Interscience, NY, 1987, Supra.

In the targeting vector, mutation polδ and/or pol ε genes were inserted between a positive gene and a negative gene. Hygromycin resistant gene was used as the positive gene while diphtheria toxin was used as the negative gene (Terada R., at al., Nature Biotech., 20: 1030-1034, 2002).

For Pol mutations, a base mutation was introduced into the proofreading activity sites of polδ to delete proofreading activity (D at position 320 and E at position 322 of SER ID NO. 48 are substituted with alanine (A)) (Morrison A. & Sugino Al, Mol. Gen. Genet. 242: 289-296, 1994: Goldsby R. E., et al., Pro. Natl. Acad. Sci. USA, 99: 15560-15565, 2002).

(3. Introduction of Vectors into Callus Cells)

Vectors are introduced into callus cells by techniques described in, for example, “Shokubutsu Baiotekunoroji II Plant Biotechnology II] ”, Yasuyuki & Kanji Ooyama, eds., Tokyo Kagakudojtn, 1991. In Example 5, vectors are introduced into callus calls by an electroporation method, an Agrobacterium method, or the like. Culture is carried out in DMEM medium (Flow Laboratory) containing hygromycin (100 μg/ml, Invitrogen).

(4. Recovery of Recombinant Cells)

After culture in the presence of hygromycin, recombinant cells are recovered (Terada R., et al., Nature Biotech., 20: 1030-1034, 2002).

(5. Confirmation of Homologous Recombinants)

Genomic DNA is extracted from recombinants. Whether or not mutant pol is successfully introduced into the ES cells is determined by Southern blotting and/or PCR (“Gintagettingu no Saishingijyutsu [Up-to-date Gene Targeting Technology]”, Takeshi Yagi, ad., Special issue, Jikken Igaku [Experimental Medicine], 2000, 4).

(6. Production of Plant Bodies)

Plant bodies are produced in methods described in, for example, “Shokubutsu Baiotekunoroji II [Plant Biotechnology II]”, Yasuyuki & Kanji Ooyama, eds., Tokyo Kagakudojin, 1991; and “Shokubutsu Soshikibaiyo no Gijyutsu [Plant Tissue Culture Technique]”, Masayuki Takeuti, Tetsuo Nakajima, & Riki Kotani, eds., Asakura Shoten, 1988. In Example 5, callus is differentiated into a plant body. Thereafter, monoploid cells derived from anther, seed, or the like and/or homo diploid cells prepared by crossbreeding plants, and the like are used to confirm properties of pol mutation (mutator mutation) using techniques well known in the art (Maki, H. et al., J. Bacteriology, 153(3), 1361-1367, 1983; Miller, J. H., 1992, A Short course in bacterial genetics, Cold Spring Harber Laboratory Press. Cold Spring Harber, N.Y.).

(Results)

It is observed that plants obtained in Example 5 having mutations can obtain low-temperature resistance and disease resistance (e.g., rice blast, etc.) rapidly as compared to plants obtained by conventional techniques. The modified cells had substantially the same growth rate as that of naturally-occurring cells, however, the mutation rate of the modified call was two or more per generation, which is significantly different from that of conventional mutations.

### Example 6

### Demonstration in Arabidopsis thaliana

Next, Arabidopsis thaliana was used to produce a mutant organism.

(Methods and Materials)

(polδ cDNA Cloning)

polδ (At1g42120) (SEQ ID NO.: 90 (nucleic acid sequence) and SEQ ID No.: 91 (amino acid sequence)) were amplified by PCR using the following primers from total mRNA derived from a root of Arabidopsis thaliana and subcloned in pBluescript SK2 (TOYOBO).

| | | |
|---|---|---|
| Xbal-42120-F: | 5′-CTGAGTCTAGATTTCCCGCCATGGAAATCG-3′ | (SEQ ID NO.: 82) |
| 2g42120-Sacl-R: | 5′-AGCAACGAGCTCTTATGATTGGTTTATCTG-3′ | (SEQ ID NO.: 83) |

(Production of mutant-type polδ gene polδ (D316A) (SEQ ID NO.: 92 (nucleic acid sequence) and SEQ ID NO.: 93 (amino acid sequence)))

A point mutation was induced using the following primers to change amino acid 316 in polδ cDNA from D to A.

| | | |
|---|---|---|
| 2g42120-D316A-F: | 5′-ATTTGCTGTCGATAATATCAGATTTCTTGG-3′ | (SEQ ID NO.: 84) |
| 2g42120R: | 5′-GAGTGAGGATTTGTACATGATCTGAAGG-3′ | (SEQ ID NO.: 85) |

(Production of Vector for Transformation)

A binary plasmid which consistently expresses a gene in plants was produced by modifying pBI121 (CLONTECH). The β-glucuronidase gone of pBI121 was extracted using restriction enzymes XbaI and SacI, and was substituted with Polδ (D316A) (hereinafter referred to as polδ (D316A)).

As a vector used as a control for transformation, the above-described pBI121 (hereinafter referred to as GUS) and pBI121 with GYP substituting for the β-glucuronidase gene (hereinafter referred to as GTP) were produced.

(Production of Callus)

Seeds of Arabidopsis thaliana (ectype: Columbia) were disseminated on germination medium, followed by low temperature treatment at 4° C. for 2 or 3 days. Thereafter, the plate was transferred into an incubator (22° C.). The seeds were grown in dark place for 10 days. The elongated hypocotyl was out into about 1-cm length pieces, which were in turn placed on CIM medium for 10 days. A callus was obtained.

(Transformation of callus using Agrobacterium)

Agrobacterium pMP90 containing a binary plasmid having GUS or GTP or polδ (D316A) was inoculated into LB medium supplemented with 50 mg/L kanamycin, followed by shaking culture at 28° C. for 2 days. 1.4 ml of Agrobacterium culture (OD600=about 0.8) was centrifuged in a bench-top centrifuge for 5 minutes to collect the bacteria. The bacteria were suspended in 1 ml of AIM (described below). Callused hypocotyl fragments were transferred into a 60-mm petri dish containing 5 ml of AIM. 1 ml of the Agrobacterium suspension was added to the dish, followed by shaking culture at room temperature for about 20 minutes. The calli were placed on a sterilized filter to remove the extra moisture content, and thereafter, was transferred to a now CIM plate. Three days later, the transformed calli were transferred into a 60-mm Petri dish containing AIM. The dish was rotated at 60 rpm for 25 minutes, followed by washing 5 times.

After washing, the calli were placed on a filter to remove the moisture content, and were grown in CIM medium containing 50 mg/L carbenicillin and 50 mg/L kanamycin (described below) (the CIM medium was prepared by the present inventors).

Note that the transformation rate of the callus was 95% or more (only calli into which GTP was introduced were measured and the presence or absence of GTP fluorescence was examined).

(Subculture of Calli and Screening of Mutants)

The calli were transferred into new CIM plates every 10 days. It this case, one callus was divided into two. One half was placed in a CIM plate for subculture, while the other half was placed in a plate for screening for resistant mutants under various conditions.

200 mM or 300 mM NaCl were added to the screening plate. Subculture and screening were performed every 10 days.

(Composition of medium)

| Germination medium (1 Liter): | | |
|---|---|---|
| Murashige Minimal Organic Medium (GIBCO BRL) | ½ | package |
| sucrose | 10 | g |
| Gelllan Gum (Wako Pure Chemical Industries) | 5 | g |
| (CIM (1 Liter)) | | |
| Gamborg's B5 Medium Salt Mixture | 1 | package |
| (Nihon Pharmaceutical) | | |
| glucose | 20 | g |
| myoinositol | 100 | mg |
| 5% Mes-KOH (pH 5.7) | 10 | ml |
| Gelllan Gum | 5 | g |
| After autoclaving, the following materials were added: | | |
| thiamin hydrochloride | 20 | mg |
| nicotinic acid | 1 | mg |
| pyridoxine hydrochloride | 1 | mg |
| biotin | 10 | mg |
| 2,4-D | 0.5 | mg |
| kinetin | 0.05 | mg |
| (AIM (1 Liter)) | | |
| Gamborg's B5 Medium Salt Mixture | 1 | package |
| (Nihon Pharmaceutical) | | |
| glucose | 20 | g |
| 5% Mes-KOH (pH 5.7) | 10 | ml |

(Results)

As a result, the above-described genes used (GFP, GUS (control for transformation), polδ (D316A)) each had a transformation rate of 95% or more (only individuals into which GTP was introduced were measured; and the presence or absence of GTP fluorescence was examined).

(Conditions for Evolution)

The plants obtained in this example were exposed to conditions for altering the following hereditary traits.

Screening mutants was performed under the following conditions.
1) 37° C. The plate was placed in an incubator at 37° C.
2)200 mM NaCl 200 mM NaCl was added to the medium, and the plant was grown at 22° C.
3)300 mM NaCl 300 mM NaCl was added to the medium, and the plant was grown at 22° C.

(Results of Screening Mutants)

The result of each treatment will be described below. Numerals in the table below indicate: the number of calli which grew like non-treated callus (resistant)/the number of calli which did not grow well but did not die (weakly resistant)/the number of dead calli (susceptible) in this order from the left.

| Treatment | GFP | polδ |
|---|---|---|
| 37° C. | 0/24/27 | 1/18/10 |
| 200 mM NaCl | 0/20/145 | 0/58/112 |
| 300 mM NaCl | 0/0/165 | 0/4/146 |

As described above, the number of plants which became resistant to high temperature treatment was increased. In addition, for salt concentration, the number of calli (polδ) resistant to 200 mM NaCl was greater than that of the control. Therefore, it was revealed that the method of the present invention could confer resistance to a high salt concentration to plants. Particularly, in the case of 300 mM NaCl, the control could not acquire resistance, while the method of the present invention could confer resistance.

### Example 7

### Serial Resistance Experiment Using Arabidopsis thaliana

Next, it was determined whether or not a hereditary trait, such as resistance, was propagated over generations. Conditions for this experiment were the same as used in Example 6.

The number of individuals are described below.

TABLE 5
Results of salt resistance experiment using Arabidopsis
thaliana callus
<Number of calli tested>

| Type of plasmid | 200 mM NaCl | 300 mM NaCl |
|---|---|---|
| Mutant polδ | 75 | 75 |
| GTP | 96 | 95 |
| GUS | 75 | 68 |

Screening Method

A callus was produced. The callus, which grew to a certain degree, was divided into two. One half was grown to the original size in normal medium, while the other half was cultured in selective medium to test the acquisition of resistance. When the callus in the normal medium grew well, one half was transferred to normal medium while the other half was transferred into selective medium for second screening. A total of 6 screenings were performed. In the case of 300 mM NaCl, no resistant callus was obtained. Therefore, all experiments were performed with respect to 200 mM NaCl.

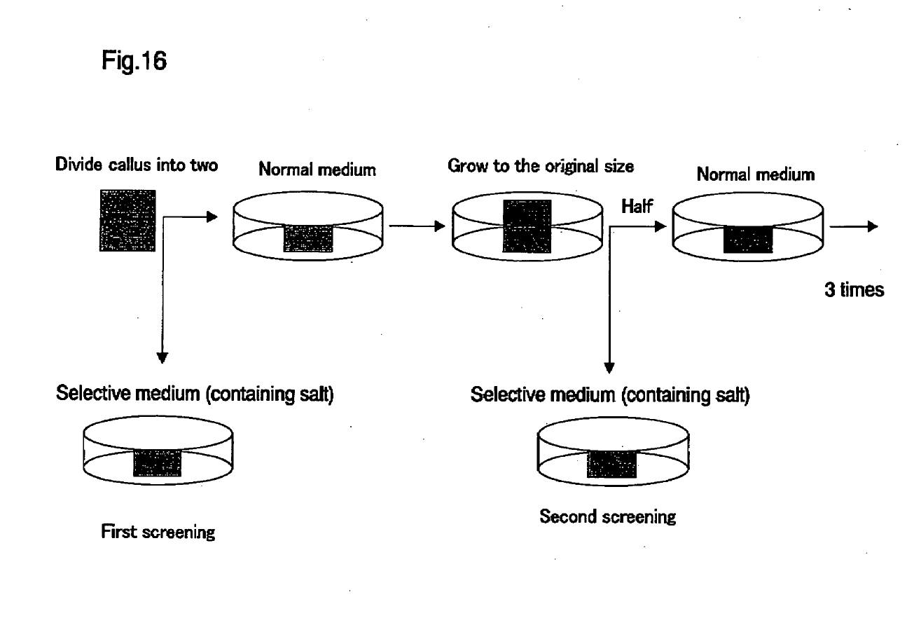

FIG. 16 schematically shows the experiment.

Discontinuous Experiments

The number of resistant callus, which discontinuously occurred in the 6 screenings, was counted. A callus, which acquired resistance once but lost it, was considered to be pseudopositive.

TABLE 6
The results of the discontinuous experiment

| | Number of 200 mM NaCl |
|---|---|
| Type of plasmid | resistant calli |
| Mutant polδ | 8 |
| GTP | 6 |
| GUS | 6 |

Continuous Experiment

To remove pseudopositive results, the number of calli, which continuously acquired resistance, was counted. The number of calli, which had resistance in up to the 6th screening, is shown below.

TABLE 7

| | Number of 200 mM NaCl |
|---|---|
| Type of plasmid | resistant calli |
| Mutant polδ | 1 |
| GTP | 0 |
| GUS | 0 |

Thus, only the strain having mutant polδ continuously exhibited resistance. This strain maintained resistance beyond the 6th generation. It was revealed that the present invention is superior over conventional techniques in terms of stability as well as the conversion rate of hereditary traits.

### Example 8

### Experiment Using ES Cell

Next, it was determined whether or not the present invention can be applied to ES cells. The procedure is shown below.

Preparation of ES Cells

An ES cell line (TT-2 call) derived from C57BL/6 and CBA FP mouse embryos (prepared by the Yagi's laboratory of Osaka University in accordance with a typical protocol) was cultured and multiplied on feeder calls in ES cell culture medium (ESM) (DMEM containing 20% FBS, 0.1 nM NEAA, 1 mM pyruvic acid, LIF (ESGRO®, Amrad), and mercaptoethanol).

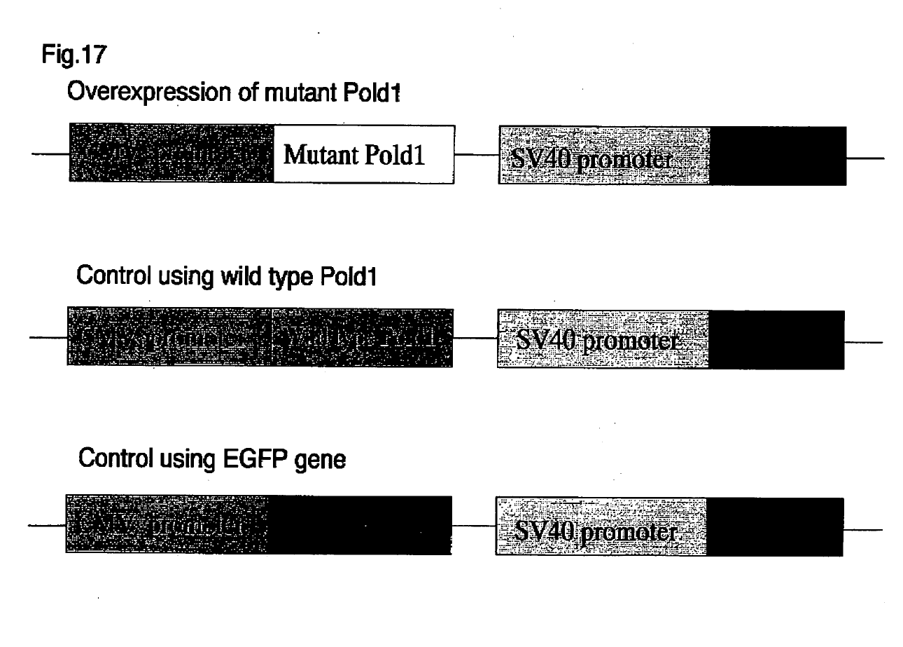

Introduced vectors (FIG. 17) were prepared as follows. cDNA of mutant Pold1, normal Pold1, or EGTP gene was incorporated into pcDNA 3.1(+), which is a protein expression vector. Restriction enzyme digestion was performed to obtain linear DNA fragments, which were in turn used for genes to be introduced. The multiplied ES cells were removed using 0.25% trypsin solution. The ES cell were placed in cuvettes at a rate of 2.0×10⁶ ES cells/cuvette. The cells were mixed with 100 μl of 25 nM vector DNA solution, followed by electroporation for gene introduction.

After electroporation, the calls were cultured using ESM for 48 hours. Thereafter, the cells were cultured in ESM medium supplemented with G418 (final concentration: 200 μg/mL) (SIGMA). Thereby, the gene introduced cells were sectioned. Culture was performed on gelatin-coated plates. Thereafter, the ES cells were cultured in the presence of Penicillin-Streptomycin (a 100-fold dilution of a commercially available product (GIBCO)).

6TG Assay

ES calls, which were multiplied and trypsin-treated (2.5%, GIBCO) were disseminated on a 10-cm dish to 5.0×10⁶ cells/dish. Resistant colonies were sectioned in the presence of 6-TG (final concentration: 2 μg/ml; Sigma, hybridoma tested) and G418. In this sectioning, the cells were cultured on a gelatin-coated dish (0.1% gelatin solution was placed in a FALCON 353003 cell culture dish, followed by incubation at 37° C. for 30 minutes (gelatin available from SIGMA)). Culture medium was exchanged once every two days. The day on which the cells were disseminated is regarded as Day 0. The number of colonies was counted on Day 11. Only colonies, which multiplied well and grew, were counted.

Results of Experiment

There were two lots of mutant Pold1 designated #1 and #2, for which electroporation were separately performed. In either case, six 10-cm dishes were used and the appearance of resistant colonies was counted. The number of colonies is shown below. (0×6 represents six dishes on which no colonies appeared)

| | |
|---|---|
| Mutant Pold1 | 3 × 1, 1 × 2, 0 × 9 |
| Wildtype Pold1 | 0 × 6 |
| EGTP control | 0 × 6 |

According to the results of this example, growing colonies were observed only in the case of mutant Pold1. For the obtained colonies, the HGPRT gone, which was a target for mutation, could be partially sequenced to confirm the introduction of a mutation.

Thus, it was revealed that overexpression of the mutant Pold1 gene facilitated introduction of a mutation into mouse ES cells. Therefore, it was demonstrated that it was possible to regulate the conversion rate of hereditary traits in ES cells, and the rate and stability were increased.

### Example 9

### Gram-Positive Bacteria

In this example, as an exemplary gram-positive bacterium, Bacillus subtilis was used as a host cell, into which a mutation was introduced. In the mutation, aspartic acid and glutamic acid at positions 425 and 427, respectively, were mutated in polymerase C set forth in SEQ ID No.: 15. This polymerase C mutant was introduced into Bacillus subtilis via a plasmid (pHY300PLK, TAKARA). Thereafter, the bacterium was exposed under conditions for evolution.

After production of the mutant, for example, an intermediate high temperature (e.g., 42° C.) was gradually increased to 50° C. or more.

Bacillus subtilis is a type of sail bacteria which has been extensively studied. The growth temperature thereof is 20 to 50° C. (the bacterium doubles at pH 6 to 7 in 30 minutes).

As described above, under the conditions for evolution, some Bacillus strains of this example could live at as high as 55° C. The strain could maintain the property in subcultures.

Therefore, it was revealed that it was possible to regulate the rate of evolution of a bacterium which has a DNA replicating mechanism different from that of E. coli.

### Example 10

### Isolation of Genes

In Example 10, genes playing a role in changing hereditary traits are isolated. Organisms acquiring the drug resistance of Example 1 are isolated. Thereafter, the sequence of a gene involved in drug resistance is determined in original organisms before modification and the modified organisms. As a result, it is found that gyrase (or topoisopolmerase II) subunit A and topoisomerase IV genes are modified. These sequences are amplified by PCR using appropriate primers and full-length genes are isolated. From the original and modified genes, polypeptides are synthesized and activity thereof is measured. As a result, it is found that the activity is certainly changed. Thus, it is demonstrated that the method of the present invention can rapidly introduce mutations at the gene level.

### Example 11

### Isolation of New Product Substances

In Example 11, new product substances obtained by modifications are isolated. Organisms acquiring the drug resistance of Example 1 are isolated. Thereafter, a substance which is not present in an original organism before modification but is present in the modified organism, is identified by chromatography analysis (e.g., HPLC, etc.). The new product substance Is isolated. As a result, gyrase (or topoisopolmerase II) subunit A and topoisomerase IV gene products are found to be new product substances. Thus, it is demonstrated that the method of the present invention is actually useful in production of new product substances.

### Example 12

### Other Methods of Modifying Error-Prone Frequency

Instead of the above-described mutations, it is possible to introduce a mutation which impairs the activity of a polymerase portion of polymerases δ and ε to reduce the accuracy of DNA replication.

### Example 13

### Relationship Between Error-Prone Frequency and the Rate of Evolution)

As a control, conventional methods (radiation, chemical treatment, etc.) of introducing mutations were carried out in experiments for acquisition by yeast of drug resistance, alcohol resistance, and high temperature resistance as described in Example1. As a result, the speed of resistance acquisition by the present invention was significantly higher than by conventional techniques. When both experiments were started at the same time, resistant strains could be obtained by the present invention earlier than conventional techniques.

In Example 13, methods having mutation rates which varied stepwise were used to compare the times required for acquisition of resistance. As a result, the rates of evolution could be obtained.

According to the present invention, desired traits can be conferred to organisms rapidly and with substantially no adverse effect, compared to conventional methods. In addition, according to the present invention, hereditary traits of organisms can be modified by easy manipulations. Thereby, it is possible to efficiently obtain useful organisms, genes, gene products, metabolites, and the like, which cannot be obtained by conventional methods.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

1. A method for regulating a conversion rate of a hereditary trait of a cell, comprising the step of:
(a) regulating an error-prone frequency of gene replication of the cell.

2. A method according to claim 1, wherein at least two kinds of error-prone frequency agents playing a role in the gene replication are present.

3. A method according to claim 2, wherein at least about 30% of the error-prone frequency agents have a lesser error-prone frequency.

4. A method according to claim 1, wherein the agents playing a role in the gene replication have heterogeneous error-prone frequencies.

5. A method according to claim 1, wherein the agent having the lesser error-prone frequency is substantially error-free.

6. A method according to claim 2, wherein the error-prone frequencies are different from each other by at least 10¹.

7. A method according to claim 2, wherein the error-prone frequencies are different from each other by at least 10².

8. A method according to claim 2, wherein the error-prone frequencies are different from each other by at least 10³.

9. A method according to claim 1, wherein the step of regulating the error-prone frequency comprises regulating an error-prone frequency of at least one agent selected from the group consisting of a repair agent capable of removing abnormal bases and a repair agent capable of repairing mismatched base pairs, the agents being present in the cell.

10. A method according to claim 1, wherein the step of regulating the error-prone frequency comprises providing a difference in the number of errors between one strand and the other strand of double-stranded genomic DNA in the call.

11. A method according to claim 1, wherein the step of regulating the error-prone frequency comprises regulating an error-prone frequency of a DNA polymerase of the cell.

12. A method according to claim 11, wherein the DNA polymerase has a proofreading function.

13. A method according to claim 11, wherein the DNA polymerase comprises at least one polymerase selected from the group consisting of DNA polymerase α, DNA polymerase β, DNA polymerase γ, DNA polymerase δ, and DNA polymerase ε of eukaryotic cells, and corresponding DNA polymerases thereto.

14. A method according to claim 1, wherein the step of regulating the error-prone frequency comprises regulating proofreading activity of at least one polymerase selected from the group consisting of DNA polymerase δ and DNA polymerase ε of eukaryotic cells, and corresponding DNA polymerases thereto.

15. A method according to claim 1, wherein the regulating the error-prone frequency comprises regulating a proofreading activity of DNA polymerase δ of a prokaryotic cell or DNA polymerase corresponding thereto.

16. A method according to claim 1, wherein the regulating the error-prone frequency comprises introducing a DNA polymerase variant into the cell.

17. A method according to claim 16, wherein the introducing the DNA polymerase variant into the cell is performed with a method selected from the group consisting of homologous recombination and transformation using gene introduction or a plasmid.

18. A method according to claim 1, wherein the regulating the error-prone frequency comprises introducing a variant of DNA polymerase δ of a prokaryotic cell or DNA polymerase corresponding thereto.

19. A method according to claim 18, wherein the variant of DNA polymerase δ of a prokaryotic cell or DNA polymerase corresponding thereto comprises a mutation which deletes a proofreading activity thereof.

20. A method according to claim 1, wherein the step of regulating the error-prone frequency comprises increasing the error-prone frequency higher than that of a wild type of the cell.

21. A method according to claim 12, wherein the proofreading function of the DNA polymerase is lower than that of a wild type of the DNA polymerase.

22. A method according to claim 12, wherein the proofreading function of the DNA polymerase provides at least one mismatched base in a base sequence, the number of the at least one mismatched base being greater by at least one than that of a wild type of the DMA polymerase.

23. A method according to claim 12, wherein the proofreading function of the DNA polymerase provides at least one mismatched base in a base sequence.

24. A method according to claim 12, wherein the proofreading function of the DNA polymerase provides at least two mismatched bases.

25. A method according to claim 12, wherein the proofreading function of the DNA polymerase provides at least one mismatched base in a base sequence at a rate of 10⁻⁶.

26. A method according to claim 12, wherein the proofreading function of the DNA polymerase provides at least one mismatched base in a base sequence at a rate of 10⁻³.

27. A method according to claim 12, wherein the proofreading function of the DNA polymerase provides at least one mismatched base in a base sequence at a rate of 10⁻².

28. A method according to claim 1, wherein the cell is a gram-positive or eukaryotic call.

29. A method according to claim 1, wherein the cell is a eukaryotic cell.

30. A method according to claim 1, wherein the cell is a unicellular or multicellular organism.

31. A method according to claim 1, wherein the cell is an animal, plant, fungus, or yeast cell.

32. A method according to claim 1, wherein the cell is a mammalian cell.

33. A method according to claim 1, wherein after conversion of the hereditary trait, the cell has substantially the same growth as that of a wild type of the cell.

34. A method according to claim 1, wherein the cell naturally has at least two kinds of polymerases.

35. A method according to claim 1 wherein the cell naturally has at least two kinds of polymerases, the at least two kinds of polymerases having a different error-prone frequency.

36. A method according to claim 1, wherein the call has at least two kinds of polymerases, one of the at least two kinds of polymerases is involved in an error-prone frequency of a lagging strand, and another of the at least two kinds of polymerases is involved in an error-prone frequency of a leading strand.

37. A method according to claim 1, wherein the cell has resistance to an environment, the resistance being not possessed by the cell before the conversion.

38. A method according to claim 37, wherein the environment comprises, as a parameter, at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the call, chemical agents, antibiotics, natural substances, mental stress, and physical stress, or a combination thereof.

39. A method according to claim 1, wherein the cell includes a cancer cell.

40. A method according to claim 2, wherein the cell constitutes a tissue.

41. A method according to claim 1, wherein the cell constitutes an organism.

42. A method according to claim 1, further comprising:
differentiating the cell to a tissue or an organism after conversion of the hereditary trait of the cell.

43. A method according to claim 1, wherein the error-prone frequency is regulated under a predetermined condition.

44. A method according to claim 43, wherein the error-prone frequency is regulated by regulating at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

45. A method for producing a cell having a regulated hereditary trait, comprising the step of:
(a) regulating an error-prone frequency of gene replication of the cell; and
(b) reproducing the resultant cell.

46. A method according to claim 45, further comprising:
screening for the reproduced cell having a desired trait.

47. A method according to claim 45, wherein at least two kinds of error-prone frequency agents playing a role in the gene replication are present.

48. A method according to claim 45, wherein at least about 30% of the error-prone frequency agents have a lesser error-prone frequency.

49. A method according to claim 45, wherein the agents playing a role in the gene replication have heterogeneous error-prone frequencies.

50. A method according to claim 45, wherein the agent having the lesser error-prone frequency is substantially error-free.

51. A method according to claim 45, wherein the error-prone frequencies are different from each other by at least 10¹.

52. A method according to claim 45, wherein the error-prone frequencies are different from each other by at least 10².

53. A method according to claim 45, wherein the error-prone frequencies are different from each other by at least 10³.

54. A method according to claim 45, wherein the step of regulating the error-prone frequency comprises regulating an error-prone frequency of at least one agent selected from the group consisting of a repair agent capable of removing abnormal bases and a repair agent capable of repairing mismatched base pairs, the agents being present in the cell.

55. A method according to claim 45, wherein the step of regulating the error-prone frequency comprises providing a difference in the number of errors between one strand and the other strand of double-stranded genomic DNA in the cell.

56. A method according to claim 45, wherein the step of regulating the error-prone frequency comprises regulating an error-prone frequency of a DNA polymerase of the cell.

57. A method according to claim 56, wherein the DNA polymerase has a proofreading function.

58. A method according to claim 56, wherein the DNA polymerase comprises at least one polymerase selected from the group consisting of DNA polymerase α, DNA polymerase β, DNA polymerase γ, DNA polymerase δ, and DNA polymerase ε of eukaryotic cells, and corresponding DNA polymerases thereto.

59. A method according to claim 45, wherein the step of regulating the error-prone frequency comprises regulating proofreading activity of at least one polymerase selected from the group consisting of DNA polymerase δ and DNA polymerase ε of eukaryotic cells, and corresponding DNA polymerases thereto.

60. A method according to claim 45, wherein the regulating the error-prone frequency comprises regulating a proofreading activity of DNA polymerase δ of a prokaryotic cell or DNA polymerase corresponding thereto.

61. A method according to claim 45, wherein the regulating the error-prone frequency comprises introducing a DNA polymerase variant into the cell.

62. A method according to claim 61, wherein the introducing the DNA polymerase variant into the cell is performed with a method selected from the group consisting of homologous recombination and transformation using gene introduction or a plasmid.

63. A method according to claim 45, wherein the regulating the error-prone frequency comprises introducing a variant of DNA polymerase δ of a prokaryotic cell or DNA polymerase corresponding thereto.

64. A method according to claim 63, wherein the variant of DNA polymerase δ of a prokaryotic call or DNA polymerase corresponding thereto comprises a mutation which deletes only a proofreading activity thereof.

65. A method according to claim 45, wherein the step of regulating the error-prone frequency comprises increasing the error-prone frequency higher than that of a wild type of the cell.

66. A method according to claim 57, wherein the proofreading function of the DNA polymerase is lower than that of a wild type of the DNA polymerase.

67. A method according to claim 57, wherein the proofreading function of the DNA polymerase provides at least one mismatched base in a base sequence, the number of the at least one mismatched base being greater by at least one than that of a wild type of the DNA polymerase.

68. A method according to claim 57, wherein the proofreading function of the DNA polymerase provides at least one mismatched base in a base sequence.

69. A method according to claim 57, wherein the proofreading function of the DNA polymerase provides at least two mismatched bases.

70. A method according to claim 57, wherein the proofreading function of the DNA polymerase provides at least one mismatched base in a base sequence at a rate of 10⁻⁶.

71. A method according to claim 57, wherein the proofreading function of the DNA polymerase provides at least one mismatched base in a base sequence at a rate of 10⁻³.

72. A method according to claim 57, wherein the proofreading function of the DNA polymerase provides at least one mismatched base in a bass sequence at a rate of 10⁻².

73. A method according to claim 45, wherein the cell is a gram-positive or eukaryotic cell.

74. A method according to claim 45, wherein the cell is a eukaryotic cell.

75. A method according to claim 45, wherein the cell is a unicellular or multicellular organism.

76. A method according to claim 45, wherein the cell is an animal, plant, fungus, or yeast cell.

77. A method according to claim 45, wherein the cell is a mammalian cell.

78. A method according to claim 45, wherein after conversion of the hereditary trait, the cell has substantially the same growth as that of a wild type of the cell.

79. A method according to claim 45, wherein the cell naturally has at least two kinds of polymerases.

80. A method according to claim 45, wherein the cell naturally has at least two kinds of polymerases, the at least two kinds of polymerases having a different error-prone frequency.

81. A method according to claim 45, wherein the cell has at least two kinds of polymerases, one of the at least two kinds of polymerases is involved in an error-prone frequency of a lagging strand, and another of the at least two kinds of polymerases to involved in an error-prone frequency of a leading strand.

82. A method according to claim 45, wherein the cell has resistance to an environment, the resistance being not possessed by the cell before the conversion.

83. A method according to claim 82, wherein the environment comprises, as a parameter, at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

84. A method according to claim 45, wherein the cell includes a cancer call.

85. A method according to claim 45, wherein the cell constitutes a tissue.

86. A method according to claim 45, wherein the cell constitutes an organism.

87. A method according to claim 45, further comprising:
differentiating the cell to a tissue or an organism after conversion of the hereditary trait of the cell.

88. A method according to claim 45, wherein the error-prone frequency is regulated under a predetermined condition.

89. A method according to claim 88, wherein the error-prone frequency is regulated by regulating at least one agent selected from the group consisting of temperature, humidity, pH, salt concentration, nutrients, metal, gas, organic solvent, pressure, atmospheric pressure, viscosity, flow rate, light intensity, light wavelength, electromagnetic waves, radiation, gravity, tension, acoustic waves, cells other than the cell, chemical agents, antibiotics, natural substances, mental stress, and physical stress, or a combination thereof.

90. A method for producing an organism having a regulated hereditary trait, comprising the steps of:
(a) regulating the error-prone frequency of gene replication of the organism; and
(b) reproducing the resultant organism.

91. A cell having a regulated hereditary trait, produced by a method according to claim 90.

92. A cell according to claim 91, wherein the cell has substantially the same growth as that of a wild type of the cell.

93. An organism having a regulated hereditary trait, produced by a method according to claim 90.

94. An organism according to claim 93, wherein the organism has substantially the same growth as that of a wild type of the organism.

95. A method for producing a nucleic acid molecule encoding a gene having a regulated hereditary trait, comprising the steps of:
(a) changing an error-prone frequency of gene replication of an organism;
(b) reproducing the resultant organism;
(c) identifying a mutation in the organism; and
(d) producing a nucleic acid molecule encoding a gene having the identified mutation.

96. A nucleic acid molecule, produced by a method according to claim 95.

97. A method for producing a polypeptide encoded by a gene having a regulated hereditary trait, comprising the steps of:
(a) changing an error-prone frequency of gene replication of an organism;
(b) reproducing the resultant organism;
(a) identifying a mutation in the organism; and
(d) producing a polypeptide encoded by a gene having the identified mutation.

98. A polypeptide, produced by a method according to claim 97.

99. A method for producing a metabolite of an organism having a regulated hereditary trait, comprising the steps of:
(a) changing an error-prone frequency of gene replication of an organism;
(b) reproducing the resultant organism;
(c) identifying a mutation in the organism; and
(d) producing a metabolite having the identified mutation.

100. A metabolite, produced by a method according to claim 99.

101. A nucleic acid molecule for regulating a hereditary trait of an organism, comprising:
a nucleic acid sequence encoding a DNA polymerase having a regulated error-prone frequency.

102. A nucleic acid molecule according to claim 101, wherein the DNA polymerase is DNA polymerase δ or ε of eukaryotic organisms, or DNA polymerase corresponding thereto of gram-positive bacteria.

103. A nucleic acid molecule according to claim 101, wherein the DNA polymerase is a variant of DNA polymerase δ or ε of eukaryotic organisms, or DNA polymerase Corresponding thereto of gram-positive bacteria, the variant comprising a mutation which deletes only a proofreading activity thereof.

104. A nucleic acid molecule according to claim 101, wherein the DNA polymerase is a variant of DNA polymerase δ of eukaryotic organisms, or DNA polymerase corresponding thereto of gram-positive bacteria, the variant comprising a mutation which deletes only a proofreading activity thereof.

105. A vector, comprising a nucleic acid molecule according to claim 101.

106. A cell, comprising a nucleic acid molecule according to claim 101.

107. A cell according to claim 106, wherein the cell is a eukaryotic cell.

108. A cell according to claim 107, wherein the eukaryotic cell is selected from the group consisting of plants, animals, and yeasts.

109. A cell according to claim 106, wherein the cell is a gram-positive bacterial cell.

110. A cell according to claim 106, wherein the cell is used for regulating a conversion rate of a hereditary trait.

111. An organism, comprising a nucleic acid molecule according to claim 101.

112. A product substance, produced by a call according to claim 106 or a part thereof.

113. A nucleic acid molecule, contained in a cell according to claim 106 or a part thereof.

114. A nucleic acid molecule according to claim 113, encoding a gene involved in the regulated hereditary trait.

115. A method for testing a drug, comprising the steps of:
testing an effect of the drug using a cell according to claim 106 as a model of disease;
testing an effect to the drug using a wild type of the cell as a control; and
comparing the model of disease and the control.

116. A method for testing a drug, comprising the steps of:
testing an effect of the drug using an organism according to claim 111 as a model of disease;
testing an effect to the drug using a wild type of the organism as a control; and
comparing the model of disease and the control.

117. A set of at least two kinds of polymerases for use in regulating a conversion rate of a hereditary trait of an organism, wherein the polymerizes have a different error-prone frequency.

118. A set according to claim 117, wherein one of the at least two kinds of polymerases is involved in an error-prone frequency of a lagging strand, and another of the at least two kinds of polymerases is involved in an error-prone frequency of a leading strand.

119. A set according to claim 117, wherein the set of polymerases are derived from the same species.

120. A set of at least two kinds of polymerases for use in producing an organism having a regulated hereditary trait, wherein the polymerases have a different error-crone frequency.

121. A set according to claim 120, wherein one of the at least two kinds of polymerases is involved in an error-prone frequency of a lagging strand, and another of the at least two kinds of polymerases is involved in an error-prone frequency of a leading strand.

122. A set according to claim 121, wherein the set of polymerases are derived from the same organism species.

123. Use of at least two kinds of polymerases for regulating a conversion rate of a hereditary trait of an organism, wherein the polymerases have a different error-prone frequency.

124. Use of at least two kinds of polymerases for producing an organism having a regulated hereditary trait, wherein the polymerases have a different error-prone frequency.


SEQUENCE LISTING (95 sequences)

SEQ ID NO: 1 - DNA, 3551 bp, Saccharomyces cerevisiae
acgcgtaactttttattctataaaatgttcaatgaggacatctgctattcgcttatgaagaacaaacactcagtactactgatctaaggcaattttcaaggataaaggaaaatagatattgagcacttgctattaagcattaatctttatacatatacgcacagcaatgagtgaaaaaagatcccttcccatggttgatgtgaagatcgatgacgaggatactccccagttggaaaagaaaatcaaacggcaatcaatagatcatggtgttggaagtgaacctgtttcaacaatagagattattccgagtgattcttttcgaaaatataatagtcaaggcttcaaagcaaaggatacagatttaatgggtacgcaattagagtctacttttgaacaagagctatcgcaaatggaacatgatatggccgaccaagaagagcatgacctgtcatcattcgagcgtaagaaacttccaaccgattttgacccaagtttgtatgatatttctttccaacaaattgatgcggaacagagcgtactgaatggtatcaaagatgaaaatacatctaccgtggtaaggttttttggtgtcactagtgaaggacactctgtactttgtaatgttacagggttcaagaactatctttacgtcccagcgcccaattcttccgacgctaacgatcaggagcaaatcaacaagtttgtgcactatttaaacgaaacatttgaccacgctattgattcgattgaagttgtatctaaacagtctatctggggttattccggagataccaaattaccattctggaaaatatacgtcacctatccgcatatggtcaacaaactgcgtactgcgtttgaaagaggtcatctttcattcaactcgtggttttctaacggcacgactacttatgataacattgcctacactttaaggttaatggtagattgtggaattgtcggtatgtcctggataacattaccaaaaggaaagtattcgatgattgagcctaataacagagtttcctcttgtcagttggaagtttcaattaattatcgtaacctaatagcacatcctgctgagggtgattggtctcatacagctccattgcgtatcatgtcctttgatatcgagtgtgctggtaggattggcgtctttccggaacctgaatacgatcccgtcatccaaattgccaacgttgtgagtattgctggcgctaagaaaccattcattcgtaatgtgtttactctgaatacatgctcacccataacaggttcaatgattttttcccacgccactgaagaggaaatgttgagcaattggcgtaactttatcatcaaagttgatcctgatgttatcattggttataatactacaaattttgatatcccttatcttttaaaccgtgcaaaggcgctaaaggtgaatgatttcccatattttggaaggttaaaaaccgttaagcaagaaattaaagagtctgtgttctcttcgaaggcttatggtacaagagaaaccaaaaatgtcaatattgacggccgattacagttggatcttttgcaatttattcagcgtgagtataaactaagatcctacacgttgaatgcagtctctgcgcactttttaggtgaacagaaggaggatgtacattatagcatcatttctgatctacaaaatggcgatagtgaaacaagaagaaggttggccgtttactgtttgaaagacgcctacctgcctttaaggcttatggaaaaactaatggcgttagttaactatacagaaatggctcgtgttacaggtgtgccattttcatatttactagctcgtggtcaacaaattaaagttgtttctcaactatttcgaaagtgcctggagattgatactgtgatacctaacatgcaatctcaggcctctgatgaccaatatgagggtgccactgttattgagcctattcgtggttattacgatgtaccgattgcaactttggatttcaattctttatatccaagtattatgatggcgcacaacctatgttatacaacactttgtaacaaagctactgtagagagattgaatcttaaaattgacgaagactacgtcataacacctaatggagattattttgttaccacaaaaagaaggcgtggtatattaccaattattctggatgaattaataagtgctagaaaacgcgctaaaaaagatctgagagatgagaaggatccattcaaaagagatgttttaaatggtagacaattggctttgaagatttcagctaactctgtctatggttttacaggagcgacggtgggtaaattgccatgtttagccatttcttcatctgttactgcttatggtcgtaccatgattttaaaaactaaaaccgcagtccaagaaaaatattgtataaagaatggttataagcacgatgccgttgtggtttacggtgacactgattccgttatggtaaagtttggtacaacagatttaaaggaagctatggatcttggtaccgaagctgccaaatatgtctccactctattcaaacatccgattaacttagaatttgaaaaagcatacttcccttaccttttgataaataaaaagcgttatgcaggtttattctggactaatcctgacaagtttgacaagttggaccaaaaaggccttgcttctgtccgtcgtgattcctgttccttggtttctattgttatgaataaagttttaaagaaaattttaattgaaagaaatgtagatggtgctttagcttttgtcagagaaactatcaatgatattctgcataatagagtagatatttcaaagttgattatatcaaagacgttagccccaaattacacaaatccacagccgcacgccgttttggctgaacgtatgaagaggagagagggcgttggtccaaatgttggtgatcgtgtggactatgtcattatcggtggtaatgataaactttacaatagagcagaagatccattatttgtactagaaaacaatattcaagtggattcgcgctattatttaactaatcaattacaaaatccaatcattagtattgttgcacctattattggcgacaaacaggcgaacggtatgttcgttgtgaaatccattaaaattaacacaggctctcaaaaaggaggcttgatgagctttattaaaaaagttgaggcttgtaaaagttgtaaaggtccgttgaggaaaggtgaaggccctctttgttcaaactgtctagcaaggtctggagaattatacataaaggcattatacgatgtcagagatttagaggaaaaatactcaagattatggacacaatgccaaaggtgcgctggtaacttacatagtgaagttttgtgttcaaataagaactgtgacattttttatatgcgggttaaggttaaaaaagagctgcaggagaaagtagaacaattaagcaaatggtaaaaaacgatagggtggcacatcatattaggattaagaaaggctaacaactttttgcatgttggtggatatatatgtatatataaatagatac

SEQ ID NO: 2 - protein, 1097 aa, Saccharomyces cerevisiae
MSEKRSLPMVDVKIDDEDTPQLEKKIKRQSIDHGVGSEPVSTIEIIPSDSFRKYNSQGFKAKDTDLMGTQLESTFEQELSQMEHDMADQEEHDLSSFERKKLPTDFDPSLYDISFQQIDAEQSVLNGIKDENTSTVVRFFGVTSEGHSVLCNVTGFKNYLYVPAPNSSDANDQEQINKFVHYLNETFDHAIDSIEVVSKQSIWGYSGDTKLPFWKIYVTYPHMVNKLRTAFERGHLSFNSWFSNGTTTYDNIAYTLRLMVDCGIVGMSWITLPKGKYSMIEPNNRVSSCQLEVSINYRNLIAHPAEGDWSHTAPLRIMSFDIECAGRIGVFPEPEYDPVIQIANVVSIAGAKKPFIRNVFTLNTCSPITGSMIFSHATEEEMLSNWRNFIIKVDPDVIIGYNTTNFDIPYLLNRAKALKVNDFPYFGRLKTVKQEIKESVFSSKAYGTRETKNVNIDGRLQLDLLQFIQREYKLRSYTLNAVSAHFLGEQKEDVHYSIISDLQNGDSETRRRLAVYCLKDAYLPLRLMEKLMALVNYTEMARVTGVPFSYLLARGQQIKVVSQLFRKCLEIDTVIPNMQSQASDDQYEGATVIEPIRGYYDVPIATLDFNSLYPSIMMAHNLCYTTLCNKATVERLNLKIDEDYVITPNGDYFVTTKRRRGILPIILDELISARKRAKKDLRDEKDPFKRDVLNGRQLALKISANSVYGFTGATVGKLPCLAISSSVTAYGRTMILKTKTAVQEKYCIKNGYKHDAVVVYGDTDSVMVKFGTTDLKEAMDLGTEAAKYVSTLFKHPINLEFEKAYFPYLLINKKRYAGLFWTNPDKFDKLDQKGLASVRRDSCSLVSIVMNKVLKKILIERNVDGALAFVRETINDILHNRVDISKLIISKTLAPNYTNPQPHAVLAERMKRREGVGPNVGDRVDYVIIGGNDKLYNRAEDPLFVLENNIQVDSRYYLTNQLQNPIISIVAPIIGDKQANGMFVVKSIKINTGSQKGGLMSFIKKVEACKSCKGPLRKGEGPLCSNCLARSGELYIKALYDVRDLEEKYSRLWTQCQRCAGNLHSEVLCSNKNCDIFYMRVKVKKELQEKVEQLSKW

SEQ ID NO: 3 - DNA, 7505 bp, Saccharomyces cerevisiae
cgctctgccctagttggaatgccatcgaaccacgaggatcttggaatggaaaaccaccgccactgccatttacgtttgcattcatattcgcattaccttggccattcacgggcgtgttcctcggaatttgcattggtcgttgctgttgtggagtgtgggaaaagcgatcgttaacgccattctgctcacttgttgcatatgcgtacggattataagacatcttggtatgggcctttggttttcgttttctgctattgatactcagtagcgaggtcttacaatcgaaaagtcaaaaagatgagttgtagtataaaacaacagctctggtgtgcaatatggatcttgatacagagtctcggatatgctgttttagcactgagaaaaagtaatagtaacactgtcagtgttcgtcaaaggcccaagtttattgtcatttgaattgtcagaatggtttattttcaggtagggtaaccagaacgcgtaagtttcttgcatcttttaccattttaactggaagaggacctatcaaaaagagcatatgatgatgaaagagcacattctatcaagataacactctcaggggacaagtatatgatgtttggcaagaaaaaaaacaacggaggatcttccactgcaagatattcagctggcaacaagtacaacacactctcaaataattatgcgcttagcgcgcaacagctcttaaatgctagtaagatcgatgacatcgattcgatgatgggatttgaaagatacgtaccgccgcaatacaatggcaggtttgatgcgaaggatatagatcagattccaggccgcgtagggtggctgacgaacatgcacgcaacgctggtctctcaggaaaccttatccagtggtagtaatggcggcggcaattcgaatgacggagaacgtgtaacgaccaaccaaggtatttccggagttgacttctactttttagatgaagagggtgggagcttcaagtcgacagttgtctatgacccatacttctttattgcgtgtaacgatgaatcaagagtaaatgatgtggaggaactagtgaaaaaatatctggaatcttgtctcaaaagcttacaaatcattagaaaggaagatcttaccatggacaatcaccttttagggctgcagaagacacttattaagttatcatttgtaaattccaatcagttattcgaggccaggaaactcctgaggccaatcttgcaggataatgccaataataatgtgcaaagaaatatatataacgttgctgcaaatggctcggaaaaagttgacgccaaacatctgatcgaagatatcagggaatatgatgtgccgtatcatgtccgagtatctatagacaaggacattagagtcggtaaatggtataaggtaactcaacagggattcattgaagatactaggaaaattgcatttgccgaccctgtggtaatggcatttgatatagaaaccacgaagccgcctttaaaattcccggattccgccgtagatcaaataatgatgatttcgtatatgatcgatggggaaggttttttgataacaaatagggagataatctctgaggatattgaagactttgagtatacaccgaaaccggagtatcctggttttttcaccatatttaacgaaaacgatgaagtggcgcttctacaaaggttttttgaacatataagagatgtacgacccactgttatatccaccttcaatggtgactttttcgattggccttttatacataacagaagtaagattcacggcttggacatgttcgatgaaattggtttcgctccagatgctgaaggtgagtacaagtcctcatactgctctcacatggattgtttccgttgggtgaagcgtgattcttatttaccacaaggttcccagggtttaaaagctgttactcaatctaagctaggttataacccaattgaactggatcccgaattaatgacgccgtatgcatttgaaaagccacagcacctttccgaatattctgtttccgatgcagtcgctacgtattacctttacatgaaatatgttcatccttttatcttttccctttgtactattattcctttgaacccggatgaaacattgagaaagggtaccggtactttgtgtgaaatgttgttgatggttcaagcttatcaacataatattcttctaccaaataagcatacagatcccattgagaggttctatgatggacatcttctagaatccgagacttacgtgggtggacatgtggagtcattagaagctggtgtttttaggagtgatttgaagaatgaattcaagatagatccttctgccattgatgaattattacaagaattaccagaagctttgaaatttagtgtggaagttgaaaataagtccagtgtagataaagtaacgaattttgaggaaataaaaaaccagataacgcagaaattattagagttgaaggaaaacaatataagaaacgaactacctttgatctatcatgtagatgtcgcctctatgtacccaaacatcatgactacaaatagactacaaccagatagtatcaaagcagagcgcgattgtgctagttgcgattttaatagacccggaaaaacctgtgcaagaaagttaaaatgggcttggagaggagaattctttcccagtaagatggatgagtataacatgatcaagcgtgcattacaaaatgagacttttcccaacaaaaacaagttttctaaaaagaaagttttgacatttgatgaactaagttacgcagaccaagttatccacataaaaaaacgtttaactgaatattcaaggaaagtttatcatagggttaaagtatcagaaattgtcgaacgagaagccattgtctgccaaagagaaaatccattctacgtcgataccgtgaaatcctttcgtgataggcgttacgaattcaaaggtttagccaagacttggaagggaaatctgtccaaaattgacccatctgataagcatgcgagagacgaggccaaaaagatgattgtgctttatgactcattacaattagctcacaaagttattttgaattcgttttatgggtatgttatgaggaaaggctctcgttggtattccatggaaatggcggggattacgtgtttaacaggtgccacgatcattcaaatggcgagagctttagtagaaagggtaggaagaccattagaattagatactgatggtatttggtgtatcttaccaaaatctttccctgaaacttacttttttacattagaaaatggtaaaaagctttatctctcctacccatgttccatgctgaattacagagttcaccaaaagtttacgaatcaccaataccaagaattaaaagacccattgaactatatatatgagacgcacagtgaaaacacgatttttttcgaagttgacggaccatataaggccatgattttgcctagttccaaggaagaaggaaaaggtataaagaaaagatatgctgtcttcaatgaagacggctcacttgctgaactgaaaggttttgaattgaagaggcgtggtgaattacaactaataaaaaattttcaaagtgatattttcaaggtctttttggaaggtgatacattagaaggatgttacagtgctgtagcaagcgtatgtaaccgttggttagatgttcttgattcacatggtcttatgttagaagatgaagacttggtcagtttgatttgtgaaaatagaagtatgtcaaaaactttaaaggaatatgaagggcaaaaatctacttctattacgacggcaaggagattgggggattttttgggtgaagatatggtaaaagataaaggtctacaatgtaaatatattattagttcaaaacctttcaatgcacctgttactgaacgagccattccagtcgcaatattttcagcggacattcccatcaaaaggtcttttctgaggcgatggacattagatccatctttggaagatctggatatcagaaccataatcgattggggttattatagagaaagacttggatctgctatacaaaagataattactattccagcagcattacaaggggtttccaatcctgttccaagggttgaacatccagattggctaaaaagaaaaatcgctacaaaggaggataagtttaagcagacttcactaaccaaatttttttcgaagacaaagaatgtaccaacaatgggcaagataaaagatatcgaggatttgtttgaaccaactgtagaagaagataacgccaaaattaaaattgcaagaactactaaaaagaaagccgtatccaagaggaaaagaaatcagcttacaaatgaagaagatccactagtattgccctcggagattccttccatggacgaggactatgttgggtggctaaattatcaaaaaattaaatggaaaatccaagcaagagatagaaagcgtcgagaccaattatttggtaatacaaacagctcccgtgaaagaagtgcactaggaagtatgattaggaagcaagctgaatcatatgcgaactccacttgggaggtcttacaatacaaggattccggtgagccaggggttttggaagtatttgtaacaattaatggcaaagtccagaacatcaccttccatataccaaaaactatttatatgaaattcaaatctcaaacaatgccgctacaaaagattaagaattgccttattgaaaaatcttctgcatcgttaccaaataatcccaaaacgtctaatccagcaggcggtcagctattcaaaattactctaccggaatctgtctttctggaagaaaaggaaaactgcactagtatcttcaacgatgaaaatgtacttggtgtatttgagggcactatcactcctcatcaaagagcgatcatggatttgggagcttcggtaacgttccgctcaaaagcaatgggtgcgttaggcaagggaatacagcagggttttgaaatgaaggatctttcaatggcggaaaatgaaaggtatctgagtggattttcaatggacattggctatttactacatttcccaacatcaattgggtatgaatttttttcattattcaagtcatggggagatactattactatattagttttgaaaccatccaaccaggctcaggaaataaatgcctcatcattaggacaaatatacaaacaaatgtttgaaaaaaagaaaggtaaaatagaaacatattcttacttggttgatattaaagaagatatcaattttgagtttgtatattttacagatatctcaaaattgtacagaagactatcacaggaaactactaaattaaaagaagaaagaggtctgcagtttttactcttgttacaatctccgtttatcactaagctcttaggcacaatccggcttctaaaccagatgcccattgttaagctttccttgaatgaagttcttctaccccaattgaactggcaaccgacattattgaagaaacttgttaaccacgttttatccagtggttcgtggatttctcacttgatcaagttatcccagtatagtaacattccaatctgtaatttgaggctggatagtatggattatattattgatgttctttatgcaagaaaactaaaaaaagagaacatcgtgctttggtggaatgagaaagctccacttccagatcatggaggcattcaaaatgattttgatttaaatacatcatggataatgaatgattcagaatttcccaaaattaataactcaggtgtgtatgacaatgtagttctcgatgttggtgttgataatttaacagtgaacacaattttgacatcagcattaatcaatgatgctgaaggcagtgatctagttaacaataatatgggtatagatgacaaagatgccgttattaactcgccatctgaattcgtgcacgacgccttttctaatgacgctttgaatgttttaagaggtatgttaaaggagtggtgggatgaggccctaaaagaaaattcaaccgcagatttgttggtaaattccctggcaagttgggttcaaaacccgaatgcgaaactattcgacggattactaagatatcacgttcataacttaacaaaaaaagccttacttcaattagtaaatgaatttagtgcacttggctcaactattgtatatgcagacaggaatcaaattctaataaagacaaacaagtactcacctgaaaactgttacgcctacagccaatatatgatgaaggcagttagaacaaatccaatgtttagttatctggacttaaatatcaaacgttattgggatctgctaatatggatggataagtttaattttagtggattagcatgtattgaaatagaggaaaaggaaaatcaggattataccgctgtttcgcaatggcaactaaagaagtttctgtcaccaatatatcagcccgaatttgaggattggatgatgatcatattggatagtatgctaaagacaaagcagagctatctaaaattgaattcagggacgcaaagacctacccaaatagttaatgtaaaaaaacaagataaggaagatagtgttgaaaactcgttgaacggattttctcaccttttttccaaaccactaatgaaaagagtcaaaaagctttttaaaaaccagcaagagttcattttagatcctcagtatgaggcagactatgttattcctgttcttcctggttcccatctgaatgtgaaaaatccccttctagaacttgtcaaatcactctgccatgtcatgttactttcaaagagtacaattttagaaatcaggaccctgagaaaagaactgctgaagatatttgaattgcgtgagtttgctaaagtagcggaattcaaagatccaagtttgagtctcgtggtgccggattttttatgtgaatactgttttttcatttctgatattgacttttgtaaggcagctcctgaatctattttttcatgcgtcagatgtcacaaagcctttaatcaagtattgttgcaagaacacctgattcaaaaactacgttctgatatcgaatcctatttaattcaagatttgagatgctccagatgtcataaagtgaaacgtgactatatgagtgcccactgtccatgtgccggcgcgtgggaaggaactctccccagagaaagcattgttcaaaagttaaatgtgtttaagcaagtagccaagtattacggttttgatatattattgagttgtattgctgatttgaccatatgagtaagcagtatataacgcgaggttcaatggcctctttaccatgaaaaaaaaaaaaaaaaaaaaaaaaaggtaaggaaaaagagtattttcaattcgtttctgaacatataaatataaataaccgaaaaattagcccttgaacataattaacactcttctttgatatttaaatcacaagtacttttcttttattttcttcttaatacttttggaaataaaatgaatgtgaccactccggaagttgc

SEQ ID NO: 4 - protein, 2222 aa, Saccharomyces cerevisiae
MMFGKKKNNGGSSTARYSAGNKYNTLSNNYALSAQQLLNASKIDDIDSMMGFERYVPPQYNGRFDAKDIDQIPGRVGWLTNMHATLVSQETLSSGSNGGGNSNDGERVTTNQGISGVDFYFLDEEGGSFKSTVVYDPYFFIACNDESRVNDVEELVKKYLESCLKSLQIIRKEDLTMDNHLLGLQKTLIKLSFVNSNQLFEARKLLRPILQDNANNNVQRNIYNVAANGSEKVDAKHLIEDIREYDVPYHVRVSIDKDIRVGKWYKVTQQGFIEDTRKIAFADPVVMAFDIETTKPPLKFPDSAVDQIMMISYMIDGEGFLITNREIISEDIEDFEYTPKPEYPGFFTIFNENDEVALLQRFFEHIRDVRPTVISTFNGDFFDWPFIHNRSKIHGLDMFDEIGFAPDAEGEYKSSYCSHMDCFRWVKRDSYLPQGSQGLKAVTQSKLGYNPIELDPELMTPYAFEKPQHLSEYSVSDAVATYYLYMKYVHPFIFSLCTIIPLNPDETLRKGTGTLCEMLLMVQAYQHNILLPNKHTDPIERFYDGHLLESETYVGGHVESLEAGVFRSDLKNEFKIDPSAIDELLQELPEALKFSVEVENKSSVDKVTNFEEIKNQITQKLLELKENNIRNELPLIYHVDVASMYPNIMTTNRLQPDSIKAERDCASCDFNRPGKTCARKLKWAWRGEFFPSKMDEYNMIKRALQNETFPNKNKFSKKKVLTFDELSYADQVIHIKKRLTEYSRKVYHRVKVSEIVEREAIVCQRENPFYVDTVKSFRDRRYEFKGLAKTWKGNLSKIDPSDKHARDEAKKMIVLYDSLQLAHKVILNSFYGYVMRKGSRWYSMEMAGITCLTGATIIQMARALVERVGRPLELDTDGIWCILPKSFPETYFFTLENGKKLYLSYPCSMLNYRVHQKFTNHQYQELKDPLNYIYETHSENTIFFEVDGPYKAMILPSSKEEGKGIKKRYAVFNEDGSLAELKGFELKRRGELQLIKNFQSDIFKVFLEGDTLEGCYSAVASVCNRWLDVLDSHGLMLEDEDLVSLICENRSMSKTLKEYEGQKSTSITTARRLGDFLGEDMVKDKGLQCKYIISSKPFNAPVTERAIPVAIFSADIPIKRSFLRRWTLDPSLEDLDIRTIIDWGYYRERLGSAIQKIITIPAALQGVSNPVPRVEHPDWLKRKIATKEDKFKQTSLTKFFSKTKNVPTMGKIKDIEDLFEPTVEEDNAKIKIARTTKKKAVSKRKRNQLTNEEDPLVLPSEIPSMDEDYVGWLNYQKIKWKIQARDRKRRDQLFGNTNSSRERSALGSMIRKQAESYANSTWEVLQYKDSGEPGVLEVFVTINGKVQNITFHIPKTIYMKFKSQTMPLQKIKNCLIEKSSASLPNNPKTSNPAGGQLFKITLPESVFLEEKENCTSIFNDENVLGVFEGTITPHQRAIMDLGASVTFRSKAMGALGKGIQQGFEMKDLSMAENERYLSGFSMDIGYLLHFPTSIGYEFFSLFKSWGDTITILVLKPSNQAQEINASSLGQIYKQMFEKKKGKIETYSYLVDIKEDINFEFVYFTDISKLYRRLSQETTKLKEERGLQFLLLLQSPFITKLLGTIRLLNQMPIVKLSLNEVLLPQLNWQPTLLKKLVNHVLSSGSWISHLIKLSQYSNIPICNLRLDSMDYIIDVLYARKLKKENIVLWWNEKAPLPDHGGIQNDFDLNTSWIMNDSEFPKINNSGVYDNVVLDVGVDNLTVNTILTSALINDAEGSDLVNNNMGIDDKDAVINSPSEFVHDAFSNDALNVLRGMLKEWWDEALKENSTADLLVNSLASWVQNPNAKLFDGLLRYHVHNLTKKALLQLVNEFSALGSTIVYADRNQILIKTNKYSPENCYAYSQYMMKAVRTNPMFSYLDLNIKRYWDLLIWMDKFNFSGLACIEIEEKENQDYTAVSQWQLKKFLSPIYQPEFEDWMMIILDSMLKTKQSYLKLNSGTQRPTQIVNVKKQDKEDSVENSLNGFSHLFSKPLMKRVKKLFKNQQEFILDPQYEADYVIPVLPGSHLNVKNPLLELVKSLCHVMLLSKSTILEIRTLRKELLKIFELREFAKVAEFKDPSLSLVVPDFLCEYCFFISDIDFCKAAPESIFSCVRCHKAFNQVLLQEHLIQKLRSDIESYLIQDLRCSRCHKVKRDYMSAHCPCAGAWEGTLPRESIVQKLNVFKQVAKYYGFDILLSCIADLTI

SEQ ID NO: 5 - protein, 12 aa, Escherichia coli
QIVLDTETTGMN

SEQ ID NO: 6 - protein, 12 aa, Haemophilus influenzae
QIVLDTETTGMN

SEQ ID NO: 7 - protein, 12 aa, Salmonella typhimurium
QIVLDTETTGMN

SEQ ID NO: 8 - protein, 12 aa, Vibrio cholerae
IVVLDTETTGMN

SEQ ID NO: 9 - protein, 12 aa, Pseudomonas aeruginosa
SVVLDTETTGMP

SEQ ID NO: 10 - protein, 12 aa, Neisseria meningitidis
QIILDTETTGLY

SEQ ID NO: 11 - protein, 12 aa, Chlamydia trachomatis
FVCLDCETTGLD

SEQ ID NO: 12 - protein, 12 aa, Streptomyces coelicolor
LAAFDTETTGVD

SEQ ID NO: 13 - protein, 12 aa, Shigella flexneri 2a str. 301
QIVLDTETTGMN

SEQ ID NO: 14 - protein, 12 aa, Staphylococcus aureus
YVVFDVETTGLS

SEQ ID NO: 15 - protein, 12 aa, Bacillus subtilis
YVVFDVETTGLS

SEQ ID NO: 16 - protein, 12 aa, Mycoplasma pulmonis
YVVYDIETTGLS

SEQ ID NO: 17 - protein, 12 aa, Mycoplasma genitalium
FVIFDIETTGLH

SEQ ID NO: 18 - protein, 12 aa, Mycoplasma pneumoniae
FVIFDIETTGLH

SEQ ID NO: 19 - protein, 12 aa, Saccharomyces cerevisiae
IMSFDIECAGRI

SEQ ID NO: 20 - protein, 12 aa, Saccharomyces cerevisiae
VMAFDIETTKPP

SEQ ID NO: 21 - protein, 12 aa, Mus musculus
VLSFDIECAGRK

SEQ ID NO: 22 - protein, 12 aa, Mus musculus
VLAFDIETTKLP

SEQ ID NO: 23 - protein, 12 aa, Homo sapiens
VLSFDIECAGRK

SEQ ID NO: 24 - protein, 12 aa, Homo sapiens
VLAFDIETTKLP

SEQ ID NO: 25 - protein, 12 aa, Oryza sativa
ILSFDIECAGRK

SEQ ID NO: 26 - protein, 12 aa, Arabidopsis thaliana
VLSFDIECAGRK

SEQ ID NO: 27 - protein, 12 aa, Arabidopsis thaliana
VCAFDIETVKLP

SEQ ID NO: 28 - protein, 12 aa, Rattus norvegicus
VLSFDIECAGRK

SEQ ID NO: 29 - protein, 12 aa, Bos taurus
VLSFDIECAGRK

SEQ ID NO: 30 - protein, 12 aa, Glycine max
ILSFDIECAGRK

SEQ ID NO: 31 - protein, 12 aa, Drosophila melanogaster
ILSFDIECAGRK

SEQ ID NO: 32 - protein, 12 aa, Drosophila melanogaster
VLAFDIETTKLP

SEQ ID NO: 33 - DNA, 36 bp, Artificial Sequence
  Description of Artificial SequenceMutated pol delta
atcatgtcctttgctatcgcttgtgctggtaggatt

SEQ ID NO: 34 - protein, 12 aa, Artificial Sequence
  Description of Artificial SequenceMutated pol delta
IMSFAIACAGRI

SEQ ID NO: 35 - DNA, 36 bp, Artificial Sequence
  Description of Artificial SequenceMutated pol epsilon
gtaatggcatttgctatagctaccacgaagccgcct

SEQ ID NO: 36 - protein, 12 aa, Artificial Sequence
  Description of Artificial SequenceMutated pol epsilon
VMAFAIATTKPP

SEQ ID NO: 37 - DNA, 30 bp, Artificial Sequence
  Description of Artificial SequencePrimer
cccgagctcatgagtgaaaaaagatccctt

SEQ ID NO: 38 - DNA, 30 bp, Artificial Sequence
  Description of Artificial SequencePrimer
cccgcggccgcttaccatttgcttaattgt

SEQ ID NO: 39 - DNA, 30 bp, Artificial Sequence
  Description of Artificial SequencePrimer
cccgagctcatgatgtttggcaagaaaaaa

SEQ ID NO: 40 - DNA, 30 bp, Artificial Sequence
  Description of Artificial SequencePrimer
cccgcggccgctcatatggtcaaatcagca

SEQ ID NO: 41 - DNA, 1592 bp, Escherichia coli
gaattcaaatacaaaaaaaccgcaaaattaaaaatcttgcggctctctgaactcattttcatgagtgaatagtggcggaacggacgggactcgaacccgcgaccccctgcgtgacaggcaggtattcaaccgactgaactaccgctccgcgttgtgttccgttgggaacgaggcgaatagttacgaattgcctcgacctcgtcaacggtttttctatcttttgaatcgtttgctgcaaaaatcgcccaagtcgctatttttagcgcctttcacaggtatttatgctcgccagaggcaacttccgcctttcttctgcaccagatcgagacgggcttcatgagctgcaatctcttcatctgtcgcaaaaacaacgcgtaacttacttgcctgacgtacaatgcgctgaattgttgcttcaccttgttgctgttgtgtctctccttccatcgcaaaagccatcgacgtttgaccaccggtcatcgccagataaacttccgcaaggatctgggcatcgagtaatgccccgtgcagcgttcgtttactgttatctatttcgtagcgagcacataacgcatcgaggctgttgcgcttaccgggaaacattttcctcgccaccgcaaggctatcggtgaccttacagaaagtattggtcttcggaatatcgcgcttaagcaacgaaaactcgtagtccataaagccgatatcgaacgctgcgttatggatcaccaactccgcgccgcgaatatagtccatgaactcatcggctacttcggcaaacgtgggcttatcgagcaaaaattcatcggcaataccatgtacgccaaaggcttccggatccaccagccgatcgggtttgagataaacatggaagttattgcccgtcaggcgacggttcaccacttcaacggcaccaatctcaatgatcttgtggccttcatagtgcgcaccaatctggttcataccggtggtttcggtatcgagaacgatctggcgtgtaattgcagtgctcatagcggtcatttatgtcagacttgtcgttttacagttcgattcaattacaggaagtctaccagagatgcttaaacaggtagaaattttcaccgatggttcgtgtctgggcaatccaggacctgggggttacggcgctattttacgctatcgcggacgcgagaaaacctttagcgctggctacacccgcaccaccaacaaccgtatggagttgatggccgctattgtcgcgctggaggcgttaaaagaacattgcgaagtcattttgagtaccgacagccagtatgtccgccagggtatcacccagtggatccataactggaaaaaacgtggctggaaaaccgcagacaaaaaaccagtaaaaaatgtcgatctctggcaacgtcttgatgctgcattggggcagcatcaaatcaaatgggaatgggttaaaggccatgccggacacccggaaaacgaacgctgtgatgaactggctcgtgccgcggcgatgaatcccacactggaagatacaggctaccaagttgaagtttaagcctgtggtttacgacattgccgggtggctccaaccgcctagcgaattc

SEQ ID NO: 42 - protein, 243 aa, Escherichia coli
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMAFAMEGETQQQQGEATIQRIVRQASKLRVVFATDEEIAAHEARLDLVQKKGGSCLWRA

SEQ ID NO: 43 - DNA, 4866 bp, Bacillus subtilis
ggtaccgcttcacttatgatgtttttagggagggatactgtcttaatggaacagttatcagtaaacagaaggcagtttcaaattcttctgcagcagattaatatgacagatgataccttcatgacatactttgaacatggcgagattaaaaagctgacaattcacaaagcttctaagtcttggcattttcattttcaatttaaatctttgctgccttttcaaatatatgacacattaacaacgaggctgacgcaatcgtttgcccacatagcaaaagtgacatcttcaattgaagttcaggatgccgaggtcagtgaaagtatcgttcaagactactggtcacgctgcattgaagaactgcagggcatttcgccgccgattatcagtcttttaaaccagcaaaaaccgaagctgaagggcaataaactgattgtcaaaaccaaaacagatacagaagcggctgcgctaaagaacaaatacagttccatgattcaagcagaataccgtcaatttggctttccggatcttcagcttgatgctgaaatatttgtatccgagcaagaagttcaaaagtttcgggagcaaaagcttgcggaagaccaagagcgggctatgcaggccttgattgaaatggagaagaaagataaagaaagtgatgaagaccaagcaccatctggtcctcttgttatcggttatcaaattaaagataacgaggaaatccgaacacttgacagcatcatggacgaagaacggagaattacggtccaaggttatgtgtttgatgtggagacgcgcgagctgaagagcgggcgcacgctgtgtatcttcaaaattacagactatacaaatagtattttgatcaaaatgtttgcacgtgaaaaagaagatgcggcgctgatgaagtctctgaaaaaaggaatgtgggtaaaagcacgcggaagcattcaaaatgatacatttgtcagagaccttgtcatgatcgcaaatgacgtaaacgaaataaaagcaaaaacccgtgaagattcagcacctgaaggagaaaaaagagtggaattgcatcttcattccccaatgagccaaatggatgctgttacgggtatcggaaagcttgtcgaacaggcgaaaaaatgggggcatgaggccatcgctttgaccgaccatgctgtcgttcaatccttccctgatgcgtattctgcggccaaaaagcatggaattaaaatgatttacgggatggaagcgaatctcgtggatgatggcgtgccaattgcttataatgccgcacatcgtctgctcgaagaagaaacatatgttgtttttgacgttgagacgacaggattgtctgctgtatacgataccattattgagctggctgcagtaaaagtaaaaggcggagaaattattgataaatttgaggcgtttgcgaacccgcatcgtccgctttccgccacaatcatagagctgacagggatcacagatgatatgctacaagacgctccggatgtcgtagatgtaataagagatttcagagaatggattggcgatgatattcttgtcgctcataatgcaagctttgatatgggattcttaaatgtagcctataaaaaacttcttgaagtcgaaaaagctaaaaacccagtcattgatacgcttgaacttggacgttttctctatccggaatttaagaaccaccggttgaacacactttgtaaaaagtttgatatcgagctcacacagcatcaccgtgcgatctatgatactgaggcaaccgcttatttgcttctgaaaatgctgaaagacgcagctgaaaaaggtattcagtaccatgatgagttgaatgaaaatatgggtcagtccaatgcttatcaaagatcaagaccgtatcatgcaacattacttgccgtgaacagcacgggacttaaaaatttatttaagcttgtgtcactttctcatattcattatttttacagagtgccgcgtattccgagatctcagcttgagaaatacagggaagggcttctgatcggttctgcttgtgacaggggagaggtttttgagggaatgatgcaaaaatcgcctgaagaggtggaagatatcgcgtcattctatgattaccttgaggttcagccgcctgaagtgtatcgtcacttgctggagcttgaactggtccgtgatgaaaaagcgctgaaagaaattattgcgaatatcacgaagctgggggaaaagcttaataaaccggttgttgctacgggaaatgttcattacttgaatgatgaggataaaatctacagaaagattttaatatcctcacaaggcggggcaaatccgctgaataggcatgaactgccgaaagtgcatttcagaacgacagacgaaatgcttgaagctttttctttcttaggtgaagaaaaagcgaaggagatcgtagtcaccaatacccaaaaggttgcttctttagttgatgacatcaagccgattaaagatgatttatatacgccgaaaatcgaaggcgctgatgaagagatcagagaaatgagctatcagcgtgcaagaagcatttacggggaagagctgcctgaaattgtcgaagcgcggattgaaaaagagttaaagagtattattggccacggatttgctgttatttacttgatctctcacaaacttgtaaaacgttcactagatgacgggtatctcgttggttcccgtggttccgtaggatcttcattagttgcgacacttactgagattactgaggtaaacccgctgccgccgcactatgtttgtcctgagtgccagcattctgagttctttaatgacggttctgtcggttctggttttgacctgcctgacaagacatgccctcattgcggaacgcctttgaaaaaagacggccatgatattccatttgaaacgttcttaggatttaaaggggacaaagtacctgatatcgatttgaacttctcaggggaatatcagccgcaagcacacaattacacaaaagtattgttcggagaagacaatgtatatcgtgcgggaacaataggcacggtggcagaaaaaacagcctacggttatgtaaaaggctatgccggagacaacaatcttcatatgcgcggtgccgaaatagatcggctcgtacagggatgcacaggtgtaaaacgtacaactggacagcaccctggcggtattatcgtagttccggattatatggatatttatgatttttcaccgatccagttcccggcagatgccacaggttcagagtggaaaacgactcattttgatttccactccatccatgacaacctgttaaaacttgatattctcggacacgatgacccgactgttattcggatgcttcaagacttaagcggaatagatccgaaaacaattccgacggatgatcctgaagtgatgaagatcttccagggaaccgaatcactcggtgtgactgaagaacagattggctgtaaaacgggcactcttggaattcctgaattcggaacccgatttgtccggcagatgcttgaagatacaaagccgaccactttttctgagctcgttcagatttcaggcttgtctcacggaactgatgtatggcttggcaatgcacaggagctcatccacaataatatttgtgagctgagtgaggttatcggctgccgtgatgacattatggtttatttaatctatcaaggccttgagccgtcccttgcctttaaaatcatggaattcgtgcgtaaaggaaaaggattaacgcctgaatgggaagaagaaatgaaaaataacaatgtcccagactggtatattgattcctgtaaaaagattaaatacatgttcccgaaagcccacgccgcggcatatgtcttaatggcagtccgcattgcttactttaaagtgcatcatgctcttttgtattatgcggcttattttaccgttcgtgcagatgactttgatattgatacaatgatcaagggctctacagcaatcagagcggtaatggaggatataaacgctaaaggacttgatgcttcaccgaaggaaaagaaccttctgactgttttagaattagcgcttgagatgtgtgagagaggctattcattccaaaaagtcgatttatatcgctccagcgccacagagtttattattgacggcaacagtcttattccgccgtttaactctattccagggttagggacgaacgctgctttgaacattgtaaaagctcgcgaagaaggcgaattcctctcaaaagaagatttgcaaaagagagggaaagtatcaaaaacgattttagagtacttagatcgccatggctgtctggagtcactgcctgatcaaaaccaattgtcactgttctaatatggaaagcagaatttctcagaaattctgcttctatgcatacataagcgcaaaaagtgccatcgtaatattagagtttctgtcacttgcttaggtatgaaggtaagcgtatatccatttgcaataaaaatatggttatggtatagttttattggaaatgctaacgattaccgaggcaaagagtggggaaacccgctcttttgtattgaacaggagaattttgtctcgacatgttcatcgtttactttttagcccctgctcttttgaagcagggtttttatgcagagtgacgagacgaatatgagatcgacagcacaaggaggaagaacatgagcaaaaaagtgactgacaccgttcaagaaatggctcagccaatcgtagacagccttcagctggaactcgttgacattgaatttgtcaaagagggccaaagctggttccttcgcgtgtttattgattccgatgacggtgtggatattgaggaatgtgccaaagtaagcgaagctt

SEQ ID NO: 44 - protein, 1437 aa, Bacillus subtilis
MEQLSVNRRQFQILLQQINMTDDTFMTYFEHGEIKKLTIHKASKSWHFHFQFKSLLPFQIYDTLTTRLTQSFAHIAKVTSSIEVQDAEVSESIVQDYWSRCIEELQGISPPIISLLNQQKPKLKGNKLIVKTKTDTEAAALKNKYSSMIQAEYRQFGFPDLQLDAEIFVSEQEVQKFREQKLAEDQERAMQALIEMEKKDKESDEDQAPSGPLVIGYQIKDNEEIRTLDSIMDEERRITVQGYVFDVETRELKSGRTLCIFKITDYTNSILIKMFAREKEDAALMKSLKKGMWVKARGSIQNDTFVRDLVMIANDVNEIKAKTREDSAPEGEKRVELHLHSPMSQMDAVTGIGKLVEQAKKWGHEAIALTDHAVVQSFPDAYSAAKKHGIKMIYGMEANLVDDGVPIAYNAAHRLLEEETYVVFDVETTGLSAVYDTIIELAAVKVKGGEIIDKFEAFANPHRPLSATIIELTGITDDMLQDAPDVVDVIRDFREWIGDDILVAHNASFDMGFLNVAYKKLLEVEKAKNPVIDTLELGRFLYPEFKNHRLNTLCKKFDIELTQHHRAIYDTEATAYLLLKMLKDAAEKGIQYHDELNENMGQSNAYQRSRPYHATLLAVNSTGLKNLFKLVSLSHIHYFYRVPRIPRSQLEKYREGLLIGSACDRGEVFEGMMQKSPEEVEDIASFYDYLEVQPPEVYRHLLELELVRDEKALKEIIANITKLGEKLNKPVVATGNVHYLNDEDKIYRKILISSQGGANPLNRHELPKVHFRTTDEMLEAFSFLGEEKAKEIVVTNTQKVASLVDDIKPIKDDLYTPKIEGADEEIREMSYQRARSIYGEELPEIVEARIEKELKSIIGHGFAVIYLISHKLVKRSLDDGYLVGSRGSVGSSLVATLTEITEVNPLPPHYVCPECQHSEFFNDGSVGSGFDLPDKTCPHCGTPLKKDGHDIPFETFLGFKGDKVPDIDLNFSGEYQPQAHNYTKVLFGEDNVYRAGTIGTVAEKTAYGYVKGYAGDNNLHMRGAEIDRLVQGCTGVKRTTGQHPGGIIVVPDYMDIYDFSPIQFPADATGSEWKTTHFDFHSIHDNLLKLDILGHDDPTVIRMLQDLSGIDPKTIPTDDPEVMKIFQGTESLGVTEEQIGCKTGTLGIPEFGTRFVRQMLEDTKPTTFSELVQISGLSHGTDVWLGNAQELIHNNICELSEVIGCRDDIMVYLIYQGLEPSLAFKIMEFVRKGKGLTPEWEEEMKNNNVPDWYIDSCKKIKYMFPKAHAAAYVLMAVRIAYFKVHHALLYYAAYFTVRADDFDIDTMIKGSTAIRAVMEDINAKGLDASPKEKNLLTVLELALEMCERGYSFQKVDLYRSSATEFIIDGNSLIPPFNSIPGLGTNAALNIVKAREEGEFLSKEDLQKRGKVSKTILEYLDRHGCLESLPDQNQLSLF

SEQ ID NO: 45 - protein, 1081 aa, Arabidopsis thaliana
MNRSGISKKRPPPSNTPPPAGKHRATGDSTPSPAIGTLDDEFMMEEDVFLDETLLYGDEDEESLILRDIEERESRSSAWARPPLSPAYLSNSQIFQQLEIDSIIAESHKELLPGSSGQAPIIRMFGVTREGNSVCCFVHGFEPYFYIACPPGMGPDDISNFHQSLEGRMRESNKNAKVPKFVKRIEMVQKRSIMYYQQQKSQTFLKITVALPTMVASCRGILDRGLQIDGLGMKSFQTYESNILFVLRFMVDCDIVGGNWIEVPTGKYKKNARTLSYCQLEFHCLYSDLISHAAEGEYSKMAPFRVLSFDIECAGRKGHFPEAKHDPVIQIANLVTLQGEDHPFVRNVMTLKSCAPIVGVDVMSFETEREVLLAWRDLIRDVDPDIIIGYNICKFDLPYLIERAATLGIEEFPLLGRVKNSRVRVRDSTFSSRQQGIRESKETTIEGRFQFDLIQAIHRDHKLSSYSLNSVSAHFLSEQKEDVHHSIITDLQNGNAETRRRLAVYCLKDAYLPQRLLDKLMFIYNYVEMARVTGVPISFLLARGQSIKVLSQLLRKGKQKNLVLPNAKQSGSEQGTYEGATVLEARTGFYEKPIATLDFASLYPSIMMAYNLCYCTLVTPEDVRKLNLPPEHVTKTPSGETFVKQTLQKGILPEILEELLTARKRAKADLKEAKDPLEKAVLDGRQLALKISANSVYGFTGATVGQLPCLEISSSVTSYGRQMIEQTKKLVEDKFTTLGGYQYNAEVIYGDTDSVMVQFGVSDVEAAMTLGREAAEHISGTFIKPIKLEFEKVYFPYLLINKKRYAGLLWTNPQQFDKMDTKGIETVRRDNCLLVKNLVTESLNKILIDRDVPGAAENVKKTISDLLMNRIDLSLLVITKGLTKTGDDYEVKSAHGELAERMRKRDAATAPNVGDRVPYVIIKAAKGAKAYERSEDPIYVLQNNIPIDPNYYLENQISKPLLRIFEPVLKNASKELLHGSHTRSISITTPSNSGIMKFAKKQLSCVGCKVPIRYFVQWNTMRKLQGKRSRVILQKRVSRYAAWLSLKRFLGGCGHSARSVKALFIKMSCAPVEIVQYFTGE

SEQ ID NO: 46 - protein, 2154 aa, Arabidopsis thaliana
MSGRRCDRRLNVQKVSAADELETKLGFGLFSQGETRLGWLLTFASSSWEDADTGKTFSCVDLFFVTQDGSSFKTKYKFRPYLYAATKDNMELEVEAYLRRRYERQVADIQIVHKEDLYLKNHLSGLQKKYLKVSFDTVQQLVEVKRDLLHIVERNLAKFNALEAYESILSGKREQRPQDCLDSVVDLREYDVPYHVRFAIDNDVRSGQWYNVSISSTDVILEKRTDLLQRAEVRVCAFDIETVKLPLKFPDAEYDQIMMISYMVDGQGFLITNRECVGKDIEDLEYTPKPEFEGYFKVTNVTNEVELLRKWFSHMQELKPGIYVTYNGDFFDWPFIERRASHHGIKMNEELGFRCDQNQGECRAKFVCHLDCFSWVKRDSYLPQGSQGLKAVTKVKLGYDPLEVNPEDMVRFAMEKPQTMASYSVSDAVATYYLYMTYVHPFVFSLATIIPMVPDEVLRKGSGTLCEMLLMVEAYKANVVCPNKNQADPEKFYQGKLLESETYIGGHVECLQSGVFRSDIPTSFKLDASAYQQLIDNLGRDLEYAITVEGKMRMDSVSNFDEVKEVIREKLEKLRDDPIREEGPLIYHLDVAAMYPNIILTNRLQPPSIVTDEVCTACDFNGPEKTCLRKLEWVWRGVTFKGNKSEYYHLKKQIESESVDAGANMQSSKPFLDLPKVEQQSKLKERLKKYCQKAYSRVLDKPITEVREAGICMRENPFYVDTVRSFRDRRYEYKTLNKVWKGKLSEAKASGNLIKIQEAHDMVVVYDSLQLAHKCILNSFYGYVMRKGARWYSMEMAGVVTYTGAKIIQNARLLIERIGKPLELDTDGIWCALPGSFPENFTFKTIDMKKFTISYPCVILNVDVAKNNSNDQYQTLVDPVRKTYNSRSECSIEFEVDGPYKAMIIPASKEEGILIKKRYAVFNHDGTIAELKGFEMKRRGELKLIKVFQAELFDKFLHGSTLEECYSAVAAVANRWLDLLEGQGKDIADSELLDYISESSTMSKSLADYGQQKSCAVTTAKRLADFLGDTMVKDKGLRCQYIVAREPEGTPVSERAVPVAIFQTDDPEKKFYLQKWCKISSYTGIRSIIDWMYYKQRLHSAIQKVITIPAAMQKVANPVLRVRHPYWLEKKVCDKFRQGKIVDMFSSANKDHSTTQDNVVADIEEFCKENRPSVKGPKPVARSFEVDRNHSEGKQQESWDPEFHDISLQNVDKNVDYQGWLELEKRKWKMTLTNKKKRRFDDLKPCNQIDAHKINKKVCKGRVGVGSYFRRPEEALTSSYLQIIQLVQSPQSGQFFAWVVVEGLMLKIPLTIPRVFYINSKASIAGNFTGKCINKILPHGKPCYNLMEARHLHNTHILLLVNIQEDQFIKESKKLAALLADPEIEGIYETKMPLEFSAICQIGCVCKIEDTAKHRNTQDGWKLGELHRITTTECRYLENSIPLVYLYHSTSTGRAVYVLYCHASKLMSVVVVNPYGDKELLSSALERQFRDRCQELSPEPFSWDGILFQVEYVDHPEAATKFLQKALCEYREENCGATVAVIECPDFNTTKEGVKALEDFPCVRIPFNDDDNSYQPVSWQRPAAKIAVLRCASAIQWLDRRIAQSRYAHVPLGNFGRDWLTFTVDIFLSRALRDQQQVLWVSDNGVPDLGDINNEETFLADELQETSLLFPGAYRKVSVELKVHRLAVNALLKSDLVSEMEGGGFLGVNSRGSSLNDNGSFDENNGCAQAFRVLKQLIKRLLHDACNSGNIYADSILQHLSWWLRSPSSKLHDPALHLMLHKVMQKVFALLLTDLRRLGAIIIYADFSKVIIDTGKFDLSAAKTYCESLLTVMGSRDIFKLILLEPVHYWHSLLFMDQHNYAGIRATGDEISGNEVTIEPKWSVARHLPEYIQKDFIIIVATFIFGPWKFALEKKRGSAESLEAEMVEYLKEQIGTRFISMIVEKIGNIRSHIKDINVSDASWASGQAPKGDYTFEFIQIITAVLALDQNVQQDVLVMRKILLKYIKVKECAAEAEFIDPGPSFILPNVACSNCGAYRDLDFCRDSALLTEKEWSCADPQCVKIYDKEQIESSIIQMVRQRERMYQLQDLVCNRCNQVKAAHLTEQCECSGSFRCKESGSDFHKRIEIFLDIAKRQKFRLLEECISWILFATSC

SEQ ID NO: 47 - DNA, 3706 bp, Oryza sativa
ctctcttcccgcgttcccctctccctccccctcccccctctccggcgatgagctcaggcggacgcggcggcaagcggcgaggggcgccgcccccggggccatccggggcggcggcgaagcgggcccaccccggtggcaccccgcagccgcctccgcccgccgcgacggcggcggcgcccgtggcggaggaggaggacatgatggacgaggacgtcttcctcgacgagaccatcctggcggaggacgaggaggcgttgctgctgctcgaccgggacgaggccctcgcctcacgcctctcccgctggaggcgccccgcgctccccgccgacctagcgtccggctgctcgcgcaatgttgcttttcagcagctggagatagattatgttattggtgagagccacaaagtactgctccccaactcatctggtcctgcagctatactcaggatatttggcgtaactagagaaggtcacagtgtatgctgccaagtgcatggatttgagccatatttttacatcagttgtccaatggggatgggccctgatgatatttcacgcttccaccaaacactagaggggaggatgaaggattcaaataggaacagcaacgtgccaaggtttgtgaagagaatcgaacttgtgcagaagcagacaatcatgcattaccaaccacagcaatctcagcctttcctcaagatagtggttgctttgccaacaatggttgctagttgtcgcggcatcctggaaaggggcataacaattgaaggccttggttcgaagagttttctgacatatgaaagcaacattctttttgcacttcgcttcatgattgactgcaatattgttggtggtaattggattgaagttcctgccggaaagtatatgaaggcagctcgtatcatgtcctattgtcagctagagttggattgcctatattcggatttggtaagccatgctgctgaaggagaacattctaagatggctccatttcgcatattaagttttgatattgaatgtgccggtcgcaaaggtcacttcccagaaccaactcatgatcccgttattcagatagctaacttggtcacccttcaaggagaaggacaaccttttgtacgcaatgttatgacgcttaaatcatgttctcccattgttggagttgatgttatgtcatttgacacagagagggatgttctacttgcttggagggatttcatacgtgaagtggaccctgatattattattggatacaatatctgcaaatttgacttaccctatcttattgagagagctgaagttcttaagatagtagagtttccaatacttggacgaatcagaaatagtcgtgttcgtgtccgtgacacaactttctcttcaaggcaatatggtatgcgtgaaagtaaagatgtagcagtggaaggaagagtacaatttgatcttctgcaggctatgcaacgggattacaagcttagttcttattcattaaactctgtatctgcacatttcctcggggagcaaaaagaggatgttcatcactcaattatatctgatcttcaaaatgggaattcagagacacgaagacggcttgcggtttattgtttgaaggatgcctatcttccacaacgactgctagataagttgatgtatatctacaactatgtggaaatggcaagagtcactggagttcccatttcatttcttctttcaaggggacagagcattaaggtcctctcacagctactcaggaaagcaaaacagaaaaaccttgttataccaaatataaagggtcaagcgtctggacaggatacctttgaaggtgcaactgttttggaggcaagggctggattttatgagaaacccattgcgactttggactttgcttcgttgtatccatccatcatgatggcatataacctatgctactgtactttggtcccccctgaggatgcccgcaaactcaacctgcctccagaaagtgtcaacaaaaccccatctggtgaaacatttgtgaaaccagatgtgcaaaagggtatacttcctgaaatccttgaagaattgttggctgctcggaaaagggcgaaagcagatttgaaggaagcaaaggatccatttgaaagggccgttcttgatggtcgtcagcttgccctaaaaataagcgcaaactctgtctatggttttactggagcgactgttggtcaattaccttgtttagaaatttcttcaagtgtgaccagctatggtcgacagatgattgaacatacaaaaaagcttgttgaagataaattcacgacacttggaggctatgagcacaatgcagaggtcatctatggagatactgattctgtaatggtacagttcggtgtttctactgttgaggacgcaatgaagctaggaagagaagctgcagactacattagtggaacatttattaagcccatcaagcttgagtttgagaagatctatttcccttatctactgattagcaagaagagatatgctggtttgtactggacaaatcctgagaaatttgacaaaatggacacgaaaggtattgaaacagtgagaagggacaactgtttattagtaaagaacctggttactgagtgccttcataaaatactagtggacagagatgttcctggtgcagttcaatatgtcaagaacaccatttctgatctactaatgaaccgtgtagacttatctcttctagttataacaaagggtttgactaaaacaggagaggattatgctgtcaaagctgcccatgtggagcttgctgagagaatgcgaaagagggacgctgctactgctcctactgttggtgaccgggttccttatgttataatcaaagcagcaaaaggggcaaaggcatatgagaggtcagaagatcctatttatgttttggataataacataccaatagatccccaatactaccttgagaaccaaatcagcaaaccacttttgaggatctttgagccgattctgaagaatgccagtagagagctgcttcatggaagtcacaccagggctgtttcaatctcaactccttcaaatagtggaataatgaaatttgcaaagaaacaattgacatgcctcggatgcaaagcagttataagtggttccaatcaaacgctttgctttcattgcaagggaagagaagcagagttatactgcaaaactgtaggaaacgtttctgagctggagatgctctttgggaggctctggacgcagtgccaggagtgccaaggctcccttcatcaggacgttctctgcacaagccgggattgtcctattttctaccgccgaagaaaggcgcagaaggatatggctgaagctagagtacagcttcaacgttgggacttctgagtcctctcatactgacggagtactactttccccaaatattgcgaaaccattactgtgaggcacgccattgcgggatcatgtgattgcatcttcatgcatgatggctctggcttgtttagttggatcggctgaaatagctttgttctacggtcagtttgttgtatttttaggtggtaggttatctgtacctctagccgctaacagggtaatctagttgcttcccttggtgcattgatgcagccatgtgtaaggtagataaacaattttttttcatcatcttttaacttcatgaggtgattgaggctgagaagcacccattcaagaaaaaaaaaaaaaaaaaa

SEQ ID NO: 48 - protein, 1105 aa, Oryza sativa
MSSGGRGGKRRGAPPPGPSGAAAKRAHPGGTPQPPPPAATAAAPVAEEEDMMDEDVFLDETILAEDEEALLLLDRDEALASRLSRWRRPALPADLASGCSRNVAFQQLEIDYVIGESHKVLLPNSSGPAAILRIFGVTREGHSVCCQVHGFEPYFYISCPMGMGPDDISRFHQTLEGRMKDSNRNSNVPRFVKRIELVQKQTIMHYQPQQSQPFLKIVVALPTMVASCRGILERGITIEGLGSKSFLTYESNILFALRFMIDCNIVGGNWIEVPAGKYMKAARIMSYCQLELDCLYSDLVSHAAEGEHSKMAPFRILSFDIECAGRKGHFPEPTHDPVIQIANLVTLQGEGQPFVRNVMTLKSCSPIVGVDVMSFDTERDVLLAWRDFIREVDPDIIIGYNICKFDLPYLIERAEVLKIVEFPILGRIRNSRVRVRDTTFSSRQYGMRESKDVAVEGRVQFDLLQAMQRDYKLSSYSLNSVSAHFLGEQKEDVHHSIISDLQNGNSETRRRLAVYCLKDAYLPQRLLDKLMYIYNYVEMARVTGVPISFLLSRGQSIKVLSQLLRKAKQKNLVIPNIKGQASGQDTFEGATVLEARAGFYEKPIATLDFASLYPSIMMAYNLCYCTLVPPEDARKLNLPPESVNKTPSGETFVKPDVQKGILPEILEELLAARKRAKADLKEAKDPFERAVLDGRQLALKISANSVYGFTGATVGQLPCLEISSSVTSYGRQMIEHTKKLVEDKFTTLGGYEHNAEVIYGDTDSVMVQFGVSTVEDAMKLGREAADYISGTFIKPIKLEFEKIYFPYLLISKKRYAGLYWTNPEKFDKMDTKGIETVRRDNCLLVKNLVTECLHKILVDRDVPGAVQYVKNTISDLLMNRVDLSLLVITKGLTKTGEDYAVKAAHVELAERMRKRDAATAPTVGDRVPYVIIKAAKGAKAYERSEDPIYVLDNNIPIDPQYYLENQISKPLLRIFEPILKNASRELLHGSHTRAVSISTPSNSGIMKFAKKQLTCLGCKAVISGSNQTLCFHCKGREAELYCKTVGNVSELEMLFGRLWTQCQECQGSLHQDVLCTSRDCPIFYRRRKAQKDMAEARVQLQRWDF

SEQ ID NO: 49 - DNA, 3427 bp, Glycine max
tgattaccctcccactccacactctccgctgtctctccctcccaattccgatgagcaacaacgcctcccggaagcgcgcgccgccgcctccgtcccaacctccgccggcgaacaagccctaatgactcaggaagaagagttcatggacgaagacgtgttcataaacgaaaccctcgtctccgaggacgaagaatccctcattctccgcgacattgagcagcgccaggccctcgccaaccgcctctccaagtggacacgtcctcctctctccgccggctacgtcgcccaatctcgtagcgtcctttttcagcagctagagattgattacgtgattgcagagagtcacggggagttgctgccgaactcgtctggacctgtcgccattatcagaatatttggagttactaaggaaggacacagtgtttgttgcaatgttcatgggtttgaaccatatttctacatctgttgccctcctggaatgggtccagatgatatctcccattttcatcaaactctcgagggaaggatgagagaagccaatagaaacagtaatgtgggaaaattcgttcgccgtattgaaatggtgcagagaaggagtattatgtactatcagcaatccaattcccaaccctttctcaaaattgtagttgcactcccaacaatggttgccagctgccgtggtattcttgataggggtattcaacttgatggtctgggaatgaagagcttcttgacttatgaaagcaatgtactttttgcccttcgcttcatgattgattgtaacatagttggtggaaattggattgggattcctgccggaaaatataagaaaacagcgaaaagcttgtcttactgccagttagagtttgattgcttgtattctgaattgattagtcatgctccagaaggggaatattcaaagatggctccgtttcgcattttgagttttgacatcgagtgtgctggtcgtaaaggtcattttcctgagcctacccatgatcctgttatccagattgctaatttggttactttacaaggagaagaccagccatttattcgtaatgtgatgacccttaaatcatgttctcctatcgttggtgttgatgtgatgccatttgaaacagaaagagaagtcctgctggcttggagggattttattcgtgaagtggaccctgatattattattggatacaacatttgcaaatttgacttgccatatcttattgagagagctttgaacctgaagatagcagaatttccaattctgggtcgtatcaggaacagtagagttcgagtaaaggatacaactttctcatcaaggcagtacggaaccagggaaagtaaagaagttgcagtagaagggagagttacgtttgatttactccaggttatgcaaagagactacaaattaagttcttattcactgaattctgtgtcatcacacttcctttctgagcagaaagaggatgttcatcattcaattatatccgatcttcagaatggaaatgcagaaactaggaggcgccttgctgtgtattgtttgaaggatgcatatctccctcagcggcttttggataaattgatgttcatttacaattatgtggagatggctcgagtaacaggtgtcccaatttcttttctactttccagaggccaaagcattaaggtactttctcaacttcttaggagggcaaggcagaagaatctggtcattcctaatgccaaacaggctgggtctgaacaaggaacatttgaaggtgccactgtattggaggcaagggctggattttatgaaaaaccaattgctactttagattttgcatccttgtatccatctattatgatggcctataacttatgttattgcactctggtgatccctgaagatgctcgcaagctcaacatacctccagagtctgtgaacagaactccatctggtgaaacatttgttaaatcaaatttgcagaagggaatacttcctgaaatacttgaagagctattaacagcccgtaaaagggcaaaagcagacttaaaggaggccaaggatcccctggagaaggcagtgctagatggtagacagctagccctgaagattagtgccaattctgtgtatgggtttacaggggctaccattggtcagttaccatgtttagagatatcatcgagtgtaacaagctatggtcgacaaatgatcgagcacacgaaaaaacttgtggaagataaatttacgacacttaatggctatgaacacaatgccgaggtaatatatggagacacagattcagtcatggtacaatttggtgtttctgctgtagaagaggctatgaacttggggagagaagctgctgaacatattagtggaactttcacaaaacccatcaaactagaatttgagaaggtttactatccatatctcctgattagcaagaagagatatgctggtttgttttggacaaaaccagacaactttgacaaaatggacactaaaggtattgaaacagttcgaagagacaattgtttattggtcaaaaacctggtgaacgattgccttcacaaaatattgattgacagggacattcctggggcagtccagtatgtcaagaatgcaatttcagatcttctcatgaatcgtatggacttatcacttctggttattacaaagggtttaacgaagacaggagatgattacgaagtaaaggcagctcatgttgaacttgctgaaaggatgcgcaagcgagatgctgccactgctccaaatgttggagacagagtaccatatgttattattaaagctgcaaaaggtgcaaaggcatatgagagatcagaggatcctatctatgtgctagagaacaacatacccatagatcctcattactatcttgagaatcaaattagcaagccaattctgagaatttttgagccaattctgaagaatgctagcaaagagcttctccatggaagtcatacaagatctatttctatttctacaccgtcaaacagtggcatattgagatttgctaagaaacagctacctgcattggttgtaaagctttacttggcaagggttatcacactctctgttcacattgcaaaggaagggaggctgagctgtactgtaaaacagtatctcaagtgtctgagctggagatgctttttgggaggttgtggacacagtgtcaggagtgccaaggttcacttcatcaggatgttctctgcaccagtcgggattgtccaattttctatcgacgaaaaaaggcacagaaagatatgggtgaagcaaagttgcaattggacagatggaacttctaagttttgccaagaatttgaccttgcggatctcttcgaaccaatggacacaaatacaatctggtgtttgccacaatcctgacatttgtaatgtgagtaaaagcccacaatttgtttactg

SEQ ID NO: 50 - protein, 1088 aa, Glycine max
MTQEEEFMDEDVFINETLVSEDEESLILRDIEQRQALANRLSKWTRPPLSAGYVAQSRSVLFQQLEIDYVIAESHGELLPNSSGPVAIIRIFGVTKEGHSVCCNVHGFEPYFYICCPPGMGPDDISHFHQTLEGRMREANRNSNVGKFVRRIEMVQRRSIMYYQQSNSQPFLKIVVALPTMVASCRGILDRGIQLDGLGMKSFLTYESNVLFALRFMIDCNIVGGNWIGIPAGKYKKTAKSLSYCQLEFDCLYSELISHAPEGEYSKMAPFRILSFDIECAGRKGHFPEPTHDPVIQIANLVTLQGEDQPFIRNVMTLKSCSPIVGVDVMPFETEREVLLAWRDFIREVDPDIIIGYNICKFDLPYLIERALNLKIAEFPILGRIRNSRVRVKDTTFSSRQYGTRESKEVAVEGRVTFDLLQVMQRDYKLSSYSLNSVSSHFLSEQKEDVHHSIISDLQNGNAETRRRLAVYCLKDAYLPQRLLDKLMFIYNYVEMARVTGVPISFLLSRGQSIKVLSQLLRRARQKNLVIPNAKQAGSEQGTFEGATVLEARAGFYEKPIATLDFASLYPSIMMAYNLCYCTLVIPEDARKLNIPPESVNRTPSGETFVKSNLQKGILPEILEELLTARKRAKADLKEAKDPLEKAVLDGRQLALKISANSVYGFTGATIGQLPCLEISSSVTSYGRQMIEHTKKLVEDKFTTLNGYEHNAEVIYGDTDSVMVQFGVSAVEEAMNLGREAAEHISGTFTKPIKLEFEKVYYPYLLISKKRYAGLFWTKPDNFDKMDTKGIETVRRDNCLLVKNLVNDCLHKILIDRDIPGAVQYVKNAISDLLMNRMDLSLLVITKGLTKTGDDYEVKAAHVELAERMRKRDAATAPNVGDRVPYVIIKAAKGAKAYERSEDPIYVLENNIPIDPHYYLENQISKPILRIFEPILKNASKELLHGSHTRSISISTPSNSGILRFAKKQLPALVVKLYLARVITLSVHIAKEGRLSCTVKQYLKCLSWRCFLGGCGHSVRSAKVHFIRMFSAPVGIVQFSIDEKRHRKIWVKQSCNWTDGTSKFCQEFDLADLFEPMDTNTIWCLPQS

SEQ ID NO: 51 - DNA, 3435 bp, Homo sapiens
acggcggcgtaggctgtggcgggaaacgctgtttgaagcgggatggatggcaagcggcggccaggcccagggcccggggtgcccccaaagcgggcccgtgggggcctctgggatgatgatgatgcacctcggccatcccaattcgaggaggacctggcactgatggaggagatggaggcagaacacaggctgcaggagcaggaggaggaggagctgcagtcagtcctggagggggttgcagacgggcaggtcccaccatcagccatagatcctcgctggcttcggcccacaccaccagcgctggacccccagacagagcccctcatcttccaacagttggagattgaccattatgtgggcccagcgcagcctgtgcctggggggcccccaccatcccacggctccgtgcctgtgctccgcgccttcggggtcaccgatgaggggttctctgtctgctgccacatccacggcttcgctccctacttctacaccccagcgccccctggtttcgggcccgagcacatgggtgacctgcaacgggagctgaacttggccatcaaccgggacagtcgcggggggagggagctgactgggccggccgtgctggctgtggaactgtgctcccgagagagcatgtttgggtaccacgggcacggcccctccccgttcctgcgcatcaccgtggcgctgccgcgcctcgtggccccggcccgccgtctcctggaacagggcatccgtgtggcaggcctgggcacgcccagcttcgcgccctacgaggccaacgtcgactttgagatccggttcatggtggacacggacatcgtcggctgcaactggctggagctcccagccgggaaatacgccctgaggctgaaggagaaggctacgcagtgccagctggaggcggacgtgctgtggtctgacgtggtcagtcacccaccggaagggccatggcagcgcattgcgcccttgcgcgtgctcagcttcgatatcgagtgcgccggccgcaaaggcatcttccctgagcctgagcgggaccctgtcatccagatctgctcgctgggcctgcgctggggggagccggagcccttcctacgcctggcgctcaccctgcggccctgtgcccccatcctgggtgccaaggtgcagagctacgagaaggaggaggacctgctgcaggcctggtccaccttcatccgtatcatggaccccgatgtgatcaccggttacaacatccagaacttcgaccttccgtacctcatctctcgggcccagaccctcaaggtacaaacattccctttcctgggccgtgtggccggcctttgctccaacatccgggactcttcattccagtccaagcagacgggccggcgggacaccaaggttgtcagcatggtgggccgcgtgcagatggacatgctgcaggtgctgctgcgggagtacaagctccgctcctacacgctcaatgccgtgagcttccacttcctgggcgagcagaaggaggacgtgcagcacagcatcatcaccgacctgcagaatgggaacgaccagacccgccgccgcctggctgtgtactgcctaaaggatgcttacctgccactgcggctgctggagcggctcatggtgctggtgaacgccgtggagatggcgagggtcactggcgtgcccctcagctacctgctcagtcgtggccagcaggtcaaggtcgtatcccagctgttgcggcaggccatgcacgaggggctgctgatgcccgtggtgaagtcagagggcggcgaggactacacgggagccactgtcattgagcccctcaaagggtactacgacgtccccatcgccaccctggacttctcctcgctgtacccgtccatcatgatggcccacaacctgtgttacaccacactccttcggcccgggactgcacagaaactgggcctgactgaggatcagttcatcaggacccccaccggggacgagtttgtgaagacctcagtgcgtaaggggctgctgccccagatcctggagaacctgctcagtgcccggaagagggccaaggccgagctggccaaggagacagaccccctccggcgccaggtcctggatggacggcagctggcgctgaaggtgagcgccaactccgtatacggcttcactggcgcccaggtgggcaagttgccgtgcctggagatctcacagagcgtcacggggttcggacgtcagatgatcgagaaaaccaagcagctggtggagtctaagtacacagtggagaatggctacagcaccagcgccaaggtggtgtatggtgacactgactccgtcatgtgccgattcggcgtgtcctcggtggctgaggcgatggccctgggcggggaggccgcggactgggtgtcaggtcacttcccgtcgcccatccggctggagtttgagaaggtctacttcccatacctgcttatcagcaagaagcgctacgcgggcctgctcttctcctcccggcccgacgcccacgaccgcatggactgcaagggcctggaggcggtgcgcagggacaactgccccctcgtggccaacctggtcactgcctcactgcgccgcctgctcatcgaccgagaccctgagggcgcggtggctcacgcacaggacgtcatctcggacctgctgtgcaaccgcatcgatatctcccagctggtcatcaccaaggagctgacccgcgcggcctccgactatgccggcaagcaggcccacgtggagctggccgagaggatgaggaagcgggaccccgggagtgcgcccagcctgggcgaccgcgtcccctacgtgatcatcagtgccgccaagggtgtggccgcctacatgaagtcggaggacccgctgttcgtgctggagcacagcctgcccattgacacgcagtactacctggagcagcagctggccaagcccctcctgcgcatcttcgagcccatcctgggcgagggccgtgccgaggctgtgctactgcggggggaccacacgcgctgcaagacggtgctcacgggcaaggtgggcggcctcctggccttcgccaaacgccgcaactgctgcattggctgccgcacagtgctcagccaccagggagccgtgtgtgagttctgccagccccgggagtctgagctgtatcagaaggaggtatcccatctgaatgccctggaggagcgcttctcgcgcctctggacgcagtgccagcgctgccagggcagcctgcacgaggacgtcatctgcaccagccgggactgccccatcttctacatgcgcaagaaggtgcggaaggacctggaagaccaggagcagctcctgcggcgcttcggaccccctggacctgaggcctggtgaccttgcaagcatcccatggggcgggggcgggaccagggagaattaataaagttctggacttttgctaca

SEQ ID NO: 52 - protein, 1107 aa, Homo sapiens [sequence omitted]

SEQ ID NO: 53 - DNA, 6912 bp, Homo sapiens
cgccaaatttctcccctgaagcagaggtggtagccaacggctccatgtctctgaggagcggcgggcggcggcgcgcggacccaggcgcggatggcgaggccagcagggatgatggcgccacttcctcagtttcggcactcaagcgcctggaacggagtcagtggacggataagatggatttgcggtttggttttgagcggctgaaggagcctggtgagaagacaggctggctcattaacatgcatcctaccgagattttagatgaagataagcgcttaggcagtgcagtggattactactttattcaagatgacggaagcagatttaaggtggctttgccctataaaccgtatttctacattgcgaccagaaagggttgtgagcgagaagtttcatcttttctctccaagaagtttcagggcaaaattgcaaaagtggagactgtccccaaagaggatctggacttgccaaatcacttggtgggtttgaagcgaaattacatcaggctgtccttccacactgtggaggatcttgtcaaagtgaggaaggagatctcccctgccgtgaagaagaacagggagcaggatcacgccagcgacgcgtacacagctctgctttccagtgttctgcagaggggcggtgtcattactgatgaagaggaaacctctaagaagatagctgaccagttggacaacattgtggacatgcgcgagtacgatgttccctaccacatccgcctctccattgacctgaagatccacgtggctcattggtacaatgtcagataccgaggaaatgcttttccggtagaaatcacccgccgagatgaccttgttgaacgacctgaccctgtggttttggcatttgacattgagacgaccaaactgcccctcaagtttcctgatgctgagacagaccagattatgatgatttcctacatgatcgatggccagggctacctcatcaccaacagggagattgtttcagaagatattgaagattttgagttcacccccaagccagaatatgaaggccccttttgtgtcttcaatgaacccgatgaggctcatctgatccaaaggtggtttgaacacgtccaggagaccaaacccaccatcatggtcacctacaacggggacttttttgactggccatttgtggaggcccgggcagcagtccacggtctgagcatgcagcaggagataggcttccagaaggacagccagggggagtacaaggcgccccagtgcatccacatggactgcctcaggtgggtgaagagggacagttaccttcctgtgggcagtcataatctcaaggcggccgccaaggccaagctaggctatgatcccgtggagctagacccggaggacatgtgccggatggccacggagcagccccagactctggccacgtattctgtgtcagatgctgtcgccacttactacctgtacatgaagtacgtccacccattcatctttgctctgtgcaccattattcccatggagcccgacgaggtgctgcggaagggctctggcactctgtgtgaggccttgctgatggtgcaggccttccacgccaacatcatcttccccaacaagcaagagcaggagttcaataagctgacggacgacggacacgtgctggactctgagacctacgtcgggggccacgtggaggccctcgagtctggggttttccgcagcgatatcccttgccggtttaggatgaatcctgccgcctttgacttcctgctgcagcgggttgagaagaccttgcgccacgcccttgaggaagaggagaaagtgcctgtggagcaagtcaccaactttgaagaggtgtgtgatgagattaagagcaagcttgcctccctgaaggacgttcccagccgcatcgagtgtccactcatctaccacctggacgtgggggccatgtaccccaacatcatcctgaccaaccgcctgcagccctctgccatggtggacgaagccacctgtgctgcctgtgacttcaataagcctggagcaaactgccagcggaagatggcctggcagtggaggggcgagttcatgccagccagtcgcagcgaataccatcggatccagcaccagctggagtcagagaagttcccccccttgttcccagaggggccagctcgggcctttcatgaactgtcccgcgaggaacaggcgaaatacgagaagagaaggctggcggattactgccggaaagcctacaagaagatccacatcaccaaggtggaagagcgtctcaccaccatctgccagcgggaaaactccttctacgtggacaccgtgcgtgccttccgggacaggcgttacgagttcaaagggctccacaaggtgtggaaaaagaagctctcggcggccgtggaggtgggcgacgcggctgaggtgaagcgctgcaagaacatggaggtgctgtatgactcgctgcagctggcccacaagtgcatcctgaactccttctatggctatgtcatgcgcaagggggctcgctggtactccatggagatggctggcatcgtctgcttcacaggggccaacatcatcacccaggcacgggagctgatcgagcagattgggaggcccttagagctggacacagatggtatatggtgcgtcctgcccaacagcttcccagaaaattttgtcttcaagacgaccaatgtgaagaagcccaaagtgaccatctcctacccaggcgccatgttgaacatcatggtcaaggaaggcttcaccaatgaccagtaccaggagctggctgagccgtcctcactcacctacgtcacccgctcagagaacagcatcttttttgaggttgatgggccctaccttgccatgattcttccagcctccaaggaagaaggcaagaaattgaagaagaggtatgctgtgttcaatgaagacggttctctggctgagctcaagggctttgaggtcaaacgccgcggggaactgcagctgattaagatcttccaatcctcggtgtttgaggccttcctcaagggcagcacgctggaagaggtgtatggctctgtagccaaggtggctgactactggctggacgtgctgtacagcaaggcagccaacatgcctgactctgagctattcgagctcatctctgagaaccgttccatgtctcggaagctggaagattacggggagcagaagtctacatccatcagcacagcaaagcgcctggccgagttcctgggagaccagatggtcaaggatgcagggctgagttgccgctacatcatctcccgcaagcccgagggctcccctgtcacggagagggccatcccacttgccattttccaagcagagcccacggtgaggaagcactttctccggaaatggctcaagagctcttcccttcaagactttgatattcgagcaattctggattgggactactacattgagcggctgggaagcgccatccagaagatcatcaccatccctgcggccctgcagcaggtaaagaacccagtgccacgtgtcaaacaccccgactggctgcacaaaaaactgctggagaagaatgatgtctacaagcagaagaagatcagtgagctcttcaccctggagggcaggagacaggtcacgatggccgaggcctcagaagacagtccgaggccaagtgctcctgacatggaggacttcggcctcgtaaagctgcctcacccagcagcccctgtcactgtgaagaggaagcgagttctttgggagagccaggaggagtcccaggacctcacgccgactgtgccctggcaggaaatcttggggcagcctcccgccctgggaaccagccaggaggaatggcttgtctggctccggttccacaagaagaagtggcagctgcaggcccggcagcgcctcgcccgcaggaagaggcagcgtctggagtcggcagagggtgtgctcaggcccggggccatccgggatggtcctgccacggggctggggagcttcttgcgaagaactgcccgcagcatcctggaccttccgtggcagattgtgcagatcagcgagaccagccaggccggcctgttcaggctgtgggcgctcgttggcagtgacttgcactgcatcaggctgagcatcccccgtgtgttctacgtgaaccagcgagtcgctaaagcggaggagggtgcttcgtatcgcaaggtaaatcgggtccttcctcgctccaacatggtctacaatctctatgagtattcagtgccagaggacatgtaccaggaacacatcaacgagatcaacgctgagctgtcagcgccagacatcgagggcgtatatgagactcaggttccgttactgttccgggccctggtgcacctgggctgtgtgtgtgtggtcaataaacagctggtgaggcacctttcaggctgggaagcagagacctttgctcttgagcacctggagatgcgctctctggcccagttcagctacctggaaccagggagtatccgccatatctacctgtaccaccacgcacaggcccacaaagcgctcttcgggatcttcatcccctcacagcgcagggcatccgtctttgtgctggacactgtgcgcagcaaccagatgcccagccttggcgccctgtactcagcagagcacggcctcctcctggagaaggtgggccctgagctcctgccaccccccaaacacaccttcgaagttcgggcagaaactgacctgaagaccatctgcagagccatccagcgattcctgctcgcctacaaggaggagcgccgggggcccacactcatcgctgttcagtccagctgggagctgaagaggctggccagtgaaattcctgtcttggaggaattcccactggtgcctatctgtgtggctgacaagatcaactatggggtcctggactggcagcgccatggagcccggcgcatgatccgtcactacctcaacctggacacctgcctgtcgcaggccttcgagatgagcaggtactttcacattcccattgggaacctaccagaggacatctccacattcggctccgacctcttctttgcccgccacctccagcgccacaaccacctgctctggctgtcccctacagcccgccctgacctgggtggaaaggaggctgatgacaactgtcttgtcatggagttcgatgaccaagccactgttgagatcaacagttcaggctgttactccacagtgtgtgtggagctggaccttcagaacctggccgtcaacaccattctccagtctcaccatgtcaacgacatggagggggccgacagcatggggatcagcttcgacgtgatccagcaggcctccctggaggacatgatcacgggtggtcaggctgccagtgccccggccagctacgatgagacagccctgtgctctaacaccttcaggatcctgaagagcatggtcgtgggctgggtgaaggagatcacccagtaccacaacatctatgcagacaaccaggtgatgcacttctaccgctggcttcggtcgccatcctctctgcttcatgaccctgccctgcaccgcacactccacaacatgatgaagaagctcttcctgcagctcatcgctgagttcaagcgcctggggtcatcagtcatctacgccaacttcaaccgcatcatcctctgtacaaagaagcgccgtgtggaagatgccatcgcttacgtggagtacatcaccagcagcatccattcaaaggagaccttccattctctgacaatttctttctctcgatgctgggaatttcttctctggatggatccatctaactatggcggaatcaaaggaaaagtttcatctcgtattcactgtggactgcaagactcccagaaagcagggggagcagaggatgagcaggaaaatgaggacgatgaggaggaaagagatggggaggaggaggaagaggcggaggaatccaacgtggaggatttactggaaaacaactggaacattttgcagtttttgccacaggcagcctcctgccagaactacttcctcatgattgtttcagcgtacatcgtggccgtgtaccactgcatgaaggacgggctgaggcgcagtgctccagggagcacccccgtgaggaggaggggggccagccagctctcccaggaggccgagggggcggtcggagcccttcccggaatgatcaccttctctcaggattatgtcgcaaatgagctcactcagagcttcttcaccatcactcagaagattcagaagaaagtcacaggctctcggaactccactgagctctcagagatgtttcctgtcctccccggttcccacttgctgctcaataaccctgccctggagttcatcaaatacgtgtgcaaggtgctgtccctggacaccaacatcacaaaccaggtgaataagctgaaccgagacctgcttcgcctggtggatgtcggcgagttctccgaggaggcccagttccgagacccctgccgctcctacgtgcttcctgaggtcatctgccgcagctgtaacttctgccgcgacctggacctgtgtaaagactcttccttctcagaggatggggcggtcctgcctcagtggctctgctccaactgtcaggcgccctacgactcctctgccatcgagatgacgctggtggaagttctacagaagaagctgatggccttcaccctgcaggacctggtctgcctgaagtgccgcggggtgaaggagaccagcatgcctgtgtactgcacgtgcgcgggagacttcgccctcaccatccacacccaggtcttcatggaacagatcggaatattccggaacattgcccagcactacggcatgtcgtacctcctggagaccctggagtggctgctgcagaagaacccacagctgggccattagccagccc

SEQ ID NO: 54 - protein, 2286 aa, Homo sapiens
MSLRSGGRRRADPGADGEASRDDGATSSVSALKRLERSQWTDKMDLRFGFERLKEPGEKTGWLINMHPTEILDEDKRLGSAVDYYFIQDDGSRFKVALPYKPYFYIATRKGCEREVSSFLSKKFQGKIAKVETVPKEDLDLPNHLVGLKRNYIRLSFHTVEDLVKVRKEISPAVKKNREQDHASDAYTALLSSVLQRGGVITDEEETSKKIADQLDNIVDMREYDVPYHIRLSIDLKIHVAHWYNVRYRGNAFPVEITRRDDLVERPDPVVLAFDIETTKLPLKFPDAETDQIMMISYMIDGQGYLITNREIVSEDIEDFEFTPKPEYEGPFCVFNEPDEAHLIQRWFEHVQETKPTIMVTYNGDFFDWPFVEARAAVHGLSMQQEIGFQKDSQGEYKAPQCIHMDCLRWVKRDSYLPVGSHNLKAAAKAKLGYDPVELDPEDMCRMATEQPQTLATYSVSDAVATYYLYMKYVHPFIFALCTIIPMEPDEVLRKGSGTLCEALLMVQAFHANIIFPNKQEQEFNKLTDDGHVLDSETYVGGHVEALESGVFRSDIPCRFRMNPAAFDFLLQRVEKTLRHALEEEEKVPVEQVTNFEEVCDEIKSKLASLKDVPSRIECPLIYHLDVGAMYPNIILTNRLQPSAMVDEATCAACDFNKPGANCQRKMAWQWRGEFMPASRSEYHRIQHQLESEKFPPLFPEGPARAFHELSREEQAKYEKRRLADYCRKAYKKIHITKVEERLTTICQRENSFYVDTVRAFRDRRYEFKGLHKVWKKKLSAAVEVGDAAEVKRCKNMEVLYDSLQLAHKCILNSFYGYVMRKGARWYSMEMAGIVCFTGANIITQARELIEQIGRPLELDTDGIWCVLPNSFPENFVFKTTNVKKPKVTISYPGAMLNIMVKEGFTNDQYQELAEPSSLTYVTRSENSIFFEVDGPYLAMILPASKEEGKKLKKRYAVFNEDGSLAELKGFEVKRRGELQLIKIFQSSVFEAFLKGSTLEEVYGSVAKVADYWLDVLYSKAANMPDSELFELISENRSMSRKLEDYGEQKSTSISTAKRLAEFLGDQMVKDAGLSCRYIISRKPEGSPVTERAIPLAIFQAEPTVRKHFLRKWLKSSSLQDFDIRAILDWDYYIERLGSAIQKIITIPAALQQVKNPVPRVKHPDWLHKKLLEKNDVYKQKKISELFTLEGRRQVTMAEASEDSPRPSAPDMEDFGLVKLPHPAAPVTVKRKRVLWESQEESQDLTPTVPWQEILGQPPALGTSQEEWLVWLRFHKKKWQLQARQRLARRKRQRLESAEGVLRPGAIRDGPATGLGSFLRRTARSILDLPWQIVQISETSQAGLFRLWALVGSDLHCIRLSIPRVFYVNQRVAKAEEGASYRKVNRVLPRSNMVYNLYEYSVPEDMYQEHINEINAELSAPDIEGVYETQVPLLFRALVHLGCVCVVNKQLVRHLSGWEAETFALEHLEMRSLAQFSYLEPGSIRHIYLYHHAQAHKALFGIFIPSQRRASVFVLDTVRSNQMPSLGALYSAEHGLLLEKVGPELLPPPKHTFEVRAETDLKTICRAIQRFLLAYKEERRGPTLIAVQSSWELKRLASEIPVLEEFPLVPICVADKINYGVLDWQRHGARRMIRHYLNLDTCLSQAFEMSRYFHIPIGNLPEDISTFGSDLFFARHLQRHNHLLWLSPTARPDLGGKEADDNCLVMEFDDQATVEINSSGCYSTVCVELDLQNLAVNTILQSHHVNDMEGADSMGISFDVIQQASLEDMITGGQAASAPASYDETALCSNTFRILKSMVVGWVKEITQYHNIYADNQVMHFYRWLRSPSSLLHDPALHRTLHNMMKKLFLQLIAEFKRLGSSVIYANFNRIILCTKKRRVEDAIAYVEYITSSIHSKETFHSLTISFSRCWEFLLWMDPSNYGGIKGKVSSRIHCGLQDSQKAGGAEDEQENEDDEEERDGEEEEEAEESNVEDLLENNWNILQFLPQAASCQNYFLMIVSAYIVAVYHCMKDGLRRSAPGSTPVRRRGASQLSQEAEGAVGALPGMITFSQDYVANELTQSFFTITQKIQKKVTGSRNSTELSEMFPVLPGSHLLLNNPALEFIKYVCKVLSLDTNITNQVNKLNRDLLRLVDVGEFSEEAQFRDPCRSYVLPEVICRSCNFCRDLDLCKDSSFSEDGAVLPQWLCSNCQAPYDSSAIEMTLVEVLQKKLMAFTLQDLVCLKCRGVKETSMPVYCTCAGDFALTIHTQVFMEQIGIFRNIAQHYGMSYLLETLEWLLQKNPQLGH

SEQ ID NO: 55 - DNA, 3390 bp, Mus musculus
atcttgtggcgggaaaagctgtttgaggcgatggattgtaagcggcgacaaggaccaggccctggggtgcccccaaagcgggctcgagggcacctctgggatgaggacgagccttcgccgtcgcagtttgaggcgaacctggcactgctggaggaaatagaggctgagaaccggctgcaggaggcagaggaggagctgcagctgcccccagagggcaccgtgggtgggcagttttccactgcagacattgaccctcggtggcggcggcccaccctacgtgccctggaccccagcacggagcccctcatcttccagcagctggagattgaccactatgtgggctcagcaccacccctgccagaacggcccctgccatcccggaactcagtgcccatactgagggcctttggggtcaccgatgaaggcttctccgtctgctgccacatacagggctttgccccctacttctacacccccgcgcctcctggttttggggccgagcacctgagtgagctgcagcaggagctgaacgcagccatcagccgggaccagcgcggtgggaaggagctctcagggccggcagtgctggcaatagagctatgctcccgtgagagcatgtttgggtaccacggtcatggcccttctccatttctccgcatcaccctggcactaccccgccttatggcaccagcccgccgccttctggaacagggtgtccgagtgccaggcctgggcaccccgagcttcgcaccctacgaagccaacgtggactttgagatccggttcatggtggatgctgacattgtgggatgcaactggttggagctgccagctggaaagtacgttcggagggcggagaagaaggccaccctgtgtcagctggaggtggacgtgctgtggtcagatgtgatcagtcacccaccggaggggcagtggcagcgcattgcacccctgcgtgtgcttagcttcgacatcgagtgtgctggccgaaaaggcatcttccctgagcctgagcgtgaccccgtgatccagatctgttctctggggctgcgctggggggagccggagccattcttgcgtctggcactcacgctgcggccctgtgcccccatcctgggtgccaaagtgcagagctatgagcgggaagaagacctgctccaggcctgggccgacttcatccttgccatggaccctgacgtgatcaccggctacaacattcagaactttgacctcccatacctcatctctcgggcacaggccctaaaggtggaccgcttccctttcctgggccgcgtgactggtctccgctccaacatccgtgactcctccttccaatcaaggcaggtcggccggcgggacagtaaggtgatcagcatggtgggtcgcgttcagatggatatgctgcaggtgctgcttcgggaacacaagctccgctcctacacgctcaacgctgtgagtttccacttcctgggcgagcagaaggaggacgttcagcacagcatcatcaccgacctgcagaatgggaacgaacagacgcgccgccgcctggccgtgtactgcctgaaggacgcctttctgccactccgactactagagcgccttatggtgctggtgaacaatgtggagatggcgcgtgtcaccggtgtaccccttgggtacctgctcacccggggccagcaggtcaaggtcgtgtctcagctgctgcgccaggccatgcgccaggggctgctgatgcctgtggtgaagaccgagggcggtgaggactacacgggagccacagtcattgagcccctcaaagggtactatgacgtccccattgccaccctggacttctcctccttgtacccatccatcatgatggcccataatctgtgctacaccacgctgctccgacctggggctgcccagaagctgggccttaaaccagatgagttcatcaagacacccactggggatgagtttgtgaagtcatctgtacggaagggcctcctgccccagatcctggagaatctgctgagtgcccgcaagagggccaaggctgagctggctcaggagacggaccccctgcggcgacaggtcttggacggccggcaactggcactaaaagtgagtgccaactccgtatatggcttcactggtgcccaggtgggcaagctgccatgtttggaaatctcccagagtgtcactgggttcgggcggcagatgattgagaaaaccaagcagcttgtggagtccaagtacaccgtggaaaatggctacgatgccaacgccaaggtagtctacggtgacacggactctgtgatgtgccggtttggcgtctcctctgtggctgaagcaatgtctctggggcgggaggctgcaaactgggtatccagtcacttcccatcacccatccggctggagttcgagaaggtttacttcccatacctgctcatcagcaagaagcgctatgctggcctgctcttctcctcccgctctgatgcccatgacaaaatggactgcaagggcctggaggctgtgcgcagggacaactgtcccctggtggccaacctcgttacatcctccctgcgccggatcctcgtggaccgggaccctgatggggcagtagcccatgccaaggacgtcatctcggacctgctgtgcaaccgcatagacatctcccagctggtcatcaccaaagagttgacccgcgcagcagcagactatgctggcaagcaggctcacgtggagctggctgagaggatgaggaagcgcgaccccggcagtgcgcccagcctgggtgaccgagtcccctatgtgatcattggtgctgctaagggtgtggccgcctacatgaagtcggaggaccccctgtttgtgctggagcacagcctgcccatcgacactcagtactacctggagcagcagctggccaagccgctcttgcgcatctttgagcccatcctgggtgagggccgtgcagagtctgtgctgctgcgcggtgaccacacacgatgcaagactgtgctcaccagcaaggtgggcggcctcttggccttcaccaagcgccgcaactgttgcattggctgccgctccgtaatcgaccatcaaggagccgtgtgtaagttctgtcagccacgggagtcggagctctctcagaaggaggtgtcacacctgaatgccttggaagaacggttctctcgcctctggacacagtgtcaacgctgccagggcagcttgcatgaggacgtcatctgtaccagccgtgactgtcccatcttctacatgcgcaagaaggtgcgcaaggacctggaagaccaggaacggctgctgcagcgctttggaccgcccggccctgaggcctggtgacctgacacgggacaaggaataaagttcagatctttgctaaaa

SEQ ID NO: 56 - protein, 1105 aa, Mus musculus
MDCKRRQGPGPGVPPKRARGHLWDEDEPSPSQFEANLALLEEIEAENRLQEAEEELQLPPEGTVGGQFSTADIDPRWRRPTLRALDPSTEPLIFQQLEIDHYVGSAPPLPERPLPSRNSVPILRAFGVTDEGFSVCCHIQGFAPYFYTPAPPGFGAEHLSELQQELNAAISRDQRGGKELSGPAVLAIELCSRESMFGYHGHGPSPFLRITLALPRLMAPARRLLEQGVRVPGLGTPSFAPYEANVDFEIRFMVDADIVGCNWLELPAGKYVRRAEKKATLCQLEVDVLWSDVISHPPEGQWQRIAPLRVLSFDIECAGRKGIFPEPERDPVIQICSLGLRWGEPEPFLRLALTLRPCAPILGAKVQSYEREEDLLQAWADFILAMDPDVITGYNIQNFDLPYLISRAQALKVDRFPFLGRVTGLRSNIRDSSFQSRQVGRRDSKVISMVGRVQMDMLQVLLREHKLRSYTLNAVSFHFLGEQKEDVQHSIITDLQNGNEQTRRRLAVYCLKDAFLPLRLLERLMVLVNNVEMARVTGVPLGYLLTRGQQVKVVSQLLRQAMRQGLLMPVVKTEGGEDYTGATVIEPLKGYYDVPIATLDFSSLYPSIMMAHNLCYTTLLRPGAAQKLGLKPDEFIKTPTGDEFVKSSVRKGLLPQILENLLSARKRAKAELAQETDPLRRQVLDGRQLALKVSANSVYGFTGAQVGKLPCLEISQSVTGFGRQMIEKTKQLVESKYTVENGYDANAKVVYGDTDSVMCRFGVSSVAEAMSLGREAANWVSSHFPSPIRLEFEKVYFPYLLISKKRYAGLLFSSRSDAHDKMDCKGLEAVRRDNCPLVANLVTSSLRRILVDRDPDGAVAHAKDVISDLLCNRIDISQLVITKELTRAAADYAGKQAHVELAERMRKRDPGSAPSLGDRVPYVIIGAAKGVAAYMKSEDPLFVLEHSLPIDTQYYLEQQLAKPLLRIFEPILGEGRAESVLLRGDHTRCKTVLTSKVGGLLAFTKRRNCCIGCRSVIDHQGAVCKFCQPRESELSQKEVSHLNALEERFSRLWTQCQRCQGSLHEDVICTSRDCPIFYMRKKVRKDLEDQERLLQRFGPPGPEAW

SEQ ID NO: 57 - DNA, 7119 bp, Mus musculus
gccaaattctccccggagcctgagggagctttggagcgtcgcaatggtcctgaggaacagtggacggaggcaccccgagccgggcgcggatggcgaaggcagccgggatgatggtccctcttcctcagtctcagcactcaagcgtctggaacggagccagtggacagacaagatggacttacggtttggtttcgaaaggctgaaagagcctggagaaaggactggctggctgatcaacatgcaccctactgagatcttagatgaagacaaacgcttagtcagcgcggtggattactacttcattcaagatgatggaagcagatttaaggtggccttgccctatatgccgtatttctacattgcagcgagaaagggttgtgatcgagaagtttcatcttttctatccaagaagtttcagggaaaaattgcaaagttagagaatgtgcccaaagaagatctggacttgccaaatcacttggtgggcttgaagcggagttacatcaagctgtccttccacactgtggaggaccttgtcaaagtgagaaaggagatctctcctgctgtgaagaagaaccgagagcaggaccatgctagtgatgagtatacaacaatgctctccagtattctgcaaggtggcagtgtaattactgatgaggaggaaacctctaagaagatagctgaccaattggacaacatagtggacatgcgggagtatgatgttccctaccacattcgcctctccattgacctcagaatccatgtggcccactggtacaatgttagatttcgaggaaatgcttttcctgtggaaatcacccgacgagatgatcttgtggaacgacctgaccctgtggttttggcatttgacatcgagacgaccaaactgcctctcaaattccctgatgctgagaccgatcagatcatgatgatctcctatatgattgatggccagggctacctcatcactaacagggagattgtttcagaagatattgaagattttgagttcacccctaagccagaatatgaagggcccttttgtgttttcaatgaacccgacgaggtccatctgatccagagatggtttgagcatatccaggagaccaaacctaccattatggtcacctacaatggggatttttttgactggccatttgtggaggctagggcagcaattcatggcctcagcatgtaccaggagataggcttccagaaggatagccagggggaatataaggcaccacagtgcatccacatggactgcctcaggtgggtgaagagggacagttaccttcctgtgggcagtcataatctcaaggcagctgccaaggccaaacttggctatgaccctgtagagctggaccctgaggacatgtgtcgtatggccactgaacagccccagactctggccacttactcagtgtcagatgctgtggctacttactacctgtacatgaaatacgtccaccccttcatattcgccctgtgcaccattattcccatggaacctgatgaggtgctgcggaagggctccgggacactgtgtgaagccttgctgatggtgcaagctttccatgccaacattatcttccccaataagcaagagcaggagttcaacaagctgacagatgatggccacgtgctagatgctgagacctacgttgggggccacgtggaggcactagagtctggtgtcttcagaagtgatatcccctgccggtttaggatgaatcctgcagcctttgatttcctgctgcaacgagtcgagaagactatgcgccacgccattgaagaagaagagaaggtgcctgtggaacaagccaccaactttcaagaggtgtgtgagcagattaagaccaagctcacctccctaaaagatgttcctaacagaattgaatgtcctctaatctatcatctagatgtgggggccatgtatcctaacataattcttaccaaccgcctacagccttctgccatagtggatgaggccacctgtgctgcctgtgacttcaataagcctggagcaagttgtcagaggaagatggcctggcagtggaggggagaattcatgccagccagtcgcagtgaataccatcggattcagcatcagctggagtcggagaagtttccccctttgtttccagaggggccagcacgggcctttcacgagctgtcccgtgaagaacaggctaaatatgagaagaggaggctggcagattattgccggaaagcctataagaagatccatgtgaccaaggtagaagaacgtctaactaccatctgccagcgggaaaactcattttatgtggacacagtgcgggccttcagagacaggcgctatgagttcaaaggactgcacaaggtgtggaagaagaagctctcggcagctgtagaggtgggcgatgcatcagaggtgaagcgctgcaagaacatggagatcctttacgattcactgcagctggctcacaagtgcatcctgaactccttctacggctatgtcatgcgcaaaggagctcgctggtattccatggagatggctggtatcgtctgctttacaggagccaacatcatcacccaagcaagagaactgattgagcagatcgggaggcctttagaattggacacggacggaatatggtgcgtcctacccaatagctttcctgaaaattttgtcatcaagacaaccaatgcgaagaaacccaaactgaccatctcctatcctggtgccatgttgaacatcatggtcaaggaaggctttaccaaccaccagtaccaggaactaacagagccttcgtctctcacctatgtcacccactctgagaatagtatcttttttgaagtcgatggaccataccttgctatgatccttccagcctccaaggaagaaggcaagaagctgaagaaaagatatgctgtgttcaatgaagatggttccttggctgaactgaaaggttttgaggtgaaacgccgaggggagttgcagctgattaaaatattccagtcctcagtttttgaggccttcctcaagggcagcacactggaggaagtgtatggctcggtggccaaagtggctgactactggctagatgtgctctatagcaaggctgctaatatgcccgattctgaattgtttgagctgatttctgagaaccgctccatgtctcggaagctggaagattacggggagcagaagtctacatccatcagcacagcaaagcgcctggctgagttcctgggagaccagatggtcaaagatgctggactgagctgccgctatatcatctcccgaaagccagaggggtctcctgtcactgagagggccattccacttgccattttccaagcagagcctacagtgaggaaacattttctccggaaatggctaaagagttcatcacttcaagactttgatattcggacaattctggactgggactactacatagagaggctggggagtgccatccagaaaatcatcaccatccccgcagctctgcagcaggtgaagaacccagttccacgtgtcaaacatccagactggctacacaaaaaactactagagaagaatgatatctacaaacagaagaagatcagtgagctctttgtgcttgaaggaaagagacagattgtgatggcccaggcttcagaaaacagtctgagtctctgcactccagacatggaggacattggactcacaaagccacaccactctacagtcccagttgctactaagaggaagcgagtctgggagacccaaaaggagtctcaggatattgcactaactgtgccctggcaagaggtcttagggcagcctccctcccttggaaccacacaggaagagtggttggtctggctccagttccacaagaaaaagtggcagctgcaggcccaacagcgcctagccctcaggaagaagcaacgcttagagtcagcagaagatatgccaaggcttgggcctatccgagaggagccttccacaggactggggagctttttgcgaaggactgcccgcagcatcatggaccttccatggcagataatacagatcagtgagaccagacaggctggtctgttccggctgtgggctatcattggcaatgacttgcactgcatcaagctgagtatccctcgagtattctatgtaaaccagcgggttgccaaagcagaggatggacctgcatatcggaaggtgaatcgggggctcttccttcgttccaacattgtctacaatctctatgagtattcagtaccagaggacatgtaccaagaacacatcaacgagatcaacactgagttgtcagtaccagacattgagggcgtgtatgagacacaggtcccattgttattccgggccctcgtgcagctgggctgtgtgtgtgtggtcaacaagcagctgacaaggcacctttcgggctgggaagctgaaacttttgccctcgagcaccttgaaatgcgttctctggcccagttcagctacttggaaccagggagtatccgccatatctacctgtaccatcacactcagggccacaaggcactctttggggtctttatcccctcacagcgaagagcatctgtgtttgtgttggatactgtacgaagcaaccaaatgccagggctcagtgccctgtactcatcagaacacagcctgctgctggacaaggtggaccccaagctcctgcctcccccaaaacacacctttgaagttcgtgctgaaaccaacctggagactatctgcagagccatccagcgcttcctgcttgcctacaaggaagagcgccgagggcccacactcatcgctgtccagtctagctgggagctgtgtaggctgaccagtgagattccagtcttagaagagttcccactagtgcctatccgagtggctgacaagatcagctatgcagtcctagactggcagcgccatggagctcgccgaatgatccggcactacctcaatttagacttgtgcctgtcgcaggcctttgagatgagcaggtacttccacatccctgttggaaacctgccggaagacatctccatctttggctcagacctcttttttgcacgccacctccagcaccataaccacctgctttggctatcccctacctctcggcctgacctgggtgggaaggaagctgatgacaaccgccttgtcatggagtttgatgaccgagccactgtggagatcaatagttctggctgttactctactgtgtgcgtggaactggacattcaaaatctggcagtcaacaccatcctccagtcccatcatgtcaatgacatggagggggctggcagcatgggcatcagcttcgatgtgatccagcaggcctccctagaggacatggtaacaggcaatcaagctgccagtgccctggccaactacgatgagacagccctctgctctagtaccttcaggatcctgaagagcatggtggttggctgggtaaaggaaatcacacagtaccacaacatctatgctgacaaccaggtaatgcacttctaccgctggctccagtcaccgtgctctctgctccacgacccagcccttcaccggacgctgcacaatatgatgaagaagctcttcctgcagctcattgctgagttcaagcgcctggggtcatcagtcgtctatgccaacttcaatcgcatcattctctgtacaaagaagcgccgaatagaggatgcccttgcctatgtggaatatattaccaacagcatccattctaaagagatcttccattccctgaccatctctttctctcgatgctgggaattccttctctggatggatccatccaactatggtggaatcaaaggaaaagttccatctagtattcactgtggacaggtaaaagagcaagactcccaggcaagagaggaaactgatgaagaggaggaggacaaggaaaaggacgaggaggaagagggcatgggagagtccgaggttgaggacttactggagaacaactggaacattctacagttcttgccccaggcagcctcttgccagagctacttcctcatgattgtttcagcatacatcgtagctgtgtaccaaagcatgaaggaggagttgagacacagtgccccgggcagtacccctgtgaagaggaagggggccagccagttctcccaggagtctgaaggggcaactggatctcttcctggaatgatcactttctctcaagattatgtggcaaatgagctcactcagagcttcttcaccattactcagaaaattcagaagaaagtcacaggttctcggaacaccactgagccctcagagatgttccccgtcctccctggttcacacttgctgctcaataatcctgctctggagttcatcaaatatgtgtgcaaggtactatctcaggatacaaacatcacaaatcaggtgaataagctgaacagagaccttcttcgcctggtagacgttggtgaattctctgaggaggcccagttcagagacccctgccactcctacgtgctccctgaggtaatctgccacagctgtaatttctgccgagacctggacctgtgcaaagattcctctttctctcaggatggagccatcctgcctcagtggctctgctccaattgtcaagccccctatgactcctctgccattgagtcagccttggtggaagccctgcagaggaaactgatggccttcacacttcaggacctggtatgcctcaagtgccgtggtatgaaagagacccatatgcctgtgtactgcagctgcgcaggggactttactctcaccatccgcactgaggtcttcatggaacagattagaatcttccagaacattgccaagtactacagcatgtcatatctccaggagaccatagaatggctgttacagacaagccctgtatcaaactgttagcaagcctaggctaaagacactttggtatcccacacctactgcctgctccaaaaggcagaaccactgaccaccttgcttttccaaactcatgagcacagcccagaaggaacagaagacttctgctaacgtcatcatgccataaacagacagaagcagggaatggctctatccctagctgcctgctaagtaaacacggttttgaagcgtctgaaaaaaaaaaa

SEQ ID NO: 58 - protein, 2284 aa, Mus musculus
MVLRNSGRRHPEPGADGEGSRDDGPSSSVSALKRLERSQWTDKMDLRFGFERLKEPGERTGWLINMHPTEILDEDKRLVSAVDYYFIQDDGSRFKVALPYMPYFYIAARKGCDREVSSFLSKKFQGKIAKLENVPKEDLDLPNHLVGLKRSYIKLSFHTVEDLVKVRKEISPAVKKNREQDHASDEYTTMLSSILQGGSVITDEEETSKKIADQLDNIVDMREYDVPYHIRLSIDLRIHVAHWYNVRFRGNAFPVEITRRDDLVERPDPVVLAFDIETTKLPLKFPDAETDQIMMISYMIDGQGYLITNREIVSEDIEDFEFTPKPEYEGPFCVFNEPDEVHLIQRWFEHIQETKPTIMVTYNGDFFDWPFVEARAAIHGLSMYQEIGFQKDSQGEYKAPQCIHMDCLRWVKRDSYLPVGSHNLKAAAKAKLGYDPVELDPEDMCRMATEQPQTLATYSVSDAVATYYLYMKYVHPFIFALCTIIPMEPDEVLRKGSGTLCEALLMVQAFHANIIFPNKQEQEFNKLTDDGHVLDAETYVGGHVEALESGVFRSDIPCRFRMNPAAFDFLLQRVEKTMRHAIEEEEKVPVEQATNFQEVCEQIKTKLTSLKDVPNRIECPLIYHLDVGAMYPNIILTNRLQPSAIVDEATCAACDFNKPGASCQRKMAWQWRGEFMPASRSEYHRIQHQLESEKFPPLFPEGPARAFHELSREEQAKYEKRRLADYCRKAYKKIHVTKVEERLTTICQRENSFYVDTVRAFRDRRYEFKGLHKVWKKKLSAAVEVGDASEVKRCKNMEILYDSLQLAHKCILNSFYGYVMRKGARWYSMEMAGIVCFTGANIITQARELIEQIGRPLELDTDGIWCVLPNSFPENFVIKTTNAKKPKLTISYPGAMLNIMVKEGFTNHQYQELTEPSSLTYVTHSENSIFFEVDGPYLAMILPASKEEGKKLKKRYAVFNEDGSLAELKGFEVKRRGELQLIKIFQSSVFEAFLKGSTLEEVYGSVAKVADYWLDVLYSKAANMPDSELFELISENRSMSRKLEDYGEQKSTSISTAKRLAEFLGDQMVKDAGLSCRYIISRKPEGSPVTERAIPLAIFQAEPTVRKHFLRKWLKSSSLQDFDIRTILDWDYYIERLGSAIQKIITIPAALQQVKNPVPRVKHPDWLHKKLLEKNDIYKQKKISELFVLEGKRQIVMAQASENSLSLCTPDMEDIGLTKPHHSTVPVATKRKRVWETQKESQDIALTVPWQEVLGQPPSLGTTQEEWLVWLQFHKKKWQLQAQQRLALRKKQRLESAEDMPRLGPIREEPSTGLGSFLRRTARSIMDLPWQIIQISETRQAGLFRLWAIIGNDLHCIKLSIPRVFYVNQRVAKAEDGPAYRKVNRGLFLRSNIVYNLYEYSVPEDMYQEHINEINTELSVPDIEGVYETQVPLLFRALVQLGCVCVVNKQLTRHLSGWEAETFALEHLEMRSLAQFSYLEPGSIRHIYLYHHTQGHKALFGVFIPSQRRASVFVLDTVRSNQMPGLSALYSSEHSLLLDKVDPKLLPPPKHTFEVRAETNLETICRAIQRFLLAYKEERRGPTLIAVQSSWELCRLTSEIPVLEEFPLVPIRVADKISYAVLDWQRHGARRMIRHYLNLDLCLSQAFEMSRYFHIPVGNLPEDISIFGSDLFFARHLQHHNHLLWLSPTSRPDLGGKEADDNRLVMEFDDRATVEINSSGCYSTVCVELDIQNLAVNTILQSHHVNDMEGAGSMGISFDVIQQASLEDMVTGNQAASALANYDETALCSSTFRILKSMVVGWVKEITQYHNIYADNQVMHFYRWLQSPCSLLHDPALHRTLHNMMKKLFLQLIAEFKRLGSSVVYANFNRIILCTKKRRIEDALAYVEYITNSIHSKEIFHSLTISFSRCWEFLLWMDPSNYGGIKGKVPSSIHCGQVKEQDSQAREETDEEEEDKEKDEEEEGMGESEVEDLLENNWNILQFLPQAASCQSYFLMIVSAYIVAVYQSMKEELRHSAPGSTPVKRKGASQFSQESEGATGSLPGMITFSQDYVANELTQSFFTITQKIQKKVTGSRNTTEPSEMFPVLPGSHLLLNNPALEFIKYVCKVLSQDTNITNQVNKLNRDLLRLVDVGEFSEEAQFRDPCHSYVLPEVICHSCNFCRDLDLCKDSSFSQDGAILPQWLCSNCQAPYDSSAIESALVEALQRKLMAFTLQDLVCLKCRGMKETHMPVYCSCAGDFTLTIRTEVFMEQIRIFQNIAKYYSMSYLQETIEWLLQTSPVSNC

SEQ ID NO: 59 - DNA, 3325 bp, Rattus norvegicus
aggcgatggatggtaaacggcggcaagcgcccagctctggggtgcccccaaagcgggcttgcaagggcctctgggatgaagatgagccgtcacagtttgaggagaacctggcgctgctggaggagatagaggccgagaatcggctgcaggaggccgaggaggagctgcagctgccccctgagggcattgtgggtgggcagttttccactgcagacattgacccacggtggctgcggcccaccccacttgccctggaccccagcacggagcccctcatcttccagcagctggagattgaccactatgtgggcacatcacctcccctgccagaaggaccccccgcatctcgtaactcagtgcccatactgagggcctttggggtcaccgatgagggcttctccgtctgctgccacatccacggctttgccccctacttctacacccctgcacctccgggttttggggctgagcacctgagtgaactacagcgggagctgaatgcagccatcagccgggaccagcgtggtggaaaggagctctcggggccggcagtgctagctatagagctgtgctcccgtgagagcatgtttgggtaccatggccacggcccttctccctttctccgcatcaccctggcactaccccgcctgatggcgccagcccgccgcctcctggaacagggtatccgagtgccaggcctgggcaccccgagctttgcaccctatgaagccaatgtggactttgagatccggttcatggtggatgctgacattgtgggatgcaactggttggagctcccagctggaaagtacgttcggagggcagagaagaaggctacactgtgtcagctggaggtggatgtgctgtggtcagacgtgatcagtcacccaccagaagggcagtggcagcgcatcgcacccctgcgtgtgcttagcttcgacatcgagtgcgctggccgaaaaggcatcttccctgagcctgagcgtgaccccgtgatccagatctgttctctggggctgcgctggggtgagccagagcccttcttgcgcctagcactcacgctgcggccttgcgcccccatcctgggtgccaaagtacagagctatgaacgggaagaagacctgctccaggcctgggccactttcatcctcgccatggaccctgacgtgatcaccggctacaacattcagaactttgacctcccctacctcatctctcgggcacaaaccttaaaggtggaccgattccctttcctgggccgtgtgactggtctccgctccaacatccgtgactcctccttccaatcaaggcaggtgggccggcgggacagtaaggtggtcagcatggtgggtcgcgttcagatggatatgctgcaggtgctgcttcgggagtacaagctccgctcctacacgctcaacgctgtgagcttccacttcctgggtgagcagaaggaggacgtacagcacagcatcatcactgacctacagaatgggaatgaacagacgcgtcgccgcctggccgtgtactgcctgaaggatgcctttctgcctcttcgcctactcgagcgccttatggtgctggtgaacaatgtggagatggcgcgtgtcactggtgtaccccttgggtacctgctcagccgaggccagcaggtcaaggtcgtgtctcagctgctgcgccaggccatgcgcgaggggctgctgatgcctgtggtgaagacggagggaggtgaggactacacgggagccactgtcattgagcccctcaaagggtactatgatgtccccattgccaccctggacttctcctctctgtacccatccatcatgatggcccacaatctgtgctacaccacattgctacggcctggggctgcccagaagttgggccttaaaccagatgagttcatcaagacacccactggggatgagtttgtgaaggcatctgtgcggaagggcctcctgccccaaatcctggagaatctgctgagtgcccggaagagggccaaggctgagctggctcaggagacggaccccctgcggcgacaggtcttggatggacggcagctggcactaaaagtgagtcccaactctgtgtatggcttcactggtgcccaggtgggcaagctgccgtgtttggagatctcccagagtgtcactgggttcgggcggcagatgattgagaaaaccaagcagctagtggagaccaagtacactctggaaaatggctacgatgctaatgccaaggtggtctacggtgacactgactctgtgatgtgccgatttggtgtctcctctgtggctgaggcaatgtctctggggcgggaggctgcaaactgggtatccagtcacttcccatcacccatccggctggagttcgagaaggtttactttccctacctgctcatcagcaagaagcgctatgccggcctactcttctcctcccgctctgatgcccatgacagaatggactgcaagggcctggaggctgtgcgtagggacaactgtcccctggtggccaaccttgtcacatcctctctgcgcagaatcctcgtggatcgggaccctgatggtgcagtagcccatgcaaaggatgtcatctcggacctgctgtgcaaccgcatagacatctcccaactggtcatcaccaaagagttgacccgcgcagcagcagactacgcgggcaagcaggctcatgtggagctggctgagaggatgaggaagcgtgaccccggcagtgcgcccaacttgggcgaccgagtaccctacgtgatcattggtgctgccaagggtgtggccgcctacatgaagtcggaggaccccctgtttgtgctggagcacagcctgcccattgatactcagtactacctggagcagcagctggccaagccgctattgcgcatctttgagcccattctgggtgagggccgcgcggagtcagtgctgctgcgcggtgaccacacacgctgcaaaaccgtgctcaccagcaaggtgggcggccttctggccttcaccaagcgccgaaactcttgtattggctgccgctccgtaatcgaccatcaaggagccgtgtgtaagttctgtcagccacgggagtctgagctctatcagaaggaggtgtcacacctgaatgccctggaggaacgtttctcgcgcctctggacacagtgccagcgctgccagggcagcttgcacgaggatgtcatctgtaccagccgcgactgtcccatcttctacatgcgcaagaaggtgcgcaaggacctggaggaccaggaacggctgctgcagcgctttggacctcctggccctgaggcctggtgacctgacaa

SEQ ID NO: 60 - protein, 1103 aa, Rattus norvegicus
MDGKRRQAPSSGVPPKRACKGLWDEDEPSQFEENLALLEEIEAENRLQEAEEELQLPPEGIVGGQFSTADIDPRWLRPTPLALDPSTEPLIFQQLEIDHYVGTSPPLPEGPPASRNSVPILRAFGVTDEGFSVCCHIHGFAPYFYTPAPPGFGAEHLSELQRELNAAISRDQRGGKELSGPAVLAIELCSRESMFGYHGHGPSPFLRITLALPRLMAPARRLLEQGIRVPGLGTPSFAPYEANVDFEIRFMVDADIVGCNWLELPAGKYVRRAEKKATLCQLEVDVLWSDVISHPPEGQWQRIAPLRVLSFDIECAGRKGIFPEPERDPVIQICSLGLRWGEPEPFLRLALTLRPCAPILGAKVQSYEREEDLLQAWATFILAMDPDVITGYNIQNFDLPYLISRAQTLKVDRFPFLGRVTGLRSNIRDSSFQSRQVGRRDSKVVSMVGRVQMDMLQVLLREYKLRSYTLNAVSFHFLGEQKEDVQHSIITDLQNGNEQTRRRLAVYCLKDAFLPLRLLERLMVLVNNVEMARVTGVPLGYLLSRGQQVKVVSQLLRQAMREGLLMPVVKTEGGEDYTGATVIEPLKGYYDVPIATLDFSSLYPSIMMAHNLCYTTLLRPGAAQKLGLKPDEFIKTPTGDEFVKASVRKGLLPQILENLLSARKRAKAELAQETDPLRRQVLDGRQLALKVSPNSVYGFTGAQVGKLPCLEISQSVTGFGRQMIEKTKQLVETKYTLENGYDANAKVVYGDTDSVMCRFGVSSVAEAMSLGREAANWVSSHFPSPIRLEFEKVYFPYLLISKKRYAGLLFSSRSDAHDRMDCKGLEAVRRDNCPLVANLVTSSLRRILVDRDPDGAVAHAKDVISDLLCNRIDISQLVITKELTRAAADYAGKQAHVELAERMRKRDPGSAPNLGDRVPYVIIGAAKGVAAYMKSEDPLFVLEHSLPIDTQYYLEQQLAKPLLRIFEPILGEGRAESVLLRGDHTRCKTVLTSKVGGLLAFTKRRNSCIGCRSVIDHQGAVCKFCQPRESELYQKEVSHLNALEERFSRLWTQCQRCQGSLHEDVICTSRDCPIFYMRKKVRKDLEDQERLLQRFGPPGPEAW

SEQ ID NO: 61 - DNA, 3451 bp, Bos taurus
agtcaggggtcacggcggcgtgggctgtggcgggaaacactgtttgaagcgggatggatggtaagcggcgaccaggcccggggcctggggtgcccccaaagcgggcccgtgggggcctctgggatgaggatgaggcataccggccctcgcagttcgaggaggagctggcgctgatggaggagatggaagcagagcgcaggctgcaggagcaggaggaggaggagctgcagtcggccctggaggcggcggacgggcaattctccccaacggccatagatgcccgctggcttcggcccgccccgcccgccttggacccccagatggagcctctcatcttccagcagttggagatcgaccattacgtggccccagcgcggcccctgcctggggcgcccccgccatcccaggactcagttcccatcctccgcgccttcggggtcaccaacgagggggtctccgtctgctgccacatccatggctttgcaccctacttctacaccccagcgccccctggttttggacctgagcacctgagcgagctgcagcgggagctgagtgcagccatcagccgggaccagcgcgggggcaaggagctcaccgggccggccgtgctggcggtagagctgtgctcccgggagagcatgttcgggtaccatgggcacggcccctccccgtttctgcgtatcaccttggcactgccccgcctcatggcacctgcccgccgcctcctggagcagggcatccgcctggccggcctcggcacccccagctttgcgccctacgaggccaacgttgactttgagatccggttcatggtggacacggacatcgtgggctgcaactggctggagctcccagccgggaaatacatcctgaggccggaggggaaggccactctgtgtcagctggaggccgacgtgctgtggtcagacgtgatcagccacccgccggaaggagagtggcagcgaatcgcccctctgcgcgtgctcagcttcgacatcgagtgcgctggtcgcaaaggcatcttccctgagcccgagcgggaccccgtgatccagatctgctcactgggcctgcgctggggcgagccggagcccttcctgcgcctggcgctcaccctgcggccctgcgcccccatcctgggcgccaaggtgcagagctatgagcgggaggaggacctgctccaggcctggtcgaccttcatccgcatcatggatcccgatgtgatcaccggctacaatatccagaactttgaccttccctacctcatctcccgggcccagaccctcaaggtgccaggcttccccttgctgggccgtgtgattggcctccgctccaacatccgggagtcgtccttccagtccaggcagactggccggcgggacagcaaggtggtcagcatggtgggccgcgtgcagatggacatgctgcaggtgctgctgcgggagtacaagctccggtcctacacgctcaatgccgtgagcttccacttcctgggcgagcagaaggaggacgtgcagcacagcatcatcacagacctgcagaacggtaacgaccagacgcgccgccgcctggccgtgtactgcctcaaggacgccttcctacccctgcggctgctggagcggctcatggtgctggtgaacgccatggagatggcgcgcgtcaccggcgtgcccctcggctacctgctcagccgcggccagcaggtcaaggtcgtgtcccagctgctgcgacaggccatgcgccaggggctgttgatgcccgtggtgaagacggagggtggtgaggactataccggggccacggtcatcgagccgctgaaagggtactacgacgttcccatcgccaccttggacttctcctcgctgtacccgtccatcatgatggcccacaacctgtgctacaccacactcctgcggcccggggccgcccagaaactgggcctgaccgaggatcagttcatcaagacgcccacgggggacgagtttgtgaaggcatcggtgcggaaggggctgctcccccagatcctggaaaacctgctcagcgcccggaagagggccaaggccgagctggccaaggagacagaccccctacggcggcaagtgttggacgggcgccagctggcgctgaaagtgagtgctaactctgtgtacggcttcactggcgcccaggtgggcaggctcccgtgcctggaaatctcacagagtgtcaccgggttcgggcgccagatgattgagaagacaaagcagcttgtggagaccaagtacacggtggaaaacggctacagcaccagcgccaaggtggtgtatggtgacacagactcggtcatgtgccgctttggcgtctcatccgtggctgaggcgatggctttgggacgggaggctgcagactgggtgtccggccacttcccctcgcccatccggctagagtttgagaaagtctacttcccctacctgctcatcagcaagaagcgttacgcaggcctgctcttctcctcccggccggacgcccacgaccgcatggactgcaagggcctggaggccgtgcgcagggacaactgccccctggtggccaacctcgtcaccgcctcgctgcgccgcctgctcatcgaccgagacccctcgggcgccgtggctcatgcacaggacgtcatctccgatctgctgtgtaatcgcattgacatctcgcagctggtcattaccaaggagctgactcgcgctgccgccgattacgcgggcaagcaggcccacgtggagctggccgagaggatgaggaagcgggaccccgggagcgcgcccagcctgggcgaccgcgtcccctacgtgatcatcagcgctgccaagggcgtggccgcctacatgaagtccgaggaccccctgttcgtactggagcacagcctgcccatcgacacgcagtactacctggagcagcagctcgccaagccgctcctgcgcatcttcgagcccatcctgggcgagggccgtgccgaggctgtgctgctgcgcggggaccacactcgctgcaagacggtgctcacggggaaggtgggcggcctcctggccttcgccaaacgccggaactgctgcatcggctgccgcactgtcctcagccaccagggagccgtgtgcaagttctgccagccccgggagtcagagctgtaccagaaggaggtgtcccacctgagtgccctggaggagcgattctcacgcctgtggacgcagtgccagcgctgccagggcagcctgcacgaggacgtcatctgcaccagccgggactgtcccatcttctacatgcgcaagaaggtgcggaaggacctggaggaccaggagcggctgctgcggcgctttggaccccccggcccagaggcttggtgacctctgacctcaacgaacttcccaccttgggggcgcgggggggacagacggggaattaataaagctcaggccttttg

SEQ ID NO: 62 - protein, 1106 aa, Bos taurus
MDGKRRPGPGPGVPPKRARGGLWDEDEAYRPSQFEEELALMEEMEAERRLQEQEEEELQSALEAADGQFSPTAIDARWLRPAPPALDPQMEPLIFQQLEIDHYVAPARPLPGAPPPSQDSVPILRAFGVTNEGVSVCCHIHGFAPYFYTPAPPGFGPEHLSELQRELSAAISRDQRGGKELTGPAVLAVELCSRESMFGYHGHGPSPFLRITLALPRLMAPARRLLEQGIRLAGLGTPSFAPYEANVDFEIRFMVDTDIVGCNWLELPAGKYILRPEGKATLCQLEADVLWSDVISHPPEGEWQRIAPLRVLSFDIECAGRKGIFPEPERDPVIQICSLGLRWGEPEPFLRLALTLRPCAPILGAKVQSYEREEDLLQAWSTFIRIMDPDVITGYNIQNFDLPYLISRAQTLKVPGFPLLGRVIGLRSNIRESSFQSRQTGRRDSKVVSMVGRVQMDMLQVLLREYKLRSYTLNAVSFHFLGEQKEDVQHSIITDLQNGNDQTRRRLAVYCLKDAFLPLRLLERLMVLVNAMEMARVTGVPLGYLLSRGQQVKVVSQLLRQAMRQGLLMPVVKTEGGEDYTGATVIEPLKGYYDVPIATLDFSSLYPSIMMAHNLCYTTLLRPGAAQKLGLTEDQFIKTPTGDEFVKASVRKGLLPQILENLLSARKRAKAELAKETDPLRRQVLDGRQLALKVSANSVYGFTGAQVGRLPCLEISQSVTGFGRQMIEKTKQLVETKYTVENGYSTSAKVVYGDTDSVMCRFGVSSVAEAMALGREAADWVSGHFPSPIRLEFEKVYFPYLLISKKRYAGLLFSSRPDAHDRMDCKGLEAVRRDNCPLVANLVTASLRRLLIDRDPSGAVAHAQDVISDLLCNRIDISQLVITKELTRAAADYAGKQAHVELAERMRKRDPGSAPSLGDRVPYVIISAAKGVAAYMKSEDPLFVLEHSLPIDTQYYLEQQLAKPLLRIFEPILGEGRAEAVLLRGDHTRCKTVLTGKVGGLLAFAKRRNCCIGCRTVLSHQGAVCKFCQPRESELYQKEVSHLSALEERFSRLWTQCQRCQGSLHEDVICTSRDCPIFYMRKKVRKDLEDQERLLRRFGPPGPEAW

SEQ ID NO: 63 - DNA, 3457 bp, Drosophila melanogaster
ctctaacgtgcctaccaacaaaagcgcgccttattttttggcatcgctcttgtcttatggatttgcaagtaacatttcaccaaggtacccagaatggatggcaagcgcaagtttaatggaacctccaatggacatgccaagaagcccaggaatcctgatgacgatgaggaaatgggctttgaggcggagctggccgccttcgagaactccgaggacatggaccagactctgctaatgggcgatggacccgagaaccaaacgaccagtgagcgttggtcccgtccgccgcccccagaactagatccctccaagcacaacttggagtttcagcagctggacgtggaaaactatttgggacagccgttgccgggaatgccaggtgcccaaataggacccgtgccggtggtccgaatgtttggtgtcaccatggagggtaactctgtgtgctgccatgtgcatggtttctgtccatacttctacatagaggcgcccagtcaattcgaggagcaccattgcgagaaactacaaaaagccttggatcaaaaggttattgccgatattcgcaacaacaaagataatgtccaggaggctgtgcttatggtggaactggtggagaagctgaacatccatggatacaatggagacaagaagcagaggtacatcaaaatatcggttacgctgcccagatttgtggctgcggcctcacgtctcctcaaaaaggaagtgatcatgtcggagattgacttccaggactgtcgcgcctttgagaataacatagactttgacattcgcttcatggtggacactgatgtggtgggttgcaattggatagagcttcccatgggtcactggcgaataaggaacagtcacagcaagccgttgcctgaatcccgctgccagattgaagtagacgtggccttcgacagatttatatcccacgagcccgaaggtgaatggtccaaggtggctcccttccggatcctctcctttgatattgaatgcgctggtcgcaaaggaatatttccggaagccaaaatagatccagtcatccagatagccaatatggtgataaggcagggagaacgagaacctttcattaggaatgtctttaccctaaatgaatgcgctccaatcataggcagccaggtgttgtgccacgacaaggagacccagatgctggacaagtggtctgcctttgtccgggaagttgacccggatattttgaccggatataatatcaacaactttgacttcccctatttgcttaaccgagcagctcacttgaaggtcaggaactttgagtatttgggcaggattaagaacattcgttcggtgatcaaggaacagatgttgcagtcgaagcagatgggtcgcagggaaaaccagtacgttaattttgagggtagagttcccttcgatctcctctttgtcctgctgcgcgactacaaactacgctcgtacactctcaacgctgtgagctatcactttctgcaggagcaaaaggaggatgtgcatcatagcattatcacagatcttcagaatggagacgagcagacacgtcgccgttcggccatgtactgcctaaaggatgcctacttaccgcttagattgctggagaagttaatggccattgttaactacatggagatggccagggtgacgggtgtgccactggagtccttgctcacccgcggacaacagataaaggttttaagtcaattgctgcgcaaggccaaaaccaagggattcatcatgccctcgtacacctctcagggatcggatgaacagtatgaaggagcgactgtgattgaaccaaaacgtggctactatgcggaccccatctccacgctggatttcgcctctctgtatccaagtataatgatggcgcataatttgtgctacaccaccttggttttgggtggaactcgtgagaagctgcggcagcaggagaacctgcaggacgatcaagtggaacgtacgcctgcaaacaactactttgtgaagtctgaggtgcgtcgtggtctgctccctgagattctggaatctcttttggcggccagaaagcgtgccaaaaatgacctaaaagtggaaacagatccgtttaaaagaaaggtcctggatggcagacagctggcgctgaagatttcggctaattccgtgtacggatttactggcgcacaggttggaaagttgccatgcttagagatctcgggcagcgtcaccgcctacggtcgtaccatgatcgagatgacgaaaaacgaagtggaatcccattacacacaggccaatggctacgagaacaatgcagtggtcatctacggcgacactgattctgtgatggttaatttcggagtaaaaactctggagcgcagcatggagctgggacgcgaggctgccgaactggtcagttccaagttcgtgcatcctattaaattggaattcgagaaagtttactatccttacctgctgattaacaagaaacgctatgcgggattatactttacgcgcccagatacctacgataaaatggattgcaagggcatagaaaccgtgaggagagataactctccgctggtggccaacctgatgaactcctgcctgcagaaactactcatcgaaagggatcccgatggtgcagttgcctatgtgaaacaggtgatagccgatctcctctgcaatcgcatcgacatctcgcacttggtcataaccaaggagttggccaaaacggattacgcagccaaacaggcacacgttgagctggccgccaagatgaagaaaagagatcccggtacggcgcccaaactgggggatcgagttccctatgtgatctgtgcggcagccaaaaacacacccgcttaccagaaggccgaggatccgctgtatgtgctggaaaacagcgtgcccatcgatgccacttactacctggaacagcagctgtctaagccgctgctaaggatctttgaacctattttgggcgacaatgccgagtcaattttgttaaaaggagaacacacgcgcacacgaactgtggtaacatccaaagtgggtggacttgctggatttatgaccaagaaaacgtcgtgtttgggctgcaaatccctgatgcccaagggctacgaacaggcctgtctgtgtccacactgcgagccacgaatgagtgagctgtatcagaaggaggtgggtgcgaagagggaactggaggagaccttctctcgcctgtggaccgagtgccagcgatgccaggaatccttgcacgaggaggttatctgctccaacagagattgccccatcttctacatgcgacagaaggttcgcatggatctggacaatcaggagaagcgggtgttgcgattcggcctggccgagtggtaaccattgcatgagtttactgaattgtttaatcctataatttaataattatattactagaagttattaaaaaaaaaaaaaaaaaaaa

SEQ ID NO: 64 - protein, 1092 aa, Drosophila melanogaster
MDGKRKFNGTSNGHAKKPRNPDDDEEMGFEAELAAFENSEDMDQTLLMGDGPENQTTSERWSRPPPPELDPSKHNLEFQQLDVENYLGQPLPGMPGAQIGPVPVVRMFGVTMEGNSVCCHVHGFCPYFYIEAPSQFEEHHCEKLQKALDQKVIADIRNNKDNVQEAVLMVELVEKLNIHGYNGDKKQRYIKISVTLPRFVAAASRLLKKEVIMSEIDFQDCRAFENNIDFDIRFMVDTDVVGCNWIELPMGHWRIRNSHSKPLPESRCQIEVDVAFDRFISHEPEGEWSKVAPFRILSFDIECAGRKGIFPEAKIDPVIQIANMVIRQGEREPFIRNVFTLNECAPIIGSQVLCHDKETQMLDKWSAFVREVDPDILTGYNINNFDFPYLLNRAAHLKVRNFEYLGRIKNIRSVIKEQMLQSKQMGRRENQYVNFEGRVPFDLLFVLLRDYKLRSYTLNAVSYHFLQEQKEDVHHSIITDLQNGDEQTRRRSAMYCLKDAYLPLRLLEKLMAIVNYMEMARVTGVPLESLLTRGQQIKVLSQLLRKAKTKGFIMPSYTSQGSDEQYEGATVIEPKRGYYADPISTLDFASLYPSIMMAHNLCYTTLVLGGTREKLRQQENLQDDQVERTPANNYFVKSEVRRGLLPEILESLLAARKRAKNDLKVETDPFKRKVLDGRQLALKISANSVYGFTGAQVGKLPCLEISGSVTAYGRTMIEMTKNEVESHYTQANGYENNAVVIYGDTDSVMVNFGVKTLERSMELGREAAELVSSKFVHPIKLEFEKVYYPYLLINKKRYAGLYFTRPDTYDKMDCKGIETVRRDNSPLVANLMNSCLQKLLIERDPDGAVAYVKQVIADLLCNRIDISHLVITKELAKTDYAAKQAHVELAAKMKKRDPGTAPKLGDRVPYVICAAAKNTPAYQKAEDPLYVLENSVPIDATYYLEQQLSKPLLRIFEPILGDNAESILLKGEHTRTRTVVTSKVGGLAGFMTKKTSCLGCKSLMPKGYEQACLCPHCEPRMSELYQKEVGAKRELEETFSRLWTECQRCQESLHEEVICSNRDCPIFYMRQKVRMDLDNQEKRVLRFGLAEW

SEQ ID NO: 65 - DNA, 9064 bp, Drosophila melanogaster
ctgcagcttgggaaaatacttttggacaccccaaaaaaagttaagcgcgatattttcccaccgtgaccatgacaaccactgtgcgttcgaaaggctctctctctctctctctctttcgcgcaaatcaaaaacacaaacaggtttatgtgtgcggagagtgtgtgcgacagagagcggcgatatggaactgaaacgactgcaatgtttttatattccggcaacgcatttcgcataaattacaaattacacagcataagtgaatgcaagtgcaggggcggcagtcaaatggccagctgcacccagaaaaagggcaataagattcgggataacaaaacttgatggcgttcccgattttcccggacaagggagcgtatatgtatgtacacacaaaaaaaaaacttaaccagccttgcataacgaaacacgtgcaataaaaatatgactgattgtcaacctctgctgcaacttaattgctgccgcagaggtaaaactgaaaaacataaaaggggggcgacaagtgcagcaagcgaaaaaataaagaactcaaagagcgcatgcgcgccgctctcccactctctctccctctctctctgtcgctccactgcgctggaatcttacaactcgtgtaggtgagccggatttttatgatgatgcgcctgtgtgcgtgactgcatgatgccattgcagcggagaactagtagaaaaaagttcacatttcagcagttggaaaacacatggccaacaggccaactcaagtggccagcagctgtccttatattgtcagcaaataggtcatttaatgcccattacacgaaaattatagctaaaatggtcaagctgtgatgaaataaacataaatattatattttatgatttcatcagatttttagcatttttttttttaatttgtgttaggtagaactacaaagctaagaataattgaggatttctaggtaaaacttatattcttaaaaccatttaataattttcttgttttcttttatttgtagtaacattttaaaattggcgccaaacgtgttactttacagtgctgtgcaacagccaaatgtcagcattctctgcaacgcgttagcacatttctgagacgtttgcagatttttggcggcaacaagttatttacatttatttattttatttctgctaaacagcacggaaatgtccgactccggcaaaggcaaagtgctgcaaaatacgggtaaattcgtcagcgagaatcgcacagaaggcgtgagtggtcaaagttcgtggatttcacgctgaacaagggatttttcaatcttatccacaggacgacttcttcaatgaggcgggctatcgtcaatcccgggagaacgataaaatcgattcgaaatatggcttcgatcgggttaaggacagccaggagcggacgggctacctcatcaacatgcattcggtaagttaggaagcccataaaacgttgaaaatcatatccaataatggctatgccaattgcagaacgaagttttggatgaggacagaagattgattgctgccttggacctgttcttcatccaaatggatggttcccgcttcaaatgcacggtggcctatcagccatatttactcatccgaccagaggataatatgcatctggaagtggcgcgatttctgggtcgcaagtattccggccagatttctggactggagcacataaccaaagaagatttggatctgcccaatcatctatccggtttgcagcagcagtacataaaactttcgtttctcaatcagacagccatgaccaaggttagaagggaactcatgtccgcggtgaagagaaatcaggagcgacagaaatccaacacatactacatgcaaatgctggccacctcgctggcccaatcctccgcaggttccgaggatgccacattgggtaagaggcagcaggattacatggattgtattgtggacataagggagcatgatgtgccttaccacgtcagagtgtccatcgatttgcgcatcttttgtggacagtggtacaatatcaggtgcagaagtggcgtggaattgcctacgatcacctgccgaccggatattctggacagacccgaacccgtggtcctggcctttgatatagaaaccactaagctgccccttaagtttcccgatgcccagacggatcaggttatgatgatctcgtacatgatcgatggtcagggttatctgataaccaatcgtgagattatatcatccaatgtggacgattttgagtacactcccaagccggaattcgagggtaactttatagtattcaacgaagagaacgagatgcagctgctccagcgcttcttcgatcacatcatggaggtgcgtccccacatcattgttacatacaacggcgacttcttcgattggcccttcgtggagacgcgtgctgcagtgtacgatctggacatgaagcaagagattggcttctccaagctacgggatggcaattatctaagtcgccctgccatacacatggattgcctatgttgggtgaaacgagattcttatttacctgttggatctcaaggcttaaaggcggtggccaaggctaaattacgctatgatcctgtggaactcgatccggaggatatgtgccgcatggccgtggaacagccccaagtgctggccaattactctgtatccgatgcggtggccacatactatctgtacatgaagtatgtgcatccatttatcttcgccctaaatacgattattcccatggaacccgatgagatcctaagaaagggttccggcacactctgtgaaacgttgctgatggtggaggcttaccatgcccagattgtgtatcccaacaagcatcagagtgagctgaataagctctccaacgagggacacgtactggattcggaaacctatgtgggtggtcatgtggaggctttggaatcgggtgttttccgggcggacataccatgccgttttcgtctagatcctgctatggtcaagcaactgcaggagcaggttgatgcagttctgcgccacgctatcgaagtggaggaaggcataccgctcgagaaggtcttgaatctggatgaagtgcggcaggagattgtgcaggggctacagggtctgcacgatatacccaatcgcttggagcagccggtcatctatcacttggatgtgggtgccatgtaccccaacattattttgaccaatcgcctgcagccctcggcaatggttagtgacttagattgtgccgcctgtgacttcaacaagccaggagttcggtgcaaacgttccatggactggttgtggcgcggcgagatgttgcccgcctccaggaacgagtttcagcgcattcagcagcagctggagaccgagaagtttccaccccttttccctggcggaccacagcgagcctttcacgagctctccaaggaggatcaggcggcgtacgagaagaaacgtctgacggattactgccgcaaggcttacaagaagaccaagctaaccaaattggaaacgcgcacttcgaccatctgccagaaggagaacagcttctatgtggacacggtgcgagcttttcgcgatcgtcgctacgagtacaaaggactaaccaaagtggcaaaagcatcggtgaatgctgcggtggcttcgggagacgcggcagagatcaaggcagccaagggcagggaggtgctctacgattccctgcagttggcccacaagtgcatcctgaactccttctatggctacgtgatgaggagaggagcccgttggcattccatgcctatggccggcattgtgtgcctcacgggctcgaatattatcaccaaggcgagggaaattatcgagcgagttggtcgaccactcgaattggacactgatggtatatggtgcatattgcctggctcctttccgcaggagtttaccattcacacgagtcatgagaagaaaaagaagattaacatatcatatccgaatgcagtgctaaacactatggttaaagatcattttaccaacgatcagtaccacgagttgaggaaggataaggaaaacaatctacccaaatacgatattcgagatgagaactctatattcttcgaggtggatggaccctaccttgccatggtgttacccgctgccaaagaggagggcaagaagctgaagaaaagatatgcggtctttaatttcgatggcacactggctgaactcaagggattcgaggtgaagcgacgcggtgaactgcagctgatcaaaaatttccagagttccgtcttcgaagctttcctcgctggtagcacacttgaggaatgctatgcatctgtggccaaggtggcggattactggcttgatgtactctacagcagaggatcaaatctacccgactcggagctattcgaacttatttcggagaacaagagcatgtccaagaagcttgaggagtatggcgcccaaaagagtacgtccatctccacggccaagcgattggctgagttcctgggcgagcagatggtaaaggatgcgggtctggcttgtaagtacattatttcgaagaaacccgaaggagcacccgtcaccgagagagctattcccttggccattttccaatccgaaccgagcgtgaggcgacatcacctgcgtcgctggcttaaggacaacaccatgggcgatgcggatatacgcgatgtgctcgattggaactactacatagagagattgggtgggaccattcagaagatcataaccataccggcggcactgcagggactggccaatccagtgcccagagttcagcatccggattggttgcacaagaaaatgctggagaagaacgatgtgctcaagcagcgtcgcatcaatgagatgttcaccagcagacccaaaccgaaacctctagccacagaggaggacaagctggccgatatggaagatttggctggtaaagatggcggtgagggtgctgcaggctgtccgatagtcaccaagagaaagagaatccagctggaggagcacgatgaggaggaggcacagccgcaggccaccacttggcgtcaggccttgggcgctccaccgcccatcggtgaaaccagaaagaccatcgttgagtgggtgagatttcagaagaagaaatggaaatggcagcaggatcagcgccagcgtaatcgccaggcgagcaagcgaactcgaggcgaggatccacgctacactgggcggttccttagacgtgcacaacgcaccctgttggaccagccgtggcagatcgtacagttggtgcccgtcgacgacctgggccacttcactgtgtgggccttaatcggcgaagagttgcacaagatcaagttgacggtaccgaggattttctatgttaatcagcgaagtgctgctcctccagaggagggtcaactttggcgcaaggtcaatcgagttctgccacgatccagacctgttttcaatctctatcgatatagtgtgcccgaacagctcttccgggataactcgctgggcatgctggcggatctggcgacgcccgacattgagggcatatacgagacgcagatgacgttggaatttcgcgccctcatggacatgggctgcatttgcggtgtccagcgcgaggaggcacgtcgcttggcccaattggccaccaaggatctggaaacatttagcatcgagcagctggaacagggccccagactcaggtcaaatatttggctagcgccaacaatcgattgcgcaaaatctacttgtatcagcataacacaccgacggccaagaaggagatctgtgtcactgatcccaatgcctagcaagaaggcatttgtttttgccttggacacagtgcgtgccaatcaaatgccgaacatgaggcaattgtataccgccgagcgtttggccctgctcaagaatctgacggcagaggagcaagataaaattcctgtagaggattacacatttgaggttctcattgaggtggatgtcaaacaaatttaccggcacatacagcgggcactgaccacctacaaacaggagcatcagggaccacccaccattctgtgccttcaaacggcgctgtcggcgcgtaaactcagcctggccatgccgatcctgctggagtttccccaggctcagattcatatctccgatgacgctagtttgctttctggccttgattggcagcgacagggctccagggcagtgatacgccactttctgaatctgaacaatgttcttgatttgatgttggatcagtgtcgctactttcatgtgcccattggcaatatgccgccggatactgtgcttttcggagcggatcttttcttcgctcgcttgctgcagcggcataactttgtgctgtggtggtcggcgagtaccagaccagatttgggtggccgggaggcggacgacagccggctgttggcggaattcgaggagagcattagtgtggtgcaaaacaaggccggtttctatccggatgtttgcgtggagctggctctggatagcctggcggtgagtgccctgctccaatcgactaggattcaggaaatggaaggcgcctcatctgccattacgttcgatgtgatgccgcaggtctcgctggaggagatgattggcactgttccggcggccaccttgccgagttatgatgaaacggccctctgttccgccgccttccgcgttatgcgctccatggtgaatggttggttgcgagaggtatccatcaataggaacatcttctcggacttccagatcgtgcacttctatcgatgggtgcgctccagtaatgcactactctatgatcctgctttgagaagatctctgaataatctgatgaggaagatgttcttgcgcattatagcagagttcaagagattgggcgccaccattatctatgcggactttaacaggattatccttagttcgggtaagaaaaccgtttccgatgccctgggctatgtggactacattgtgcaaagcttgaggaacaaggagatgttccactccatccaactgagcttcgagcaatgctggaactttatgctctggatggaccaggcaaatttctcgggaattaggggaaagctaccaaagggaatcgatgagacagtgtcgtcaatagtttccactaccatgatacgggattctgaacgcaatcaagatgacgacgaggatgaagaagaggattcggaaaaccgtgatccagtggagagcaacgaggccgagcaggatcaagaggatgagctgtccctggagctcaactggacaattggcgaacatctgcccgatgaaaacgagtgccgcgaaaagtttgaatccctgctgaccctctttatgcaatctttggccgaaaagaagaccaccgagcaggccatcaaggatatctcgcactgcgcgttcgactttatcctgaaactgcacaaaaactacggcaagggcaagcccagcccgggcctagaacttatccgcactctgatcaaggcgttgagtgtggacaaaacgctggcggagcagatcaacgagttgcgccgaaatatgctgcgtctggtggggattggtgagttctcggacttggctgagtgggaggatccctgcgacagtcacatcatcaacgaggtcatctgcaaagcgtgtaatcactgcagggacctggatctctgcaaggacaagcatcgcgccatgaaagatggagtgtgagttacacaaatcagtacacataatttaccacaaataattgattaatgttggatttttcagacccgtttggctgtgtgcccagtgctatgtggcctatgataacgaggagatcgaaatgagaatgctggatgcactgcagcgcaagatgatgtcctatgtgctgcaggatttgcgctgttcgcgctgcagcgagatcaagcgcgagaatctggcagagttctgcacttgcgctggcaactttgtgcccctcatcagcgggaaggacatccagacactgctgggcacattcaacaaggtggctgccaaccacaagatgcagttgctccagcagactgttcatcaggcgctgaccacgccacgctaggacctagtttgttgttgttttctagatcgtagggcttaaatatattgtatttataatggaatttaattcgattttaatgagttttgagtttatgatgtcgcacaagacgaatgtctgtgttaaggaatggacgcgctttataattcaatgagattcacacacttttagtggctttcgcatacgaatcgcttgttgttttcccgattttattggttttttttgttgacttgcccgcggtttttgggggcgcacaggcgaaatcagcagctgaacttaaagcaattagactaactcattcgcgaagagcgatctctactgtggggcctgggtgatgggatcgaccttaacatcggggaactggaattcggggaacttcagcatgtcggtcttgccatcgctgccaaactgcttggccacacggtccagttcggtcttcagctcccgctcaatatcgggattggagtccaccagcttgccaccgctgcaggtgagcaaaacaaggattatgtcgaggcacaccaacggatgaaggagccagaacttacgcgctcttctgcttgtactcgcgcactttgtccagaaacagctgctggatgggatcggaggccttgttcagggcaggggcaacgattccgaagttacgacgggcctctgtgcgcaggacacgcatgccactcagcagggattgcgacagcatctggaattgatagatccatgttagaatagcaataaacggccttcttcatatgtaaccttaaaaaggttatgtaatacaattgtttgtttcgacgtttcccaatcggttttcaagccgactttgctagcaactatatcttgtattcaaattgtattccctcaatcgatttttatgtattttaaatcttgttttcaccttattttcctttgcaaatgctaactttcgtgcggaaaagtgacaattgtcagttcacaatggcagttggtgttagtgatgtgcgcgtgatgggtgtatgcgatactatcgtatgtaagctt

SEQ ID NO: 66 - protein, 2220 aa, Drosophila melanogaster
MSDSGKGKVLQNTGKFVSENRTEGDDFFNEAGYRQSRENDKIDSKYGFDRVKDSQERTGYLINMHSNEVLDEDRRLIAALDLFFIQMDGSRFKCTVAYQPYLLIRPEDNMHLEVARFLGRKYSGQISGLEHITKEDLDLPNHLSGLQQQYIKLSFLNQTAMTKVRRELMSAVKRNQERQKSNTYYMQMLATSLAQSSAGSEDATLGKRQQDYMDCIVDIREHDVPYHVRVSIDLRIFCGQWYNIRCRSGVELPTITCRPDILDRPEPVVLAFDIETTKLPLKFPDAQTDQVMMISYMIDGQGYLITNREIISSNVDDFEYTPKPEFEGNFIVFNEENEMQLLQRFFDHIMEVRPHIIVTYNGDFFDWPFVETRAAVYDLDMKQEIGFSKLRDGNYLSRPAIHMDCLCWVKRDSYLPVGSQGLKAVAKAKLRYDPVELDPEDMCRMAVEQPQVLANYSVSDAVATYYLYMKYVHPFIFALNTIIPMEPDEILRKGSGTLCETLLMVEAYHAQIVYPNKHQSELNKLSNEGHVLDSETYVGGHVEALESGVFRADIPCRFRLDPAMVKQLQEQVDAVLRHAIEVEEGIPLEKVLNLDEVRQEIVQGLQGLHDIPNRLEQPVIYHLDVGAMYPNIILTNRLQPSAMVSDLDCAACDFNKPGVRCKRSMDWLWRGEMLPASRNEFQRIQQQLETEKFPPLFPGGPQRAFHELSKEDQAAYEKKRLTDYCRKAYKKTKLTKLETRTSTICQKENSFYVDTVRAFRDRRYEYKGLTKVAKASVNAAVASGDAAEIKAAKGREVLYDSLQLAHKCILNSFYGYVMRRGARWHSMPMAGIVCLTGSNIITKAREIIERVGRPLELDTDGIWCILPGSFPQEFTIHTSHEKKKKINISYPNAVLNTMVKDHFTNDQYHELRKDKENNLPKYDIRDENSIFFEVDGPYLAMVLPAAKEEGKKLKKRYAVFNFDGTLAELKGFEVKRRGELQLIKNFQSSVFEAFLAGSTLEECYASVAKVADYWLDVLYSRGSNLPDSELFELISENKSMSKKLEEYGAQKSTSISTAKRLAEFLGEQMVKDAGLACKYIISKKPEGAPVTERAIPLAIFQSEPSVRRHHLRRWLKDNTMGDADIRDVLDWNYYIERLGGTIQKIITIPAALQGLANPVPRVQHPDWLHKKMLEKNDVLKQRRINEMFTSRPKPKPLATEEDKLADMEDLAGKDGGEGAAGCPIVTKRKRIQLEEHDEEEAQPQATTWRQALGAPPPIGETRKTIVEWVRFQKKKWKWQQDQRQRNRQASKRTRGEDPRYTGRFLRRAQRTLLDQPWQIVQLVPVDDLGHFTVWALIGEELHKIKLTVPRIFYVNQRSAAPPEEGQLWRKVNRVLPRSRPVFNLYRYSVPEQLFRDNSLGMLADLATPDIEGIYETQMTLEFRALMDMGCICGVQREEARRLAQLATKDLETFSIEQLEQGPRLRSNIWLAPTIDCAKSTCISITHRRPRRRSVSLIPMPSKKAFVFALDTVRANQMPNMRQLYTAERLALLKNLTAEEQDKIPVEDYTFEVLIEVDVKQIYRHIQRALTTYKQEHQGPPTILCLQTALSARKLSLAMPILLEFPQAQIHISDDASLLSGLDWQRQGSRAVIRHFLNLNNVLDLMLDQCRYFHVPIGNMPPDTVLFGADLFFARLLQRHNFVLWWSASTRPDLGGREADDSRLLAEFEESISVVQNKAGFYPDVCVELALDSLAVSALLQSTRIQEMEGASSAITFDVMPQVSLEEMIGTVPAATLPSYDETALCSAAFRVMRSMVNGWLREVSINRNIFSDFQIVHFYRWVRSSNALLYDPALRRSLNNLMRKMFLRIIAEFKRLGATIIYADFNRIILSSGKKTVSDALGYVDYIVQSLRNKEMFHSIQLSFEQCWNFMLWMDQANFSGIRGKLPKGIDETVSSIVSTTMIRDSERNQDDDEDEEEDSENRDPVESNEAEQDQEDELSLELNWTIGEHLPDENECREKFESLLTLFMQSLAEKKTTEQAIKDISHCAFDFILKLHKNYGKGKPSPGLELIRTLIKALSVDKTLAEQINELRRNMLRLVGIGEFSDLAEWEDPCDSHIINEVICKACNHCRDLDLCKDKHRAMKDGCYVAYDNEEIEMRMLDALQRKMMSYVLQDLRCSRCSEIKRENLAEFCTCAGNFVPLISGKDIQTLLGTFNKVAANHKMQLLQQTVHQALTTPR

SEQ ID NO: 67 - DNA, 26 bp, Artificial Sequence
  primer
gactagtggctatcttgtggcgggaa

SEQ ID NO: 68 - DNA, 26 bp, Artificial Sequence
  primer
ggaattccttgtcccgtgtcaggtca

SEQ ID NO: 69 - DNA, 24 bp, Artificial Sequence
  primer
cagaactttgccctcccatacctc

SEQ ID NO: 70 - DNA, 3318 bp, Mus musculus
atggattgtaagcggcgacaaggaccaggccctggggtgcccccaaagcgggctcgagggcacctctgggatgaggacgagccttcgccgtcgcagtttgaggcgaacctggcactgctggaggaaatagaggctgagaaccggctgcaggaggcagaggaggagctgcagctgcccccagagggcaccgtgggtgggcagttttccactgcagacattgaccctcggtggcggcggcccaccctacgtgccctggaccccagcacggagcccctcatcttccagcagctggagattgaccactatgtgggctcagcaccacccctgccagaagggcccctgccatcccggaactcagtgcccatactgagggcctttggggtcaccgatgaaggcttctccgtctgctgccacatacagggctttgccccctacttctacacccccgcgcctcctggttttggggccgagcacctgagtgagctgcagcaggagctgaacgcagccatcagccgggaccagcgcggtgggaaggagctctcagggccggcagtgctggcaatagagctatgctcccgtgagagcatgtttgggtaccacggtcatggcccttctccatttctccgcatcaccctggcactaccccgccttatggcaccagcccgccgccttctggaacagggtgtccgagtgccaggcctgggcaccccgagcttcgcaccctacgaagccaacgtggactttgagatccggttcatggtggatgctgacattgtgggatgcaactggttggagctgccagctggaaagtacgttcggagggcggagaagaaggccaccctgtgtcagctggaggtggacgtgctgtggtcagatgtgatcagtcacccaccggaggggcagtggcagcgcattgcacccctgcgtgtgcttagcttcgacatcgagtgtgctggccgaaaaggcatcttccctgagcctgagcgtgaccccgtgatccagatctgttctctggggctgcgctggggggagccggagccattcttgcgtctggcactcacgctgcggccctgtgcccccatcctgggtgccaaagtgcagagctatgagcgggaagaagacctgctccaggcctgggccgacttcatccttgccatggaccctgacgtgatcaccggctacaacattcagaactttgccctcccatacctcatctctcgggcacaggccctaaaggtggaccgcttccctttcctgggccgcgtgactggtctccgctccaacatccgtgactcctccttccaatcaaggcaggtcggccggcgggacagtaaggtgatcagcatggtgggtcgcgttcagatggatatgctgcaggtgctgcttcgggaacacaagctccgctcctacacgctcaacgctgtgagtttccacttcctgggcgagcagaaggaggacgttcagcacagcatcatcaccgacctgcagaatgggaacgaacagacgcgccgccgcctggccgtgtactgcctgaaggacgcctttctgccactccgactactagagcgccttatggtgctggtgaataatgtggagatggcgcgtgtcacgggtgtaccccttgggtacctgctcacccggggccagcaggtcaaggtcgtgtctcagctgctgcgccaggccatgcgccaggggctgctgatgcctgtggtgaagaccgagggcagtgaggactacacgggagccacagtcattgagcccctcaaagggtactatgacgtccccattgccaccctggacttctcctccttgtacccatccatcatgatggcccataatctgtgctacaccacgctgctccgacctggggctgcccagaagctgggccttaaaccagatgagttcatcaagacacccactggggatgagtttgtgaagtcatctgtacggaagggcctcctgccccagatcctggagaatctgctgagtgcccgcaagagggccaaggctgagctggctcaggagacggaccccctgcggcgacaggtcttggacggccggcaactggcactaaaagtgagtgccaactccgtatatggcttcactggtgcccaggtgggcaagctgccatgtttggagatctcccagagtgtcactgggttcgggcggcagatgattgagaaaaccaagcagcttgtggagtccaagtacaccgtggaaaatggctacgatgccaacgccaaggtagtctacggtgacacggactctgtgatgtgccggtttggcgtctcctctgtggctgaagcaatgtctctggggcgggaggctgcaaactgggtatccagtcacttcccatcacccatccggctggagttcgagaaggtttacttcccatacctgctcatcagcaagaagcgctatgctggcctgctcttctcctcccgctctgatgcccatgacaaaatggactgcaagggcctggaggctgtgcgcagggacaactgtcccctggtggccaacctcgttacatcctctctgcgccggatcctcgtggaccgggaccctgatggggcagtagcccatgccaaggacgtcatctcggacctgctgtgcaaccgcatagacatctcccagctggtcatcaccaaagagttgacccgcgcagcagcagactatgctggcaagcaggctcacgtggagctggctgagaggatgaggaagcgcgaccccggcagtgcgcccagcctgggtgaccgagtcccctatgtgatcattggtgctgctaagggtgtggccgcctacatgaagtcggaggaccccctgtttgtgctggagcacagcctgcccatcgacactcagtactacctggagcagcagctggccaagccgctcttgcgcatctttgagcccatcctgggtgagggccgtgcagagtctgtgctgctgcgcggtgaccacacacgatgcaagactgtgctcaccagcaaggtgggcggcctcttggccttcaccaagcgccgcaactgttgcattggctgccgctccgtaatcgaccatcaaggagccgtgtgtaagttctgtcagccacgggagtcggagctctatcagaaggaggtgtcacacctgaatgccttggaagaacggttctctcgcctctggacacagtgtcaacgctgccagggcagcttgcatgaggacgtcatctgtaccagccgtgactgtcccatcttctacatgcgcaagaaggtgcgcaaggacctggaagaccaggaacggctgctgcagcgctttggaccgcccggccctgaggcctggtga

SEQ ID NO: 71 - DNA, 29 bp, Artificial Sequence
  primer
tccccgcggctgcagaggattttccacag

SEQ ID NO: 72 - DNA, 28 bp, Artificial Sequence
  primer
ggactagtatggtatgcacaacagcctc

SEQ ID NO: 73 - DNA, 29 bp, Artificial Sequence
  primer
tccccgcggagtggttgtgggagacttac

SEQ ID NO: 74 - DNA, 28 bp, Artificial Sequence
  primer
ggactagtcagtcccacagtcccaaagt

SEQ ID NO: 75 - DNA, 18 bp, Artificial Sequence
  primer
ctgagagtgatgaggttc

SEQ ID NO: 76 - DNA, 21 bp, Artificial Sequence
  primer
ctaatcgccatcttccagcag

SEQ ID NO: 77 - DNA, 20 bp, Artificial Sequence
  primer
gctcgacgttgtcactgaag

SEQ ID NO: 78 - DNA, 20 bp, Artificial Sequence
  primer
ccaacgctatgtcctgatag

SEQ ID NO: 79 - DNA, 20 bp, Artificial Sequence
  primer
gctcgacgttgtcactgaag

SEQ ID NO: 80 - DNA, 20 bp, Artificial Sequence
  primer
ccaacgctatgtcctgatag

SEQ ID NO: 81 - DNA, 5725 bp, Mus musculus
agtggttgtgggagacttaccgtcctcttgtctctggagagtgccttctaccatgtcatcagaagggctgtcatcttggtccccgatttcttccacatcattgtcctgagcatcagcagcgtctccaatggggcagtttaatttgaggcaatacttactcttcaactggccgttactttcacctggactagacacatcgcgcttctgagaacacactttatccaaaaaccggataccgaaatcctgcccggactacatcatcaagttgatgtcctcctttttcacgaaaatctttgtggtgtacctgtagttcagcacctcttcaaatatgtgggagcagatgaaatctatctctatgatggacaagctgtccagttctagcttcttgaaaagtttcttttttttttctttcaattttttattaggtatttagctcatttacatttccaatgctataccaaaagtcccccatacccacccacccccactcccctacccgctcactccacctttttggccctggcgttcccctgttctggggcatataaagtttgtgtgtccaatgggcctctctttccagtgatggccgactaggccatcttttgatacatatgcagctagagtcaagagctccggggtactggttagttcataatgttgatccacctatagggttgcagatccctttagctccttgggtactttctctagctcctccattgggagccctgtgatccatccattagctgactgtgggcatccacttctgtgtttgctaggccccggcatagtctcacaagagacagctacatctgggtcctttcgataaaatcttgctagtgtatgcaatggtgtcagcgtttggatgctgattatggggtggatccctggataaggcagtctctacatggtccatcctttcatctcagctccaaactttgtctctgtaactccttccaagggtgttttgttcccacttctaaggaggggcatagtgtccacacttcagtcttcttttttcatgagtttcatgtgtttaggaaattgtatcttatatcttgggtatcctaggttttgggctaatatccacttatcagcgagtacatattgtgtgagttcctttgtgaatgtgttacctcactcaggaagatgccctccaggtccatccatttggctaggaatttcataaattcattctttttaatagctgagtagtactccattgtgtagatgtaccacattttctgtatccattcctctgttgaggggcatctgggttctttccagcttctggctattataaataaggctgctatgaacatagtggagcatgtgtccttcttaccagttggggcatctcctggatatatgcccaggagaggtattcctggatcctccggtagtactatgtccaattttctaaggaaccgccagatggatttccagagtggttgtacaagcctgcaatcccaccaacaatggaggagtgttcctctttctccacatcctcgccagcatctgctgtcacctgaatttttgatcttagccattctgactggtgtgaggtggaatctcagggttgttttgatttgcatttccctgatgattaaggatgttgaacattttttcaggtgcttctctgccattcggtattcctcaggtgagaattctttgttcagttctgagccccatttttttaatggggttatttgattttctgaagtccaccttcttgagttctttatatatgttggatattagtcccctatctgatttaggataggtaaagatcctttcccaatctgttggtggtctctttgtcttattgacagtgtcttttgccttgcagaaactttggagtttcattaggtcccatttgtcaattctcgatcttacagcacaagccattgctgttctgttcaggaatttttcccctgtgcccatatcttcaaggcttttccccactttctcctctataagtttcagtgtctctggttttatgtgaagttctttgatccatttagatttgaccttagtacaaggagataagtatggatcgattcgcattcttctacatgataacaaccagttgtgccagcaccatttgttgaaaatgctgtctttcttccactggatggttttagctcccttgtcgaagatcaagtgaccataggtgtgtgggttcatttctgggtcttcaattctattccattggtctacttgtctgtctctataccagtaccatgcagtttttaccacaattgctctgtagtaaagctttaggtcaggcatggtgattccaccagaggttcttttatccttgagaagagtttttgctatcctaggttttttgttattccagatgaatttgcaaattgctccttctaattcgttgaagaattgagttggaattttgatggggattgcattgaatctgtagattgcttttggcaagatagccatttttacaatattgatcctgccaatccatgagcatgggagatctttccatcttctgagatcttctttaatttctttcttcagagacttgaagtttttatcatacatatctttcacttcctcagttagagtcacgccgagatattttatattatttgtgactattgagaagggtgttgtttccctaatttctttctcagactgtttattctttgtgtagagaaaggccattgacttgtttgagttaattttatatccagctacttcaccaaagctgtttatcaggtttaggagttctctggtggaatttttagggtcacttatatatactatcatatcatctgcgaaaagtgatattttgacttcctcttttccaatttgtatccccttgatctccttttgttgtcgaattgctcagcactgcagtattcttaacaaagttgattctcattagaaaataaagctgccatatgaccatctaacacttagtaactgggaaaatttgatttgatctggtgatttgtcacagcaataacaaagtaactatcacaatggctgaaacaagtctgctcacttggaatcaacactttttgtcccaggagcaacagagtcttcagtatcaggggcctaaatttagagatgaaggtttggacctgtagcctgaacctcttgcagaagtgatgtattaaaaggctgatcaacaatggtcgctacaagagtactccaggccagccaatccaacaagcccaataccttgaaggatgaaacccatgcccaagatcacttcctgtgcctggtggctgggcctctactgagtgccacaaagtagaagaaaaaactctagatgctagtgttcctctaagcatgaataactgatatatattcatgctgctctacctcagatattcagacttttccccctggttttcagtaaatcaagttggatcactccactcttctctctcctcctcttctcatttcctctgttcagcctcccctcccagattctccagtctgaccacgtttctaaacagtttgctaatttatcttggcagctgaggcagctatgggagccacagcaggcagagagcacaggagactcagggttctcagcagggctctcagcagggctctcagcagggctgttgtgggtgatccttggattctctatcctccaggaacagtttcactgttccagaagatggtgatgattctgacgactgagatgcacgtgtggtggtgtctctggaggctaaagtgcaggtgccaagtggttccaaatagctgggtgcagtttgtctcaggggcacctctagagggtcagcctgtttctttcagcctctagagggcagagatgagtcccagaccttgctctctacattatcctagcctttctgtgccctttgtgaggcgcctgcagttaagaacgtgtccagcagggggcgcagtgtacatgggtgttctccagtaacagtcttggctgggaaggagagctagattccaggttcttaaaatactgttttcctgtacatacctaagtaatctttacattggaaaacaaacaaatagtaaagctctgttgttgttgttgataagtagcaaattaatttagggggagcagtttggggggctgatgggtcttgttctgccattcaggctgggctgaagctcctagggtctgaagatgctcctgtctcagactccaaagcatgggactgcatacagaagcgtgcaacagtgctaggcttccaatcaggtttcacattttgcttctagacagaaatataatttcctgaaacctttcgttttgaaatgatgtcattattgggcccccgcactcattgctgctgacgcccccctcccactatccctcgggctgaaatgtcctgattgggtgtcaacgctcatttgcactgatgctccccccttaatccctggagctgaaatgtcctgattgggtgccaaaattttttccactgatgcctctcccatcccattagcactaggactgaaatgtcatgattgggcgcaaaaactaatttccaattatgcccggcccctccgattatccctggtgctgagatgtcatgattggggccaacactcatttctgctgatggccctcctctcccattagacctggacctgaatccgtgtcatgattggacgccaacactcatttccactgatgccccggccctcccattagtcctgaggctgaacccctgtcatgattgggctccaatcctcatttcaggtgatgctcagctcctcccatcagccctgtggctgaatctctgtcacgattgggccccaaccctcatttctgctgaggcccagcccctcccattagccctggggctgcatccctgtcaccactagacaccaacactcatttctgctgatgccccgcccatgccattacccttgggcctaaatccctgtcatgattgaactccaacccacatttctcctgataccctgactcccattatccttaggcttaatccgtgtcataattgggcgccaacactgatttcctctgatgccctgcccctcccattagcccgggactgaatccctgtcataattggacttgaaccctcatttttgctgaggcccagcgcctcccatttgtcctcgggctgatccctgtcatgattgggccccaaccctcatttcggctgaggccccactcctccaattagccctgaggctgaatccatgtcatgagtaggcaccaactctcattaacactgatgcgctgccccttacattagccctggggctgaatccctgtcatgattgggccccaacactcatttctgctgatggccctcctctcccattagacctgaggctgaatccgtgtcatgattgggcaccaaaccgcaattccactaaagccccacccctcccattaccatcctgccgaaaccatgtcttgtcatgattgggccccaaccctcctttccactgatgcccccccctcccttaagccctcctgctgagaccacatcttgattgggcactaacactcatttccgctgatgcccaccactcccatttgccctgggacttaaaccctgtcgtgattgggtgtcaaccctcatttccggtgatgccccgcctcttcctataaatcctggcgctcaaatactgtggtcggtgggcaggaacagccatttggatcactgcctgcagcctagcggttgagctgctctggcgatcatctgttctgaggtactttgggactgtgggactg

SEQ ID NO: 82 - DNA, 30 bp, Artificial Sequence
  primer
ctgagtctagatttcccgccatggaaatcg

SEQ ID NO: 83 - DNA, 30 bp, Artificial Sequence
  primer
agcaacgagctcttatgattggtttatctg

SEQ ID NO: 84 - DNA, 30 bp, Artificial Sequence
  primer
atttgctgtcgataatatcagatttcttgg

SEQ ID NO: 85 - DNA, 28 bp, Artificial Sequence
  primer
gagtgaggatttgtacatgatctgaagg

SEQ ID NO: 86 - DNA, 3357 bp, Mus musculus
  CDS (24)..(3341)
ggcgggaaaagctgtttgaggcgatggattgtaagcggcgacaaggaccaggccctggggtgcccccaaagcgggctcgagggcacctctgggatgaggacgagccttcgccgtcgcagtttgaggcgaacctggcactgctggaggaaatagaggctgagaaccggctgcaggaggcagaggaggagctgcagctgcccccagagggcaccgtgggtgggcagttttccactgcagacattgaccctcggtggcggcggcccaccctacgtgccctggaccccagcacggagcccctcatcttccagcagctggagattgaccactatgtgggctcagcaccacccctgccagaagggcccctgccatcccggaactcagtgcccatactgagggcctttggggtcaccgatgaaggcttctccgtctgctgccacatacagggctttgccccctacttctacacccccgcgcctcctggttttggggccgagcacctgagtgagctgcagcaggagctgaacgcagccatcagccgggaccagcgcggtgggaaggagctctcagggccggcagtgctggcaatagagctatgctcccgtgagagcatgtttgggtaccacggtcatggcccttctccatttctccgcatcaccctggcactaccccgccttatggcaccagcccgccgccttctggaacagggtgtccgagtgccaggcctgggcaccccgagcttcgcaccctacgaagccaacgtggactttgagatccggttcatggtggatgctgacattgtgggatgcaactggttggagctgccagctggaaagtacgttcggagggcggagaagaaggccaccctgtgtcagctggaggtggacgtgctgtggtcagatgtgatcagtcacccaccggaggggcagtggcagcgcattgcacccctgcgtgtgcttagcttcgacatcgagtgtgctggccgaaaaggcatcttccctgagcctgagcgtgaccccgtgatccagatctgttctctggggctgcgctggggggagccggagccattcttgcgtctggcactcacgctgcggccctgtgcccccatcctgggtgccaaagtgcagagctatgagcgggaagaagacctgctccaggcctgggccgacttcatccttgccatggaccctgacgtgatcaccggctacaacattcagaactttgacctcccatacctcatctctcgggcacaggccctaaaggtggaccgcttccctttcctgggccgcgtgactggtctccgctccaacatccgtgactcctccttccaatcaaggcaggtcggccggcgggacagtaaggtgatcagcatggtgggtcgcgttcagatggatatgctgcaggtgctgcttcgggaacacaagctccgctcctacacgctcaacgctgtgagtttccacttcctgggcgagcagaaggaggacgttcagcacagcatcatcaccgacctgcagaatgggaacgaacagacgcgccgccgcctggccgtgtactgcctgaaggacgcctttctgccactccgactactagagcgccttatggtgctggtgaataatgtggagatggcgcgtgtcacgggtgtaccccttgggtacctgctcacccggggccagcaggtcaaggtcgtgtctcagctgctgcgccaggccatgcgccaggggctgctgatgcctgtggtgaagaccgagggcagtgaggactacacgggagccacagtcattgagcccctcaaagggtactatgacgtccccattgccaccctggacttctcctccttgtacccatccatcatgatggcccataatctgtgctacaccacgctgctccgacctggggctgcccagaagctgggccttaaaccagatgagttcatcaagacacccactggggatgagtttgtgaagtcatctgtacggaagggcctcctgccccagatcctggagaatctgctgagtgcccgcaagagggccaaggctgagctggctcaggagacggaccccctgcggcgacaggtcttggacggccggcaactggcactaaaagtgagtgccaactccgtatatggcttcactggtgcccaggtgggcaagctgccatgtttggagatctcccagagtgtcactgggttcgggcggcagatgattgagaaaaccaagcagcttgtggagtccaagtacaccgtggaaaatggctacgatgccaacgccaaggtagtctacggtgacacggactctgtgatgtgccggtttggcgtctcctctgtggctgaagcaatgtctctggggcgggaggctgcaaactgggtatccagtcacttcccatcacccatccggctggagttcgagaaggtttacttcccatacctgctcatcagcaagaagcgctatgctggcctgctcttctcctcccgctctgatgcccatgacaaaatggactgcaagggcctggaggctgtgcgcagggacaactgtcccctggtggccaacctcgttacatcctctctgcgccggatcctcgtggaccgggaccctgatggggcagtagcccatgccaaggacgtcatctcggacctgctgtgcaaccgcatagacatctcccagctggtcatcaccaaagagttgacccgcgcagcagcagactatgctggcaagcaggctcacgtggagctggctgagaggatgaggaagcgcgaccccggcagtgcgcccagcctgggtgaccgagtcccctatgtgatcattggtgctgctaagggtgtggccgcctacatgaagtcggaggaccccctgtttgtgctggagcacagcctgcccatcgacactcagtactacctggagcagcagctggccaagccgctcttgcgcatctttgagcccatcctgggtgagggccgtgcagagtctgtgctgctgcgcggtgaccacacacgatgcaagactgtgctcaccagcaaggtgggcggcctcttggccttcaccaagcgccgcaactgttgcattggctgccgctccgtaatcgaccatcaaggagccgtgtgtaagttctgtcagccacgggagtcggagctctatcagaaggaggtgtcacacctgaatgccttggaagaacggttctctcgcctctggacacagtgtcaacgctgccagggcagcttgcatgaggacgtcatctgtaccagccgtgactgtcccatcttctacatgcgcaagaaggtgcgcaaggacctggaagaccaggaacggctgctgcagcgctttggaccgcccggccctgaggcctggtgacctgacacgggacaag

SEQ ID NO: 87 - protein, 1105 aa, Mus musculus
MDCKRRQGPGPGVPPKRARGHLWDEDEPSPSQFEANLALLEEIEAENRLQEAEEELQLPPEGTVGGQFSTADIDPRWRRPTLRALDPSTEPLIFQQLEIDHYVGSAPPLPEGPLPSRNSVPILRAFGVTDEGFSVCCHIQGFAPYFYTPAPPGFGAEHLSELQQELNAAISRDQRGGKELSGPAVLAIELCSRESMFGYHGHGPSPFLRITLALPRLMAPARRLLEQGVRVPGLGTPSFAPYEANVDFEIRFMVDADIVGCNWLELPAGKYVRRAEKKATLCQLEVDVLWSDVISHPPEGQWQRIAPLRVLSFDIECAGRKGIFPEPERDPVIQICSLGLRWGEPEPFLRLALTLRPCAPILGAKVQSYEREEDLLQAWADFILAMDPDVITGYNIQNFDLPYLISRAQALKVDRFPFLGRVTGLRSNIRDSSFQSRQVGRRDSKVISMVGRVQMDMLQVLLREHKLRSYTLNAVSFHFLGEQKEDVQHSIITDLQNGNEQTRRRLAVYCLKDAFLPLRLLERLMVLVNNVEMARVTGVPLGYLLTRGQQVKVVSQLLRQAMRQGLLMPVVKTEGSEDYTGATVIEPLKGYYDVPIATLDFSSLYPSIMMAHNLCYTTLLRPGAAQKLGLKPDEFIKTPTGDEFVKSSVRKGLLPQILENLLSARKRAKAELAQETDPLRRQVLDGRQLALKVSANSVYGFTGAQVGKLPCLEISQSVTGFGRQMIEKTKQLVESKYTVENGYDANAKVVYGDTDSVMCRFGVSSVAEAMSLGREAANWVSSHFPSPIRLEFEKVYFPYLLISKKRYAGLLFSSRSDAHDKMDCKGLEAVRRDNCPLVANLVTSSLRRILVDRDPDGAVAHAKDVISDLLCNRIDISQLVITKELTRAAADYAGKQAHVELAERMRKRDPGSAPSLGDRVPYVIIGAAKGVAAYMKSEDPLFVLEHSLPIDTQYYLEQQLAKPLLRIFEPILGEGRAESVLLRGDHTRCKTVLTSKVGGLLAFTKRRNCCIGCRSVIDHQGAVCKFCQPRESELYQKEVSHLNALEERFSRLWTQCQRCQGSLHEDVICTSRDCPIFYMRKKVRKDLEDQERLLQRFGPPGPEAW

SEQ ID NO: 88 - DNA, 3318 bp, Mus musculus; identical to SEQ ID NO: 70
  CDS (1)..(3318)

SEQ ID NO: 89 - protein, 1105 aa, Mus musculus
MDCKRRQGPGPGVPPKRARGHLWDEDEPSPSQFEANLALLEEIEAENRLQEAEEELQLPPEGTVGGQFSTADIDPRWRRPTLRALDPSTEPLIFQQLEIDHYVGSAPPLPEGPLPSRNSVPILRAFGVTDEGFSVCCHIQGFAPYFYTPAPPGFGAEHLSELQQELNAAISRDQRGGKELSGPAVLAIELCSRESMFGYHGHGPSPFLRITLALPRLMAPARRLLEQGVRVPGLGTPSFAPYEANVDFEIRFMVDADIVGCNWLELPAGKYVRRAEKKATLCQLEVDVLWSDVISHPPEGQWQRIAPLRVLSFDIECAGRKGIFPEPERDPVIQICSLGLRWGEPEPFLRLALTLRPCAPILGAKVQSYEREEDLLQAWADFILAMDPDVITGYNIQNFALPYLISRAQALKVDRFPFLGRVTGLRSNIRDSSFQSRQVGRRDSKVISMVGRVQMDMLQVLLREHKLRSYTLNAVSFHFLGEQKEDVQHSIITDLQNGNEQTRRRLAVYCLKDAFLPLRLLERLMVLVNNVEMARVTGVPLGYLLTRGQQVKVVSQLLRQAMRQGLLMPVVKTEGSEDYTGATVIEPLKGYYDVPIATLDFSSLYPSIMMAHNLCYTTLLRPGAAQKLGLKPDEFIKTPTGDEFVKSSVRKGLLPQILENLLSARKRAKAELAQETDPLRRQVLDGRQLALKVSANSVYGFTGAQVGKLPCLEISQSVTGFGRQMIEKTKQLVESKYTVENGYDANAKVVYGDTDSVMCRFGVSSVAEAMSLGREAANWVSSHFPSPIRLEFEKVYFPYLLISKKRYAGLLFSSRSDAHDKMDCKGLEAVRRDNCPLVANLVTSSLRRILVDRDPDGAVAHAKDVISDLLCNRIDISQLVITKELTRAAADYAGKQAHVELAERMRKRDPGSAPSLGDRVPYVIIGAAKGVAAYMKSEDPLFVLEHSLPIDTQYYLEQQLAKPLLRIFEPILGEGRAESVLLRGDHTRCKTVLTSKVGGLLAFTKRRNCCIGCRSVIDHQGAVCKFCQPRESELYQKEVSHLNALEERFSRLWTQCQRCQGSLHEDVICTSRDCPIFYMRKKVRKDLEDQERLLQRFGPPGPEAW

SEQ ID NO: 90 - DNA, 3684 bp, Arabidopsis thaliana
agcgattccttagcagaaaggcgctccatttctctggcgtaaaccaaaggagatccttgaactgtttcctgcaccattgctcttaaaacccttctccggcacgaattcttccaaccctgcttcaccaccggaacattgagacaaaatctcgacggtgacgctgaggttgaaaaaaccaatcgaaccgcagacgtaccaggaaccgaaccatgtatcaacgccattgaagaagaagaagaagaagaaggtgaaaaacgaaagattgagaatttgtttgctttgagcaaccaaacctcaggaaaaaagagttaaggtgggagtgtctggttcaaccggtttatatccggttcaaattaaacctcttacagttaaccgggttttgtgtttggttcgattgttcataaaagaaagaagactcttgtcgtcgattagtgccaaagttgaaagttgaaaccttttctcagaattttctgctcagttctgagtttttttttcccgccatggaaatcgactccgagaaaattcacgaaaggaagcaatccgattacaattcgctggtacgaactctattactttatcgacttgtagtgaaagacaaatgtaatcattcgtggtggtgactgtttctacttataagtgtacgggctagggtttgttatctgattctgagtttttgcaattgaagcaggatgagagattcgagatacagaaggagatgtacagaggtcagcaatacagtcagatttactttgctcgtcttcatctcatgagaacacttctctactctcttgctcctacttggaaatctcatttgcctggtcagtgcttttgtttctctcatatttagcacaacaacgaagagcagtttttgagaattttcttgggttagatataattaggtgaaatcagtgatttttagggatttttgctatcttatggattacagttgagaaagattgctagtattgtttaaattatagatctgaatgtgaatttcatttttgcagtgtgtaaggttttgggacttgaaaaaggaaaagaatgcataattgtgggaaccttgttcaaacacatgaagcttaaaccttgtgttctcgatgaatattctaaagaggttggtttttattaacctctactgtttttttgagctatgtctatgctgaatcaatctgagtatatttaacataatgcagaggtcagttactccgcttgttaaaccacataactttatgcatcctgatgataatctgatcctcgaagacgagagtgggagagttaagcttgctggttccgcactttcacctgcgatttatgtgacaggtattgcaaatgggttcttactgtttttactgtatgattttttccttctttacaatgtggcaaatcttagagattttgatcaagctttcctctcttaaaagatgggttctttaagaaaattaacgttgaagcctcccgtgcattgtaggtgttgttgttgcactgcatgggaaggaaactaatgctggtgaattctttgttgaggatgtactagaagctggtttaccacctcagattgagcggcctatcgatctacgtaagtctagctatgttctcttccttttgctaacctcatggctcaatcatttctataagcaatctctcatgatacatccatattgcatctgcagaggaagataaatatgtcgtgttattgtcgggcctttgtattggaagcaaatcggctaatcccctgcagtttcagcttcttgttgaccatataactgggcatctcggagatgaggaggttcaaatctcttaacttgcaggttgttcaacatatttctttccttaatttatactttatggtttgaacaggaacaaggccttgcagcacagatagttcatgtagtaattgctggaaactcttttgaatttccccgcaaactcattaatggccaggtacttataacttttgttgctgatatattctcagatacagttccagtaattatctgccccagttatgtcttatgatctttattggttgatctttgtagaacttggcctcgaaagatcaatcgacactgtatgagcccatcaaagagcttgatatcatgttaagccaggtcagttaactggatctacgtgtgtgttatcgatatctattgagatgaaagttcaaactcctgtttttttttttgtggattgtttttagatagctgcaggagtttcagtagatatcatgccaggcacgaatgatccagctaacttcgcattgcctcagcaggtctgcaaatacataagaaacattcaaaatcccgcattttgtatcgataactctgattcataggcccttctcttttgttcagcctctgaatagatgtcttttccctggatcttcaccttataacaccttcagatcatgtacaaatcctcactcatttgatgtcgataatatcaggtatgattattattaatagttgaatacaatctctctgattttacaacgataaaattcttgggtttatctgactgaaaacctcatatgggggcattttgcagatttcttggaacttctggtcagaacatcgatgaccttggcaagtactcagaggctaagagcaagcttgattttgtggaaagaacgctgaggtggagacatcttgccccaactgcacctaatacactcggtaagaattctccttgccctgcaagattacttttttgaactaagcccataaaaaaatgatcctttgagttctatttggttttgattcacttgcgtacaggttgttatcctttcaccgatagagaccctttcttgattgaaacctgcccgcatgtctacttcgtcgggaatcaagataaatatgacaaccgtttgataaagggtaaaagcaccttacacagagattagaaataacattctcttttgtcaaacatcaggctttaacttttcttgggtaaatatgaatgctgcagggtcagaagggcagcttgtccggttgatctgcattcctaagttctgtgagaccggtattgctgttgcggtgagtttaaaatttgagcagaatttgagaccatttaccctcatagattgcagattctaaatctcaaaatcaccatgtctatttcgcaggtgaacctaagaaatctggaatgtcacactttaagctttagcactcagataaaccaatcataacattgagttgctactttggtagattatttcctgtcttgaagatgtaatgttgagctttttcagtaacacactcctatgttctaaccaaatgtttgttaaaaatcctttttcttgagtggaacttccaaatctttggatatattggtaatgctcattgttttgtcctaattttctaaaaatctcgacacgagttcttaggtagtcacataaaggacaaaaagggccgaccagatagtgtcgtggtcgttggtcagaagaacgtgaaaagactgcaaaaataatcttaaaaaaagcaacaagtgcacagaatctcatgcaaatgtctctctctctcttctcaacggctatatccatccacacttattacattataaaattaattaaatgcaataatgtaacgcattatattctccaacggtccattttcccgcatttccctaacctttcctttataacgcaaaacagtttcatcttctacacttaacactttaatcctctct

SEQ ID NO: 91 - protein, 440 aa, Arabidopsis thaliana
MEIDSEKIHERKQSDYNSLDERFEIQKEMYRGQQYSQIYFARLHLMRTLLYSLAPTWKSHLPVCKVLGLEKGKECIIVGTLFKHMKLKPCVLDEYSKERSVTPLVKPHNFMHPDDNLILEDESGRVKLAGSALSPAIYVTGVVVALHGKETNAGEFFVEDVLEAGLPPQIERPIDLQEDKYVVLLSGLCIGSKSANPLQFQLLVDHITGHLGDEEEQGLAAQIVHVVIAGNSFEFPRKLINGQNLASKDQSTLYEPIKELDIMLSQIAAGVSVDIMPGTNDPANFALPQQPLNRCLFPGSSPYNTFRSCTNPHSFDVDNIRFLGTSGQNIDDLGKYSEAKSKLDFVERTLRWRHLAPTAPNTLGCYPFTDRDPFLIETCPHVYFVGNQDKYDNRLIKGSEGQLVRLICIPKFCETGIAVAVNLRNLECHTLSFSTQINQS

SEQ ID NO: 92 - DNA, 3684 bp, Arabidopsis thaliana
agcgattccttagcagaaaggcgctccatttctctggcgtaaaccaaaggagatccttgaactgtttcctgcaccattgctcttaaaacccttctccggcacgaattcttccaaccctgcttcaccaccggaacattgagacaaaatctcgacggtgacgctgaggttgaaaaaaccaatcgaaccgcagacgtaccaggaaccgaaccatgtatcaacgccattgaagaagaagaagaagaagaaggtgaaaaacgaaagattgagaatttgtttgctttgagcaaccaaacctcaggaaaaaagagttaaggtgggagtgtctggttcaaccggtttatatccggttcaaattaaacctcttacagttaaccgggttttgtgtttggttcgattgttcataaaagaaagaagactcttgtcgtcgattagtgccaaagttgaaagttgaaaccttttctcagaattttctgctcagttctgagtttttttttcccgccatggaaatcgactccgagaaaattcacgaaaggaagcaatccgattacaattcgctggtacgaactctattactttatcgacttgtagtgaaagacaaatgtaatcattcgtggtggtgactgtttctacttataagtgtacgggctagggtttgttatctgattctgagtttttgcaattgaagcaggatgagagattcgagatacagaaggagatgtacagaggtcagcaatacagtcagatttactttgctcgtcttcatctcatgagaacacttctctactctcttgctcctacttggaaatctcatttgcctggtcagtgcttttgtttctctcatatttagcacaacaacgaagagcagtttttgagaattttcttgggttagatataattaggtgaaatcagtgatttttagggatttttgctatcttatggattacagttgagaaagattgctagtattgtttaaattatagatctgaatgtgaatttcatttttgcagtgtgtaaggttttgggacttgaaaaaggaaaagaatgcataattgtgggaaccttgttcaaacacatgaagcttaaaccttgtgttctcgatgaatattctaaagaggttggtttttattaacctctactgtttttttgagctatgtctatgctgaatcaatctgagtatatttaacataatgcagaggtcagttactccgcttgttaaaccacataactttatgcatcctgatgataatctgatcctcgaagacgagagtgggagagttaagcttgctggttccgcactttcacctgcgatttatgtgacaggtattgcaaatgggttcttactgtttttactgtatgattttttccttctttacaatgtggcaaatcttagagattttgatcaagctttcctctcttaaaagatgggttctttaagaaaattaacgttgaagcctcccgtgcattgtaggtgttgttgttgcactgcatgggaaggaaactaatgctggtgaattctttgttgaggatgtactagaagctggtttaccacctcagattgagcggcctatcgatctacgtaagtctagctatgttctcttccttttgctaacctcatggctcaatcatttctataagcaatctctcatgatacatccatattgcatctgcagaggaagataaatatgtcgtgttattgtcgggcctttgtattggaagcaaatcggctaatcccctgcagtttcagcttcttgttgaccatataactgggcatctcggagatgaggaggttcaaatctcttaacttgcaggttgttcaacatatttctttccttaatttatactttatggtttgaacaggaacaaggccttgcagcacagatagttcatgtagtaattgctggaaactcttttgaatttccccgcaaactcattaatggccaggtacttataacttttgttgctgatatattctcagatacagttccagtaattatctgccccagttatgtcttatgatctttattggttgatctttgtagaacttggcctcgaaagatcaatcgacactgtatgagcccatcaaagagcttgatatcatgttaagccaggtcagttaactggatctacgtgtgtgttatcgatatctattgagatgaaagttcaaactcctgtttttttttttgtggattgtttttagatagctgcaggagtttcagtagatatcatgccaggcacgaatgatccagctaacttcgcattgcctcagcaggtctgcaaatacataagaaacattcaaaatcccgcattttgtatcgataactctgattcataggcccttctcttttgttcagcctctgaatagatgtcttttccctggatcttcaccttataacaccttcagatcatgtacaaatcctcactcatttgctgtcgataatatcaggtatgattattattaatagttgaatacaatctctctgattttacaacgataaaattcttgggtttatctgactgaaaacctcatatgggggcattttgcagatttcttggaacttctggtcagaacatcgatgaccttggcaagtactcagaggctaagagcaagcttgattttgtggaaagaacgctgaggtggagacatcttgccccaactgcacctaatacactcggtaagaattctccttgccctgcaagattacttttttgaactaagcccataaaaaaatgatcctttgagttctatttggttttgattcacttgcgtacaggttgttatcctttcaccgatagagaccctttcttgattgaaacctgcccgcatgtctacttcgtcgggaatcaagataaatatgacaaccgtttgataaagggtaaaagcaccttacacagagattagaaataacattctcttttgtcaaacatcaggctttaacttttcttgggtaaatatgaatgctgcagggtcagaagggcagcttgtccggttgatctgcattcctaagttctgtgagaccggtattgctgttgcggtgagtttaaaatttgagcagaatttgagaccatttaccctcatagattgcagattctaaatctcaaaatcaccatgtctatttcgcaggtgaacctaagaaatctggaatgtcacactttaagctttagcactcagataaaccaatcataacattgagttgctactttggtagattatttcctgtcttgaagatgtaatgttgagctttttcagtaacacactcctatgttctaaccaaatgtttgttaaaaatcctttttcttgagtggaacttccaaatctttggatatattggtaatgctcattgttttgtcctaattttctaaaaatctcgacacgagttcttaggtagtcacataaaggacaaaaagggccgaccagatagtgtcgtggtcgttggtcagaagaacgtgaaaagactgcaaaaataatcttaaaaaaagcaacaagtgcacagaatctcatgcaaatgtctctctctctcttctcaacggctatatccatccacacttattacattataaaattaattaaatgcaataatgtaacgcattatattctccaacggtccattttcccgcatttccctaacctttcctttataacgcaaaacagtttcatcttctacacttaacactttaatcctctct

SEQ ID NO: 93 - protein, 440 aa, Arabidopsis thaliana
MEIDSEKIHERKQSDYNSLDERFEIQKEMYRGQQYSQIYFARLHLMRTLLYSLAPTWKSHLPVCKVLGLEKGKECIIVGTLFKHMKLKPCVLDEYSKERSVTPLVKPHNFMHPDDNLILEDESGRVKLAGSALSPAIYVTGVVVALHGKETNAGEFFVEDVLEAGLPPQIERPIDLQEDKYVVLLSGLCIGSKSANPLQFQLLVDHITGHLGDEEEQGLAAQIVHVVIAGNSFEFPRKLINGQNLASKDQSTLYEPIKELDIMLSQIAAGVSVDIMPGTNDPANFALPQQPLNRCLFPGSSPYNTFRSCTNPHSFAVDNIRFLGTSGQNIDDLGKYSEAKSKLDFVERTLRWRHLAPTAPNTLGCYPFTDRDPFLIETCPHVYFVGNQDKYDNRLIKGSEGQLVRLICIPKFCETGIAVAVNLRNLECHTLSFSTQINQS

SEQ ID NO: 94 - DNA, 454 bp, Mus musculus
ctgcagaggattttccacagtataattagagattggatgtggggaagaattatgtttttttttttctttttggtaatcttatggttgggtatgctttattttctaattgatttgaaagaggatatagaaacaaaagacaggagaaaaataatctggccttctgatacttatttgaaggctttctaatttcccaactctaaccccaagctctccgttttactgtttagtattctaggctggcagtttgagtctgtaccaggcaaaaaacgttccaaatcaagatagacaggatggagaaccaatcacagagctggatttcctttcaaattctaccaatggctattgtgcaggagactttgaactcacaaagaaaggcggggccaagacttaagcgttaaaaatcaccaccaagccagcctcccagcagcagtaaagaggctgttgtgcataccat

SEQ ID NO: 95 - DNA, 5725 bp, Mus musculus; identical to SEQ ID NO: 81